(19) United States
(12) Patent Application Publication
(10) Pub. No.: US 2025/0333523 A1
(43) Pub. Date: Oct. 30, 2025

(54) Anti-IL18 Binding Protein Antibodies and Methods of Use Thereof

(71) Applicant: Alector LLC, South San Francisco, CA (US)

(72) Inventors: Farah Riad Itani, South San Francisco, CA (US); Angie Grace Yee, San Francisco, CA (US); Yong Wang, Alviso, CA (US); Wei Li, Pacifica, CA (US); Wei-Hsien Ho, Belmont, CA (US); Marina Roell, Concord, CA (US); Seung-Joo Lee, South San Francisco, CA (US); Spencer Liang, San Mateo, CA (US); Li Zhou, San Jose, CA (US); Belvin Gong, Pleasanton, CA (US)

(73) Assignee: Alector LLC, South San Francisco, CA (US)

(21) Appl. No.: 19/259,368

(22) Filed: Jul. 3, 2025

Related U.S. Application Data
(63) Continuation of application No. PCT/US24/10432, filed on Jan. 5, 2024.
(60) Provisional application No. 63/478,793, filed on Jan. 6, 2023.
(60) Provisional application No. 63/515,493, filed on Jul. 25, 2023.
(60) Provisional application No. 63/615,158, filed on Dec. 27, 2023.

(51) Int. Cl. C07K 16/28; A61K 39/00; A61K 39/395; A61P 35/00

(52) U.S. Cl. CPC C07K 16/2866; A61K 39/39558; A61P 35/00; A61K 2039/507; C07K 2317/24; C07K 2317/33; C07K 2317/34; C07K 2317/76; C07K 2317/92

(57) ABSTRACT

The present disclosure is generally directed to compositions that include monovalent antibodies, e.g., monoclonal monovalent antibodies that specifically bind a IL18BP polypeptide, e.g., a mammalian IL18BP polypeptide or human IL18BP polypeptide, and use of such compositions in treating an individual in need thereof.

This application is a continuation of International Application No. PCT/US2024/010432, filed on Jan. 5, 2024, which claims the benefit of priority to U.S. Provisional Patent Application Nos. 63/615,158, filed Dec. 27, 2023, 63/515,493, filed Jul. 25, 2023, and 63/478,793, filed Jan. 6, 2023, each of which is hereby incorporated by reference in its entirety for any purpose.

### SEQUENCE LISTING

The instant application contains a Sequence Listing which has been submitted electronically in XML file format and is hereby incorporated by reference in its entirety. Said XML copy, created on Dec. 18, 2023, is named 2023-12-18_01209-0015-62US_SL.xml and is 254,583 bytes in size.

### FIELD OF THE PRESENT DISCLOSURE

The present disclosure relates to anti-IL18BP antibodies and uses (e.g., therapeutic uses) of such antibodies.

### BACKGROUND OF THE PRESENT DISCLOSURE

Interluekin-18 (IL-18), initially described as an interferon-γ (IFNγ)-inducing factor, is an early signal in the development of T-lymphocyte helper type 1 (Th1) responses. Interleukin-18 binding protein (IL18BP) binds IL-18 and neutralizes its activity, thus acting as a natural inhibitor of IL-18-induced interferon-γ (IFNγ) and suppressing the Th1 response (Novick et al, 1999, Immunity, 10:127-136). IL18BP is constitutively secreted and has an exceptionally high affinity for IL-18 (˜400 pM). It is present in the serum of healthy humans at a 20-fold molar excess compared to IL-18, and thus competes with cell surface receptors for IL-18 to act as a natural anti-inflammatory and an immunosuppressive molecule.

The human IL18BP gene encodes for at least four distinct isoforms derived from mRNA splice variants: two isoforms of mouse IL18BP have been identified: IL18BPa, b, c, d (Kim et al, 2000, PNAS, 97:1190-1195). IL18BPa is the most widely expressed isoform which binds mature (but not pro-) IL-18 with an affinity substantially higher than IL-18 receptor α.

IL-18 and IL18BP are prevalent in the tumor microenvironment (TME). IL18BP is upregulated in various tumors and limits the anti-tumor activity of IL-18 (Zhou et al, 2020, Nature, 583:609). In a tumor mouse model, mIL-18 administration had no effect on tumor growth in wild-type animals; however, it significantly inhibited tumor growth in IL18 bp^{-/-} mice, suggesting that IL-18BP expression is common in cancer and that it may act as a soluble immune checkpoint.

Anti-IL18BP antibodies have been previously described in, e.g., WO2002/092008, WO2015/032932, WO2016/139297, WO2019/213686, WO2023/178192.

There is a need for novel therapeutic anti-IL18BP antibodies that are effective at treating various diseases, disorders, and conditions, such as cancer.

All references cited herein, including patent applications and publications, are hereby incorporated by reference in their entirety.

### SUMMARY OF THE PRESENT DISCLOSURE

The present disclosure is generally directed to anti-IL18BP antibodies and methods of using such antibodies. The disclosure includes multiple embodiments, including, but not limited to, the following embodiments.

Embodiment 1 is an isolated anti-interleukin-18-binding protein (IL18BP) antibody, wherein the anti-IL18BP antibody blocks binding of IL-18 to IL18BP.

Embodiment 2 is the anti-IL18BP antibody of embodiment 1, wherein the anti-IL18BP antibody increases expression levels of interferon-gamma (IFNγ) in peripheral blood mononuclear cells.

Embodiment 3 is the anti-IL18BP antibody of embodiment 1 or 2, wherein the anti-IL18BP antibody increases expression of IFNγ in natural killer cells.

Embodiment 4 is the anti-IL18BP antibody of any one of embodiments 1-3, wherein the anti-IL18BP antibody increases levels of inflammatory cytokines in serum.

Embodiment 5 is the anti-IL18BP antibody of any one of embodiments 1-4, wherein the anti-IL18BP antibody increases IFNγ levels in serum.

Embodiment 6 is the anti-IL18BP antibody of any one of embodiments 1-5, wherein the anti-IL18BP antibody increases IL-10 levels in serum.

Embodiment 7 is the anti-IL18BP antibody of any one of embodiments 1-6, wherein the anti-IL18BP antibody increases IL-6 levels in serum.

Embodiment 8 is the anti-IL18BP antibody of any one of embodiments 1-7, wherein the anti-IL18BP antibody increases tumor necrosis factor alpha (TNFα) levels in serum.

Embodiment 9 is the anti-IL18BP antibody of any one of embodiments 1-8, wherein the anti-IL18BP antibody decreases the percentage of CD3+ T cells in vivo.

Embodiment 10 is the anti-IL18BP antibody of any one of embodiments 1-9, wherein the anti-IL18BP antibody decreases the percentage of CD19+ B cells in vivo.

Embodiment 11 is the anti-IL18BP antibody of any one of embodiments 1-10, wherein the anti-IL18BP antibody decreases the percentage of CD11b+ cells in vivo.

Embodiment 12 is the anti-IL18BP antibody of any one of embodiments 1-11, wherein the anti-IL18BP antibody increases the percentage of macrophages in vivo.

Embodiment 13 is the anti-IL18BP antibody of any one of embodiments 1-12, wherein the anti-IL18BP antibody competes with one or more antibodies selected from BP-10, BP-02, BP-03, BP-04, BP-05, BP-06, BP-07, BP-08, BP-09, BP-10, BP-11, BP-12, BP-13, BP-14, BP-15, BP-16, BP-04.01, BP-04.02, BP-04.03, BP-04.04, BP-04.05, BP-04.06, BP-04.07, BP-04.08, BP-04.09, BP-04.10, BP-04.11, BP-04.12, BP-04.13, BP-04.14, BP-04.15, BP-04.16, BP-04.17, BP-04.18, BP-04.19, BP-04.20, BP-04.21, BP-04.22, BP-04.23, BP-04.24, BP-04.25, BP-04.26, BP-04.27, BP-04.28, BP-04.29, BP-04.30, BP-04.31, and BP-04.32, and any combination thereof for binding to IL18BP.

Embodiment 14 is the anti-IL18BP antibody of any one of embodiments 1-13, wherein the anti-IL18BP antibody comprises a light chain variable domain and a heavy chain variable domain, wherein the light chain variable domain, the heavy chain variable domain, or both comprise at least one, at least two, at least three, at least four, at least five, or six HVRs selected from HVR-L1, HVR-L2, HVR-L3, HVR-H1, HVR-H2, and HVR-H3 of an antibody selected from: BP-01, BP-02, BP-03, BP-04, BP-05, BP-06, BP-07, BP-08, BP-09, BP-10, BP-11, BP-12, BP-13, BP-14, BP-15, BP-16, BP-04.01, BP-04.02, BP-04.03, BP-04.04, BP-04.05, BP-04.06, BP-04.07, BP-04.08, BP-04.09, BP-04.10, BP-04.11, BP-04.12, BP-04.13, BP-04.14, BP-04.15, BP-04.16, BP-04.17, BP-04.18, BP-04.19, BP-04.20, BP-04.21, BP-04.22, BP-04.23, BP-04.24, BP-04.25, BP-04.26, BP-04.27, BP-04.28, BP-04.29, BP-04.30, BP-04.31, and BP-04.32.

Embodiment 15 is the anti-IL18BP antibody of embodiment 14, wherein:
a. the HVR-H1 comprises an amino acid sequence selected from the group consisting of SEQ ID NOs: 60, 61, 62, 63, 64, 65, 66, 67, 68, 69, 70, 167, 168, and 169;
b. the HVR-H2 comprises an amino acid sequence selected from the group consisting of SEQ ID NOs: 71, 72, 73, 74, 75, 76, 77, 78, 79, 80, 81, 82, 83, 84, 85, 86, 170, 171, 172, 173, 174, 175, 176, 177, and 178;
c. the HVR-H3 comprises an amino acid sequence selected from the group consisting of SEQ ID NOs: 87, 88, 89, 90, 91, 92, 93, 94, 95, 96, 97, 98, 99, 100, 179, 180, 181, 182, 183, 184, 185, 186, 187, and 188;
d. the HVR-L1 comprises an amino acid sequence selected from the group consisting of SEQ ID NOs: 101, 102, 103, 104, 105, 106, 107, 108, 109, 110, 111, and 247;
e. the HVR-L2 comprises an amino acid sequence selected from the group consisting of SEQ ID NOs: 112, 113, 114, 115, 116, 117, 118, 119, 120, 121, and 248; and/or
f. the HVR-L3 comprises an amino acid sequence selected from the group consisting of SEQ ID NOs: 122, 123, 124, 125, 126, 127, 128, 129, 130, 131, 132, 133, and 249.

Embodiment 16 is the anti-IL18BP antibody of embodiment 14 or 15, wherein:
a. the HVR-H1 comprises the amino acid sequence of SEQ ID NO:60; the HVR-H2 comprises the amino acid sequence of SEQ ID NO:71; the HVR-H3 comprises the amino acid sequence of SEQ ID NO: 87; the HVR-L1 comprises the amino acid sequence of SEQ ID NO: 101; the HVR-L2 comprises the amino acid sequence of SEQ ID NO: 112; and the HVR-L3 comprises the amino acid sequence of SEQ ID NO: 122; or
b. the HVR-H1 comprises the amino acid sequence of SEQ ID NO:61; the HVR-H2 comprises the amino acid sequence of SEQ ID NO:72; the HVR-H3 comprises the amino acid sequence of SEQ ID NO: 88; the HVR-L1 comprises the amino acid sequence of SEQ ID NO: 102; the HVR-L2 comprises the amino acid sequence of SEQ ID NO: 113; and the HVR-L3 comprises the amino acid sequence of SEQ ID NO: 123; or
c. the HVR-H1 comprises the amino acid sequence of SEQ ID NO:61; the HVR-H2 comprises the amino acid sequence of SEQ ID NO: 73; the HVR-H3 comprises the amino acid sequence of SEQ ID NO: 88; the HVR-L1 comprises the amino acid sequence of SEQ ID NO: 103; the HVR-L2 comprises the amino acid sequence of SEQ ID NO: 113; and the HVR-L3 comprises the amino acid sequence of SEQ ID NO: 123; or
d. the HVR-H1 comprises the amino acid sequence of SEQ ID NO:62; the HVR-H2 comprises the amino acid sequence of SEQ ID NO: 74; the HVR-H3 comprises the amino acid sequence of SEQ ID NO: 89; the HVR-L1 comprises the amino acid sequence of SEQ ID NO: 104; the HVR-L2 comprises the amino acid sequence of SEQ ID NO: 114; and the HVR-L3 comprises the amino acid sequence of SEQ ID NO: 124; or
e. the HVR-H1 comprises the amino acid sequence of SEQ ID NO:61; the HVR-H2 comprises the amino acid sequence of SEQ ID NO: 75; the HVR-H3 comprises the amino acid sequence of SEQ ID NO: 90; the HVR-L1 comprises the amino acid sequence of SEQ ID NO: 105; the HVR-L2 comprises the amino acid sequence of SEQ ID NO: 113; and the HVR-L3 comprises the amino acid sequence of SEQ ID NO: 123; or
f. the HVR-H1 comprises the amino acid sequence of SEQ ID NO:63; the HVR-H2 comprises the amino acid sequence of SEQ ID NO: 76; the HVR-H3 comprises the amino acid sequence of SEQ ID NO: 91; the HVR-L1 comprises the amino acid sequence of SEQ ID NO: 106; the HVR-L2 comprises the amino acid sequence of SEQ ID NO: 115; and the HVR-L3 comprises the amino acid sequence of SEQ ID NO: 125; or
g. the HVR-H1 comprises the amino acid sequence of SEQ ID NO:61; the HVR-H2 comprises the amino acid sequence of SEQ ID NO:77; the HVR-H3 comprises the amino acid sequence of SEQ ID NO: 88; the HVR-L1 comprises the amino acid sequence of SEQ ID NO: 103; the HVR-L2 comprises the amino acid sequence of SEQ ID NO:113; and the HVR-L3 comprises the amino acid sequence of SEQ ID NO: 123; or
h. the HVR-H1 comprises the amino acid sequence of SEQ ID NO:64; the HVR-H2 comprises the amino acid sequence of SEQ ID NO: 78; the HVR-H3 comprises the amino acid sequence of SEQ ID NO: 92; the HVR-L1 comprises the amino acid sequence of SEQ ID NO: 107; the HVR-L2 comprises the amino acid sequence of SEQ ID NO: 116; and the HVR-L3 comprises the amino acid sequence of SEQ ID NO: 126; or
i. the HVR-H1 comprises the amino acid sequence of SEQ ID NO:65; the HVR-H2 comprises the amino acid sequence of SEQ ID NO: 79; the HVR-H3 comprises the amino acid sequence of SEQ ID NO: 93; the HVR-L1 comprises the amino acid sequence of SEQ ID NO: 101; the HVR-L2 comprises the amino acid sequence of SEQ ID NO: 116; and the HVR-L3 comprises the amino acid sequence of SEQ ID NO: 127; or
j. the HVR-H1 comprises the amino acid sequence of SEQ ID NO:66; the HVR-H2 comprises the amino acid sequence of SEQ ID NO:80; the HVR-H3 comprises the amino acid sequence of SEQ ID NO: 94; the HVR-L1 comprises the amino acid sequence of SEQ ID NO: 108; the HVR-L2 comprises the amino acid sequence of SEQ ID NO: 117; and the HVR-L3 comprises the amino acid sequence of SEQ ID NO: 128; or
k. the HVR-H1 comprises the amino acid sequence of SEQ ID NO:66; the HVR-H2 comprises the amino acid sequence of SEQ ID NO:81; the HVR-H3 comprises the amino acid sequence of SEQ ID NO: 95; the HVR-L1 comprises the amino acid sequence of SEQ ID NO: 109; the HVR-L2 comprises the amino acid sequence of SEQ ID NO: 118; and the HVR-L3 comprises the amino acid sequence of SEQ ID NO: 129; or
l. the HVR-H1 comprises the amino acid sequence of SEQ ID NO:67; the HVR-H2 comprises the amino acid sequence of SEQ ID NO:82; the HVR-H3 comprises the amino acid sequence of SEQ ID NO: 96; the HVR-L1 comprises the amino acid sequence of SEQ ID NO: 101; the HVR-L2 comprises the amino acid sequence of SEQ ID NO: 116; and the HVR-L3 comprises the amino acid sequence of SEQ ID NO: 130; or
m. the HVR-H1 comprises the amino acid sequence of SEQ ID NO:68; the HVR-H2 comprises the amino acid sequence of SEQ ID NO:83; the HVR-H3 comprises the amino acid sequence of SEQ ID NO: 97; the HVR-L1 comprises the amino acid sequence of SEQ ID NO: 101; the HVR-L2 comprises the amino acid sequence of SEQ ID NO: 119; and the HVR-L3 comprises the amino acid sequence of SEQ ID NO: 131; or
n. the HVR-H1 comprises the amino acid sequence of SEQ ID NO:69; the HVR-H2 comprises the amino acid sequence of SEQ ID NO:84; the HVR-H3 comprises the amino acid sequence of SEQ ID NO: 98; the HVR-L1 comprises the amino acid sequence of SEQ ID NO: 110; the HVR-L2 comprises the amino acid sequence of SEQ ID NO: 120; and the HVR-L3 comprises the amino acid sequence of SEQ ID NO: 132; or
o. the HVR-H1 comprises the amino acid sequence of SEQ ID NO: 70; the HVR-H2 comprises the amino acid sequence of SEQ ID NO:85; the HVR-H3 comprises the amino acid sequence of SEQ ID NO: 99; the HVR-L1 comprises the amino acid sequence of SEQ ID NO: 111; the HVR-L2 comprises the amino acid sequence of SEQ ID NO: 121; and the HVR-L3 comprises the amino acid sequence of SEQ ID NO: 133; or
p. the HVR-H1 comprises the amino acid sequence of SEQ ID NO: 70; the HVR-H2 comprises the amino acid sequence of SEQ ID NO:86; the HVR-H3 comprises the amino acid sequence of SEQ ID NO: 100; the HVR-L1 comprises the amino acid sequence of SEQ ID NO: 111; the HVR-L2 comprises the amino acid sequence of SEQ ID NO: 121; and the HVR-L3 comprises the amino acid sequence of SEQ ID NO: 133; or
q. the HVR-H1 comprises the amino acid sequence of SEQ ID NO: 167; the HVR-H2 comprises the amino acid sequence of SEQ ID NO: 170; the HVR-H3 comprises the amino acid sequence of SEQ ID NO: 179; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
r. the HVR-H1 comprises the amino acid sequence of SEQ ID NO: 168; the HVR-H2 comprises the amino acid sequence of SEQ ID NO: 171; the HVR-H3 comprises the amino acid sequence of SEQ ID NO: 180; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO: 249, or
s. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 181; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
t. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 182; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
u. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
v. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
w. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 185; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
x. the HVR-H1 comprises the amino acid sequence of SEQ ID NO: 169; the HVR-H2 comprises the amino acid sequence of SEQ ID NO: 1704; the HVR-H3 comprises the amino acid sequence of SEQ ID NO: 185; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
y. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO: 249, or
z. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
aa. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 185; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
bb. the HVR-H1 comprises the amino acid sequence of SEQ ID NO: 169; the HVR-H2 comprises the amino acid sequence of SEQ ID NO: 176; the HVR-H3 comprises the amino acid sequence of SEQ ID NO: 186; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
cc. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 187; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
dd. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 188; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
ee, the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 182; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
ff. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
gg. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 184; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
hh. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
ii. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
jj. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 184; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
kk. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
ll. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 186; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
mm. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
nn. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO: 249, or
oo. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 185; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
pp. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 186; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
qq. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
rr. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 184; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
ss. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
tt. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 186; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
uu. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
vv. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249.

Embodiment 17 is an isolated anti-IL18BP antibody, wherein the anti-IL18BP antibody comprises a heavy chain variable region (VH) and a light chain variable region (VL), wherein the heavy chain variable region (VH) comprises:
a. an HVR-H1 comprising an amino acid sequence chosen from any one of SEQ ID NOs: 60, 61, 62, 63, 64, 65, 66, 67, 68, 69, 70, 167, 168, and 169;
b. an HVR-H2 comprising an amino acid sequence chosen from any one of SEQ ID NOs: 71, 72, 73, 74, 75, 76, 77, 78, 79, 80, 81, 82, 83, 84, 85, 86, 170, 171, 172, 173, 174, 175, 176, 177, and 178; and
c. an HVR-H3 comprising an amino acid sequence chosen from any one of SEQ ID NOs: 87, 88, 89, 90, 91, 92, 93, 94, 95, 96, 97, 98, 99, 100, 179, 180, 181, 182, 183, 184, 185, 186, 187, and 188.

Embodiment 18 is the antibody of embodiment 17, wherein the light chain variable region comprises:
a. an HVR-L1 comprising an amino acid sequence chosen from any one of SEQ ID NOs: 101, 102, 103, 104, 105, 106, 107, 108, 109, 110, 111, and 247;
b. an HVR-L2 comprising an amino acid sequence chosen from any one of SEQ ID NOs: 112, 113, 114, 115, 116, 117, 118, 119, 120, 121, and 248; and
c. an HVR-L3 comprising an amino acid sequence chosen from any one of SEQ ID NOs: 122, 123, 124, 125, 126, 127, 128, 129, 130, 131, 132, 133, and 249.

Embodiment 19 is the antibody of embodiment 17 or 18, wherein the antibody comprises a VH that is at least 90%, at least 95%, at least 97%, or at least 99% identical to an amino acid sequence selected from any one of SEQ ID NOs: 29, 30, 31, 32, 33, 34, 35, 36, 37, 38, 39, 40, 41, 42, 43, 44, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, and 165.

Embodiment 20 is the antibody of any one of embodiments 17-19, wherein the antibody comprises a VL that is at least 90%, at least 95%, at least 97%, or at least 99% identical to an amino acid sequence selected from any one of SEQ ID NOs: 45, 46, 47, 48, 49, 50, 51, 52, 53, 54, 55, 56, 57, 58, 59, and 166.

Embodiment 21 is the antibody of any one of embodiments 17-20, wherein the VH comprises 1, 2, 3, 4, 5, 6, 7, 8, 9, or 10 amino acid substitutions, insertions, and/or deletions compared to an amino acid sequence selected from any one of SEQ ID NOs: 29, 30, 31, 32, 33, 34, 35, 36, 37, 38, 39, 40, 41, 42, 43, 44, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, and 165.

Embodiment 22 is the antibody of any one of embodiments 17-21, wherein the VL comprises 1, 2, 3, 4, 5, 6, 7, 8, 9, or 10 amino acid substitutions, insertions, and/or deletions compared to an amino acid sequence selected from any one of SEQ ID NOs: 45, 46, 47, 48, 49, 50, 51, 52, 53, 54, 55, 56, 57, 58, 59, and 166.

Embodiment 23 is the antibody of any one of embodiments 17-22, wherein the antibody comprises a VH comprising an amino acid sequence selected from any one of SEQ ID NOs: 29, 30, 31, 32, 33, 34, 35, 36, 37, 38, 39, 40, 41, 42, 43, 44, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, and 165.

Embodiment 24 is the antibody of any one of embodiments 17-23, wherein the antibody comprises a VL comprising an amino acid sequence selected from any one of SEQ ID NOs: 45, 46, 47, 48, 49, 50, 51, 52, 53, 54, 55, 56, 57, 58, 59, and 166.

Embodiment 25 is an isolated anti-IL18BP antibody, wherein the anti-IL18BP antibody comprises:
a. an HVR-H1 comprising the amino acid sequence of SEQ ID NO:60; an HVR-H2 comprising the amino acid sequence of SEQ ID NO:71; an HVR-H3 comprising the amino acid sequence of SEQ ID NO:87; an HVR-L1 comprising the amino acid sequence of SEQ ID NO: 101; an HVR-L2 comprising the amino acid sequence of SEQ ID NO: 112; and an HVR-L3 comprising the amino acid sequence of SEQ ID NO: 122;
b. an HVR-H1 comprising the amino acid sequence of SEQ ID NO:61; an HVR-H2 comprising the amino acid sequence of SEQ ID NO: 72; an HVR-H3 comprising the amino acid sequence of SEQ ID NO:88; an HVR-L1 comprising the amino acid sequence of SEQ ID NO: 102; an HVR-L2 comprising the amino acid sequence of SEQ ID NO: 113; and an HVR-L3 comprising the amino acid sequence of SEQ ID NO: 123;
c. an HVR-H1 comprising the amino acid sequence of SEQ ID NO:61; an HVR-H2 comprising the amino acid sequence of SEQ ID NO: 73; an HVR-H3 comprising the amino acid sequence of SEQ ID NO:88; an HVR-L1 comprising the amino acid sequence of SEQ ID NO: 103; an HVR-L2 comprising the amino acid sequence of SEQ ID NO: 113; and an HVR-L3 comprising the amino acid sequence of SEQ ID NO: 123;
d. an HVR-H1 comprising the amino acid sequence of SEQ ID NO:62; an HVR-H2 comprising the amino acid sequence of SEQ ID NO:74; an HVR-H3 comprising the amino acid sequence of SEQ ID NO:89; an HVR-L1 comprising the amino acid sequence of SEQ ID NO: 104; an HVR-L2 comprising the amino acid sequence of SEQ ID NO: 114; and an HVR-L3 comprising the amino acid sequence of SEQ ID NO: 124;
e. an HVR-H1 comprising the amino acid sequence of SEQ ID NO:61; an HVR-H2 comprising the amino acid sequence of SEQ ID NO: 75; an HVR-H3 comprising the amino acid sequence of SEQ ID NO:90; an HVR-L1 comprising the amino acid sequence of SEQ ID NO: 105; an HVR-L2 comprising the amino acid sequence of SEQ ID NO: 113; and an HVR-L3 comprising the amino acid sequence of SEQ ID NO: 123;
f. an HVR-H1 comprising the amino acid sequence of SEQ ID NO:63; an HVR-H2 comprising the amino acid sequence of SEQ ID NO: 76; an HVR-H3 comprising the amino acid sequence of SEQ ID NO:91; an HVR-L1 comprising the amino acid sequence of SEQ ID NO: 106; an HVR-L2 comprising the amino acid sequence of SEQ ID NO: 115; and an HVR-L3 comprising the amino acid sequence of SEQ ID NO: 125;
g. an HVR-H1 comprising the amino acid sequence of SEQ ID NO:61; an HVR-H2 comprising the amino acid sequence of SEQ ID NO:77; an HVR-H3 comprising the amino acid sequence of SEQ ID NO:88; an HVR-L1 comprising the amino acid sequence of SEQ ID NO: 103; an HVR-L2 comprising the amino acid sequence of SEQ ID NO: 113; and an HVR-L3 comprising the amino acid sequence of SEQ ID NO:123;
h. an HVR-H1 comprising the amino acid sequence of SEQ ID NO:64; an HVR-H2 comprising the amino acid sequence of SEQ ID NO: 78; an HVR-H3 comprising the amino acid sequence of SEQ ID NO:92; an HVR-L1 comprising the amino acid sequence of SEQ ID NO: 107; an HVR-L2 comprising the amino acid sequence of SEQ ID NO: 116; and an HVR-L3 comprising the amino acid sequence of SEQ ID NO:126;
i. an HVR-H1 comprising the amino acid sequence of SEQ ID NO:65; an HVR-H2 comprising the amino acid sequence of SEQ ID NO: 79; an HVR-H3 comprising the amino acid sequence of SEQ ID NO:93; an HVR-L1 comprising the amino acid sequence of SEQ ID NO: 101; an HVR-L2 comprising the amino acid sequence of SEQ ID NO: 116; and an HVR-L3 comprising the amino acid sequence of SEQ ID NO: 127;
j. an HVR-H1 comprising the amino acid sequence of SEQ ID NO:66; an HVR-H2 comprising the amino acid sequence of SEQ ID NO:80; an HVR-H3 comprising the amino acid sequence of SEQ ID NO:94; an HVR-L1 comprising the amino acid sequence of SEQ ID NO: 108; an HVR-L2 comprising the amino acid sequence of SEQ ID NO: 117; and an HVR-L3 comprising the amino acid sequence of SEQ ID NO: 128;
k. an HVR-H1 comprising the amino acid sequence of SEQ ID NO:66; an HVR-H2 comprising the amino acid sequence of SEQ ID NO:81; an HVR-H3 comprising the amino acid sequence of SEQ ID NO:95; an HVR-L1 comprising the amino acid sequence of SEQ ID NO: 109; an HVR-L2 comprising the amino acid sequence of SEQ ID NO: 118; and an HVR-L3 comprising the amino acid sequence of SEQ ID NO: 129;
l. an HVR-H1 comprising the amino acid sequence of SEQ ID NO:67; an HVR-H2 comprising the amino acid sequence of SEQ ID NO:82; an HVR-H3 comprising the amino acid sequence of SEQ ID NO:96; an HVR-L1 comprising the amino acid sequence of SEQ ID NO: 101; an HVR-L2 comprising the amino acid sequence of SEQ ID NO: 116; and an HVR-L3 comprising the amino acid sequence of SEQ ID NO:130;
m. an HVR-H1 comprising the amino acid sequence of SEQ ID NO:68; an HVR-H2 comprising the amino acid sequence of SEQ ID NO:83; an HVR-H3 comprising the amino acid sequence of SEQ ID NO:97; an HVR-L1 comprising the amino acid sequence of SEQ ID NO: 101; an HVR-L2 comprising the amino acid sequence of SEQ ID NO: 119; and an HVR-L3 comprising the amino acid sequence of SEQ ID NO: 131;
n. an HVR-H1 comprising the amino acid sequence of SEQ ID NO:69; an HVR-H2 comprising the amino acid sequence of SEQ ID NO:84; an HVR-H3 comprising the amino acid sequence of SEQ ID NO:98; an HVR-L1 comprising the amino acid sequence of SEQ ID NO: 110; an HVR-L2 comprising the amino acid sequence of SEQ ID NO: 120; and an HVR-L3 comprising the amino acid sequence of SEQ ID NO: 132;
o. an HVR-H1 comprising the amino acid sequence of SEQ ID NO: 70; an HVR-H2 comprising the amino acid sequence of SEQ ID NO:85; an HVR-H3 comprising the amino acid sequence of SEQ ID NO:99; an HVR-L1 comprising the amino acid sequence of SEQ ID NO: 111; an HVR-L2 comprising the amino acid sequence of SEQ ID NO: 121; and an HVR-L3 comprising the amino acid sequence of SEQ ID NO:133;
p. an HVR-H1 comprising the amino acid sequence of SEQ ID NO: 70; an HVR-H2 comprising the amino acid sequence of SEQ ID NO:86; an HVR-H3 comprising the amino acid sequence of SEQ ID NO:100; an HVR-L1 comprising the amino acid sequence of SEQ ID NO: 111; an HVR-L2 comprising the amino acid sequence of SEQ ID NO: 121; and an HVR-L3 comprising the amino acid sequence of SEQ ID NO: 133;
q. the HVR-H1 comprises the amino acid sequence of SEQ ID NO: 167; the HVR-H2 comprises the amino acid sequence of SEQ ID NO: 170; the HVR-H3 comprises the amino acid sequence of SEQ ID NO: 179; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
r. the HVR-H1 comprises the amino acid sequence of SEQ ID NO: 168; the HVR-H2 comprises the amino acid sequence of SEQ ID NO: 171; the HVR-H3 comprises the amino acid sequence of SEQ ID NO: 180; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
s. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 181; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249;
t. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 182; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
u. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
v. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
w. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 185; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249;
x. the HVR-H1 comprises the amino acid sequence of SEQ ID NO: 169; the HVR-H2 comprises the amino acid sequence of SEQ ID NO: 1704; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
y. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
z. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
aa. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
bb. the HVR-H1 comprises the amino acid sequence of SEQ ID NO: 169; the HVR-H2 comprises the amino acid sequence of SEQ ID NO: 176; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
cc. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 187; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
dd, the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 188; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
ee, the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 182; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
ff. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
gg. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
hh. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
ii. the HVR-H1 comprises the amino acid sequence of SEQ ID NO: 169; the HVR-H2 comprises the amino acid sequence of SEQ ID NO:177; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
jj. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
kk. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 185; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249;
ll. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
mm. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
nn. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 184; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249;
oo. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
pp. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
qq. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
rr. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
ss. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
tt. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
uu. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249; or
vv. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO: 248; and the HVR-L3 comprises the amino acid sequence of SEQ ID NO:249.

Embodiment 26 is the antibody of embodiment 25, wherein the antibody comprises:
a. the HVRs of embodiment 25.a, and further comprises a VH that is at least 90%, at least 95%, at least 97%, or at least 99% identical to the amino acid sequence of SEQ ID NO:29;
b. the HVRs of embodiment 25.b, and further comprises a VH that is at least 90%, at least 95%, at least 97%, or at least 99% identical to the amino acid sequence of SEQ ID NO:30;
c. the HVRs of embodiment 25.c, and further comprises a VH that is at least 90%, at least 95%, at least 97%, or at least 99% identical to the amino acid sequence of SEQ ID NO:31;
d. the HVRs of embodiment 25.d, and further comprises a VH that is at least 90%, at least 95%, at least 97%, or at least 99% identical to the amino acid sequence of SEQ ID NO:32;
e. the HVRs of embodiment 25.e, and further comprises a VH that is at least 90%, at least 95%, at least 97%, or at least 99% identical to the amino acid sequence of SEQ ID NO:33;
f. the HVRs of embodiment 25.f, and further comprises a VH that is at least 90%, at least 95%, at least 97%, or at least 99% identical to the amino acid sequence of SEQ ID NO:34;
g. the HVRs of embodiment 25.g, and further comprises a VH that is at least 90%, at least 95%, at least 97%, or at least 99% identical to the amino acid sequence of SEQ ID NO:35;
h. the HVRs of embodiment 25.h, and further comprises a VH that is at least 90%, at least 95%, at least 97%, or at least 99% identical to the amino acid sequence of SEQ ID NO:36;
i. the HVRs of embodiment 25.i, and further comprises a VH that is at least 90%, at least 95%, at least 97%, or at least 99% identical to the amino acid sequence of SEQ ID NO:37;
j. the HVRs of embodiment 25.j, and further comprises a VH that is at least 90%, at least 95%, at least 97%, or at least 99% identical to the amino acid sequence of SEQ ID NO:38;
k. the HVRs of embodiment 25.k, and further comprises a VH that is at least 90%, at least 95%, at least 97%, or at least 99% identical to the amino acid sequence of SEQ ID NO:39;
l. the HVRs of embodiment 25.1, and further comprises a VH that is at least 90%, at least 95%, at least 97%, or at least 99% identical to the amino acid sequence of SEQ ID NO:40;
m. the HVRs of embodiment 25.m, and further comprises a VH that is at least 90%, at least 95%, at least 97%, or at least 99% identical to the amino acid sequence of SEQ ID NO:41;
n. the HVRs of embodiment 25.n, and further comprises a VH that is at least 90%, at least 95%, at least 97%, or at least 99% identical to the amino acid sequence of SEQ ID NO:42;
o. the HVRs of embodiment 25.0, and further comprises a VH that is at least 90%, at least 95%, at least 97%, or at least 99% identical to the amino acid sequence of SEQ ID NO:43;
p. the HVRs of embodiment 25.p, and further comprises a VH that is at least 90%, at least 95%, at least 97%, or at least 99% identical to the amino acid sequence of SEQ ID NO:44;
q. the HVRs of embodiment 25.q, and further comprises a VH that is at least 90%, at least 95%, at least 97%, or at least 99% identical to the amino acid sequence of SEQ ID NO: 134;
r. the HVRs of embodiment 25.r, and further comprises a VH that is at least 90%, at least 95%, at least 97%, or at least 99% identical to the amino acid sequence of SEQ ID NO: 135;
s. the HVRs of embodiment 25.s, and further comprises a VH that is at least 90%, at least 95%, at least 97%, or at least 99% identical to the amino acid sequence of SEQ ID NO: 136;
t. the HVRs of embodiment 25.t, and further comprises a VH that is at least 90%, at least 95%, at least 97%, or at least 99% identical to the amino acid sequence of SEQ ID NO: 137;
u. the HVRs of embodiment 25.u, and further comprises a VH that is at least 90%, at least 95%, at least 97%, or at least 99% identical to the amino acid sequence of SEQ ID NO: 138;
v. the HVRs of embodiment 25.v, and further comprises a VH that is at least 90%, at least 95%, at least 97%, or at least 99% identical to the amino acid sequence of SEQ ID NO: 139;
w. the HVRs of embodiment 25.w, and further comprises a VH that is at least 90%, at least 95%, at least 97%, or at least 99% identical to the amino acid sequence of SEQ ID NO: 140;
x. the HVRs of embodiment 25.x, and further comprises a VH that is at least 90%, at least 95%, at least 97%, or at least 99% identical to the amino acid sequence of SEQ ID NO: 141;
y. the HVRs of embodiment 25.y, and further comprises a VH that is at least 90%, at least 95%, at least 97%, or at least 99% identical to the amino acid sequence of SEQ ID NO: 142;
z. the HVRs of embodiment 25.z, and further comprises a VH that is at least 90%, at least 95%, at least 97%, or at least 99% identical to the amino acid sequence of SEQ ID NO: 143;
aa. the HVRs of embodiment 25.aa, and further comprises a VH that is at least 90%, at least 95%, at least 97%, or at least 99% identical to the amino acid sequence of SEQ ID NO: 144;
bb. the HVRs of embodiment 25.bb, and further comprises a VH that is at least 90%, at least 95%, at least 97%, or at least 99% identical to the amino acid sequence of SEQ ID NO: 145;
cc. the HVRs of embodiment 25.cc, and further comprises a VH that is at least 90%, at least 95%, at least 97%, or at least 99% identical to the amino acid sequence of SEQ ID NO: 146;
dd, the HVRs of embodiment 25.dd, and further comprises a VH that is at least 90%, at least 95%, at least 97%, or at least 99% identical to the amino acid sequence of SEQ ID NO: 147;
ee, the HVRs of embodiment 25.cc, and further comprises a VH that is at least 90%, at least 95%, at least 97%, or at least 99% identical to the amino acid sequence of SEQ ID NO: 148;
ff. the HVRs of embodiment 25.ff, and further comprises a VH that is at least 90%, at least 95%, at least 97%, or at least 99% identical to the amino acid sequence of SEQ ID NO: 149;
gg. the HVRs of embodiment 25.gg, and further comprises a VH that is at least 90%, at least 95%, at least 97%, or at least 99% identical to the amino acid sequence of SEQ ID NO: 150;
hh. the HVRs of embodiment 25.hh, and further comprises a VH that is at least 90%, at least 95%, at least 97%, or at least 99% identical to the amino acid sequence of SEQ ID NO: 151;
ii. the HVRs of embodiment 25.ii, and further comprises a VH that is at least 90%, at least 95%, at least 97%, or at least 99% identical to the amino acid sequence of SEQ ID NO: 152;
jj. the HVRs of embodiment 25.jj, and further comprises a VH that is at least 90%, at least 95%, at least 97%, or at least 99% identical to the amino acid sequence of SEQ ID NO: 153;
kk. the HVRs of embodiment 25.kk, and further comprises a VH that is at least 90%, at least 95%, at least 97%, or at least 99% identical to the amino acid sequence of SEQ ID NO: 154;
11, the HVRs of embodiment 2511, and further comprises a VH that is at least 90%, at least 95%, at least 97%, or at least 99% identical to the amino acid sequence of SEQ ID NO: 155;
mm. the HVRs of embodiment 25.mm, and further comprises a VH that is at least 90%, at least 95%, at least 97%, or at least 99% identical to the amino acid sequence of SEQ ID NO: 156;
nn. the HVRs of embodiment 25.nn, and further comprises a VH that is at least 90%, at least 95%, at least 97%, or at least 99% identical to the amino acid sequence of SEQ ID NO: 157;
oo. the HVRs of embodiment 25.oo, and further comprises a VH that is at least 90%, at least 95%, at least 97%, or at least 99% identical to the amino acid sequence of SEQ ID NO: 158;
pp. the HVRs of embodiment 25.pp, and further comprises a VH that is at least 90%, at least 95%, at least 97%, or at least 99% identical to the amino acid sequence of SEQ ID NO: 159;
qq. the HVRs of embodiment 25.qq, and further comprises a VH that is at least 90%, at least 95%, at least 97%, or at least 99% identical to the amino acid sequence of SEQ ID NO: 160;
rr. the HVRs of embodiment 25.rr, and further comprises a VH that is at least 90%, at least 95%, at least 97%, or at least 99% identical to the amino acid sequence of SEQ ID NO: 161;
ss. the HVRs of embodiment 25.ss, and further comprises a VH that is at least 90%, at least 95%, at least 97%, or at least 99% identical to the amino acid sequence of SEQ ID NO: 162;
tt. the HVRs of embodiment 25.tt, and further comprises a VH that is at least 90%, at least 95%, at least 97%, or at least 99% identical to the amino acid sequence of SEQ ID NO: 163;
uu. the HVRs of embodiment 25.uu, and further comprises a VH that is at least 90%, at least 95%, at least 97%, or at least 99% identical to the amino acid sequence of SEQ ID NO: 164;
vv. the HVRs of embodiment 25.vv, and further comprises a VH that is at least 90%, at least 95%, at least 97%, or at least 99% identical to the amino acid sequence of SEQ ID NO: 165.

Embodiment 27 is the antibody of embodiment 25 or 26, wherein the antibody comprises:
a. the HVRs of embodiment 25.a, and further comprises a VL that is at least 90%, at least 95%, at least 97%, or at least 99% identical to the amino acid sequence of SEQ ID NO:45
b. the HVRs of embodiment 25.b, and further comprises a VL that is at least 90%, at least 95%, at least 97%, or at least 99% identical to the amino acid sequence of SEQ ID NO: 46;
c. the HVRs of embodiment 25.c, and further comprises a VL that is at least 90%, at least 95%, at least 97%, or at least 99% identical to the amino acid sequence of SEQ ID NO: 47;
d. the HVRs of embodiment 25.d, and further comprises a VL that is at least 90%, at least 95%, at least 97%, or at least 99% identical to the amino acid sequence of SEQ ID NO: 48;
e. the HVRs of embodiment 25.c, and further comprises a VL that is at least 90%, at least 95%, at least 97%, or at least 99% identical to the amino acid sequence of SEQ ID NO: 49;
f. the HVRs of embodiment 25.f, and further comprises a VL that is at least 90%, at least 95%, at least 97%, or at least 99% identical to the amino acid sequence of SEQ ID NO: 50;
g. the HVRs of embodiment 25.g, and further comprises a VL that is at least 90%, at least 95%, at least 97%, or at least 99% identical to the amino acid sequence of SEQ ID NO: 51;
h. the HVRs of embodiment 25.h, and further comprises a VL that is at least 90%, at least 95%, at least 97%, or at least 99% identical to the amino acid sequence of SEQ ID NO: 52;
i. the HVRs of embodiment 25.i, and further comprises a VL that is at least 90%, at least 95%, at least 97%, or at least 99% identical to the amino acid sequence of SEQ ID NO: 53;
j. the HVRs of embodiment 25.j, and further comprises a VL that is at least 90%, at least 95%, at least 97%, or at least 99% identical to the amino acid sequence of SEQ ID NO: 54;
k. the HVRs of embodiment 25.k, and further comprises a VL that is at least 90%, at least 95%, at least 97%, or at least 99% identical to the amino acid sequence of SEQ ID NO: 55;
l. the HVRs of embodiment 25.1, and further comprises a VL that is at least 90%, at least 95%, at least 97%, or at least 99% identical to the amino acid sequence of SEQ ID NO: 56;
m. the HVRs of embodiment 25.m, and further comprises a VL that is at least 90%, at least 95%, at least 97%, or at least 99% identical to the amino acid sequence of SEQ ID NO: 57
n. the HVRs of embodiment 25.n, and further comprises a VL that is at least 90%, at least 95%, at least 97%, or at least 99% identical to the amino acid sequence of SEQ ID NO: 58;
o. the HVRs of embodiment 25.0, and further comprises a VL that is at least 90%, at least 95%, at least 97%, or at least 99% identical to the amino acid sequence of SEQ ID NO: 59;
p. the HVRs of embodiment 25.p, and further comprises a VL that is at least 90%, at least 95%, at least 97%, or at least 99% identical to the amino acid sequence of SEQ ID NO: 59;
q. the HVRs of embodiment 25.q, and further comprises a VH that is at least 90%, at least 95%, at least 97%, or at least 99% identical to the amino acid sequence of SEQ ID NO: 134;
r. the HVRs of embodiment 25.r, and further comprises a VH that is at least 90%, at least 95%, at least 97%, or at least 99% identical to the amino acid sequence of SEQ ID NO: 135;
s. the HVRs of embodiment 25.s, and further comprises a VH that is at least 90%, at least 95%, at least 97%, or at least 99% identical to the amino acid sequence of SEQ ID NO: 136;
t. the HVRs of embodiment 25.t, and further comprises a VH that is at least 90%, at least 95%, at least 97%, or at least 99% identical to the amino acid sequence of SEQ ID NO: 137;
u. the HVRs of embodiment 25.u, and further comprises a VH that is at least 90%, at least 95%, at least 97%, or at least 99% identical to the amino acid sequence of SEQ ID NO: 138;
v. the HVRs of embodiment 25.v, and further comprises a VH that is at least 90%, at least 95%, at least 97%, or at least 99% identical to the amino acid sequence of SEQ ID NO:139;
w. the HVRs of embodiment 25.w, and further comprises a VH that is at least 90%, at least 95%, at least 97%, or at least 99% identical to the amino acid sequence of SEQ ID NO: 140;
x. the HVRs of embodiment 25.x, and further comprises a VH that is at least 90%, at least 95%, at least 97%, or at least 99% identical to the amino acid sequence of SEQ ID NO: 141;
y. the HVRs of embodiment 25.y, and further comprises a VH that is at least 90%, at least 95%, at least 97%, or at least 99% identical to the amino acid sequence of SEQ ID NO: 142;
z. the HVRs of embodiment 25.z, and further comprises a VH that is at least 90%, at least 95%, at least 97%, or at least 99% identical to the amino acid sequence of SEQ ID NO: 143;
aa. the HVRs of embodiment 25.aa, and further comprises a VH that is at least 90%, at least 95%, at least 97%, or at least 99% identical to the amino acid sequence of SEQ ID NO: 144;
bb. the HVRs of embodiment 25.bb, and further comprises a VH that is at least 90%, at least 95%, at least 97%, or at least 99% identical to the amino acid sequence of SEQ ID NO: 145;
cc. the HVRs of embodiment 25.cc, and further comprises a VH that is at least 90%, at least 95%, at least 97%, or at least 99% identical to the amino acid sequence of SEQ ID NO: 146;
dd, the HVRs of embodiment 25.dd, and further comprises a VH that is at least 90%, at least 95%, at least 97%, or at least 99% identical to the amino acid sequence of SEQ ID NO: 147;
ee, the HVRs of embodiment 25.cc, and further comprises a VH that is at least 90%, at least 95%, at least 97%, or at least 99% identical to the amino acid sequence of SEQ ID NO: 148;
ff. the HVRs of embodiment 25.ff, and further comprises a VH that is at least 90%, at least 95%, at least 97%, or at least 99% identical to the amino acid sequence of SEQ ID NO: 149;
gg. the HVRs of embodiment 25.gg, and further comprises a VH that is at least 90%, at least 95%, at least 97%, or at least 99% identical to the amino acid sequence of SEQ ID NO: 150;
hh. the HVRs of embodiment 25.hh, and further comprises a VH that is at least 90%, at least 95%, at least 97%, or at least 99% identical to the amino acid sequence of SEQ ID NO: 151;
ii. the HVRs of embodiment 25.ii, and further comprises a VH that is at least 90%, at least 95%, at least 97%, or at least 99% identical to the amino acid sequence of SEQ ID NO: 152;
jj. the HVRs of embodiment 25.jj, and further comprises a VH that is at least 90%, at least 95%, at least 97%, or at least 99% identical to the amino acid sequence of SEQ ID NO: 153;
kk. the HVRs of embodiment 25.kk, and further comprises a VH that is at least 90%, at least 95%, at least 97%, or at least 99% identical to the amino acid sequence of SEQ ID NO: 154;
ll. the HVRs of embodiment 2511, and further comprises a VH that is at least 90%, at least 95%, at least 97%, or at least 99% identical to the amino acid sequence of SEQ ID NO: 155;
mm. the HVRs of embodiment 25.mm, and further comprises a VH that is at least 90%, at least 95%, at least 97%, or at least 99% identical to the amino acid sequence of SEQ ID NO: 156;
nn. the HVRs of embodiment 25.nn, and further comprises a VH that is at least 90%, at least 95%, at least 97%, or at least 99% identical to the amino acid sequence of SEQ ID NO: 157;
00, the HVRs of embodiment 25.oo, and further comprises a VH that is at least 90%, at least 95%, at least 97%, or at least 99% identical to the amino acid sequence of SEQ ID NO: 158;
pp. the HVRs of embodiment 25.pp, and further comprises a VH that is at least 90%, at least 95%, at least 97%, or at least 99% identical to the amino acid sequence of SEQ ID NO: 159;
qq. the HVRs of embodiment 25.qq, and further comprises a VH that is at least 90%, at least 95%, at least 97%, or at least 99% identical to the amino acid sequence of SEQ ID NO: 160;
rr. the HVRs of embodiment 25.rr, and further comprises a VH that is at least 90%, at least 95%, at least 97%, or at least 99% identical to the amino acid sequence of SEQ ID NO: 161;
ss. the HVRs of embodiment 25.ss, and further comprises a VH that is at least 90%, at least 95%, at least 97%, or at least 99% identical to the amino acid sequence of SEQ ID NO: 162;
tt. the HVRs of embodiment 25.tt, and further comprises a VH that is at least 90%, at least 95%, at least 97%, or at least 99% identical to the amino acid sequence of SEQ ID NO: 163;
uu. the HVRs of embodiment 25.uu, and further comprises a VH that is at least 90%, at least 95%, at least 97%, or at least 99% identical to the amino acid sequence of SEQ ID NO: 164; or
vv. the HVRs of embodiment 25.vv, and further comprises a VH that is at least 90%, at least 95%, at least 97%, or at least 99% identical to the amino acid sequence of SEQ ID NO: 165.

Embodiment 28 is the antibody of any one of embodiments 25-27, wherein the antibody comprises:
a. the HVRs of embodiment 25.a, and further comprises a VH comprising the amino acid sequence of SEQ ID NO:29;
b. the HVRs of embodiment 25.b, and further comprises a VH comprising the amino acid sequence of SEQ ID NO:30;
c. the HVRs of embodiment 25.c, and further comprises a VH comprising the amino acid sequence of SEQ ID NO:31;
d. the HVRs of embodiment 25.d, and further comprises a VH comprising the amino acid sequence of SEQ ID NO:32;
e. the HVRs of embodiment 25.e, and further comprises a VH comprising the amino acid sequence of SEQ ID NO:33;
f. the HVRs of embodiment 25.f, and further comprises a VH comprising the amino acid sequence of SEQ ID NO:34;
g. the HVRs of embodiment 25.g, and further comprises a VH comprising the amino acid sequence of SEQ ID NO:35;
h. the HVRs of embodiment 25.h, and further comprises a VH comprising the amino acid sequence of SEQ ID NO:36;
i. the HVRs of embodiment 25.i, and further comprises a VH comprising the amino acid sequence of SEQ ID NO:37;
j. the HVRs of embodiment 25.j, and further comprises a VH comprising the amino acid sequence of SEQ ID NO:38;
k. the HVRs of embodiment 25.k, and further comprises a VH comprising the amino acid sequence of SEQ ID NO:39;
l. the HVRs of embodiment 25.1, and further comprises a VH comprising the amino acid sequence of SEQ ID NO:40;
m. the HVRs of embodiment 25.m, and further comprises a comprising the amino acid sequence of SEQ ID NO:41;
n. the HVRs of embodiment 25.n, and further comprises a VH comprising the amino acid sequence of SEQ ID NO:42;
o. the HVRs of embodiment 25.0, and further comprises a VH comprising the amino acid sequence of SEQ ID NO:43;
p. the HVRs of embodiment 25.p, and further comprises a VH comprising the amino acid sequence of SEQ ID NO:44
q. the HVRs of embodiment 25.q, and further comprises a VH comprising the amino acid sequence of SEQ ID NO: 134;
r. the HVRs of embodiment 25.r, and further comprises a VH comprising the amino acid sequence of SEQ ID NO: 135;
s. the HVRs of embodiment 25.s, and further comprises a VH comprising the amino acid sequence of SEQ ID NO: 136;
t. the HVRs of embodiment 25.t, and further comprises a VH comprising the amino acid sequence of SEQ ID NO: 137;
u. the HVRs of embodiment 25.u, and further comprises a VH comprising the amino acid sequence of SEQ ID NO: 138;
v. the HVRs of embodiment 25.v, and further comprises a VH comprising the amino acid sequence of SEQ ID NO: 139;
w. the HVRs of embodiment 25.w, and further comprises a VH comprising the amino acid sequence of SEQ ID NO: 140;
x. the HVRs of embodiment 25.x, and further comprises a VH comprising the amino acid sequence of SEQ ID NO: 141;
y. the HVRs of embodiment 25.y, and further comprises a VH comprising the amino acid sequence of SEQ ID NO: 142;
z. the HVRs of embodiment 25.z, and further comprises a VH comprising the amino acid sequence of SEQ ID NO: 143;
aa. the HVRs of embodiment 25.aa, and further comprises a VH comprising the amino acid sequence of SEQ ID NO: 144;
bb. the HVRs of embodiment 25.bb, and further comprises a VH comprising the amino acid sequence of SEQ ID NO: 145;
cc. the HVRs of embodiment 25.cc, and further comprises a VH comprising the amino acid sequence of SEQ ID NO: 146;
dd, the HVRs of embodiment 25.dd, and further comprises a VH comprising the amino acid sequence of SEQ ID NO: 147;
ee, the HVRs of embodiment 25.ee, and further comprises a VH comprising the amino acid sequence of SEQ ID NO: 148;
ff. the HVRs of embodiment 25.ff, and further comprises a VH comprising the amino acid sequence of SEQ ID NO: 149;
gg. the HVRs of embodiment 25.gg, and further comprises a VH comprising the amino acid sequence of SEQ ID NO: 150;
hh. the HVRs of embodiment 25.hh, and further comprises a VH comprising the amino acid sequence of SEQ ID NO: 151;
ii. the HVRs of embodiment 25.ii, and further comprises a VH comprising the amino acid sequence of SEQ ID NO: 152;
jj. the HVRs of embodiment 25.jj, and further comprises a VH comprising the amino acid sequence of SEQ ID NO: 153;
kk. the HVRs of embodiment 25.kk, and further comprises a VH that is at least 90%, at least 95%, at least 97%, or at least 99% identical t VH comprising o the amino acid sequence of SEQ ID NO: 154;
ll. the HVRs of embodiment 2511, and further comprises a VH comprising the amino acid sequence of SEQ ID NO: 155;
mm. the HVRs of embodiment 25.mm, and further comprises a VH comprising the amino acid sequence of SEQ ID NO: 156;
nn. the HVRs of embodiment 25.nn, and further comprises a VH comprising the amino acid sequence of SEQ ID NO: 157;
oo. the HVRs of embodiment 25.oo, and further comprises a VH comprising amino acid sequence of SEQ ID NO:158;
pp. the HVRs of embodiment 25.pp, and further comprises a VH comprising the amino acid sequence of SEQ ID NO: 159;
qq. the HVRs of embodiment 25.qq, and further comprises a VH comprising the amino acid sequence of SEQ ID NO: 160;
rr. the HVRs of embodiment 25.rr, and further comprises a VH comprising the amino acid sequence of SEQ ID NO: 161;
ss. the HVRs of embodiment 25.ss, and further comprises a VH comprising the amino acid sequence of SEQ ID NO: 162;
tt. the HVRs of embodiment 25.tt, and further comprises a VH comprising the amino acid sequence of SEQ ID NO: 163;
uu. the HVRs of embodiment 25.uu, and further comprises a VH comprising the amino acid sequence of SEQ ID NO: 164; or
vv. the HVRs of embodiment 25.vv, and further comprises a VH comprising the amino acid sequence of SEQ ID NO: 165.

Embodiment 29 is the antibody of any one of embodiments 25-28, wherein the antibody comprises:
a. the HVRs of embodiment 25.a, and further comprises a VL comprising the amino acid sequence of SEQ ID NO:45
b. the HVRs of embodiment 25.b, and further comprises a VL comprising amino acid sequence of SEQ ID NO: 46;
c. the HVRs of embodiment 25.c, and further comprises a VL comprising the amino acid sequence of SEQ ID NO: 47;
d. the HVRs of embodiment 25.d, and further comprises a VL comprising the amino acid sequence of SEQ ID NO: 48;
e. the HVRs of embodiment 25.e, and further comprises a VL comprising the amino acid sequence of SEQ ID NO: 49;
f. the HVRs of embodiment 25.f, and further comprises a VL comprising the amino acid sequence of SEQ ID NO: 50;
g. the HVRs of embodiment 25.g, and further comprises a VL comprising the amino acid sequence of SEQ ID NO: 51;
h. the HVRs of embodiment 25.h, and further comprises a VL comprising the amino acid sequence of SEQ ID NO: 52;
i. the HVRs of embodiment 25.i, and further comprises a VL comprising the amino acid sequence of SEQ ID NO: 53;
j. the HVRs of embodiment 25.j, and further comprises a VL comprising the amino acid sequence of SEQ ID NO: 54;
k. the HVRs of embodiment 25.k, and further comprises a VL comprising the amino acid sequence of SEQ ID NO: 55;
l. the HVRs of embodiment 25.1, and further comprises a VL comprising the amino acid sequence of SEQ ID NO: 56;
m. the HVRs of embodiment 25.m, and further comprises a VL comprising the amino acid sequence of SEQ ID NO: 57
n. the HVRs of embodiment 25.n, and further comprises a VL comprising the amino acid sequence of SEQ ID NO: 58;
o. the HVRs of embodiment 25.0, and further comprises a VL comprising the amino acid sequence of SEQ ID NO: 59; or
p. the HVRs of embodiment 25.p, and further comprises a VL comprising the amino acid sequence of SEQ ID NO: 59
q. the HVRs of embodiment 25.q, and further comprises a VL comprising the amino acid sequence of SEQ ID NO: 166;
r. the HVRs of embodiment 25.r, and further comprises a VL comprising the amino acid sequence of SEQ ID NO: 166;
s. the HVRs of embodiment 25.s, and further comprises a VL comprising the amino acid sequence of SEQ ID NO: 166;
t. the HVRs of embodiment 25.t, and further comprises a VL comprising the amino acid sequence of SEQ ID NO: 166;
u. the HVRs of embodiment 25.u, and further comprises a VL comprising the amino acid sequence of SEQ ID NO: 166;
v. the HVRs of embodiment 25.v, and further comprises a VL comprising the amino acid sequence of SEQ ID NO: 166;
w. the HVRs of embodiment 25.w, and further comprises a VL comprising the amino acid sequence of SEQ ID NO: 166;
x. the HVRs of embodiment 25.x, and further comprises a VL comprising the amino acid sequence of SEQ ID NO: 166;
y. the HVRs of embodiment 25.y, and further comprises a VL comprising the amino acid sequence of SEQ ID NO: 166;
z. the HVRs of embodiment 25.z, and further comprises a VL comprising the amino acid sequence of SEQ ID NO: 166;
aa. the HVRs of embodiment 25.aa, and further comprises a VL comprising the amino acid sequence of SEQ ID NO: 166;
bb. the HVRs of embodiment 25.bb, and further comprises a VL comprising the amino acid sequence of SEQ ID NO: 166;
cc. the HVRs of embodiment 25.cc, and further comprises a VL comprising the amino acid sequence of SEQ ID NO: 166;
dd, the HVRs of embodiment 25.dd, and further comprises a VL comprising the amino acid sequence of SEQ ID NO: 166;
ee, the HVRs of embodiment 25.ee, and further comprises a VL comprising the amino acid sequence of SEQ ID NO: 166;
ff. the HVRs of embodiment 25.ff, and further comprises a VL comprising the amino acid sequence of SEQ ID NO: 166;
gg. the HVRs of embodiment 25.gg, and further comprises a VL comprising the amino acid sequence of SEQ ID NO: 166;
hh. the HVRs of embodiment 25.hh, and further comprises a VL comprising the amino acid sequence of SEQ ID NO: 166;
ii. the HVRs of embodiment 25.ii, and further comprises a VL comprising the amino acid sequence of SEQ ID NO: 166;
jj. the HVRs of embodiment 25.jj, and further comprises a VL comprising the amino acid sequence of SEQ ID NO: 166;
kk. the HVRs of embodiment 25.kk, and further comprises a VL comprising the amino acid sequence of SEQ ID NO: 166;
ll. the HVRs of embodiment 25.ll, and further comprises a VL comprising the amino acid sequence of SEQ ID NO: 166;
mm. the HVRs of embodiment 25.mm, and further comprises a VL comprising the amino acid sequence of SEQ ID NO: 166;
nn. the HVRs of embodiment 25.nn, and further comprises a VL comprising the amino acid sequence of SEQ ID NO: 166;
oo. the HVRs of embodiment 25.oo, and further comprises a VL comprising the amino acid sequence of SEQ ID NO: 166;
pp. the HVRs of embodiment 25.pp, and further comprises a VL comprising the amino acid sequence of SEQ ID NO: 166;
qq. the HVRs of embodiment 25.qq, and further comprises a VL comprising the amino acid sequence of SEQ ID NO: 166;
rr. the HVRs of embodiment 25.rr, and further comprises a VL comprising the amino acid sequence of SEQ ID NO: 166;
ss. the HVRs of embodiment 25.ss, and further comprises a VL comprising the amino acid sequence of SEQ ID NO: 166;
tt. the HVRs of embodiment 25.tt, and further comprises a VL comprising the amino acid sequence of SEQ ID NO: 166;
uu. the HVRs of embodiment 25.uu, and further comprises a VL comprising the amino acid sequence of SEQ ID NO: 166; or
vv. the HVRs of embodiment 25.vv, and further comprises a VL comprising the amino acid sequence of SEQ ID NO: 166.

Embodiment 30 is the antibody of embodiment 25, wherein the antibody comprises:
a. a VH comprising the amino acid sequence of SEQ ID NO: 29 and a VL comprising the amino acid sequence of SEQ ID NO: 45;
b. a VH comprising the amino acid sequence of SEQ ID NO: 30 and a VL comprising the amino acid sequence of SEQ ID NO: 46;
c. a VH comprising the amino acid sequence of SEQ ID NO: 31 and a VL comprising the amino acid sequence of SEQ ID NO: 47;
d. a VH comprising the amino acid sequence of SEQ ID NO: 32 and a VL comprising the amino acid sequence of SEQ ID NO: 48;
e. a VH comprising the amino acid sequence of SEQ ID NO: 33 and a VL comprising the amino acid sequence of SEQ ID NO: 49;
f. a VH comprising the amino acid sequence of SEQ ID NO: 34 and a VL comprising the amino acid sequence of SEQ ID NO: 50;
g. a VH comprising the amino acid sequence of SEQ ID NO: 35 and a VL comprising the amino acid sequence of SEQ ID NO: 51;
h. a VH comprising the amino acid sequence of SEQ ID NO: 36 and a VL comprising the amino acid sequence of SEQ ID NO: 52;
i. a VH comprising the amino acid sequence of SEQ ID NO: 37 and a VL comprising the amino acid sequence of SEQ ID NO: 53;
j. a VH comprising the amino acid sequence of SEQ ID NO: 38 and a VL comprising the amino acid sequence of SEQ ID NO: 54;
k. a VH comprising the amino acid sequence of SEQ ID NO: 39 and a VL comprising the amino acid sequence of SEQ ID NO: 55;
l. a VH comprising the amino acid sequence of SEQ ID NO: 40 and a VL comprising the amino acid sequence of SEQ ID NO: 56;
m. a VH comprising the amino acid sequence of SEQ ID NO: 41 and a VL comprising the amino acid sequence of SEQ ID NO: 57;
n. a VH comprising the amino acid sequence of SEQ ID NO: 42 and a VL comprising the amino acid sequence of SEQ ID NO: 58;
o. a VH comprising the amino acid sequence of SEQ ID NO: 43 and a VL comprising the amino acid sequence of SEQ ID NO: 59;
p. a VH comprising the amino acid sequence of SEQ ID NO: 44 and a VL comprising the amino acid sequence of SEQ ID NO: 59,
q. a VH comprising the amino acid sequence of SEQ ID NO: 134 and a VL comprising the amino acid sequence of SEQ ID NO: 166;
r. a VH comprising the amino acid sequence of SEQ ID NO: 135 and a VL comprising the amino acid sequence of SEQ ID NO: 166;
s. a VH comprising the amino acid sequence of SEQ ID NO: 136 and a VL comprising the amino acid sequence of SEQ ID NO: 166;
t. a VH comprising the amino acid sequence of SEQ ID NO: 137 and a VL comprising the amino acid sequence of SEQ ID NO: 166;
u. a VH comprising the amino acid sequence of SEQ ID NO: 138 and a VL comprising the amino acid sequence of SEQ ID NO: 166;
v. a VH comprising the amino acid sequence of SEQ ID NO: 139 and a VL comprising the amino acid sequence of SEQ ID NO: 166;
w. a VH comprising the amino acid sequence of SEQ ID NO: 140 and a VL comprising the amino acid sequence of SEQ ID NO: 166;
x. a VH comprising the amino acid sequence of SEQ ID NO: 141 and a VL comprising the amino acid sequence of SEQ ID NO: 166;
y. a VH comprising the amino acid sequence of SEQ ID NO: 142 and a VL comprising the amino acid sequence of SEQ ID NO: 166;
z. a VH comprising the amino acid sequence of SEQ ID NO: 143 and a VL comprising the amino acid sequence of SEQ ID NO: 166;
aa. a VH comprising the amino acid sequence of SEQ ID NO: 144 and a VL comprising the amino acid sequence of SEQ ID NO: 166;
bb. a VH comprising the amino acid sequence of SEQ ID NO: 145 and a VL comprising the amino acid sequence of SEQ ID NO: 166;
cc. a VH comprising the amino acid sequence of SEQ ID NO: 146 and a VL comprising the amino acid sequence of SEQ ID NO: 166;
dd. a VH comprising the amino acid sequence of SEQ ID NO: 147 and a VL comprising the amino acid sequence of SEQ ID NO: 166;
ee. a VH comprising the amino acid sequence of SEQ ID NO: 148 and a VL comprising the amino acid sequence of SEQ ID NO: 166;
ff. a VH comprising the amino acid sequence of SEQ ID NO: 149 and a VL comprising the amino acid sequence of SEQ ID NO: 166;
gg. a VH comprising the amino acid sequence of SEQ ID NO: 150 and a VL comprising the amino acid sequence of SEQ ID NO: 166;
hh. a VH comprising the amino acid sequence of SEQ ID NO: 151 and a VL comprising the amino acid sequence of SEQ ID NO: 166;
ii. a VH comprising the amino acid sequence of SEQ ID NO: 152 and a VL comprising the amino acid sequence of SEQ ID NO: 166;
jj. a VH comprising the amino acid sequence of SEQ ID NO: 153 and a VL comprising the amino acid sequence of SEQ ID NO: 166;
kk. a VH comprising the amino acid sequence of SEQ ID NO: 154 and a VL comprising the amino acid sequence of SEQ ID NO: 166;
ll. a VH comprising the amino acid sequence of SEQ ID NO: 155 and a VL comprising the amino acid sequence of SEQ ID NO: 166;
mm. a VH comprising the amino acid sequence of SEQ ID NO: 156 and a VL comprising the amino acid sequence of SEQ ID NO: 166;
nn. a VH comprising the amino acid sequence of SEQ ID NO: 157 and a VL comprising the amino acid sequence of SEQ ID NO: 166;
oo. a VH comprising the amino acid sequence of SEQ ID NO: 158 and a VL comprising the amino acid sequence of SEQ ID NO: 166;
pp. a VH comprising the amino acid sequence of SEQ ID NO: 159 and a VL comprising the amino acid sequence of SEQ ID NO: 166;
qq. a VH comprising the amino acid sequence of SEQ ID NO: 160 and a VL comprising the amino acid sequence of SEQ ID NO: 166;
rr. a VH comprising the amino acid sequence of SEQ ID NO: 161 and a VL comprising the amino acid sequence of SEQ ID NO: 166;
ss. a VH comprising the amino acid sequence of SEQ ID NO: 162 and a VL comprising the amino acid sequence of SEQ ID NO: 166;
tt. a VH comprising the amino acid sequence of SEQ ID NO: 163 and a VL comprising the amino acid sequence of SEQ ID NO: 166;
uu. a VH comprising the amino acid sequence of SEQ ID NO: 164 and a VL comprising the amino acid sequence of SEQ ID NO: 166; or
vv. a VH comprising the amino acid sequence of SEQ ID NO: 165 and a VL comprising the amino acid sequence of SEQ ID NO: 166.

Embodiment 31 is the antibody of embodiment 25, wherein the antibody comprises:
a. the HVRs of embodiment 25.s, and further comprises a heavy chain that is at least 90%, at least 95%, at least 97%, or at least 99% identical to the amino acid sequence of SEQ ID NO: 190;
b. the HVRs of embodiment 25.s, and further comprises a heavy chain that is at least 90%, at least 95%, at least 97%, or at least 99% identical to the amino acid sequence of SEQ ID NO: 191;
c. the HVRs of embodiment 25.s, and further comprises a heavy chain that is at least 90%, at least 95%, at least 97%, or at least 99% identical to the amino acid sequence of SEQ ID NO: 192;
d. the HVRs of embodiment 25.s, and further comprises a heavy chain that is at least 90%, at least 95%, at least 97%, or at least 99% identical to the amino acid sequence of SEQ ID NO: 193;
e. the HVRs of embodiment 25.s, and further comprises a heavy chain that is at least 90%, at least 95%, at least 97%, or at least 99% identical to the amino acid sequence of SEQ ID NO: 194;
f. the HVRs of embodiment 25.s, and further comprises a heavy chain that is at least 90%, at least 95%, at least 97%, or at least 99% identical to the amino acid sequence of SEQ ID NO: 195;
g. the HVRs of embodiment 25.s, and further comprises a heavy chain that is at least 90%, at least 95%, at least 97%, or at least 99% identical to the amino acid sequence of SEQ ID NO: 196;
h. the HVRs of embodiment 25.s, and further comprises a heavy chain that is at least 90%, at least 95%, at least 97%, or at least 99% identical to the amino acid sequence of SEQ ID NO: 197;
i. the HVRs of embodiment 25.s, and further comprises a heavy chain that is at least 90%, at least 95%, at least 97%, or at least 99% identical to the amino acid sequence of SEQ ID NO: 198;
j. the HVRs of embodiment 25.s, and further comprises a heavy chain that is at least 90%, at least 95%, at least 97%, or at least 99% identical to the amino acid sequence of SEQ ID NO: 199;
k. the HVRs of embodiment 25.s, and further comprises a heavy chain that is at least 90%, at least 95%, at least 97%, or at least 99% identical to the amino acid sequence of SEQ ID NO: 200;
l. the HVRs of embodiment 25.s, and further comprises a heavy chain that is at least 90%, at least 95%, at least 97%, or at least 99% identical to the amino acid sequence of SEQ ID NO: 201;
m. the HVRs of embodiment 25.s, and further comprises a heavy chain that is at least 90%, at least 95%, at least 97%, or at least 99% identical to the amino acid sequence of SEQ ID NO: 202;
n. the HVRs of embodiment 25.s, and further comprises a heavy chain that is at least 90%, at least 95%, at least 97%, or at least 99% identical to the amino acid sequence of SEQ ID NO: 203;
o. the HVRs of embodiment 25.s, and further comprises a heavy chain that is at least 90%, at least 95%, at least 97%, or at least 99% identical to the amino acid sequence of SEQ ID NO: 204;
p. the HVRs of embodiment 25.u, and further comprises a heavy chain that is at least 90%, at least 95%, at least 97%, or at least 99% identical to the amino acid sequence of SEQ ID NO: 205;
q. the HVRs of embodiment 25.u, and further comprises a heavy chain that is at least 90%, at least 95%, at least 97%, or at least 99% identical to the amino acid sequence of SEQ ID NO: 206;
r. the HVRs of embodiment 25.u, and further comprises a heavy chain that is at least 90%, at least 95%, at least 97%, or at least 99% identical to the amino acid sequence of SEQ ID NO: 207;
s. the HVRs of embodiment 25.u, and further comprises a heavy chain that is at least 90%, at least 95%, at least 97%, or at least 99% identical to the amino acid sequence of SEQ ID NO: 208;
t. the HVRs of embodiment 25.u, and further comprises a heavy chain that is at least 90%, at least 95%, at least 97%, or at least 99% identical to the amino acid sequence of SEQ ID NO: 209;
u. the HVRs of embodiment 25.u, and further comprises a heavy chain that is at least 90%, at least 95%, at least 97%, or at least 99% identical to the amino acid sequence of SEQ ID NO: 210;
v. the HVRs of embodiment 25.u, and further comprises a heavy chain that is at least 90%, at least 95%, at least 97%, or at least 99% identical to the amino acid sequence of SEQ ID NO: 211;
w. the HVRs of embodiment 25.u, and further comprises a heavy chain that is at least 90%, at least 95%, at least 97%, or at least 99% identical to the amino acid sequence of SEQ ID NO: 212;
x. the HVRs of embodiment 25.u, and further comprises a heavy chain that is at least 90%, at least 95%, at least 97%, or at least 99% identical to the amino acid sequence of SEQ ID NO: 213;
y. the HVRs of embodiment 25.u, and further comprises a heavy chain that is at least 90%, at least 95%, at least 97%, or at least 99% identical to the amino acid sequence of SEQ ID NO: 214;
z. the HVRs of embodiment 25.u, and further comprises a heavy chain that is at least 90%, at least 95%, at least 97%, or at least 99% identical to the amino acid sequence of SEQ ID NO: 215;
aa. the HVRs of embodiment 25.u, and further comprises a heavy chain that is at least 90%, at least 95%, at least 97%, or at least 99% identical to the amino acid sequence of SEQ ID NO: 216;
bb. the HVRs of embodiment 25.u, and further comprises a heavy chain that is at least 90%, at least 95%, at least 97%, or at least 99% identical to the amino acid sequence of SEQ ID NO: 217;
cc. the HVRs of embodiment 25.u, and further comprises a heavy chain that is at least 90%, at least 95%, at least 97%, or at least 99% identical to the amino acid sequence of SEQ ID NO: 218;
dd, the HVRs of embodiment 25.u, and further comprises a heavy chain that is at least 90%, at least 95%, at least 97%, or at least 99% identical to the amino acid sequence of SEQ ID NO: 219;
ee, the HVRs of embodiment 25.w, and further comprises a heavy chain that is at least 90%, at least 95%, at least 97%, or at least 99% identical to the amino acid sequence of SEQ ID NO: 220;
ff. the HVRs of embodiment 25.w, and further comprises a heavy chain that is at least 90%, at least 95%, at least 97%, or at least 99% identical to the amino acid sequence of SEQ ID NO: 221;
gg. the HVRs of embodiment 25.w, and further comprises a heavy chain that is at least 90%, at least 95%, at least 97%, or at least 99% identical to the amino acid sequence of SEQ ID NO: 222;
hh. the HVRs of embodiment 25.w, and further comprises a heavy chain that is at least 90%, at least 95%, at least 97%, or at least 99% identical to the amino acid sequence of SEQ ID NO: 223;
ii. the HVRs of embodiment 25.w, and further comprises a heavy chain that is at least 90%, at least 95%, at least 97%, or at least 99% identical to the amino acid sequence of SEQ ID NO: 224;
jj. the HVRs of embodiment 25.w, and further comprises a heavy chain that is at least 90%, at least 95%, at least 97%, or at least 99% identical to the amino acid sequence of SEQ ID NO: 225;
kk. the HVRs of embodiment 25.w, and further comprises a heavy chain that is at least 90%, at least 95%, at least 97%, or at least 99% identical to the amino acid sequence of SEQ ID NO: 226;
ll. the HVRs of embodiment 25.w, and further comprises a heavy chain that is at least 90%, at least 95%, at least 97%, or at least 99% identical to the amino acid sequence of SEQ ID NO: 227;
mm. the HVRs of embodiment 25.w, and further comprises a heavy chain that is at least 90%, at least 95%, at least 97%, or at least 99% identical to the amino acid sequence of SEQ ID NO: 228;
nn. the HVRs of embodiment 25.w, and further comprises a heavy chain that is at least 90%, at least 95%, at least 97%, or at least 99% identical to the amino acid sequence of SEQ ID
oo. the HVRs of embodiment 25.w, and further comprises a heavy chain that is at least 90%, at least 95%, at least 97%, or at least 99% identical to the amino acid sequence of SEQ ID NO: 230;
pp. the HVRs of embodiment 25.w, and further comprises a heavy chain that is at least 90%, at least 95%, at least 97%, or at least 99% identical to the amino acid sequence of SEQ ID NO: 231;
qq. the HVRs of embodiment 25.w, and further comprises a heavy chain that is at least 90%, at least 95%, at least 97%, or at least 99% identical to the amino acid sequence of SEQ ID NO: 232;
rr. the HVRs of embodiment 25.w, and further comprises a heavy chain that is at least 90%, at least 95%, at least 97%, or at least 99% identical to the amino acid sequence of SEQ ID NO: 233; or
ss. the HVRs of embodiment 25.w, and further comprises a heavy chain that is at least 90%, at least 95%, at least 97%, or at least 99% identical to the amino acid sequence of SEQ ID NO: 234.

Embodiment 32 is the antibody of embodiment 25 or embodiment 31, wherein the antibody comprises:
a. the HVRs of embodiment 25.s, and further comprises a light chain that is at least 90%, at least 95%, at least 97%, or at least 99% identical to the amino acid sequence of SEQ ID NO: 189;
b. the HVRs of embodiment 25.u, and further comprises a light chain that is at least 90%, at least 95%, at least 97%, or at least 99% identical to the amino acid sequence of SEQ ID NO: 189; or
c. the HVRs of embodiment 25.w, and further comprises a light chain that is at least 90%, at least 95%, at least 97%, or at least 99% identical to the amino acid sequence of SEQ ID NO: 189.

Embodiment 33 is the antibody of embodiment 25, wherein the antibody comprises:
a. a heavy chain that is at least 90%, at least 95%, at least 97%, or at least 99% identical to the amino acid sequence of SEQ ID NO: 190 and a light chain that is at least 90%, at least 95%, at least 97%, or at least 99% identical to the amino acid sequence of SEQ ID NO: 189;
b. a heavy chain that is at least 90%, at least 95%, at least 97%, or at least 99% identical to the amino acid sequence of SEQ ID NO: 191 and a light chain that is at least 90%, at least 95%, at least 97%, or at least 99% identical to the amino acid sequence of SEQ ID NO: 189;
c. a heavy chain that is at least 90%, at least 95%, at least 97%, or at least 99% identical to the amino acid sequence of SEQ ID NO: 192 and a light chain that is at least 90%, at least 95%, at least 97%, or at least 99% identical to the amino acid sequence of SEQ ID NO: 189;
d. a heavy chain that is at least 90%, at least 95%, at least 97%, or at least 99% identical to the amino acid sequence of SEQ ID NO: 193 and a light chain that is at least 90%, at least 95%, at least 97%, or at least 99% identical to the amino acid sequence of SEQ ID NO:189;
e. a heavy chain that is at least 90%, at least 95%, at least 97%, or at least 99% identical to the amino acid sequence of SEQ ID NO: 194 and a light chain that is at least 90%, at least 95%, at least 97%, or at least 99% identical to the amino acid sequence of SEQ ID NO: 189;
f. a heavy chain that is at least 90%, at least 95%, at least 97%, or at least 99% identical to the amino acid sequence of SEQ ID NO: 195 and a light chain that is at least 90%, at least 95%, at least 97%, or at least 99% identical to the amino acid sequence of SEQ ID NO: 189;
g. a heavy chain that is at least 90%, at least 95%, at least 97%, or at least 99% identical to the amino acid sequence of SEQ ID NO: 196 and a light chain that is at least 90%, at least 95%, at least 97%, or at least 99% identical to the amino acid sequence of SEQ ID NO: 189;
h. a heavy chain that is at least 90%, at least 95%, at least 97%, or at least 99% identical to the amino acid sequence of SEQ ID NO: 197 and a light chain that is at least 90%, at least 95%, at least 97%, or at least 99% identical to the amino acid sequence of SEQ ID NO: 189;
i. a heavy chain that is at least 90%, at least 95%, at least 97%, or at least 99% identical to the amino acid sequence of SEQ ID NO: 198 and a light chain that is at least 90%, at least 95%, at least 97%, or at least 99% identical to the amino acid sequence of SEQ ID NO:189;
j. a heavy chain that is at least 90%, at least 95%, at least 97%, or at least 99% identical to the amino acid sequence of SEQ ID NO: 199 and a light chain that is at least 90%, at least 95%, at least 97%, or at least 99% identical to the amino acid sequence of SEQ ID NO: 189;
k. a heavy chain that is at least 90%, at least 95%, at least 97%, or at least 99% identical to the amino acid sequence of SEQ ID NO:200 and a light chain that is at least 90%, at least 95%, at least 97%, or at least 99% identical to the amino acid sequence of SEQ ID NO: 189;
l. a heavy chain that is at least 90%, at least 95%, at least 97%, or at least 99% identical to the amino acid sequence of SEQ ID NO:201 and a light chain that is at least 90%, at least 95%, at least 97%, or at least 99% identical to the amino acid sequence of SEQ ID NO: 189;
m. a heavy chain that is at least 90%, at least 95%, at least 97%, or at least 99% identical to the amino acid sequence of SEQ ID NO:202 and a light chain that is at least 90%, at least 95%, at least 97%, or at least 99% identical to the amino acid sequence of SEQ ID NO: 189;
n. a heavy chain that is at least 90%, at least 95%, at least 97%, or at least 99% identical to the amino acid sequence of SEQ ID NO:203 and a light chain that is at least 90%, at least 95%, at least 97%, or at least 99% identical to the amino acid sequence of SEQ ID NO: 189;
o. a heavy chain that is at least 90%, at least 95%, at least 97%, or at least 99% identical to the amino acid sequence of SEQ ID NO:204 and a light chain that is at least 90%, at least 95%, at least 97%, or at least 99% identical to the amino acid sequence of SEQ ID NO: 189;
p. a heavy chain that is at least 90%, at least 95%, at least 97%, or at least 99% identical to the amino acid sequence of SEQ ID NO:205 and a light chain that is at least 90%, at least 95%, at least 97%, or at least 99% identical to the amino acid sequence of SEQ ID NO: 189;
q. a heavy chain that is at least 90%, at least 95%, at least 97%, or at least 99% identical to the amino acid sequence of SEQ ID NO:206 and a light chain that is at least 90%, at least 95%, at least 97%, or at least 99% identical to the amino acid sequence of SEQ ID NO: 189;
r. a heavy chain that is at least 90%, at least 95%, at least 97%, or at least 99% identical to the amino acid sequence of SEQ ID NO:207 and a light chain that is at least 90%, at least 95%, at least 97%, or at least 99% identical to the amino acid sequence of SEQ ID NO: 189;
s. a heavy chain that is at least 90%, at least 95%, at least 97%, or at least 99% identical to the amino acid sequence of SEQ ID NO:208 and a light chain that is at least 90%, at least 95%, at least 97%, or at least 99% identical to the amino acid sequence of SEQ ID NO: 189;
t. a heavy chain that is at least 90%, at least 95%, at least 97%, or at least 99% identical to the amino acid sequence of SEQ ID NO:209 and a light chain that is at least 90%, at least 95%, at least 97%, or at least 99% identical to the amino acid sequence of SEQ ID NO: 189;
u. a heavy chain that is at least 90%, at least 95%, at least 97%, or at least 99% identical to the amino acid sequence of SEQ ID NO:210 and a light chain that is at least 90%, at least 95%, at least 97%, or at least 99% identical to the amino acid sequence of SEQ ID NO: 189;
v. a heavy chain that is at least 90%, at least 95%, at least 97%, or at least 99% identical to the amino acid sequence of SEQ ID NO:211 and a light chain that is at least 90%, at least 95%, at least 97%, or at least 99% identical to the amino acid sequence of SEQ ID NO: 189;
w. a heavy chain that is at least 90%, at least 95%, at least 97%, or at least 99% identical to the amino acid sequence of SEQ ID NO:212 and a light chain that is at least 90%, at least 95%, at least 97%, or at least 99% identical to the amino acid sequence of SEQ ID NO: 189;
x. a heavy chain that is at least 90%, at least 95%, at least 97%, or at least 99% identical to the amino acid sequence of SEQ ID NO:213 and a light chain that is at least 90%, at least 95%, at least 97%, or at least 99% identical to the amino acid sequence of SEQ ID NO: 189;
y. a heavy chain that is at least 90%, at least 95%, at least 97%, or at least 99% identical to the amino acid sequence of SEQ ID NO:214 and a light chain that is at least 90%, at least 95%, at least 97%, or at least 99% identical to the amino acid sequence of SEQ ID NO: 189;
z. a heavy chain that is at least 90%, at least 95%, at least 97%, or at least 99% identical to the amino acid sequence of SEQ ID NO:215 and a light chain that is at least 90%, at least 95%, at least 97%, or at least 99% identical to the amino acid sequence of SEQ ID NO: 189;
aa. a heavy chain that is at least 90%, at least 95%, at least 97%, or at least 99% identical to the amino acid sequence of SEQ ID NO:216 and a light chain that is at least 90%, at least 95%, at least 97%, or at least 99% identical to the amino acid sequence of SEQ ID NO: 189;
bb. a heavy chain that is at least 90%, at least 95%, at least 97%, or at least 99% identical to the amino acid sequence of SEQ ID NO:217 and a light chain that is at least 90%, at least 95%, at least 97%, or at least 99% identical to the amino acid sequence of SEQ ID NO: 189;
cc. a heavy chain that is at least 90%, at least 95%, at least 97%, or at least 99% identical to the amino acid sequence of SEQ ID NO:218 and a light chain that is at least 90%, at least 95%, at least 97%, or at least 99% identical to the amino acid sequence of SEQ ID NO: 189;
dd. a heavy chain that is at least 90%, at least 95%, at least 97%, or at least 99% identical to the amino acid sequence of SEQ ID NO:219 and a light chain that is at least 90%, at least 95%, at least 97%, or at least 99% identical to the amino acid sequence of SEQ ID NO: 189;
ee. a heavy chain that is at least 90%, at least 95%, at least 97%, or at least 99% identical to the amino acid sequence of SEQ ID NO:220 and a light chain that is at least 90%, at least 95%, at least 97%, or at least 99% identical to the amino acid sequence of SEQ ID NO:189;
ff. a heavy chain that is at least 90%, at least 95%, at least 97%, or at least 99% identical to the amino acid sequence of SEQ ID NO:221 and a light chain that is at least 90%, at least 95%, at least 97%, or at least 99% identical to the amino acid sequence of SEQ ID NO: 189;
gg. a heavy chain that is at least 90%, at least 95%, at least 97%, or at least 99% identical to the amino acid sequence of SEQ ID NO:222 and a light chain that is at least 90%, at least 95%, at least 97%, or at least 99% identical to the amino acid sequence of SEQ ID NO: 189;
hh. a heavy chain that is at least 90%, at least 95%, at least 97%, or at least 99% identical to the amino acid sequence of SEQ ID NO:223 and a light chain that is at least 90%, at least 95%, at least 97%, or at least 99% identical to the amino acid sequence of SEQ ID NO: 189;
ii. a heavy chain that is at least 90%, at least 95%, at least 97%, or at least 99% identical to the amino acid sequence of SEQ ID NO:224 and a light chain that is at least 90%, at least 95%, at least 97%, or at least 99% identical to the amino acid sequence of SEQ ID NO: 189;
jj. a heavy chain that is at least 90%, at least 95%, at least 97%, or at least 99% identical to the amino acid sequence of SEQ ID NO:225 and a light chain that is at least 90%, at least 95%, at least 97%, or at least 99% identical to the amino acid sequence of SEQ ID NO: 189;
kk. a heavy chain that is at least 90%, at least 95%, at least 97%, or at least 99% identical to the amino acid sequence of SEQ ID NO:226 and a light chain that is at least 90%, at least 95%, at least 97%, or at least 99% identical to the amino acid sequence of SEQ ID NO: 189;
ll. a heavy chain that is at least 90%, at least 95%, at least 97%, or at least 99% identical to the amino acid sequence of SEQ ID NO:227 and a light chain that is at least 90%, at least 95%, at least 97%, or at least 99% identical to the amino acid sequence of SEQ ID NO: 189;
mm. a heavy chain that is at least 90%, at least 95%, at least 97%, or at least 99% identical to the amino acid sequence of SEQ ID NO:228 and a light chain that is at least 90%, at least 95%, at least 97%, or at least 99% identical to the amino acid sequence of SEQ ID NO: 189;
nn. a heavy chain that is at least 90%, at least 95%, at least 97%, or at least 99% identical to the amino acid sequence of SEQ ID NO:229 and a light chain that is at least 90%, at least 95%, at least 97%, or at least 99% identical to the amino acid sequence of SEQ ID NO:189;
oo. a heavy chain that is at least 90%, at least 95%, at least 97%, or at least 99% identical to the amino acid sequence of SEQ ID NO:230 and a light chain that is at least 90%, at least 95%, at least 97%, or at least 99% identical to the amino acid sequence of SEQ ID NO: 189;
pp. a heavy chain that is at least 90%, at least 95%, at least 97%, or at least 99% identical to the amino acid sequence of SEQ ID NO:231 and a light chain that is at least 90%, at least 95%, at least 97%, or at least 99% identical to the amino acid sequence of SEQ ID NO: 189;
qq. a heavy chain that is at least 90%, at least 95%, at least 97%, or at least 99% identical to the amino acid sequence of SEQ ID NO:232 and a light chain that is at least 90%, at least 95%, at least 97%, or at least 99% identical to the amino acid sequence of SEQ ID NO: 189;
rr. a heavy chain that is at least 90%, at least 95%, at least 97%, or at least 99% identical to the amino acid sequence of SEQ ID NO:233 and a light chain that is at least 90%, at least 95%, at least 97%, or at least 99% identical to the amino acid sequence of SEQ ID NO: 189; or
ss. a heavy chain that is at least 90%, at least 95%, at least 97%, or at least 99% identical to the amino acid sequence of SEQ ID NO:234 and a light chain that is at least 90%, at least 95%, at least 97%, or at least 99% identical to the amino acid sequence of SEQ ID NO: 189.

Embodiment 34 is an isolated anti-IL18BP antibody, wherein the anti-IL18BP antibody comprises:
a. a heavy chain comprising the amino acid sequence of SEQ ID NO: 190 and a light chain comprising the amino acid sequence of SEQ ID NO: 189;
b. a heavy chain comprising the amino acid sequence of SEQ ID NO: 191 and a light chain comprising the amino acid sequence of SEQ ID NO:189;
c. a heavy chain comprising the amino acid sequence of SEQ ID NO: 192 and a light chain comprising the amino acid sequence of SEQ ID NO: 189;
d. a heavy chain comprising the amino acid sequence of SEQ ID NO: 193 and a light chain comprising the amino acid sequence of SEQ ID NO: 189;
e. a heavy chain comprising the amino acid sequence of SEQ ID NO: 194 and a light chain comprising the amino acid sequence of SEQ ID NO: 189;
f. a heavy chain comprising the amino acid sequence of SEQ ID NO: 195 and a light chain comprising the amino acid sequence of SEQ ID NO: 189;
g. a heavy chain comprising the amino acid sequence of SEQ ID NO: 196 and a light chain comprising the amino acid sequence of SEQ ID NO: 189;
h. a heavy chain comprising the amino acid sequence of SEQ ID NO: 197 and a light chain comprising the amino acid sequence of SEQ ID NO:189;
i. a heavy chain comprising the amino acid sequence of SEQ ID NO: 198 and a light chain comprising the amino acid sequence of SEQ ID NO: 189;
j. a heavy chain comprising the amino acid sequence of SEQ ID NO: 199 and a light chain comprising the amino acid sequence of SEQ ID NO:189;
k. a heavy chain comprising the amino acid sequence of SEQ ID NO:200 and a light chain comprising the amino acid sequence of SEQ ID NO: 189;
l. a heavy chain comprising the amino acid sequence of SEQ ID NO:201 and a light chain comprising the amino acid sequence of SEQ ID NO: 189;
m. a heavy chain comprising the amino acid sequence of SEQ ID NO:202 and a light chain comprising the amino acid sequence of SEQ ID NO: 189;
n. a heavy chain comprising the amino acid sequence of SEQ ID NO:203 and a light chain comprising the amino acid sequence of SEQ ID NO: 189;
o. a heavy chain comprising the amino acid sequence of SEQ ID NO:204 and a light chain comprising the amino acid sequence of SEQ ID NO: 189;
p. a heavy chain comprising the amino acid sequence of SEQ ID NO:205 and a light chain comprising the amino acid sequence of SEQ ID NO: 189;
q. a heavy chain comprising the amino acid sequence of SEQ ID NO:206 and a light chain comprising the amino acid sequence of SEQ ID NO: 189;
r. a heavy chain comprising the amino acid sequence of SEQ ID NO:207 and a light chain comprising the amino acid sequence of SEQ ID NO: 189;
s. a heavy chain comprising the amino acid sequence of SEQ ID NO:208 and a light chain comprising the amino acid sequence of SEQ ID NO: 189;
t. a heavy chain comprising the amino acid sequence of SEQ ID NO:209 and a light chain comprising the amino acid sequence of SEQ ID NO:189;
u. a heavy chain comprising the amino acid sequence of SEQ ID NO:210 and a light chain comprising the amino acid sequence of SEQ ID NO:189;
v. a heavy chain comprising the amino acid sequence of SEQ ID NO:211 and a light chain comprising the amino acid sequence of SEQ ID NO: 189;
w. a heavy chain comprising the amino acid sequence of SEQ ID NO:212 and a light chain comprising the amino acid sequence of SEQ ID NO:189;
x. a heavy chain comprising the amino acid sequence of SEQ ID NO:213 and a light chain comprising the amino acid sequence of SEQ ID NO: 189;
y. a heavy chain comprising the amino acid sequence of SEQ ID NO:214 and a light chain comprising the amino acid sequence of SEQ ID NO: 189;
z. a heavy chain comprising the amino acid sequence of SEQ ID NO:215 and a light chain comprising the amino acid sequence of SEQ ID NO: 189;
aa. a heavy chain comprising the amino acid sequence of SEQ ID NO:216 and a light chain comprising the amino acid sequence of SEQ ID NO: 189;
bb. a heavy chain comprising the amino acid sequence of SEQ ID NO:217 and a light chain comprising the amino acid sequence of SEQ ID NO:189;
cc. a heavy chain comprising the amino acid sequence of SEQ ID NO:218 and a light chain comprising the amino acid sequence of SEQ ID NO: 189;
dd. a heavy chain comprising the amino acid sequence of SEQ ID NO:219 and a light chain comprising the amino acid sequence of SEQ ID NO: 189;
ee. a heavy chain comprising the amino acid sequence of SEQ ID NO:220 and a light chain comprising the amino acid sequence of SEQ ID NO: 189;
ff. a heavy chain comprising the amino acid sequence of SEQ ID NO:221 and a light chain comprising the amino acid sequence of SEQ ID NO: 189;
gg. a heavy chain comprising the amino acid sequence of SEQ ID NO:222 and a light chain comprising the amino acid sequence of SEQ ID NO:189;
hh. a heavy chain comprising the amino acid sequence of SEQ ID NO:223 and a light chain comprising the amino acid sequence of SEQ ID NO: 189;
ii. a heavy chain comprising the amino acid sequence of SEQ ID NO:224 and a light chain comprising the amino acid sequence of SEQ ID NO: 189;
jj. a heavy chain comprising the amino acid sequence of SEQ ID NO:225 and a light chain comprising the amino acid sequence of SEQ ID NO: 189;
kk. a heavy chain comprising the amino acid sequence of SEQ ID NO:226 and a light chain comprising the amino acid sequence of SEQ ID NO: 189;
ll. a heavy chain comprising the amino acid sequence of SEQ ID NO:227 and a light chain comprising the amino acid sequence of SEQ ID NO:189;
mm. a heavy chain comprising the amino acid sequence of SEQ ID NO:228 and a light chain comprising the amino acid sequence of SEQ ID NO: 189;
nn. a heavy chain comprising the amino acid sequence of SEQ ID NO:229 and a light chain comprising the amino acid sequence of SEQ ID NO: 189;
oo. a heavy chain comprising the amino acid sequence of SEQ ID NO:230 and a light chain comprising the amino acid sequence of SEQ ID NO: 189;
pp. a heavy chain comprising the amino acid sequence of SEQ ID NO:321 and a light chain comprising the amino acid sequence of SEQ ID NO: 189;
qq. a heavy chain comprising the amino acid sequence of SEQ ID NO:232 and a light chain comprising the amino acid sequence of SEQ ID NO: 189;
rr. a heavy chain comprising the amino acid sequence of SEQ ID NO:233 and a light chain comprising the amino acid sequence of SEQ ID NO: 189; or
ss. a heavy chain comprising the amino acid sequence of SEQ ID NO:234 and a light chain comprising the amino acid sequence of SEQ ID NO: 189.

Embodiment 35 is an isolated anti-IL18BP antibody, wherein the anti-IL18BP antibody comprises a VH comprising HVR-H1, HVR-H2, and HVR-H3 and a VL comprising HVR-L1, HVR-L2, and HVR-L3 of any one of antibodies BP-01, BP-02, BP-03, BP-04, BP-05, BP-06, BP-07, BP-08, BP-09, BP-10, BP-11, BP-12, BP-13, BP-14, BP-15, BP-16, BP-04.01, BP-04.02, BP-04.03, BP-04.04, BP-04.05, BP-04.06, BP-04.07, BP-04.08, BP-04.09, BP-04.10, BP-04.11, BP-04.12, BP-04.13, BP-04.14, BP-04.15, BP-04.16, BP-04.17, BP-04.18, BP-04.19, BP-04.20, BP-04.21, BP-04.22, BP-04.23, BP-04.24, BP-04.25, BP-04.26, BP-04.27, BP-04.28, BP-04.29, BP-04.30, BP-04.31, and BP-04.32.

Embodiment 36 is the isolated antibody of embodiment 35, wherein the antibody comprises a VH and/or a VL at least 90%, at least 95%, at least 97%, or at least 99% identical to those of any one of antibodies BP-01, BP-02, BP-03, BP-04, BP-05, BP-06, BP-07, BP-08, BP-09, BP-10, BP-11, BP-12, BP-13, BP-14, BP-15, BP-16, BP-04.01, BP-04.02, BP-04.03, BP-04.04, BP-04.05, BP-04.06, BP-04.07, BP-04.08, BP-04.09, BP-04.10, BP-04.11, BP-04.12, BP-04.13, BP-04.14, BP-04.15, BP-04.16, BP-04.17, BP-04.18, BP-04.19, BP-04.20, BP-04.21, BP-04.22, BP-04.23, BP-04.24, BP-04.25, BP-04.26, BP-04.27, BP-04.28, BP-04.29, BP-04.30, BP-04.31, and BP-04.32.

Embodiment 37 is the isolated antibody of embodiment 35 or embodiment 36, wherein the antibody comprises the VH and/or the VL of any one of antibodies BP-01, BP-02, BP-03, BP-04, BP-05, BP-06, BP-07, BP-08, BP-09, BP-10, BP-11, BP-12, BP-13, BP-14, BP-15, BP-16, BP-04.01, BP-04.02, BP-04.03, BP-04.04, BP-04.05, BP-04.06, BP-04.07, BP-04.08, BP-04.09, BP-04.10, BP-04.11, BP-04.12, BP-04.13, BP-04.14, BP-04.15, BP-04.16, BP-04.17, BP-04.18, BP-04.19, BP-04.20, BP-04.21, BP-04.22, BP-04.23, BP-04.24, BP-04.25, BP-04.26, BP-04.27, BP-04.28, BP-04.29, BP-04.30, BP-04.31, and BP-04.32.

Embodiment 38 is an isolated anti-IL18BP antibody, wherein the anti-IL18BP antibody comprises:
a. a VH and VL, wherein the VH comprises the HVR-H1, HVR-H2, and HVR-H3 of the antibody BP-01 (as shown in Table 1) and the VL comprises the HVR-L1, HVR-L2, and HVR-L3 of the antibody BP-01 (as shown in Table 2);
b. VH and VL, wherein the VH comprises the HVR-H1, HVR-H2, and HVR-H3 of the antibody BP-02 (as shown in Table 1) and the VL comprises the HVR-L1, HVR-L2, and HVR-L3 of the antibody BP-02 (as shown in Table 2);
c. VH and VL, wherein the VH comprises the HVR-H1, HVR-H2, and HVR-H3 of the antibody BP-03 (as shown in Table 1) and the VL comprises the HVR-L1, HVR-L2, and HVR-L3 of the antibody BP-03 (as shown in Table 2);
d. VH and VL, wherein the VH comprises the HVR-H1, HVR-H2, and HVR-H3 of the antibody BP-04 (as shown in Table 1) and the VL comprises the HVR-L1, HVR-L2, and HVR-L3 of the antibody BP-04 (as shown in Table 2);
e. VH and VL, wherein the VH comprises the HVR-H1, HVR-H2, and HVR-H3 of the antibody BP-05 (as shown in Table 1) and the VL comprises the HVR-L1, HVR-L2, and HVR-L3 of the antibody BP-05 (as shown in Table 2);
f. VH and VL, wherein the VH comprises the HVR-H1, HVR-H2, and HVR-H3 of the antibody BP-06 (as shown in Table 1) and the VL comprises the HVR-L1, HVR-L2, and HVR-L3 of the antibody BP-06 (as shown in Table 2);
g. VH and VL, wherein the VH comprises the HVR-H1, HVR-H2, and HVR-H3 of the antibody BP-07 (as shown in Table 1) and the VL comprises the HVR-L1, HVR-L2, and HVR-L3 of the antibody BP-07 (as shown in Table 2);
h. VH and VL, wherein the VH comprises the HVR-H1, HVR-H2, and HVR-H3 of the antibody BP-08 (as shown in Table 1) and the VL comprises the HVR-L1, HVR-L2, and HVR-L3 of the antibody BP-08 (as shown in Table 2);
i. VH and VL, wherein the VH comprises the HVR-H1, HVR-H2, and HVR-H3 of the antibody BP-09 (as shown in Table 1) and the VL comprises the HVR-L1, HVR-L2, and HVR-L3 of the antibody BP-09 (as shown in Table 2);
j. VH and VL, wherein the VH comprises the HVR-H1, HVR-H2, and HVR-H3 of the antibody BP-10 (as shown in Table 1) and the VL comprises the HVR-L1, HVR-L2, and HVR-L3 of the antibody BP-10 (as shown in Table 2);
k. VH and VL, wherein the VH comprises the HVR-H1, HVR-H2, and HVR-H3 of the antibody BP-11 (as shown in Table 1) and the VL comprises the HVR-L1, HVR-L2, and HVR-L3 of the antibody BP-11 (as shown in Table 2);
l. VH and VL, wherein the VH comprises the HVR-H1, HVR-H2, and HVR-H3 of the antibody BP-12 (as shown in Table 1) and the VL comprises the HVR-L1, HVR-L2, and HVR-L3 of the antibody BP-12 (as shown in Table 2);
m. VH and VL, wherein the VH comprises the HVR-H1, HVR-H2, and HVR-H3 of the antibody BP-13 (as shown in Table 1) and the VL comprises the HVR-L1, HVR-L2, and HVR-L3 of the antibody BP-13 (as shown in Table 2);
n. VH and VL, wherein the VH comprises the HVR-H1, HVR-H2, and HVR-H3 of the antibody BP-14 (as shown in Table 1) and the VL comprises the HVR-L1, HVR-L2, and HVR-L3 of the antibody BP-14 (as shown in Table 2);
o. VH and VL, wherein the VH comprises the HVR-H1, HVR-H2, and HVR-H3 of the antibody BP-15 (as shown in Table 1) and the VL comprises the HVR-L1, HVR-L2, and HVR-L3 of the antibody BP-15 (as shown in Table 2);
p. VH and VL, wherein the VH comprises the HVR-H1, HVR-H2, and HVR-H3 of the antibody BP-16 (as shown in Table 1) and the VL comprises the HVR-L1, HVR-L2, and HVR-L3 of the antibody BP-16 (as shown in Table 2)
q. VH and VL, wherein the VH comprises the HVR-H1, HVR-H2, and HVR-H3 of the antibody BP-04.01 (as shown in Table 12) and the VL comprises the HVR-L1, HVR-L2, and HVR-L3 of the antibody BP-04.LC (as shown in Table 13);
r. VH and VL, wherein the VH comprises the HVR-H1, HVR-H2, and HVR-H3 of the antibody BP-04.02 (as shown in Table 12) and the VL comprises the HVR-L1, HVR-L2, and HVR-L3 of the antibody BP-04.LC (as shown in Table 13);
s. VH and VL, wherein the VH comprises the HVR-H1, HVR-H2, and HVR-H3 of the antibody BP-04.03 (as shown in Table 12) and the VL comprises the HVR-L1, HVR-L2, and HVR-L3 of the antibody BP-04.LC (as shown in Table 13);
t. VH and VL, wherein the VH comprises the HVR-H1, HVR-H2, and HVR-H3 of the antibody BP-04.04 (as shown in Table 12) and the VL comprises the HVR-L1, HVR-L2, and HVR-L3 of the antibody BP-04.LC (as shown in Table 13);
u. VH and VL, wherein the VH comprises the HVR-H1, HVR-H2, and HVR-H3 of the antibody BP-04.05 (as shown in Table 12) and the VL comprises the HVR-L1, HVR-L2, and HVR-L3 of the antibody BP-04.LC (as shown in Table 13);
v. VH and VL, wherein the VH comprises the HVR-H1, HVR-H2, and HVR-H3 of the antibody BP-04.06 (as shown in Table 12) and the VL comprises the HVR-L1, HVR-L2, and HVR-L3 of the antibody BP-04.LC (as shown in Table 13);
w. VH and VL, wherein the VH comprises the HVR-H1, HVR-H2, and HVR-H3 of the antibody BP-04.07 (as shown in Table 12) and the VL comprises the HVR-L1, HVR-L2, and HVR-L3 of the antibody BP-04.LC (as shown in Table 13);
x. VH and VL, wherein the VH comprises the HVR-H1, HVR-H2, and HVR-H3 of the antibody BP-04.08 (as shown in Table 12) and the VL comprises the HVR-L1, HVR-L2, and HVR-L3 of the antibody BP-04.LC (as shown in Table 13);
y. VH and VL, wherein the VH comprises the HVR-H1, HVR-H2, and HVR-H3 of the antibody BP-04.09 (as shown in Table 12) and the VL comprises the HVR-L1, HVR-L2, and HVR-L3 of the antibody BP-04.LC (as shown in Table 13);
z. VH and VL, wherein the VH comprises the HVR-H1, HVR-H2, and HVR-H3 of the antibody BP-04.10 (as shown in Table 12) and the VL comprises the HVR-L1, HVR-L2, and HVR-L3 of the antibody BP-04.LC (as shown in Table 13);
aa. VH and VL, wherein the VH comprises the HVR-H1, HVR-H2, and HVR-H3 of the antibody BP-04.11 (as shown in Table 12) and the VL comprises the HVR-L1, HVR-L2, and HVR-L3 of the antibody BP-04.LC (as shown in Table 13);
bb. VH and VL, wherein the VH comprises the HVR-H1, HVR-H2, and HVR-H3 of the antibody BP-04.12 (as shown in Table 12) and the VL comprises the HVR-L1, HVR-L2, and HVR-L3 of the antibody BP-04.LC (as shown in Table 13);
cc. VH and VL, wherein the VH comprises the HVR-H1, HVR-H2, and HVR-H3 of the antibody BP-04.13 (as shown in Table 12) and the VL comprises the HVR-L1, HVR-L2, and HVR-L3 of the antibody BP-04.LC (as shown in Table 13);
dd. VH and VL, wherein the VH comprises the HVR-H1, HVR-H2, and HVR-H3 of the antibody BP-04.14 (as shown in Table 12) and the VL comprises the HVR-L1, HVR-L2, and HVR-L3 of the antibody BP-04.LC (as shown in Table 13);
ee. VH and VL, wherein the VH comprises the HVR-H1, HVR-H2, and HVR-H3 of the antibody BP-04.15 (as shown in Table 12) and the VL comprises the HVR-L1, HVR-L2, and HVR-L3 of the antibody BP-04.LC (as shown in Table 13);
ff. VH and VL, wherein the VH comprises the HVR-H1, HVR-H2, and HVR-H3 of the antibody BP-04.16 (as shown in Table 12) and the VL comprises the HVR-L1, HVR-L2, and HVR-L3 of the antibody BP-04.LC (as shown in Table 13);
gg. VH and VL, wherein the VH comprises the HVR-H1, HVR-H2, and HVR-H3 of the antibody BP-04.17 (as shown in Table 12) and the VL comprises the HVR-L1, HVR-L2, and HVR-L3 of the antibody BP-04.LC (as shown in Table 13);
hh. VH and VL, wherein the VH comprises the HVR-H1, HVR-H2, and HVR-H3 of the antibody BP-04.18 (as shown in Table 12) and the VL comprises the HVR-L1, HVR-L2, and HVR-L3 of the antibody BP-04.LC (as shown in Table 13);
ii. VH and VL, wherein the VH comprises the HVR-H1, HVR-H2, and HVR-H3 of the antibody BP-04.19 (as shown in Table 12) and the VL comprises the HVR-L1, HVR-L2, and HVR-L3 of the antibody BP-04.LC (as shown in Table 13);
jj. VH and VL, wherein the VH comprises the HVR-H1, HVR-H2, and HVR-H3 of the antibody BP-04.20 (as shown in Table 12) and the VL comprises the HVR-L1, HVR-L2, and HVR-L3 of the antibody BP-04.LC (as shown in Table 13);
kk. VH and VL, wherein the VH comprises the HVR-H1, HVR-H2, and HVR-H3 of the antibody BP-04.21 (as shown in Table 12) and the VL comprises the HVR-L1, HVR-L2, and HVR-L3 of the antibody BP-04.LC (as shown in Table 13);
ll. VH and VL, wherein the VH comprises the HVR-H1, HVR-H2, and HVR-H3 of the antibody BP-04.22 (as shown in Table 12) and the VL comprises the HVR-L1, HVR-L2, and HVR-L3 of the antibody BP-04.LC (as shown in Table 13);
mm. VH and VL, wherein the VH comprises the HVR-H1, HVR-H2, and HVR-H3 of the antibody BP-04.23 (as shown in Table 12) and the VL comprises the HVR-L1, HVR-L2, and HVR-L3 of the antibody BP-04.LC (as shown in Table 13);
nn. VH and VL, wherein the VH comprises the HVR-H1, HVR-H2, and HVR-H3 of the antibody BP-04.24 (as shown in Table 12) and the VL comprises the HVR-L1, HVR-L2, and HVR-L3 of the antibody BP-04.LC (as shown in Table 13);
oo. VH and VL, wherein the VH comprises the HVR-H1, HVR-H2, and HVR-H3 of the antibody BP-04.25 (as shown in Table 12) and the VL comprises the HVR-L1, HVR-L2, and HVR-L3 of the antibody BP-04.LC (as shown in Table 13);
pp. VH and VL, wherein the VH comprises the HVR-H1, HVR-H2, and HVR-H3 of the antibody BP-04.26 (as shown in Table 12) and the VL comprises the HVR-L1, HVR-L2, and HVR-L3 of the antibody BP-04.LC (as shown in Table 13);
qq. VH and VL, wherein the VH comprises the HVR-H1, HVR-H2, and HVR-H3 of the antibody BP-04.27 (as shown in Table 12) and the VL comprises the HVR-L1, HVR-L2, and HVR-L3 of the antibody BP-04.LC (as shown in Table 13);
rr. VH and VL, wherein the VH comprises the HVR-H1, HVR-H2, and HVR-H3 of the antibody BP-04.28 (as shown in Table 12) and the VL comprises the HVR-L1, HVR-L2, and HVR-L3 of the antibody BP-04.LC (as shown in Table 13);
ss. VH and VL, wherein the VH comprises the HVR-H1, HVR-H2, and HVR-H3 of the antibody BP-04.29 (as shown in Table 12) and the VL comprises the HVR-L1, HVR-L2, and HVR-L3 of the antibody BP-04.LC (as shown in Table 13);
tt. VH and VL, wherein the VH comprises the HVR-H1, HVR-H2, and HVR-H3 of the antibody BP-04.30 (as shown in Table 12) and the VL comprises the HVR-L1, HVR-L2, and HVR-L3 of the antibody BP-04.LC (as shown in Table 13);
uu. VH and VL, wherein the VH comprises the HVR-H1, HVR-H2, and HVR-H3 of the antibody BP-04.31 (as shown in Table 12) and the VL comprises the HVR-L1, HVR-L2, and HVR-L3 of the antibody BP-04.LC (as shown in Table 13); or
vv. VH and VL, wherein the VH comprises the HVR-H1, HVR-H2, and HVR-H3 of the antibody BP-04.32 (as shown in Table 12) and the VL comprises the HVR-L1, HVR-L2, and HVR-L3 of the antibody BP-04.LC (as shown in Table 13).

Embodiment 39 is the antibody of any one of embodiments 25-38, wherein the anti-IL18BP antibody blocks binding of IL-18 to IL18BP.

Embodiment 40 is the antibody of any one of embodiments 25-39, wherein the antibody has one or more of the following properties:
a. the anti-IL18BP antibody increases expression levels of interferon-gamma (IFNγ) in peripheral blood mononuclear cells;
b. the anti-IL18BP antibody increases expression of IFNγ in natural killer cells;
c. the anti-IL18BP antibody increases levels of inflammatory cytokines in serum;
d. the anti-IL18BP antibody increases IFNγ levels in serum;
e. the anti-IL18BP antibody increases IL-10 levels in serum;
f. the anti-IL18BP antibody increases IL-6 levels in serum;
g. the anti-IL18BP antibody increases tumor necrosis factor alpha (TNFα) levels in serum;
h. the anti-IL18BP antibody decreases the percentage of CD3+ T cells in vivo;
i. the anti-IL18BP antibody decreases the percentage of CD19+ B cells in vivo;
j. the anti-IL18BP antibody decreases the percentage of CD11b+ cells in vivo; and
k. the anti-IL18BP antibody increases the percentage of macrophages in vivo.
l. the anti-IL18BP antibody increases free IL-18 serum levels;
m. the anti-IL18BP antibody decreases IL-18BP serum levels;
n. the anti-IL18BP antibody increases IFNγ levels in tumor lysates;
o. the anti-IL18BP antibody increases IL-Iβ levels in tumor lysates;
p. the anti-IL18BP antibody increases IL-15 levels in tumor lysates;
q. the anti-IL18BP antibody increases IL-27 levels in tumor lysates;
r. the anti-IL18BP antibody increases MIP-1α levels in tumor lysates; and
s. the anti-IL18BP antibody decreases tumor volume.

Embodiment 41 is the antibody of any one of embodiments 1-30 and 35-40, wherein the antibody is a monoclonal antibody.

Embodiment 42 is the antibody of any one of embodiments 1-41, wherein the antibody is a humanized antibody.

Embodiment 43 is the antibody of any one of embodiments 1-42, wherein the antibody is an antigen binding fragment, such as an Fab, Fab′, Fab′-SH, F(ab′)2, Fv, or scFv fragment.

Embodiment 44 is the antibody of any one of embodiments 1-43, wherein the antibody is a bispecific or multispecific antibody.

Embodiment 45 is the antibody of any one of embodiments 1-44, wherein the antibody is of the IgG class, the IgM class, or the IgA class.

Embodiment 46 is the antibody of embodiment 45, wherein the antibody is of the IgG class and is of a human IgG1, IgG2, IgG3, or IgG4 isotype or of a mouse IgG1 or IgG2 isotype.

Embodiment 47 is the antibody of any one of embodiments 1-46, wherein the antibody has a human or mouse IgG1 isotype and comprises one or more amino acid substitutions in the Fc region at an amino acid residue selected from the group consisting of N297A, D265A, D270A, L234A, L235A, G237A, P238D, L328E, E233D, G237D, H268D, P271G, A330R, C226S, C229S, E233P, L234V, L234F, L235E, P331S, P331G, S267E, L328F, A330L, M252Y, S254T, T256E, N297Q, P238S, P238A, A327Q, A327G, P329A, P329S, P329G, K322A, N325S, T394D, A330S, E430G, E430S, E430F, E430T, E345K, E345Q, E345R, E345Y, S440Y, S440W, and any combination thereof, wherein the numbering of the residues is according to EU numbering.

Embodiment 48 is the antibody of any one of embodiments 1-40 or 35-46, wherein the antibody has a human or mouse IgG2 isotype and comprises one or more amino acid substitutions in the Fc region at an amino acid residue selected from the group consisting of A330S, C127S, C214S, C219S, C220S, E345K, E345Q, E345R, E345Y, E430F, E430G, E430S, E430T, G237A, H268Q, L328F, M252Y, P331S, S254T, S267E, S440W, S440Y, T256E, V234A, V309L, and any combination thereof, wherein the numbering of the residues is according to EU numbering.

Embodiment 49 is the antibody of any one of embodiments 1-46, wherein the antibody has a human or mouse IgG4 isotype and comprises one or more amino acid substitutions in the Fc region at an amino acid residue selected from the group consisting of C127S, E318A, E345R, E430G, F234A, G237A, K322A, L235A, L235E, L236E, L243A, L328F, M252Y, P331S, S228P, S229P, S254T, S267E, S440Y, T256E, and any combination thereof, wherein the numbering of the residues is according to EU numbering.

Embodiment 50 is the antibody of any one of embodiments 1-49, wherein the antibody comprises one or more amino acid substitutions in the Fc region at a residue position selected from the group consisting of A330L, A330S, C127S, E345R, E430G, K322A, L234A, L234F, L235A, L235E, L243A, L328F, P331S, P331G, S267E, S440Y, and any combination thereof, wherein the numbering of the amino acid residues is according to EU or Kabat numbering.

Embodiment 51 is the antibody of any one of embodiments 1-50, where in the IgG Fc amino acid sequences is selected from the group consisting of SEQ ID NOs: 8-27 and 235-246.

Embodiment 52 is a pharmaceutical composition comprising the anti-IL18BP antibody of any one of embodiments 1-51 and a pharmaceutically acceptable carrier.

Embodiment 53 is an isolated nucleic acid comprising a nucleic acid sequence encoding the anti-IL18BP antibody of any one of embodiments 1-51.

Embodiment 54 is an isolated vector comprising the nucleic acid of embodiment 53.

Embodiment 55 is an isolated host cell comprising the nucleic acid of embodiment 52 or the vector of embodiment 54.

Embodiment 56 is a method of producing an anti-IL18BP antibody, comprising culturing the cell of embodiment 55 so that the antibody is produced.

Embodiment 57 is the method of embodiment 56, further comprising recovering the antibody produced by the cell.

Embodiment 58 is a method of treating a cancer in a subject in need thereof, the method comprising administering to an individual in need thereof a therapeutically effective amount of an anti-IL18BP antibody of any one of embodiments 1-51, thereby treating the cancer.

Embodiment 59 is the method of embodiment 58, further comprising administering one or more therapeutic agents.

Embodiment 60 is the method of embodiment 59, wherein at least one therapeutic agent is a checkpoint inhibitor.

Embodiment 61 is the method of embodiment 60, where the checkpoint inhibitor is selected from a PD1, PD-L1, and PD-L2 inhibitor.

Embodiment 62 is the method of embodiment 60, where in the checkpoint inhibitor is selected from an anti-PD-L1 antibody, an anti-PD-L2 antibody, and an anti-PD-1 antibody.

Embodiment 63 is the method of any one of embodiments 58-62, wherein the cancer is cancer is cholangiocarcinoma, diffuse large B cell lymphoma, glioblastoma multiform, head and neck squamous carcinoma, kidney renal clear cell carcinoma, sarcoma, bladder cancer, breast cancer, colon cancer, endometrial cancer, kidney cancer, renal cancer, pancreatic adenocarcinoma, skin cutaneous melanoma, stomach adenocarcinoma, lung cancer, non-small cell lung cancer, melanoma, lymphoma, pancreatic cancer, prostate cancer, ovarian cancer, stomach cancer, thyroid cancer, cancer of the uterus, liver cancer, cervical cancer, testicular cancer, squamous cell carcinoma, glioma, glioblastoma, adenoma, neuroblastoma, bladder carcinoma, esophageal carcinoma, or triple-negative breast carcinoma.

Embodiment 64 is a method of detecting the presence of IL18BP in a sample in vitro or an individual, the method comprising an anti-IL18bP antibody of any one of embodiments 1-51.

Embodiment 65 is the method of embodiment 64, further comprising quantification of antigen-bound anti-IL18BP antibody.

It is to be understood that one, some, or all of the properties of the various embodiments described herein may be combined to form other embodiments of the present disclosure. These and other aspects of the disclosure will become apparent to one of skill in the art. These and other aspects of the disclosure are further described by the detailed description that follows. In some aspects, provided herein is a method for detecting IL18BP in a sample comprising contacting said sample with any of the anti-IL18BP antibodies described herein.

### BRIEF DESCRIPTION OF THE DRAWINGS

FIG. 1 sets forth data showing changes in spleen weight in mice administered various anti-IL18BP antibodies of the present disclosure compared to that observed in isotype control treated mice in a macrophage activation syndrome animal model.

FIGS. 2A-2D set forth data showing changes in the percentage of various cell types from splenocytes derived from mice administered various anti-IL18BP antibodies of the present disclosure in a macrophage activation syndrome animal model, specifically T cells (FIG. 2A), B cells (FIG. 2B), CD11b+ cells (FIG. 2C), and macrophages (FIG. 2D).

FIGS. 3A-3D set forth data showing changes in cytokine levels in serum of mice administered various anti-IL18BP antibodies of the present disclosure in a macrophage activation syndrome animal model, specifically IFNgamma (FIG. 3A), IL-10 (FIG. 3B), TNFalpha (FIG. 3C), and IL-6 (FIG. 3D).

FIGS. 4A and 4B set forth data showing the effects of anti-IL18BP antibodies of the present disclosure (as monotherapy (FIG. 4A) or in combination with anti-PDL1 antibody administration (FIG. 4B) on tumor growth in E0771 syngeneic tumor model.

FIGS. 5A and 5B set forth data showing the effects of anti-IL18BP antibodies of the present disclosure (as monotherapy (FIG. 5A) or in combination with anti-PDL1 antibody administration (FIG. 5B)) on tumor growth in E0771 syngeneic tumor model.

FIGS. 6A, 6B, and 6C set forth data showing anti-IL-18BP antibodies of the present disclosure increase free IL-18 levels in serum.

FIGS. 7A and 7B set forth data showing anti-IL-18BP antibodies of the present disclosure reduced tumor volume as monotherapy or in combination with anti-PDL1 antibody in E0771 syngeneic tumor model.

FIGS. 8A, 8B, 8C, 8D, 8E, and 8F set forth data showing anti-IL-18BP antibodies of the present disclosure reduced serum levels of IL-18BP and were effective (as monotherapy) or in combination with anti0-PDL1 antibody administration at increasing levels of proinflammatory cytokines and chemokines IFNγ, IL-1β, IL-15, IL-27, and MIP-1α in tumor lysates in E0771 syngeneic tumor model.

FIGS. 9A and 9B set forth data showing anti-IL-18BP antibodies of the present disclosure reduced tumor volume as monotherapy or in combination with anti-PDL1 antibody in EMT6 animal tumor model.

### DETAILED DESCRIPTION OF THE PRESENT DISCLOSURE

The present disclosure relates to anti-IL18BP antibodies (e.g., monoclonal antibodies); methods of making and using such antibodies; pharmaceutical compositions comprising such antibodies: nucleic acids encoding such antibodies; and host cells comprising nucleic acids encoding such antibodies.

The techniques and procedures described or referenced herein are generally well understood and commonly employed using conventional methodology by those skilled in the art, such as, for example, the widely utilized methodologies such as those described in Sambrook et al. Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003); Monoclonal Antibodies: A Practical Approach (P. Shepherd and C. Dean, eds., Oxford University Press, 2000).

### Definitions

The terms “interleukin-18 binding protein” or “IL18BP” or “IL18BP polypeptide” or “IL18BP protein” are used interchangeably herein refer herein to any native IL18BP from any vertebrate source, including mammals such as primates (e.g., humans and cynomolgus monkeys (cyno)) and rodents (e.g., mice and rats), unless otherwise indicated. IL18BP is also referred to as tadekinig-alfa and interferon-γ-inducing factor. In some embodiments, the term encompasses both wild-type sequences and naturally occurring variant sequences, e.g., splice variants or allelic variants. In some embodiments, the term encompasses “full-length.” unprocessed IL18BP as well as any form of IL18BP that results from processing in the cell. In some embodiments, the IL18BP is human IL18BP. As used herein, the term “human IL18BP” refers to a polypeptide with the amino acid sequence of SEQ ID NO: 1.

The terms “anti-IL18BP antibody,” an “antibody that binds to IL18BP,” and “antibody that specifically binds IL18BP” refer to an antibody that is capable of binding IL18BP with sufficient affinity such that the antibody is useful as a diagnostic and/or therapeutic agent in targeting IL18BP. In one embodiment, the extent of binding of an anti-IL18BP antibody to an unrelated, non-IL18BP polypeptide is less than about 10% of the binding of the antibody to IL18BP as measured, e.g., by a radioimmunoassay (RIA). In certain embodiments, an antibody that binds to IL18BP has a dissociation constant (KD) of <1 μM, <100 nM, <10 nM, <1 nM, <0.1 nM, <0.01 nM, or <0.001 nM (e.g., 10⁻⁸ M or less, e.g. from 10⁻⁸ M to 10⁻¹³ M, e.g., from 10⁻⁹ M to 10⁻¹³ M). In certain embodiments, an anti-IL18BP antibody binds to an epitope of IL18BP that is conserved among IL18BP from different species.

With regard to the binding of an antibody to a target molecule, the term “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a KD for the target of about any of 10⁻⁴ M or lower, 10⁻⁵ M or lower, 10⁻⁶ M or lower, 10⁻⁷ M or lower, 10⁻⁸ M or lower, 10⁻⁹ M or lower, 10⁻¹⁰ M or lower, 10⁻¹¹ M or lower, 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In one embodiment, the term “specific binding” refers to binding where a molecule binds to a particular polypeptide or epitope on a particular polypeptide without substantially binding to any other polypeptide or polypeptide epitope.

The term “immunoglobulin” (lg) is used interchangeably with “antibody” herein. The term “antibody” herein is used in the broadest sense and specially covers monoclonal antibodies, polyclonal antibodies, multispecific antibodies (e.g., bispecific antibodies) including those formed from at least two intact antibodies, and antigen-binding antibody fragments so long as they exhibit the desired biological activity.

“Native antibodies” are usually heterotetrameric glycoproteins of about 150,000 Daltons, composed of two identical light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

For the structure and properties of the different classes of antibodies, see, e.g., Basic and Clinical Immunology. 8th Ed., Daniel P. Stites. Abba I. Terr and Tristram G. Parslow (eds.). Appleton & Lange. Norwalk. CT. 1994, page 71 and Chapter 6.

The light chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”), and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three-dimensional configurations of different classes of immunoglobulins are well known and described generally in, for example. Abbas et al., Cellular and Molecular Immunology. 4th ed. (W.B. Saunders Co., 2000).

The “variable region” or “variable domain” of an antibody, such as an anti-IL18BP antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen-binding sites.

The term “variable” refers to the fact that certain segments of the variable domains differ extensively in sequence among antibodies, such as anti-IL18BP antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest. Fifth Edition. National Institute of Health. Bethesda, MD (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody, such as a monoclonal anti-IL18BP antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, but not limited to one or more of the following methods, immunization methods of animals including, but not limited to rats, mice, rabbits, guinea pigs, hamsters and/or chickens with one or more of DNA(s), virus-like particles, polypeptide(s), and/or cell(s), the hybridoma methods. B-cell cloning methods, recombinant DNA methods, and technologies for producing human or human-like antibodies in animals that have parts or all of the human immunoglobulin loci or genes encoding human immunoglobulin sequences.

The terms “full-length antibody.” “intact antibody” or “whole antibody” are used interchangeably to refer to an antibody, such as an anti-IL18BP antibody, in its substantially intact form, as opposed to an antibody fragment. Specifically, full-length antibodies include those with 2 light chains and 2 heavy chains including an Fc region. The constant domains may be native sequence constant domains (e.g., human native sequence constant domains) or amino acid sequence variants thereof. In some cases, the intact antibody may have one or more effector functions.

The terms “monovalent antibody” or “monoarm antibody” refers to an antibody having a single antigen-binding recognition domain that is specific to a target antigen (i.e., the antibody comprises no more than one antigen-binding domain). In some embodiments, a single antigen-binding domain comprises a single variable region heavy chain polypeptide and a single variable region light chain polypeptide. An antibody that is “monovalent” for a target comprises no more than one antigen-binding domain for that target.

An “antibody fragment” refers to a molecule other than an intact antibody that comprises a portion of an intact antibody that binds the antigen to which the intact antibody binds. Examples of antibody fragments include Fab, Fab′, F(ab′)₂ and Fv fragments; diabodies; linear antibodies (see U.S. Pat. No. 5,641,870. Example 2; Zapata et al., Protein Eng. 8 (10): 1057-1062 (1995)); single-chain antibody molecules and multispecific antibodies formed from antibody fragments.

As used herein, the terms “antigen-binding domain,” “antigen-binding region,” “antigen-binding site,” and similar terms refer to the portion of antibody molecules which comprises the amino acid residues that confer on the antibody molecule its specificity for the antigen (e.g., the hypervariable regions (HVR)).

Papain digestion of antibodies, such as anti-IL18BP antibodies of the present disclosure, produces two identical antigen-binding fragments, called “Fab” fragments, and a residual “Fc” fragment, a designation reflecting the ability to crystallize readily. The Fab fragment consists of an entire light chain along with the variable region domain of the heavy chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both heavy chains held together by disulfides. The effector functions of antibodies are determined by sequences in the Fc region, the region which is also recognized by Fc receptors (FcR) found on certain types of cells.

“Functional fragments” of antibodies, such as anti-IL18BP antibodies of the present disclosure comprise a portion of an intact antibody, generally including the antigen-binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing scFv fragments with short linkers (about 5-10 residues) between the V_{H} and V_{L} domains such that inter-chain but not intra-chain pairing of the variable domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains.

As used herein, a “chimeric antibody” refers to an antibody (immunoglobulin), such as a chimeric anti-IL18BP antibody of the present disclosure, in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity. Chimeric antibodies of interest herein include PRIMATIZED® antibodies wherein the antigen-binding region of the antibody is derived from an antibody produced by, e.g., immunizing macaque monkeys with an antigen of interest. As used herein. “humanized antibody” is used a subset of “chimeric antibodies.”

“Humanized” forms of non-human (e.g., murine) antibodies, such as humanized forms of anti-IL18BP antibodies of the present disclosure, are chimeric antibodies comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one that possesses an amino-acid sequence corresponding to that of an antibody, such as an anti-IL18BP antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries and yeast-display libraries. Human antibodies can be prepared by administering the antigen to a transgenic animal that has been modified to produce such antibodies in response to antigenic challenge, but whose endogenous loci have been disabled, e.g., immunized xenomice as well as generated via a human B-cell hybridoma technology.

The term “hypervariable region,” “HVR,” or “HV,” when used herein refers to the regions of an antibody-variable domain, such as that of an anti-IL18BP antibody of the present disclosure, that are hypervariable in sequence and/or form structurally defined loops. Generally, antibodies comprise six HVRs; three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. Naturally occurring camelid antibodies consisting of a heavy chain only are functional and stable in the absence of light chain.

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J. Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework may comprise the same amino acid sequence thereof, or it may comprise pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferable those changes occur at only three, two, or one of positions 71H, 73H and 78H; for instance, the amino acid residues at those positions may by 71A, 73T and/or 78A. In one embodiment, the VL acceptor human framework is identical in sequence to the V_{L} human immunoglobulin framework sequence or human consensus framework sequence.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest. 5th Ed. Public Health Service, National Institutes of Health. Bethesda, MD (1991). Examples include for the V_{L}, the subgroup may be subgroup kappa I, kappa II, kappa III or kappa IV as in Kabat et al., supra. Additionally, for the V_{H}, the subgroup may be subgroup I, subgroup II, or subgroup III as in Kabat et al., supra.

An “amino-acid modification” at a specified position, e.g., of an anti-IL18BP antibody of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

“FV” is the minimum antibody fragment which comprises a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen-binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains, which enables the sFv to form the desired structure for antigen binding.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fe regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least 80% homology with a native sequence Fc region and/or with an Fc region of a parent polypeptide, and most preferably at least 90% homology therewith, more preferably at least 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies.

As used herein. “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

The term “compete” when used in the context of antibodies that compete for the same epitope or overlapping epitopes means competition between antibody as determined by an assay in which the antibody being tested prevents or inhibits (e.g., reduces) specific binding of a reference molecule (e.g., a ligand, or a reference antibody) to a common antigen (e.g., IL18BP or a fragment thereof). Numerous types of competitive binding assays can be used to determine if antibody competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986. J. Immunol. 137:3614-3619) solid phase direct labeled assay, solid phase direct labeled sandwich assay (see, e.g., Harlow and Lane, 1988, Antibodies, A Laboratory Manual. Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990. Scand. J. Immunol. 32:77-82). Typically, such an assay involves the use of purified antigen bound to a solid surface or cells bearing either of these, an unlabeled test antibody and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit (e.g., reduce) specific binding of a reference antibody to a common antigen by at least 20%, 30%, 40%, 50%, 60%, 70%, 80%, 85%, 90%, 95%. 97.5%, and/or near 100%.

As used herein, an “interaction” between a IL18BP polypeptide and a second polypeptide encompasses, without limitation, protein-protein interaction, a physical interaction, a chemical interaction, binding, covalent binding, and ionic binding. As used herein, an antibody “inhibits interaction” between two polypeptides when the antibody disrupts, reduces, or completely eliminates an interaction between the two polypeptides. An antibody of the present disclosure, thereof. “inhibits interaction” between two polypeptides when the antibody thereof binds to one of the two polypeptides. In some embodiments, the interaction can be inhibited by at least any of 20%, 30%, 40%, 50%, 60%, 70%, 80%, 85%, 90%, 95%. 97.5%, and/or near 100%.

The term “epitope” includes any determinant capable of being bound by an antibody. An epitope is a region of an antigen that is bound by an antibody that targets that antigen, and when the antigen is a polypeptide, includes specific amino acids that directly contact the antibody. Most often, epitopes reside on polypeptides, but in some instances, can reside on other kinds of molecules, such as nucleic acids. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of polypeptides and/or macromolecules.

An “isolated” antibody, such as an isolated anti-IL18BP antibody of the present disclosure, is one that has been identified, separated and/or recovered from a component of its production environment (e.g., naturally or recombinantly). Preferably, the isolated antibody is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the antibody will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T-cells since at least one component of the antibody's natural environment will not be present. Ordinarily, however, an isolated polypeptide or antibody will be prepared by at least one purification step.

An “isolated” nucleic acid molecule encoding an antibody, such as an anti-IL18BP antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

“Carriers” as used herein include pharmaceutically acceptable carriers, excipients, or stabilizers that are nontoxic to the cell or mammal being exposed thereto at the dosages and concentrations employed.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary to achieve the desired therapeutic result. An effective amount can be provided in one or more administrations. An effective amount is also one in which any toxic or detrimental effects of the treatment are outweighed by the therapeutically beneficial effects. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “individual” for purposes of treatment refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the individual is human.

As used herein, administration “in conjunction” or “in combination” with another compound or composition includes simultaneous administration and/or administration at different times.

Administration in conjunction or in combination also encompasses administration as a co-formulation or administration as separate compositions, including at different dosing frequencies or intervals, and using the same route of administration or different routes of administration. In some embodiments, administration in conjunction is administration as a part of the same treatment regimen.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a.” “an.” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the present disclosure described herein include “comprising.” “consisting.” and “consisting essentially of” aspects and embodiments.

### Anti-IL18BP Antibodies

Provided herein are anti-IL18BP antibodies. Antibodies provided herein are useful, e.g., for the treatment of various diseases, disorders, and conditions, such as cancer.

In one aspect, the present disclosure provides isolated (e.g., monoclonal) antibodies that bind to an epitope within a IL18BP protein or polypeptide of the present disclosure. IL18BP proteins or polypeptides of the present disclosure include, without limitation, a mammalian IL18BP protein or polypeptide, human IL18BP protein or polypeptide, mouse (murine) IL18BP protein or polypeptide, and cynomolgus IL18BP protein or polypeptide. IL18BP proteins and polypeptides of the present disclosure include naturally occurring variants of IL18BP. In some embodiments, IL18BP proteins and polypeptides of the present disclosure are soluble. In some embodiments, IL18BP proteins and polypeptides of the present disclosure are within the extracellular space. In some embodiments, IL18BP is expressed by mononuclear cells.

### IL18BP Protein

The human IL18BP gene encodes for at least four distinct isoforms derived from mRNA splice variants (IL18BPa, b, c, d); two isoforms of mouse IL18BP have been identified (Kim et al. 2000, PNAS. 97:1190-1195). IL18BPa is the most widely expressed isoform which binds mature IL-18 with an affinity substantially higher than that of IL-18 receptor α.

### Macrophage Activation Syndrome

Macrophage activation syndrome (MAS) is a term used by rheumatologists to describe a potentially life-threatening complication of systemic inflammatory disorders. Animal models of MAS provide a useful model of inflammation (McCain et al, 2018, Blood, 131:1303-1394). In the MAS model described by McCain et al, repeated TLR9 stimulation by repeated injection of cytosine guanine 1826 oligonucleotide (CpG) results in MAS-like disease in rodents, leading to macrophage activation, cytopenia, splenomegaly, and increased levels of serum cytokines, including increased IL-18 levels, IFNγ levels, and IFNγ-related gene expression, independent of lymphocyte dysfunction. When inducing MAS in IL-18BP-deficient mice, a marked increase in levels of IL-18, IFNγ, and CXCL9 is observed, which was resolved with IL-18 blockade or IFNγ blockade. Accordingly, in this MAS model, increased IL-18 levels play a significant role in driving MAS, in part by increasing IFNγ levels.

As described below, a MAS model was used to examine the effects of IL-18BP blockade by anti-IL18BP antibodies of the present disclosure on inflammation. As shown herein in a rodent MAS model, anti-IL18BP antibodies of the present disclosure reduced the percentage of CD3+ T cells; reduced the percentage of CD19+ B cells; reduced the percentage of CD11b+ cells; and increased the percentage of macrophages.

Additionally, anti-IL18BP antibodies of the present disclosure, when administered to mice in the MAS model, resulted in increased levels of inflammatory cytokines in serum, including IFNγ, Il-10, TNFα, and IL-6.

### IL-18, IL18BP, and Cancer

IL18BP is elevated in various human cancers, including, for example, cholangiocarcinoma, diffuse large B cell lymphoma, glioblastoma multiform, head and neck squamous carcinoma, kidney renal clear cell carcinoma, pancreatic adenocarcinoma, skin cutaneous melanoma, and stomach adenocarcinoma. IL18BP has been described as a secreted immune checkpoint that fundamentally alters the biological effects of IL-18 within the tumor microenvironment (TME). Both IL-18BP transcripts and protein are highly expressed in the TME and further increased by treatment with IL-18 in an IFNγ-dependent manner (Zhou et al, 2020, Nature, 583:609; Dixon and Kuchroo, 2020, Cell Research, 30:831-832).

Zhou et al showed that an IL-18 variant that retains the ability to bind the IL-18 receptor alpha and also retains full receptor signaling capacity, but that is not capable of binding to IL-18BP (and thus is not inhibited by IL-18BP, could enhance the activity of IL-18 when this IL-18 variant (referred to as ‘decoy-resistant’ IL-18 (DR-18)) was administered to mice. Specifically, DR-18 showed inhibition of tumor grown, enhanced survival, and (in some mice) complete tumor regression. In this study, the authors suggested that the anti-tumor efficacy of DR-18 is due to its independence from IL18BP, highlighting the potential of the IL-18 pathway to enhance anti-tumor immunity (Zhou et al. 2020. Nature. 583:609).

Without being bound by theory, anti-IL18BP antibodies of the present disclosure block binding of IL-18 to IL18BP, thereby increasing IL-18 availability, enhancing the activity of IL-18, and increasing anti-tumor activity. Anti-IL18BP antibody-mediated blockade of IL-18BP, subsequently unbound IL-18 in the tumor microenvironment (TME) is available to induce inflammation and bind its receptor on T cells and NK cells to promote anti-tumor immunity.

In some aspects, an anti-IL-18BP antibody of the present disclosure increases free IL-18 serum levels. In some aspects, an anti-IL18BP antibody of the present disclosure decreases IL-18BP serum levels.

In some aspects, an anti-IL-18BP antibody of the present disclosure increases levels of proinflammatory cytokines and chemokines in vivo. In some aspects, an anti-IL-18BP antibody of the present disclosure increases levels of proinflammatory cytokines and chemokines in tumor lysates (e.g., in the tumor micro-environment). In some aspects, an anti-IL-18BP antibody of the present disclosure increases levels of INFγ, IL-1B, IL-15, IL-27, and/or MIP-1α in vivo. In other aspects, an anti-IL-18BP antibody of the present disclosure increases levels of INFγ, IL-1β, IL-15, IL-27, and/or MIP-1α in tumor lysates (e.g., in the tumor micro-environment).

The present disclosure shows that reduction in IL18BP activity or expression with anti-IL18BP antibodies reduces tumor growth in a mouse tumor model.

### Exemplary Anti-IL18BP Antibodies and Certain Other Antibody Embodiments

In some aspects, provided herein are anti-IL18BP antibodies comprising at least one, two, three, four, five, or six HVRs selected from: (a) HVR-H1 comprising an amino acid sequence selected the group consisting of SEQ ID NOs: 60, 61, 62, 63, 64, 65, 66, 67, 68, 69, 70, 167, 168, and 169; (b) HVR-H2 comprising an amino acid sequence selected from the group consisting of SEQ ID NOs: 71, 72, 73, 74, 75, 76, 77, 78, 79, 80, 81, 82, 83, 84, 85, 86, 170, 171, 172, 173, 174, 175, 176, 177, and 178; (c) HVR-H3 comprising an amino acid sequence selected from the group consisting of SEQ ID NOs: 87, 88, 89, 90, 91, 92, 93, 94, 95, 96, 97, 98, 99, 100, 179, 180, 181, 182, 183, 184, 185, 186, 187, and 188; (d) HVR-L1 comprising an amino acid sequence selected from the group consisting of SEQ ID NOs: 101, 102, 103, 104, 105, 106, 107, 108, 109, 110, 111, and 247; (c) HVR-L2 comprising an amino acid sequence selected from the group consisting of SEQ ID NOs: 112, 113, 114, 115, 116, 117, 118, 119, 120, 121, and 248; and (f) HVR-L3 comprising an amino acid sequence selected from the group consisting of SEQ ID NOs: 122, 123, 124, 125, 126, 127, 128, 129, 130, 131, 132, 133, and 249.

In some aspects, provided herein are anti-IL18BP antibodies comprising at least one, at least two, or all three V_{H} HVR sequences selected from (a) HVR-H1 comprising an amino acid sequence selected the group consisting of SEQ ID NOs: 60, 61, 62, 63, 64, 65, 66, 67, 68, 69, 70, 167, 168, and 169; (b) HVR-H2 comprising an amino acid sequence selected from the group consisting of SEQ ID NOs: 71, 72, 73, 74, 75, 76, 77, 78, 79, 80, 81, 82, 83, 84, 85, 86, 170, 171, 172, 173, 174, 175, 176, 177, and 178; (c) HVR-H3 comprising an amino acid sequence selected from the group consisting of SEQ ID NOs: 87, 88, 89, 90, 91, 92, 93, 94, 95, 96, 97, 98, 99, 100, 179, 180, 181, 182, 183, 184, 185, 186, 187, and 188.

In some aspects, provided herein are anti-IL18BP antibodies comprising at least one, at least two, or all three V_{L} HVR sequences selected from (a) HVR-L1 comprising an amino acid sequence selected from the group consisting of SEQ ID NOs: 101, 102, 103, 104, 105, 106, 107, 108, 109, 110, 111, and 247; (b) HVR-L2 comprising an amino acid sequence selected from the group consisting of SEQ ID NOs: 112, 113, 114, 115, 116, 117, 118, 119, 120, 121, and 248; and (c) HVR-L3 comprising an amino acid sequence selected from the group consisting of SEQ ID NOs: 122, 123, 124, 125, 126, 127, 128, 129, 130, 131, 132, 133, and 249.

In some aspects, provided herein are anti-IL18BP antibodies comprising (a) a V_{H} domain comprising at least one, at least two, or all three V_{H} HVR sequences selected from (i) HVR-H1 comprising an amino acid sequence selected from the group consisting of SEQ ID NOs: 60, 61, 62, 63, 64, 65, 66, 67, 68, 69, 70, 167, 168, and 169; (ii) HVR-H2 comprising an amino acid sequence selected from the group consisting of SEQ ID NOs: 71, 72, 73, 74, 75, 76, 77, 78, 79, 80, 81, 82, 83, 84, 85, 86, 170, 171, 172, 173, 174, 175, 176, 177, and 178; and (iii) HVR-H3 comprising an amino acid sequence selected from the group consisting of SEQ ID NOs: 87, 88, 89, 90, 91, 92, 93, 94, 95, 96, 97, 98, 99, 100, 179, 180, 181, 182, 183, 184, 185, 186, 187, and 188; and (b) a V_{L} domain comprising at least one, at least two, or all three V_{L} HVR sequences selected from (i) HVR-L1 comprising an amino acid sequence selected from the group consisting of SEQ ID NOs: 101, 102, 103, 104, 105, 106, 107, 108, 109, 110, 111, and 247; (ii) HVR-L2 comprising an amino acid sequence selected from the group consisting of SEQ ID NOs: 112, 113, 114, 115, 116, 117, 118, 119, 120, 121, and 248; and (iii) HVR-L3 comprising an amino acid sequence selected from the group consisting of SEQ ID NOs: 122, 123, 124, 125, 126, 127, 128, 129, 130, 131, 132, 133, and 249.

In some aspects, provided herein are anti-IL18BP antibodies comprising: (a) HVR-H1 comprising the amino acid sequence of SEQ ID NO:60; (b) HVR-H2 comprising the amino acid sequence of SEQ ID NO:71; (c) HVR-H3 comprising the amino acid sequence of SEQ ID NO:87; (d) HVR-L1 comprising the amino acid sequence of SEQ ID NO: 101; (e) HVR-L2 comprising the amino acid sequence of SEQ ID NO: 112; and (f) HVR-L3 comprising the amino acid sequence of SEQ ID NO: 122; (a) HVR-H1 comprising the amino acid sequence of SEQ ID NO:61; (b) HVR-H2 comprising the amino acid sequence of SEQ ID NO: 72; (c) HVR-H3 comprising the amino acid sequence of SEQ ID NO: 88; (d) HVR-L1 comprising the amino acid sequence of SEQ ID NO: 102; (c) HVR-L2 comprising the amino acid sequence of SEQ ID NO: 113; and (f) HVR-L3 comprising the amino acid sequence of SEQ ID NO: 123; (a) HVR-H1 comprising the amino acid sequence of SEQ ID NO:61; (b) HVR-H2 comprising the amino acid sequence of SEQ ID NO: 73; (c) HVR-H3 comprising the amino acid sequence of SEQ ID NO:88; (d) HVR-L1 comprising the amino acid sequence of SEQ ID NO: 103; (c) HVR-L2 comprising the amino acid sequence of SEQ ID NO: 113; and (f) HVR-L3 comprising the amino acid sequence of SEQ ID NO: 123; (a) HVR-H1 comprising the amino acid sequence of SEQ ID NO: 62; (b) HVR-H2 comprising the amino acid sequence of SEQ ID NO: 74; (c) HVR-H3 comprising the amino acid sequence of SEQ ID NO:89; (d) HVR-L1 comprising the amino acid sequence of SEQ ID NO: 104; (c) HVR-L2 comprising the amino acid sequence of SEQ ID NO: 114; and (f) HVR-L3 comprising the amino acid sequence of SEQ ID NO: 124; (a) HVR-H1 comprising the amino acid sequence of SEQ ID NO:61; (b) HVR-H2 comprising the amino acid sequence of SEQ ID NO: 75; (c) HVR-H3 comprising the amino acid sequence of SEQ ID NO:90; (d) HVR-L1 comprising the amino acid sequence of SEQ ID NO: 105; (e) HVR-L2 comprising the amino acid sequence of SEQ ID NO: 113; and (f) HVR-L3 comprising the amino acid sequence of SEQ ID NO: 123; (a) HVR-H1 comprising the amino acid sequence of SEQ ID NO:63; (b) HVR-H2 comprising the amino acid sequence of SEQ ID NO: 76; (c) HVR-H3 comprising the amino acid sequence of SEQ ID NO:91; (d) HVR-L1 comprising the amino acid sequence of SEQ ID NO: 106; (e) HVR-L2 comprising the amino acid sequence of SEQ ID NO: 115; and (f) HVR-L3 comprising the amino acid sequence of SEQ ID NO: 125; (a) HVR-H1 comprising the amino acid sequence of SEQ ID NO:61; (b) HVR-H2 comprising the amino acid sequence of SEQ ID NO: 77; (c) HVR-H3 comprising the amino acid sequence of SEQ ID NO: 88; (d) HVR-L1 comprising the amino acid sequence of SEQ ID NO: 103; (e) HVR-L2 comprising the amino acid sequence of SEQ ID NO: 113; and (f) HVR-L3 comprising the amino acid sequence of SEQ ID NO: 123; (a) HVR-H1 comprising the amino acid sequence of SEQ ID NO:64; (b) HVR-H2 comprising the amino acid sequence of SEQ ID NO: 78; (c) HVR-H3 comprising the amino acid sequence of SEQ ID NO:92; (d) HVR-L1 comprising the amino acid sequence of SEQ ID NO: 107; (e) HVR-L2 comprising the amino acid sequence of SEQ ID NO: 116; and (f) HVR-L3 comprising the amino acid sequence of SEQ ID NO: 126; (a) HVR-H1 comprising the amino acid sequence of SEQ ID NO: 65; (b) HVR-H2 comprising the amino acid sequence of SEQ ID NO: 79; (c) HVR-H3 comprising the amino acid sequence of SEQ ID NO:93; (d) HVR-L1 comprising the amino acid sequence of SEQ ID NO: 101; (e) HVR-L2 comprising the amino acid sequence of SEQ ID NO: 116; and (f) HVR-L3 comprising the amino acid sequence of SEQ ID NO: 127; (a) HVR-H1 comprising the amino acid sequence of SEQ ID NO:66; (b) HVR-H2 comprising the amino acid sequence of SEQ ID NO:80; (c) HVR-H3 comprising the amino acid sequence of SEQ ID NO:94; (d) HVR-L1 comprising the amino acid sequence of SEQ ID NO: 108; (e) HVR-L2 comprising the amino acid sequence of SEQ ID NO: 117; and (f) HVR-L3 comprising the amino acid sequence of SEQ ID NO: 128; (a) HVR-H1 comprising the amino acid sequence of SEQ ID NO:66; (b) HVR-H2 comprising the amino acid sequence of SEQ ID NO:81; (c) HVR-H3 comprising the amino acid sequence of SEQ ID NO:95; (d) HVR-L1 comprising the amino acid sequence of SEQ ID NO: 109; (e) HVR-L2 comprising the amino acid sequence of SEQ ID NO: 118; and (f) HVR-L3 comprising the amino acid sequence of SEQ ID NO: 129; (a) HVR-H1 comprising the amino acid sequence of SEQ ID NO:67; (b) HVR-H2 comprising the amino acid sequence of SEQ ID NO:82; (c) HVR-H3 comprising the amino acid sequence of SEQ ID NO: 96; (d) HVR-L1 comprising the amino acid sequence of SEQ ID NO: 101; (e) HVR-L2 comprising the amino acid sequence of SEQ ID NO: 116; and (f) HVR-L3 comprising the amino acid sequence of SEQ ID NO: 130; (a) HVR-H1 comprising the amino acid sequence of SEQ ID NO:68; (b) HVR-H2 comprising the amino acid sequence of SEQ ID NO:83; (c) HVR-H3 comprising the amino acid sequence of SEQ ID NO:97; (d) HVR-L1 comprising the amino acid sequence of SEQ ID NO: 101; (e) HVR-L2 comprising the amino acid sequence of SEQ ID NO: 119; and (f) HVR-L3 comprising the amino acid sequence of SEQ ID NO: 131; (a) HVR-H1 comprising the amino acid sequence of SEQ ID NO: 69; (b) HVR-H2 comprising the amino acid sequence of SEQ ID NO:84; (c) HVR-H3 comprising the amino acid sequence of SEQ ID NO:98; (d) HVR-L1 comprising the amino acid sequence of SEQ ID NO: 110; (e) HVR-L2 comprising the amino acid sequence of SEQ ID NO: 120; and (f) HVR-L3 comprising the amino acid sequence of SEQ ID NO: 132; (a) HVR-H1 comprising the amino acid sequence of SEQ ID NO: 70; (b) HVR-H2 comprising the amino acid sequence of SEQ ID NO:85; (c) HVR-H3 comprising the amino acid sequence of SEQ ID NO:99; (d) HVR-L1 comprising the amino acid sequence of SEQ ID NO: 111; (e) HVR-L2 comprising the amino acid sequence of SEQ ID NO: 121; and (f) HVR-L3 comprising the amino acid sequence of SEQ ID NO: 133; and (a) HVR-H1 comprising the amino acid sequence of SEQ ID NO:70; (b) HVR-H2 comprising the amino acid sequence of SEQ ID NO:86; (c) HVR-H3 comprising the amino acid sequence of SEQ ID NO: 100; (d) HVR-L1 comprising the amino acid sequence of SEQ ID NO: 111; (e) HVR-L2 comprising the amino acid sequence of SEQ ID NO: 121; and (f) HVR-L3 comprising the amino acid sequence of SEQ ID NO: 133.

In another aspect, an anti-IL18BP antibody comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs: 29, 30, 31, 32, 33, 34, 35, 36, 37, 38, 39, 40, 41, 42, 43, and 44. In certain aspects, a V_{H} sequence having at least 90%, 91%, 92%, 93%, 94%, 95%, 96%, 97%, 98%, or 99% identity to an amino acid sequence selected from the group consisting of SEQ ID NOs: 29, 30, 31, 32, 33, 34, 35, 36, 37, 38, 39, 40, 41, 42, 43, and 44, and contains substitutions (e.g., conservative substitutions), insertions, or deletions relative to the reference sequence, but an anti-IL18BP antibody comprising that sequence retains the ability to bind to IL18BP. In certain aspects, a total of 1 to 10 amino acids have been substituted, inserted, and/or deleted in SEQ ID NO: 29, 30, 31, 32, 33, 34, 35, 36, 37, 38, 39, 40, 41, 42, 43, or 44. In certain aspects, a total of 1 to 5 amino acids have been substituted, inserted and/or deleted in SEQ ID NO: 29, 30, 31, 32, 33, 34, 35, 36, 37, 38, 39, 40, 41, 42, 43, or 44. In certain aspects, substitutions, insertions, or deletions occur in regions outside the HVRs (i.e., in the FRs). Optionally, the anti-IL18BP antibody comprises the V_{H} sequence of SEQ ID NO: 29, 30, 31, 32, 33, 34, 35, 36, 37, 38, 39, 40, 41, 42, 43, and 44, including post-translational modifications of that sequence. In a particular aspect, the V_{H} comprises one, two or three HVRs selected from: (a) HVR-H1 comprising an amino acid sequence selected from the group consisting of SEQ ID NOs: 60, 61, 62, 63, 64, 65, 66, 67, 68, 69, and 70; (b) HVR-H2 comprising an amino acid sequence selected from the group consisting of SEQ ID NOs: 71, 72, 73, 74, 75, 76, 77, 78, 79, 80, 81, 82, 83, 84, 85, and 86; and (c) HVR-H3 comprising an amino acid sequence selected from the group consisting of SEQ ID NOs: 87, 88, 89, 90, 91, 92, 93, 94, 95, 96, 97, 98, 99, and 100.

In another aspect, an anti-IL18BP antibody is provided, wherein the antibody comprises a light chain variable domain (V_{L}) having at least 90%, 91%, 92%, 93%, 94%, 95%, 96%, 97%, 98%, 99%, or 100% sequence identity to an amino acid sequence selected from the group consisting of SEQ ID NOs: 45, 46, 47, 48, 49, 50, 51, 52, 53, 54, 55, 56, 57, 58, and 59. In certain aspects, a V_{L} sequence having at least 90%, 91%, 92%, 93%, 94%, 95%, 96%, 97%, 98%, or 99% identity to an amino acid sequence selected from the group consisting of SEQ ID NOs: 45, 46, 47, 48, 49, 50, 51, 52, 53, 54, 55, 56, 57, 58, and 59, and contains substitutions (e.g., conservative substitutions), insertions, or deletions relative to the reference sequence, but an anti-IL18BP antibody comprising that sequence retains the ability to bind to IL18BP. In some aspects, a total of 1 to 10 amino acids have been substituted, inserted and/or deleted in SEQ ID NO: 45, 46, 47, 48, 49, 50, 51, 52, 53, 54, 55, 56, 57, 58, or 59. In certain aspects, a total of 1 to 5 amino acids have been substituted, inserted and/or deleted in SEQ ID NO: 45, 46, 47, 48, 49, 50, 51, 52, 53, 54, 55, 56, 57, 58, or 59. In certain aspects, the substitutions, insertions, or deletions occur in regions outside the HVRs (i.e., in the FRs). Optionally, the anti-IL18BP antibody comprises the V_{L} sequence of SEQ ID NO: 162, 164, 166, 168, 170, 172, 174, 176, 178, 180, 182, 184, 186, 188, 190, 192, 194, 196, 198, 200, 202, 204, 206, 208, or 210, including post-translational modifications of that sequence. In a particular aspect, the V_{L} comprises one, two or three HVRs selected from (a) HVR-L1 comprising an amino acid sequence selected from the group consisting of SEQ ID NOs: 101, 102, 103, 104, 105, 106, 107, 108, 109, 110, and 111; (b) HVR-L2 comprising an amino acid sequence selected from the group consisting of SEQ ID NOs: 112, 113, 114, 115, 116, 117, 118, 119, 120, and 121; and (c) HVR-L3 comprising an amino acid sequence selected from the group consisting of SEQ ID NOs: 122, 123, 124, 125, 126, 127, 128, 129, 130, 131, 132, and 133.

In some aspects, an anti-IL18BP antibody is provided, wherein the antibody comprises a V_{H} as in any of the aspects provided above, and a V_{L} as in any of the aspects provided above. In some aspects, provided herein are anti-IL18BP antibodies, wherein the antibody comprises a V_{H} as in any of the aspects provided above, and a V_{L} as in any of the aspects provided above. In one aspect, the antibody comprises the V_{H} and V_{L} sequences in SEQ ID NOs: 29, 30, 31, 32, 33, 34, 35, 36, 37, 38, 39, 40, 41, 42, 43, and 44, and SEQ ID NOs: 45, 46, 47, 48, 49, 50, 51, 52, 53, 54, 55, 56, 57, 58, and 59, respectively, including post-translational modifications of those sequences.

In some aspects, provided herein are anti-IL18BP antibodies comprising a heavy chain variable domain (V_{H}) and a light chain variable domain (V_{L}), wherein the V_{H} and V_{L} are selected from the group consisting of: V_{H} comprising the amino acid sequence of SEQ ID NO:29 and V_{L} comprising the amino acid sequence of SEQ ID NO:45; V_{H} comprising the amino acid sequence of SEQ ID NO:30 and V_{L} comprising the amino acid sequence of SEQ ID NO:46; V_{H} comprising the amino acid sequence of SEQ ID NO:31 and V_{L} comprising the amino acid sequence of SEQ ID NO:47; V_{H} comprising the amino acid sequence of SEQ ID NO:32 and V_{L} comprising the amino acid sequence of SEQ ID NO:48; V_{H} comprising the amino acid sequence of SEQ ID NO:33 and V_{L} comprising the amino acid sequence of SEQ ID NO:49; V_{H} comprising the amino acid sequence of SEQ ID NO:34 and V_{L} comprising the amino acid sequence of SEQ ID NO:50; V_{H} comprising the amino acid sequence of SEQ ID NO:35 and V_{L} comprising the amino acid sequence of SEQ ID NO:51; V_{H} comprising the amino acid sequence of SEQ ID NO:36 and V_{L} comprising the amino acid sequence of SEQ ID NO:52; V_{H} comprising the amino acid sequence of SEQ ID NO:37 and V_{L} comprising the amino acid sequence of SEQ ID NO:53; V_{H} comprising the amino acid sequence of SEQ ID NO:38 and V_{L} comprising the amino acid sequence of SEQ ID NO:54; V_{H} comprising the amino acid sequence of SEQ ID NO:39 and V_{L} comprising the amino acid sequence of SEQ ID NO:55; V_{H} comprising the amino acid sequence of SEQ ID NO:40 and V_{L} comprising the amino acid sequence of SEQ ID NO:56; V_{H} comprising the amino acid sequence of SEQ ID NO:41 and V_{L} comprising the amino acid sequence of SEQ ID NO:57; V_{H} comprising the amino acid sequence of SEQ ID NO:42 and V_{L} comprising the amino acid sequence of SEQ ID NO:58; V_{H} comprising the amino acid sequence of SEQ ID NO:43 and V_{L} comprising the amino acid sequence of SEQ ID NO:59; and V_{H} comprising the amino acid sequence of SEQ ID NO:44 and V_{L} comprising the amino acid sequence of SEQ ID NO:59.

In some aspects, an anti-IL18BP antibody of the present disclosure competitively inhibits binding of at least one reference antibody selected from anti-IL18BP antibody BP-01, BP-02, BP-03, BP-04, BP-05, BP-06, BP-07, BP-08, BP-09, BP-10, BP-11, BP-12, BP-13, BP-14, BP-15, and BP-16, and any combination thereof, for binding to IL18BP.

In some aspects, an anti-IL18BP antibody of the present disclosure binds to an epitope of human IL18BP that is the same as or overlaps with the IL18BP epitope bound by at least one reference antibody selected from anti-IL18BP antibody BP-01, BP-02, BP-03, BP-04, BP-05, BP-06, BP-07, BP-08, BP-09, BP-10, BP-11, BP-12, BP-13, BP-14, BP-15, and BP-16. Detailed exemplary methods for mapping an epitope to which an antibody binds are provided in Morris (1996) “Epitope Mapping Protocols.” in Methods in Molecular Biology vol. 66 (Humana Press. Totowa, NJ).

In some aspects, an anti-IL18BP antibody of the present disclosure competitively inhibits binding of at least one reference antibody, or binds to an epitope of human IL18BP that is the same as or overlaps with the IL18BP epitope bound by at least one reference antibody, wherein the reference antibody is an anti-IL18BP antibody comprising a heavy chain variable domain (V_{H}) and a light chain variable domain (V_{L}), wherein the V_{H} and V_{L} are selected from the group consisting of: V_{H} comprising the amino acid sequence of SEQ ID NO:29 and V_{L} comprising the amino acid sequence of SEQ ID NO: 45; V_{H} comprising the amino acid sequence of SEQ ID NO:30 and V_{L} comprising the amino acid sequence of SEQ ID NO:46; V_{H} comprising the amino acid sequence of SEQ ID NO:31 and V_{L}, comprising the amino acid sequence of SEQ ID NO:47; V_{H} comprising the amino acid sequence of SEQ ID NO:32 and V_{L} comprising the amino acid sequence of SEQ ID NO:48; V_{H} comprising the amino acid sequence of SEQ ID NO:33 and V_{L} comprising the amino acid sequence of SEQ ID NO:49; V_{H} comprising the amino acid sequence of SEQ ID NO:34 and V_{L} comprising the amino acid sequence of SEQ ID NO:50; V_{H} comprising the amino acid sequence of SEQ ID NO:35 and V_{L} comprising the amino acid sequence of SEQ ID NO:51; V_{H} comprising the amino acid sequence of SEQ ID NO:36 and V_{L} comprising the amino acid sequence of SEQ ID NO:52; V_{H} comprising the amino acid sequence of SEQ ID NO:37 and V_{L} comprising the amino acid sequence of SEQ ID NO:53; V_{H} comprising the amino acid sequence of SEQ ID NO:38 and V_{L} comprising the amino acid sequence of SEQ ID NO:54; V_{H} comprising the amino acid sequence of SEQ ID NO:39 and V_{L} comprising the amino acid sequence of SEQ ID NO:55; V_{H} comprising the amino acid sequence of SEQ ID NO:40 and V_{L} comprising the amino acid sequence of SEQ ID NO:56; V_{H} comprising the amino acid sequence of SEQ ID NO:41 and V_{L} comprising the amino acid sequence of SEQ ID NO:57; V_{H} comprising the amino acid sequence of SEQ ID NO:42 and V_{L} comprising the amino acid sequence of SEQ ID NO:58; V_{H} comprising the amino acid sequence of SEQ ID NO:43 and V_{L} comprising the amino acid sequence of SEQ ID NO:59; and V_{H} comprising the amino acid sequence of SEQ ID NO:44 and V_{L} comprising the amino acid sequence of SEQ ID NO:59.

In some aspects, provided herein are anti-IL18BP antibodies comprising at least one, two, three, four, five, or six HVRs selected from: (a) HVR-H1 comprising an amino acid sequence selected the group consisting of SEQ ID NOs: 167, 168, and 169; (b) HVR-H2 comprising an amino acid sequence selected from the group consisting of SEQ ID NOs: 170, 171, 172, 173, 174, 175, 176, 177, and 178; (c) HVR-H3 comprising an amino acid sequence selected from the group consisting of SEQ ID NOs: 179, 180, 181, 182, 183, 184, 185, 186, 187, and 188; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249.

In some aspects, provided herein are anti-IL18BP antibodies comprising at least one, at least two, or all three V_{H} HVR sequences selected from (a) HVR-H1 comprising an amino acid sequence selected the group consisting of SEQ ID NOs: 167, 168, and 169; (b) HVR-H2 comprising an amino acid sequence selected from the group consisting of SEQ ID NOs: 170, 171, 172, 173, 174, 175, 176, 177, and 178; (c) HVR-H3 comprising an amino acid sequence selected from the group consisting of SEQ ID NOs: 179, 180, 181, 182, 183, 184, 185, 186, 187, and 188.

In some aspects, provided herein are anti-IL18BP antibodies comprising at least one, at least two, or all three V_{L} HVR sequences selected from (a) HVR-L1 comprising the amino acid sequence of SEQ ID NO:247; (b) HVR-L2 comprising the amino acid sequence of SEQ ID NO:248; and (c) HVR-L3 comprising the amino acid sequence of SEQ ID NO:249.

In some aspects, provided herein are anti-IL18BP antibodies comprising (a) a V_{H} domain comprising at least one, at least two, or all three V_{H} HVR sequences selected from (i) HVR-H1 comprising an amino acid sequence selected from the group consisting of SEQ ID NOs: 167, 168, and 169; (ii) HVR-H2 comprising an amino acid sequence selected from the group consisting of SEQ ID NOs: 170, 171, 172, 173, 174, 175, 176, 177, and 178; and (iii) HVR-H3 comprising an amino acid sequence selected from the group consisting of SEQ ID NOs: 179, 180, 181, 182, 183, 184, 185, 186, 187, and 188 and (b) a V_{L} domain comprising at least one, at least two, or all three V_{L} HVR sequences selected from (i) HVR-L1 comprising the amino acid sequence of SEQ ID NO: 247; (ii) HVR-L2 comprising the amino acid sequence of SEQ ID NO: 248; and (iii) HVR-L3 comprising the amino acid sequence of SEQ ID NO: 249.

In some aspects, provided herein are anti-IL18BP antibodies comprising: (a) HVR-H1 comprising the amino acid sequence of SEQ ID NO: 167; (b) HVR-H2 comprising the amino acid sequence of SEQ ID NO: 170; (c) HVR-H3 comprising the amino acid sequence of SEQ ID NO: 179; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO: 249; (a) HVR-H1 comprising the amino acid sequence of SEQ ID NO: 168; (b) HVR-H2 comprising the amino acid sequence of SEQ ID NO: 171; (c) HVR-H3 comprising the amino acid sequence of SEQ ID NO: 180; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 171; (c) HVR-H3 comprising the amino acid sequence of SEQ ID NO: 181; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 172; (c) HVR-H3 comprising the amino acid sequence of SEQ ID NO: 182; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO: 249; (a) HVR-H1 comprising the amino acid sequence of SEQ ID NO: 169; (b) HVR-H2 comprising the amino acid sequence of SEQ ID NO: 173; (c) HVR-H3 comprising the amino acid sequence of SEQ ID NO: 183; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 173; (c) HVR-H3 comprising the amino acid sequence of SEQ ID NO: 184; (d) HVR-L1 comprising the amino acid sequence of SEQ ID NO: 247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 173; (c) HVR-H3 comprising the amino acid sequence of SEQ ID NO: 185; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO: 249; (a) HVR-H1 comprising the amino acid sequence of SEQ ID NO: 169; (b) HVR-H2 comprising the amino acid sequence of SEQ ID NO: 174; (c) HVR-H3 comprising the amino acid sequence of SEQ ID NO: 185; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 175; (c) HVR-H3 comprising the amino acid sequence of SEQ ID NO: 183; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 175; (c) HVR-H3 comprising the amino acid sequence of SEQ ID NO: 184; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO: 249; (a) HVR-H1 comprising the amino acid sequence of SEQ ID NO: 169; (b) HVR-H2 comprising the amino acid sequence of SEQ ID NO: 175; (c) HVR-H3 comprising the amino acid sequence of SEQ ID NO: 185; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 176; (c) HVR-H3 comprising the amino acid sequence of SEQ ID NO: 186; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 171; (c) HVR-H3 comprising the amino acid sequence of SEQ ID NO: 187; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO: 249; (a) HVR-H1 comprising the amino acid sequence of SEQ ID NO: 169; (b) HVR-H2 comprising the amino acid sequence of SEQ ID NO: 171; (c) HVR-H3 comprising the amino acid sequence of SEQ ID NO: 188; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 171; (c) HVR-H3 comprising the amino acid sequence of SEQ ID NO: 182; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 172; (c) HVR-H3 comprising the amino acid sequence of SEQ ID NO: 183; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO: 249; (a) HVR-H1 comprising the amino acid sequence of SEQ ID NO: 169; (b) HVR-H2 comprising the amino acid sequence of SEQ ID NO: 172; (c) HVR-H3 comprising the amino acid sequence of SEQ ID NO: 184; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 172; (c) HVR-H3 comprising the amino acid sequence of SEQ ID NO: 185; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 177; (c) HVR-H3 comprising the amino acid sequence of SEQ ID NO: 183; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO: 249; (a) HVR-H1 comprising the amino acid sequence of SEQ ID NO: 169; (b) HVR-H2 comprising the amino acid sequence of SEQ ID NO: 177; (c) HVR-H3 comprising the amino acid sequence of SEQ ID NO: 184; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 177; (c) HVR-H3 comprising the amino acid sequence of SEQ ID NO: 185; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 173; (c) HVR-H3 comprising the amino acid sequence of SEQ ID NO: 186; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO: 249; (a) HVR-H1 comprising the amino acid sequence of SEQ ID NO: 169; (b) HVR-H2 comprising the amino acid sequence of SEQ ID NO: 178; (c) HVR-H3 comprising the amino acid sequence of SEQ ID NO: 183; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 178; (c) HVR-H3 comprising the amino acid sequence of SEQ ID NO: 184; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 178; (c) HVR-H3 comprising the amino acid sequence of SEQ ID NO: 185; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO: 249; (a) HVR-H1 comprising the amino acid sequence of SEQ ID NO: 169; (b) HVR-H2 comprising the amino acid sequence of SEQ ID NO: 175; (c) HVR-H3 comprising the amino acid sequence of SEQ ID NO: 186; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 171; (c) HVR-H3 comprising the amino acid sequence of SEQ ID NO: 183; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 171; (c) HVR-H3 comprising the amino acid sequence of SEQ ID NO: 184; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO: 249; (a) HVR-H1 comprising the amino acid sequence of SEQ ID NO: 169; (b) HVR-H2 comprising the amino acid sequence of SEQ ID NO: 171; (c) HVR-H3 comprising the amino acid sequence of SEQ ID NO: 185; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 172; (c) HVR-H3 comprising the amino acid sequence of SEQ ID NO: 186; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249; (a) HVR-H1 comprising the amino acid sequence of SEQ ID NO: 169; (b) HVR-H2 comprising the amino acid sequence of SEQ ID NO: 177; (c) HVR-H3 comprising the amino acid sequence of SEQ ID NO: 186; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO: 249; and (a) HVR-H1 comprising the amino acid sequence of SEQ ID NO: 169; (b) HVR-H2 comprising the amino acid sequence of SEQ ID NO: 178; (c) HVR-H3 comprising the amino acid sequence of SEQ ID NO: 186; (d) HVR-L1 comprising the amino acid sequence of SEQ ID NO:247; (e) HVR-L2 comprising the amino acid sequence of SEQ ID NO:248; and (f) HVR-L3 comprising the amino acid sequence of SEQ ID NO:249.

In another aspect, an anti-IL18BP antibody comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs: 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, and 165. In certain aspects, a V_{H} sequence having at least 90%, 91%, 92%, 93%, 94%, 95%, 96%, 97%, 98%, or 99% identity to an amino acid sequence selected from the group consisting of SEQ ID NOs: 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, and 165, and contains substitutions (e.g., conservative substitutions), insertions, or deletions relative to the reference sequence, but an anti-IL18BP antibody comprising that sequence retains the ability to bind to IL18BP. In certain aspects, a total of 1 to 10 amino acids have been substituted, inserted, and/or deleted in SEQ ID NO: 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, or 165. In certain aspects, a total of 1 to 5 amino acids have been substituted, inserted and/or deleted in SEQ ID NO: 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, or 165. In certain aspects, substitutions, insertions, or deletions occur in regions outside the HVRs (i.e., in the FRs). Optionally, the anti-IL18BP antibody comprises the V_{H} sequence of SEQ ID NO: 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, and 165, including post-translational modifications of that sequence. In a particular aspect, the V_{H} comprises one, two or three HVRs selected from: (a) HVR-H1 comprising an amino acid sequence selected from the group consisting of SEQ ID NOs: 167, 168, and 169; (b) HVR-H2 comprising an amino acid sequence selected from the group consisting of SEQ ID NOs: 170, 171, 172, 173, 174, 175, 176, 177, and 178; and (c) HVR-H3 comprising an amino acid sequence selected from the group consisting of SEQ ID NOs: 179, 180, 181, 182, 183, 184, 185, 186, 187, and 188.

In another aspect, an anti-IL18BP antibody is provided, wherein the antibody comprises a light chain variable domain (V_{L}) having at least 90%, 91%, 92%, 93%, 94%, 95%, 96%, 97%, 98%, 99%, or 100% sequence identity to the amino acid sequence of SEQ ID NO: 166. In certain aspects, a V_{L} sequence having at least 90%, 91%, 92%, 93%, 94%, 95%, 96%, 97%, 98%, or 99% identity to the amino acid sequence of SEQ ID NO: 166, and contains substitutions (e.g., conservative substitutions), insertions, or deletions relative to the reference sequence, but an anti-IL18BP antibody comprising that sequence retains the ability to bind to IL18BP. In some aspects, a total of 1 to 10 amino acids have been substituted, inserted and/or deleted in SEQ ID NO: 166. In certain aspects, a total of 1 to 5 amino acids have been substituted, inserted and/or deleted in SEQ ID NO: 166. In certain aspects, the substitutions, insertions, or deletions occur in regions outside the HVRs (i.e., in the FRs). Optionally, the anti-IL18BP antibody comprises the V_{L} sequence of SEQ ID NO: 166, including post-translational modifications of that sequence. In a particular aspect, the V_{L} comprises one, two or three HVRs selected from (a) HVR-L1 comprising the amino acid sequence of SEQ ID NO: 247; (b) HVR-L2 comprising the amino acid sequence of SEQ ID NO: 248; and (c) HVR-L3 comprising the amino acid sequence of SEQ ID NO: 249.

In some aspects, an anti-IL18BP antibody is provided, wherein the antibody comprises a V_{H} as in any of the aspects provided above, and a V_{L} as in any of the aspects provided above. In some aspects, provided herein are anti-IL18BP antibodies, wherein the antibody comprises a V_{H} as in any of the aspects provided above, and a V_{L} as in any of the aspects provided above. In one aspect, the antibody comprises the V_{H} and V_{L} sequences in SEQ ID NOs: 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, and 165, and SEQ ID NO: 166, respectively, including post-translational modifications of those sequences.

In some aspects, provided herein are anti-IL18BP antibodies comprising a heavy chain variable domain (V_{H}) and a light chain variable domain (V_{L}), wherein the V_{H} and V_{L} are selected from the group consisting of: V_{H} comprising the amino acid sequence of SEQ ID NO: 134 and V_{L} comprising the amino acid sequence of SEQ ID NO: 166; V_{H} comprising the amino acid sequence of SEQ ID NO: 135 and V_{L} comprising the amino acid sequence of SEQ ID NO: 166; V_{H} comprising the amino acid sequence of SEQ ID NO: 136 and V_{L} comprising the amino acid sequence of SEQ ID NO: 166; V_{H} comprising the amino acid sequence of SEQ ID NO: 137 and V_{L} comprising the amino acid sequence of SEQ ID NO: 166; V_{H} comprising the amino acid sequence of SEQ ID NO: 138 and V_{L} comprising the amino acid sequence of SEQ ID NO: 166; V_{H} comprising the amino acid sequence of SEQ ID NO: 139 and V_{L} comprising the amino acid sequence of SEQ ID NO: 166; V_{H} comprising the amino acid sequence of SEQ ID NO: 140 and V_{L} comprising the amino acid sequence of SEQ ID NO: 166; V_{H} comprising the amino acid sequence of SEQ ID NO: 141 and V_{L} comprising the amino acid sequence of SEQ ID NO: 166; V_{H} comprising the amino acid sequence of SEQ ID NO: 142 and V_{L} comprising the amino acid sequence of SEQ ID NO: 166; V_{H} comprising the amino acid sequence of SEQ ID NO: 143 and V_{L} comprising the amino acid sequence of SEQ ID NO: 166; V_{H} comprising the amino acid sequence of SEQ ID NO: 144 and V_{L} comprising the amino acid sequence of SEQ ID NO: 166; V_{H} comprising the amino acid sequence of SEQ ID NO: 145 and V_{L} comprising the amino acid sequence of SEQ ID NO: 166; V_{H} comprising the amino acid sequence of SEQ ID NO: 146 and V_{L} comprising the amino acid sequence of SEQ ID NO: 166; V_{H} comprising the amino acid sequence of SEQ ID NO: 147 and V_{L} comprising the amino acid sequence of SEQ ID NO: 166; V_{H} comprising the amino acid sequence of SEQ ID NO: 148 and V_{L} comprising the amino acid sequence of SEQ ID NO: 166; V_{H} comprising the amino acid sequence of SEQ ID NO: 149 and V_{L} comprising the amino acid sequence of SEQ ID NO: 166; V_{H} comprising the amino acid sequence of SEQ ID NO: 150 and V_{L} comprising the amino acid sequence of SEQ ID NO: 166; V_{H} comprising the amino acid sequence of SEQ ID NO: 151 and V_{L} comprising the amino acid sequence of SEQ ID NO: 166; V_{H} comprising the amino acid sequence of SEQ ID NO: 152 and V_{L} comprising the amino acid sequence of SEQ ID NO: 166; V_{H} comprising the amino acid sequence of SEQ ID NO: 153 and V_{L} comprising the amino acid sequence of SEQ ID NO: 166; V_{H} comprising the amino acid sequence of SEQ ID NO: 154 and V_{L} comprising the amino acid sequence of SEQ ID NO: 166; V_{H} comprising the amino acid sequence of SEQ ID NO: 155 and V_{L} comprising the amino acid sequence of SEQ ID NO: 166; V_{H} comprising the amino acid sequence of SEQ ID NO: 156 and V_{L} comprising the amino acid sequence of SEQ ID NO: 166; V_{H} comprising the amino acid sequence of SEQ ID NO: 157 and V_{L} comprising the amino acid sequence of SEQ ID NO: 166; V_{H} comprising the amino acid sequence of SEQ ID NO: 158 and V_{L} comprising the amino acid sequence of SEQ ID NO: 166; V_{H} comprising the amino acid sequence of SEQ ID NO: 159 and V_{L} comprising the amino acid sequence of SEQ ID NO: 166; V_{H} comprising the amino acid sequence of SEQ ID NO: 160 and V_{L} comprising the amino acid sequence of SEQ ID NO: 166; V_{H} comprising the amino acid sequence of SEQ ID NO: 161 and V_{L} comprising the amino acid sequence of SEQ ID NO: 166; V_{H} comprising the amino acid sequence of SEQ ID NO: 162 and V_{L} comprising the amino acid sequence of SEQ ID NO: 166; V_{H} comprising the amino acid sequence of SEQ ID NO: 163 and V_{L} comprising the amino acid sequence of SEQ ID NO: 166; V_{H} comprising the amino acid sequence of SEQ ID NO: 164 and V_{L} comprising the amino acid sequence of SEQ ID NO: 166; and V_{H} comprising the amino acid sequence of SEQ ID NO: 165 and V_{L} comprising the amino acid sequence of SEQ ID NO: 166.

In some aspects, an anti-IL18BP antibody of the present disclosure competitively inhibits binding of at least one reference antibody selected from anti-IL18BP antibody BP-04.01, BP-04.02, BP-04.03, BP-04.04, BP-04.05, BP-04.06, BP-04.07, BP-04.08, BP-04.09, BP-04.10, BP-04.11, BP-04.12, BP-04.13, BP-04.14, BP-04.15, BP-04.16, BP-04.17, BP-04.18, BP-04.19, BP-04.20, BP-04.21, BP-04.22, BP-04.23, BP-04.24, BP-04.25, BP-04.26, BP-04.27, BP-04.28, BP-04.29, BP-04.30, BP-04.31, and BP-04.32, and any combination thereof, for binding to IL18BP.

In some aspects, an anti-IL18BP antibody of the present disclosure binds to an epitope of human IL18BP that is the same as or overlaps with the IL18BP epitope bound by at least one reference antibody selected from anti-IL18BP antibody BP-04.01, BP-04.02, BP-04.03, BP-04.04, BP-04.05, BP-04.06, BP-04.07, BP-04.08, BP-04.09, BP-04.10, BP-04.11, BP-04.12, BP-04.13, BP-04.14, BP-04.15, BP-04.16, BP-04.17, BP-04.18, BP-04.19, BP-04.20, BP-04.21, BP-04.22, BP-04.23, BP-04.24, BP-04.25, BP-04.26, BP-04.27, BP-04.28, BP-04.29, BP-04.30, BP-04.31, and BP-04.32. Detailed exemplary methods for mapping an epitope to which an antibody binds are provided in Morris (1996) “Epitopc Mapping Protocols.” in Methods in Molecular Biology vol. 66 (Humana Press. Totowa, NJ).

In some aspects, an anti-IL18BP antibody of the present disclosure competitively inhibits binding of at least one reference antibody, or binds to an epitope of human IL18BP that is the same as or overlaps with the IL18BP epitope bound by at least one reference antibody, wherein the reference antibody is an anti-IL18BP antibody comprising a heavy chain variable domain (V_{H}) and a light chain variable domain (V_{L}), wherein the V_{H} and V_{L} are selected from the group consisting of: V_{H} comprising the amino acid sequence of SEQ ID NO: 134 and V_{L} comprising the amino acid sequence of SEQ ID NO: 166; V_{H} comprising the amino acid sequence of SEQ ID NO: 135 and V_{L} comprising the amino acid sequence of SEQ ID NO: 166; V_{H} comprising the amino acid sequence of SEQ ID NO: 136 and V_{L} comprising the amino acid sequence of SEQ ID NO: 166; V_{H} comprising the amino acid sequence of SEQ ID NO: 137 and V_{L} comprising the amino acid sequence of SEQ ID NO: 166; V_{H} comprising the amino acid sequence of SEQ ID NO: 138 and V_{L} comprising the amino acid sequence of SEQ ID NO: 166; V_{H} comprising the amino acid sequence of SEQ ID NO: 139 and V_{L} comprising the amino acid sequence of SEQ ID NO: 166; V_{H} comprising the amino acid sequence of SEQ ID NO: 140 and V_{L} comprising the amino acid sequence of SEQ ID NO: 166; V_{H} comprising the amino acid sequence of SEQ ID NO: 141 and V_{L} comprising the amino acid sequence of SEQ ID NO: 166; V_{H} comprising the amino acid sequence of SEQ ID NO: 142 and V_{L} comprising the amino acid sequence of SEQ ID NO: 166; V_{H} comprising the amino acid sequence of SEQ ID NO: 143 and V_{L} comprising the amino acid sequence of SEQ ID NO: 166; V_{H} comprising the amino acid sequence of SEQ ID NO: 144 and V_{L} comprising the amino acid sequence of SEQ ID NO: 166; V_{H} comprising the amino acid sequence of SEQ ID NO: 145 and V_{L} comprising the amino acid sequence of SEQ ID NO: 166; V_{H} comprising the amino acid sequence of SEQ ID NO: 146 and V_{L} comprising the amino acid sequence of SEQ ID NO: 166; V_{H} comprising the amino acid sequence of SEQ ID NO: 147 and V_{L} comprising the amino acid sequence of SEQ ID NO: 166; V_{H} comprising the amino acid sequence of SEQ ID NO: 148 and V_{L} comprising the amino acid sequence of SEQ ID NO: 166; V_{H} comprising the amino acid sequence of SEQ ID NO: 149 and V_{L} comprising the amino acid sequence of SEQ ID NO: 166; V_{H} comprising the amino acid sequence of SEQ ID NO: 150 and V_{L} comprising the amino acid sequence of SEQ ID NO: 166; V_{H} comprising the amino acid sequence of SEQ ID NO: 151 and V_{L} comprising the amino acid sequence of SEQ ID NO: 166; V_{H} comprising the amino acid sequence of SEQ ID NO: 152 and V_{L} comprising the amino acid sequence of SEQ ID NO: 166; V_{H} comprising the amino acid sequence of SEQ ID NO: 153 and V_{L} comprising the amino acid sequence of SEQ ID NO: 166; V_{H} comprising the amino acid sequence of SEQ ID NO: 154 and V_{L} comprising the amino acid sequence of SEQ ID NO: 166; V_{H} comprising the amino acid sequence of SEQ ID NO: 155 and V_{L} comprising the amino acid sequence of SEQ ID NO: 166; V_{H} comprising the amino acid sequence of SEQ ID NO: 156 and V_{L} comprising the amino acid sequence of SEQ ID NO: 166; V_{H} comprising the amino acid sequence of SEQ ID NO: 157 and V_{L} comprising the amino acid sequence of SEQ ID NO: 166; V_{H} comprising the amino acid sequence of SEQ ID NO: 158 and V_{L} comprising the amino acid sequence of SEQ ID NO: 166; V_{H} comprising the amino acid sequence of SEQ ID NO: 159 and V_{L} comprising the amino acid sequence of SEQ ID NO: 166; V_{H} comprising the amino acid sequence of SEQ ID NO: 160 and V_{L} comprising the amino acid sequence of SEQ ID NO: 166; V_{H} comprising the amino acid sequence of SEQ ID NO: 161 and V_{L} comprising the amino acid sequence of SEQ ID NO: 166; V_{H} comprising the amino acid sequence of SEQ ID NO: 162 and V_{L} comprising the amino acid sequence of SEQ ID NO: 166; V_{H} comprising the amino acid sequence of SEQ ID NO: 163 and V_{L} comprising the amino acid sequence of SEQ ID NO: 166; V_{H} comprising the amino acid sequence of SEQ ID NO: 164 and V_{L} comprising the amino acid sequence of SEQ ID NO: 166; and V_{H} comprising the amino acid sequence of SEQ ID NO: 165 and V_{L} comprising the amino acid sequence of SEQ ID NO: 166.

In some aspects, provided herein is an isolated anti-IL18BP antibody, wherein the antibody comprises a heavy chain and a light chain, wherein the heavy chain comprises the amino acid sequence of SEQ ID NO: 190 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO: 191 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO: 192 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO: 193 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO: 194 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO: 195 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO: 196 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO: 197 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO: 198 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO: 199 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:200 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:201 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:202 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:203 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:204 and the light chain comprises the amino acid sequence of SEQ ID NO: 189.

In some aspects, provided herein is an isolated anti-IL18BP antibody, wherein the antibody comprises a heavy chain and a light chain, wherein the heavy chain comprises the amino acid sequence of SEQ ID NO:205 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:206 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:207 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:208 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:209 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:210 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:211 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:212 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:213 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:214 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:215 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:216 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:217 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:218 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:219 and the light chain comprises the amino acid sequence of SEQ ID NO: 189.

In some aspects, provided herein is an isolated anti-IL18BP antibody, wherein the antibody comprises a heavy chain and a light chain, wherein the heavy chain comprises the amino acid sequence of SEQ ID NO:220 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:221 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:222 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:223 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:224 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:225 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:226 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:227 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:228 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:229 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:230 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:231 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:232 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:233 and the light chain comprises the amino acid sequence of SEQ ID NO: 189. In some aspects, provided herein is an isolated anti-IL18BP antibody, wherein the antibody comprises a heavy chain and a light chain, wherein the heavy chain comprises the amino acid sequence of SEQ ID NO:234 and the light chain comprises the amino acid sequence of SEQ ID NO: 189.

In some aspects, the anti-IL18BP antibody according to any of the above aspects is a monoclonal antibody, including a humanized and/or human antibody. In some aspects, the anti-IL18BP antibody is an antibody fragment, e.g., a Fv, Fab, Fab′, scFv, diabody, or F(ab′)2 fragment. In some aspects, the anti-IL18BP antibody is a substantially full-length antibody, e.g., an IgG1 antibody, IgG2a antibody or other antibody class or isotype as defined herein.

In some aspects, an anti-IL18BP antibody according to any of the above aspects may incorporate any of the features, singly or in combination, as described below.

### Anti-IL18BP Antibody Binding Affinity

In some embodiments of any of the antibodies provided herein, the antibody has a dissociation constant (K_{D}) of <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M). Dissociation constants may be determined through any analytical technique, including any biochemical or biophysical technique such as ELISA, surface plasmon resonance (SPR), bio-layer interferometry (see, e.g., Octet System by ForteBio), isothermal titration calorimetry (ITC), differential scanning calorimetry (DSC), circular dichroism (CD), stopped-flow analysis, and colorimetric or fluorescent protein melting analyses. In one embodiment, Kd is measured by a radiolabeled antigen binding assay (RIA). In some embodiment, an RIA is performed with the Fab version of an antibody of interest and its antigen, for example as described in Chen et al. J. Mol. Biol. 293:865-881 (1999)). In some embodiments, K_{D} is measured using a BIACORE surface plasmon resonance assay, for example, an assay using a BIACORE-2000 or a BIACORE-3000 (BIAcore, Inc., Piscataway, NJ) is performed at 25° C. with immobilized antigen CM5 chips at ˜10 response units (RU). In some embodiments, the K_{D} is determined using a monovalent antibody (e.g., a Fab) or a full-length antibody. In some embodiments, the K_{D} is determined using a full-length antibody in a monovalent form.

### Antibody Fragments

In some embodiments of any of the antibodies provided herein, the antibody is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP404097; WO 1993/01161; Hudson et al. Nat. Med. 9:129-134 (2003). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### Chimeric and Humanized Antibodies

In some embodiments of any of the antibodies provided herein, the antibody is a chimeric antibody. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments of any of the antibodies provided herein, the antibody is a humanized antibody. Typically, a non-human antibody is humanized to reduce immunogenicity to humans, while retaining the specificity and affinity of the parental non-human antibody. In certain embodiments, a humanized antibody is substantially non-immunogenic in humans. In certain embodiments, a humanized antibody has substantially the same affinity for a target as an antibody from another species from which the humanized antibody is derived. See, e.g., U.S. Pat. No. 5,530,101, 5,693,761, 5,693,762; and 5,585,089. In certain embodiments, amino acids of an antibody variable domain that can be modified without diminishing the native affinity of the antigen-binding domain while reducing its immunogenicity are identified. See, e.g., U.S. Pat. Nos. 5,766,886 and 5,869,619. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:161 9-1633 (2008), and are further described, e.g., in U.S. Pat. Nos. 5,821,337, 7,527,791, 6,982,321, and 7,087,409. Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA 89:4285 (1992); and Presta et al., J. Immunol. 151:2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al. J. Biol. Chem. 272:10678-10684 (1997) and Rosok et al. J. Biol. Chem. 271:22611-22618 (1996)).

### Human Antibodies

In some embodiments of any of the antibodies provided herein, the antibody is a human antibody. Human antibodies can be produced using various techniques known in the art. Human antibodies are described generally in van Dijk et al. Curr. Opin. Pharmacol. 5:368-74 (2001) and Lonberg Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human MAbs with the desired specificity can be produced and selected. Certain exemplary methods are described in U.S. Pat. No. 5,545,807, EP 546073, and EP 546073. See also, for example·U.S. Pat. Nos. 6,075,181 and 6,150,584 describing XENOMOUSE™ technology: U.S. Pat. No. 5,770,429 describing HUMAB® technology: U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001 (1984) and Boerner et al. J. Immunol. 147:86 (1991)). Human antibodies generated via human B-cell hybridoma technology are also described in Li et al. Proc. Natl. Acad. Sci. USA. 1 03:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al. Histology and Histopathology 20 (3): 927-937 (2005) and Vollmers et al. Methods and Findings in Experimental and Clinical Pharmacology 27 (3): 185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

In some embodiments of any of the antibodies provided herein, the antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed) ribosome display (CAT), yeast display (Adimab), and the like. In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol. 12:433-455 (1994). For example, a variety of methods are known in the art for generating phage display libraries and screening such libraries for antibodies possessing the desired binding characteristics. See also Sidhu et al. J. Mol. Biol. 338 (2): 299-310, 2004; Lee et al. J. Mol. Biol. 340 (5): 1073-1093, 2004; Fellouse Proc. Natl. Acad. Sci. USA 101 (34): 12467-12472 (2004); and Lee et al. J. Immunol. Methods 284 (−2): 1 19-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. EMBO J. 12:725-734 (1993). Finally, naive libraries can also be made synthetically by cloning unrearranged V-gene segments from stem cells, and using PCR primers comprising random sequence to encode the highly variable HVR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and US Patent Publication Nos. 2007/0292936 and 2009/0002360. Antibodies isolated from human antibody libraries are considered human antibodies or human antibody fragments herein.

### Constant Regions Including Fc Regions

In some embodiments of any of the antibodies provided herein, the antibody comprises an Fc. In some embodiments, the Fc is a human IgG1, IgG2, IgG3, and/or IgG4 isotype. In some embodiments, the antibody is of the IgG class, the IgM class, or the IgA class.

In certain embodiments of any of the antibodies provided herein, the antibody has an IgG2 isotype. In some embodiments, the antibody contains a human IgG2 constant region. In some embodiments, the human IgG2 constant region includes an Fc region. In some embodiments, the antibody induces the one or more IL18BP activities or independently of binding to an Fc receptor. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG4 isotype. In some embodiments, the antibody contains a human IgG4 constant region. In some embodiments, the human IgG4 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has a hybrid IgG2/4 isotype. In some embodiments, the antibody includes an amino acid sequence comprising amino acids 118 to 260 according to EU numbering of human IgG2 and amino acids 261-447 according to EU numbering of human IgG4 (WO 1997/11971: WO 2007/106585).

In some embodiments, the Fc region increases clustering without activating complement as compared to a corresponding antibody comprising an Fc region that does not comprise the amino acid substitutions. In some embodiments, the antibody induces one or more activities of a target specifically bound by the antibody. In some embodiments, the antibody binds to IL18BP.

It may also be desirable to modify an anti-IL18BP antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce Antibody-dependent cell-mediated cytotoxicity. In some embodiments, the effector function is impaired by removing N-glycosylation of the Fc region (e.g., in the CH2 domain of IgG) of the antibody. In some embodiments, the effector function is impaired by modifying regions such as 233-236, 297, and/or 327-331 of human IgG as described in WO 99/58572 and Armour et al. Molecular Immunology 40:585-593 (2003); Reddy et al. J. Immunology 164:1925-1933 (2000). In other embodiments, it may also be desirable to modify an anti-IL18BP antibody of the present disclosure to modify effector function to increase finding selectivity toward the ITIM-containing FcgRIIb (CD32b) to increase clustering of IL18BP antibodies on adjacent cells without activating humoral responses including Antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Other amino acid sequence modifications.

### Antibody Variants

In some embodiments of any of the antibodies provided herein, amino acid sequence variants of the antibodies are contemplated. For example, it may be desirable to improve the binding affinity and/or other biological properties of the antibody.

### Substitution, Insertion, and Deletion Variants

In some embodiments of any of the antibodies provided herein, antibody variants having one or more amino acid substitutions are provided. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody.

TABLE A
Amino Acid Substitutions

| Original | | Preferred |
|---|---|---|
| Residue | Exemplary Substitutions | Substitutions |
| Ala (A) | Val; Leu; Ile | Va |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the polypeptide or antibody described herein, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al. J. Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One can also identify epitopes from primary amino acid sequences on the basis of hydrophilicity. These regions are also referred to as “epitopic core regions”.

In certain embodiments of the variant VH and VL sequences provided above, each HVR is unaltered.

Amino acid sequence insertions include amino- and/or carboxyl-terminal fusions ranging in length from one residue to polypeptides comprising a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Any cysteine residue outside the HVRs and not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

### Glycosylation Variants

In some embodiments of any of the antibodies provided herein, the antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. Native antibodies produced by mammalian cells typically comprise a branched, biantennary oligosaccharide that is generally attached by an N-linkage to Asn297 according to Kabat numbering of the CH2 domain of the Fc region. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the disclosure may be made in order to create antibody variants with certain improved properties.

In one embodiment, antibody variants are provided having a carbohydrate structure that lacks fucose attached (directly or indirectly) to an Fc region. See, e.g., US Patent Publication Nos. 2003/0157108 and 2004/0093621. Examples of publications related to “defucosylated” or “fucose-deficient”antibody variants include: US 2003/0157108; US 2003/0115614; US 2002/0164328; US 2004/0093621; US 2004/0132140; US 2004/0110704; US 2004/0110282; US 2004/0109865; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Led 3 CHO cells deficient in protein fucosylation (Ripka et al. Arch. Biochem. Biophys. 249:533-545 (1986); US 2003/0157108), and knockout cell lines, such as alpha-1,6-fucosyltransferase gene. FUT8, knockout CHO cells (see, e.g., Yamane-Ohnuki et al. Biotech. Bioeng. 87:614 (2004) and Kanda et al. Biotechnol. Bioeng. 94 (4): 680-688 (2006)).

### Modified Constant Regions

In some embodiments of any of the antibodies provided herein, the antibody Fc is an antibody. Fc isotypes and/or modifications. In some embodiments, the antibody Fc isotype and/or modification is capable of binding to Fc gamma receptor.

In some embodiments of any of the antibodies provided herein, the modified antibody Fc is an IgG1 modified Fc. In some embodiments, the IgG1 modified Fc comprises one or more modifications. For example, in some embodiments, the IgG1 modified Fc comprises one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA. 92:11980-11984; Alegre et al., (1994) Transplantation 57:1537-1543. 31; Xu et al., (2000) Cell Immunol. 200:16-26). G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood. 109:1185-1192), P331S (Sazinsky et al., (2008) Proc Natl Acad Sci USA 2008. 105:20167-20172), S267E, L328F, A330L, M252Y, S254T, and/or T256E, where the amino acid position is according to the EU numbering convention.

In some embodiments of any of the IgG1 modified Fc, the Fc comprises N297A mutation according to EU numbering. In some embodiments of any of the IgG1 modified Fc, the Fc comprises D265A and N297A mutations according to EU numbering. In some embodiments of any of the IgG1 modified Fc, the Fc comprises D270A mutations according to EU numbering. In some embodiments, the IgG1 modified Fc comprises L234A and L235A mutations according to EU numbering. In some embodiments of any of the IgG1 modified Fc, the Fc comprises L234A and G237A mutations according to EU numbering. In some embodiments of any of the IgG1 modified Fc, the Fc comprises L234A, L235A and G237A mutations according to EU numbering. In some embodiments of any of the IgG1 modified Fc, the Fc comprises one or more (including all) of P238D, L328E, E233, G237D, H268D, P271G and A330R mutations according to EU numbering. In some embodiments of any of the IgG1 modified Fc, the Fc comprises one or more of S267E/L328F mutations according to EU numbering. In some embodiments of any of the IgG1 modified Fc, the Fc comprises P238D, L328E, E233D, G237D, H268D, P271G and A330R mutations according to EU numbering. In some embodiments of any of the IgG1 modified Fc, the Fc comprises P238D, L328E, G237D, H268D, P271G and A330R mutations according to EU numbering. In some embodiments of any of the IgG1 modified Fc, the Fc comprises P238D, S267E, L328E, E233D, G237D, H268D, P271G and A330R mutations according to EU numbering. In some embodiments of any of the IgG1 modified Fc, the Fc comprises P238D, S267E, L328E, G237D, H268D, P271G and A330R mutations according to EU numbering. In some embodiments of any of the IgG1 modified Fc, the Fc comprises C226S, C229S, E233P, L234V, and L235A mutations according to EU numbering. In some embodiments of any of the IgG1 modified Fc, the Fc comprises L234F, L235E, and P331S mutations according to EU numbering. In some embodiments of any of the IgG1 modified Fc, the Fc comprises S267E and L328F mutations according to EU numbering. In some embodiments of any of the IgG1 modified Fc, the Fc comprises N325S and L328F mutations according to EU numbering. In some embodiments of any of the IgG1 modified Fc, the Fc comprises K322A mutation according to EU numbering. In some embodiments of any of the IgG1 modified Fc, the Fc comprises P331S mutation according to EU numbering. In some embodiments of any of the IgG1 modified Fc, the Fc comprises S267E mutations according to EU numbering. In some embodiments of any of the IgG1 modified Fc, the Fc comprises a substitute of the constant heavy 1 (CH1) and hinge region of IgG1 with CH1 and hinge region of IgG2 (amino acids 118-230 of IgG2 according to EU numbering) with a Kappa light chain.

In some embodiments of any of the IgG1 modified Fc, the Fc includes two or more amino acid substitutions that increase antibody clustering without activating complement as compared to a corresponding antibody having an Fc region that does not include the two or more amino acid substitutions. Accordingly, in some embodiments of any of the IgG1 modified Fc, the IgG1 modified Fc is an antibody comprising an Fc region, where the antibody comprises an amino acid substitution at position E430G and one or more amino acid substitutions in the Fc region at a residue position selected from: L234F, L235A, L235E, S267E, K322A, L328F, A330S, P331S, P331G, and any combination thereof according to EU numbering. In some embodiments, the IgG1 modified Fc comprises an amino acid substitution at positions E430G, L243A, L235A, and P331S according to EU numbering. In some embodiments, the IgG1 modified Fc comprises an amino acid substitution at positions E430G and P331S according to EU numbering. In some embodiments, the IgG1 modified Fc comprises an amino acid substitution at positions E430G and K322A according to EU numbering. In some embodiments, the IgG1 modified Fc comprises an amino acid substitution at positions E430G, A330S, and P331S according to EU numbering. In some embodiments, the IgG1 modified Fc comprises an amino acid substitution at positions E430G, K322A, A330S, and P331S according to EU numbering. In some embodiments, the IgG1 modified Fc comprises an amino acid substitution at positions E430G, K322A, and A330S according to EU numbering. In some embodiments, the IgG1 modified Fc comprises an amino acid substitution at positions E430G, K322A, and P331S according to EU numbering. In some embodiments, the IgG1 modified Fc comprises an amino acid substitution at positions L234A, L235A, and P331S according to EU numbering. In some embodiments, the IgG1 modified Fc comprises an amino acid substitution at positions L234A, L235A, and P331G according to EU numbering. In some embodiments, the IgG1 modified Fc comprises an amino acid substitution at positions L234A, L235A, and P329S according to EU numbering. In some embodiments, the IgG1 modified Fc comprises an amino acid substitution at positions L234A, L235A, and P329G according to EU numbering. In some embodiments, the IgG1 modified Fc comprises an amino acid substitution at positions P331S and E430G according to EU numbering. In some embodiments, the IgG1 modified Fc comprises an amino acid substitution at positions N325S and L328F according to EU numbering. In some embodiments, the IgG1 modified Fc comprises an amino acid substitution at positions S267E and L328F according to EU numbering.

In some embodiments of any of the IgG1 modified Fc, the IgG1 modified Fc may further comprise herein may be combined with an A330L mutation (Lazar et al. Proc Natl Acad Sci USA. 103:4005-4010 (2006)), or one or more of L234F, L235E, and/or P331S mutations (Sazinsky et al, Proc Natl Acad Sci USA. 105:20167-20172 (2008)), according to the EU numbering convention, to eliminate complement activation. In some embodiments of any of the IgG1 modified Fc, the IgG1 modified Fc may further comprise one or more of A330L, A330S, L234F, L235E, and/or P331S according to EU numbering. In some embodiments of any of the IgG1 modified Fc, the IgG1 modified Fc may further comprise one or more mutations to enhance the antibody half-life in human serum (e.g., one or more (including all) of M252Y, S254T, and T256E mutations according to the EU numbering convention). In some embodiments of any of the IgG1 modified Fc, the IgG1 modified Fc may further comprise one or more of E430G, E430S, E430F, E430T, E345K, E345Q, E345R, E345Y, S440Y, and/or S440W according to EU numbering.

Other aspects of the present disclosure relate to antibodies having modified constant regions (i.e., Fc regions). An antibody dependent on binding to FcgR receptor to activate targeted receptors may lose its agonist activity if engineered to eliminate FcgR binding (see, e.g., Wilson et al. Cancer Cell 19:101-113 (2011); Armour at al. Immunology 40:585-593 (2003); and White et al. Cancer Cell 27:138-148 (2015)). As such, it is thought that an anti-IL18BP antibody of the present disclosure with the correct epitope specificity can activate the target antigen, with minimal adverse effects, when the antibody has an Fc domain from a human IgG2 isotype (CH1 and hinge region) or another type of Fc domain that is capable of preferentially binding the inhibitory FcgRIIB r receptors, or a variation thereof.

In some embodiments of any of the antibodies provided herein, the modified antibody Fc is an IgG2 modified Fc. In some embodiments, the IgG2 modified Fc comprises one or more modifications. For example, in some embodiments, the IgG2 modified Fc comprises one or more amino acid substitutions (e.g., relative to a wild-type Fc region of the same isotype). In some embodiments of any of the IgG2 modified Fc, the one or more amino acid substitutions are selected from V234A (Alegre et al. Transplantation 57:1537-1543 (1994); Xu et al. Cell Immunol. 200:16-26 (2000)); G237A (Cole et al. Transplantation. 68:563-571 (1999)); H268Q, V309L, A330S, P331S (US 2007/0148167; Armour et al. Eur J Immunol 29:2613-2624 (1999); Armour et al. The Haematology Journal 1 (Suppl. 1); 27 (2000); Armour et al. The Haematology Journal 1 (Suppl.1); 27 (2000)), C219S, and/or C220S (White et al. Cancer Cell 27, 138-148 (2015)); S267E, L328F (Chu et al. Mol Immunol. 45:3926-3933 (2008)); and M252Y, S254T, and/or T256E according to the EU numbering convention. In some embodiments of any of the IgG2 modified Fc, the Fc comprises an amino acid substitution at positions V234A and G237A according to EU numbering. In some embodiments of any of the IgG2 modified Fc, the Fc comprises an amino acid substitution at positions C219S or C220S according to EU numbering. In some embodiments of any of the IgG2 modified Fc, the Fc comprises an amino acid substitution at positions A330S and P331S according to EU numbering. In some embodiments of any of the IgG2 modified Fc, the Fc comprises an amino acid substitution at positions S267E and L328F according to EU numbering.

In some embodiments of any of the IgG2 modified Fc, the Fc comprises a C127S amino acid substitution according to the EU numbering convention (White et al., (2015) Cancer Cell 27, 138-148; Lightle et al. Protein Sci. 19:753-762 (2010); and WO 2008/079246). In some embodiments of any of the IgG2 modified Fc, the antibody has an IgG2 isotype with a Kappa light chain constant domain that comprises a C214S amino acid substitution according to the EU numbering convention (White et al. Cancer Cell 27:138-148 (2015); Lightle et al. Protein Sci. 19:753-762 (2010); and WO 2008/079246).

In some embodiments of any of the IgG2 modified Fc, the Fc comprises a C220S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc comprises a C219S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc includes an IgG2 isotype heavy chain constant domain 1 (CH1) and hinge region (White et al. Cancer Cell 27:138-148 (2015)). In certain embodiments of any of the IgG2 modified Fc, the IgG2 isotype CH1 and hinge region comprise the amino acid sequence of 118-230 according to EU numbering. In some embodiments of any of the IgG2 modified Fc, the antibody Fc region comprises a S267E amino acid substitution, a L328F amino acid substitution, or both, and/or a N297A or N297Q amino acid substitution according to the EU numbering convention.

In some embodiments of any of the IgG2 modified Fc, the Fc further comprises one or more amino acid substitution at positions E430G, E430S, E430F, E430T, E345K, E345Q, E345R, E345Y, S440Y, and S440W according to EU numbering. In some embodiments of any of the IgG2 modified Fc, the Fc may further comprise one or more mutations to enhance the antibody half-life in human scrum (e.g., one or more (including all) of M252Y, S254T, and T256E mutations according to the EU numbering convention). In some embodiments of any of the IgG2 modified Fc, the Fc may further comprise A330S and P331S.

In some embodiments of any of the IgG2 modified Fc, the Fc is an IgG2/4 hybrid Fc. In some embodiments, the IgG2/4 hybrid Fc comprises IgG2 aa 118 to 260 and IgG4 aa 261 to 447. In some embodiments of any IgG2 modified Fc, the Fc comprises one or more amino acid substitutions at positions H268Q, V309L, A330S, and P331S according to EU numbering.

In some embodiments of any of the IgG1 and/or IgG2 modified Fc, the Fc comprises one or more additional amino acid substitutions selected from A330L, L234F; L235E, or P331S according to EU numbering; and any combination thereof.

In certain embodiments of any of the IgG1 and/or IgG2 modified Fc, the Fc comprises one or more amino acid substitutions at a residue position selected from C127S, L234A, L234F, L235A, L235E, S267E, K322A, L328F, A330S, P331S, E345R, E430G, S440Y, and any combination thereof according to EU numbering. In some embodiments of any of the IgG1 and/or IgG2 modified Fc, the Fc comprises an amino acid substitution at positions E430G, L243A, L235A, and P331S according to EU numbering. In some embodiments of any of the IgG1 and/or IgG2 modified Fc, the Fc comprises an amino acid substitution at positions E430G and P331S according to EU numbering. In some embodiments of any of the IgG1 and/or IgG2 modified Fc, the Fc comprises an amino acid substitution at positions E430G and K322A according to EU numbering. In some embodiments of any of the IgG1 and/or IgG2 modified Fc, the Fc comprises an amino acid substitution at positions E430G, A330S, and P331S according to EU numbering. In some embodiments of any of the IgG1 and/or IgG2 modified Fc, the Fc comprises an amino acid substitution at positions E430G, K322A, A330S, and P331S according to EU numbering. In some embodiments of any of the IgG1 and/or IgG2 modified Fc, the Fc comprises an amino acid substitution at positions E430G, K322A, and A330S according to EU numbering. In some embodiments of any of the IgG1 and/or IgG2 modified Fc, the Fc comprises an amino acid substitution at positions E430G, K322A, and P331S according to EU numbering. In some embodiments of any of the IgG1 and/or IgG2 modified Fc, the Fc comprises an amino acid substitution at positions S267E and L328F according to EU numbering. In some embodiments of any of the IgG1 and/or IgG2 modified Fc, the Fc comprises an amino acid substitution at position C127S according to EU numbering. In some embodiments of any of the IgG1 and/or IgG2 modified Fc, the Fc comprises an amino acid substitution at positions E345R, E430G and S440Y according to EU numbering.

In some embodiments of any of the antibodies provided herein, the modified antibody Fc is an IgG4 modified Fc. In some embodiments, the IgG4 modified Fc comprises one or more modifications. For example, in some embodiments, the IgG4 modified Fc comprises one or more amino acid substitutions (e.g., relative to a wild-type Fc region of the same isotype). In some embodiments of any of the IgG4 modified Fc, the one or more amino acid substitutions are selected from L235A, G237A, S229P, L236E (Reddy et al. J Immunol 164:1925-1933 (2000)), S267E, E318A, L328F, M252Y, S254T, and/or T256E according to the EU numbering convention. In some embodiments of any of the IgG4 modified Fc, the Fc may further comprise L235A, G237A, and E318A according to the EU numbering convention. In some embodiments of any of the IgG4 modified Fc, the Fc may further comprise S228P and L235E according to the EU numbering convention. In some embodiments of any of the IgG4 modified Fc, the IgG4 modified Fc may further comprise S267E and L328F according to the EU numbering convention.

In some embodiments of any of the IgG4 modified Fc, the IgG4 modified Fc comprises an S228P substitution or may be combined with an S228P mutation according to the EU numbering convention (Angal et al. Mol Immunol. 30:105-108 (1993)) and/or with one or more mutations described in (Peters et al. J Biol Chem. 287 (29): 24525-33 (2012)) to enhance antibody stabilization.

In some embodiments of any of the IgG4 modified Fc, the IgG4 modified Fc may further comprise one or more mutations to enhance the antibody half-life in human serum (e.g., one or more (including all) of M252Y, S254T, and T256E mutations according to the EU numbering convention).

In some embodiments of any of the IgG4 modified Fc, the Fc comprises L235E according to EU numbering. In certain embodiments of any of the IgG4 modified Fc, the Fc comprises one or more amino acid substitutions at a residue position selected from C127S, F234A, L235A, L235E, S267E, K322A, L328F, E345R, E430G, S440Y, and any combination thereof, according to EU numbering. In some embodiments of any of the IgG4 modified Fc, the Fc comprises an amino acid substitution at positions E430G, L243A, L235A, and P331S according to EU numbering. In some embodiments of any of the IgG4 modified Fc, the Fc comprises an amino acid substitution at positions E430G and P331S according to EU numbering. In some embodiments of any of the IgG4 modified Fc, the Fc comprises an amino acid substitution at positions E430G and K322A according to EU numbering. In some embodiments of any of the IgG4 modified Fc, the Fc comprises an amino acid substitution at position E430 according to EU numbering. In some embodiments of any of the IgG4 modified Fc, the Fc region comprises an amino acid substitution at positions E430G and K322A according to EU numbering. In some embodiments of any of the IgG4 modified Fc, the Fc comprises an amino acid substitution at positions S267E and L328F according to EU numbering. In some embodiments of any of the IgG4 modified Fc, the Fc comprises an amino acid substitution at position C127S according to EU numbering. In some embodiments of any of the IgG4 modified Fc, the Fc comprises an amino acid substitution at positions E345R, E430G and S440Y according to EU numbering.

### Other Antibody Modifications

In some embodiments of any of the antibodies, the antibody is a derivative. The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen-binding protein can have a greater circulating half-life than an antigen-binding protein that is not chemically modified. In certain embodiments, a chemically modified antigen-binding protein can have improved targeting capacity for desired cells, tissues, and/or organs. In some embodiments, a derivative antigen-binding protein is covalently modified to include one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative antigen-binding protein comprises one or more polymer, including, but not limited to, monomethoxy-polyethylene glycol, dextran, cellulose, copolymers of ethylene glycol/propylene glycol, carboxymethylcellulose, polyvinyl pyrrolidone, poly-1,3-dioxolane, poly-1,3,6-trioxane, ethylene/maleic anhydride copolymer, polyaminoacids (either homopolymers or random copolymers), poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers.

In certain embodiments, a derivative is covalently modified with polyethylene glycol (PEG) subunits. In certain embodiments, one or more water-soluble polymer is bonded at one or more specific position, for example at the amino terminus, of a derivative. In certain embodiments, one or more water-soluble polymer is randomly attached to one or more side chains of a derivative. In certain embodiments. PEG is used to improve the therapeutic capacity for an antigen-binding protein. In certain embodiments. PEG is used to improve the therapeutic capacity for a humanized antibody. Certain such methods are discussed, for example, in U.S. Pat. No. 6,133,426, which is hereby incorporated by reference for any purpose.

Peptide analogs are commonly used in the pharmaceutical industry as non-peptide drugs with properties analogous to those of the template peptide. These types of non-peptide compound are termed “peptide mimetics” or “peptidomimetics.” Fauchere. J. Adv. Drug Res., 15:29 (1986); and Evans et al. J. Med. Chem., 30: 1229 (1987), which are incorporated herein by reference for any purpose. Such compounds are often developed with the aid of computerized molecular modeling. Peptide mimetics that are structurally similar to therapeutically useful peptides can be used to produce a similar therapeutic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) can be used in certain embodiments to generate more stable peptides. In addition, constrained peptides comprising a consensus sequence or a substantially identical consensus sequence variation can be generated by methods known in the art (Rizo and Gicrasch Ann. Rev. Biochem., 61:387 (1992), incorporated herein by reference for any purpose); for example, by adding internal cysteine residues capable of forming intramolecular disulfide bridges which cyclize the peptide.

Drug conjugation involves coupling of a biological active cytotoxic (anticancer) payload or drug to an antibody that specifically targets a certain tumor marker (e.g. a polypeptide that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue OncLive Jul. 5, 2012: ADC Review on antibody-drug conjugates; and Ducry et al. Bioconjugate Chemistry 21 (1): 5-13 (2010).

### Nucleic Acids, Vectors, and Host Cells

Anti-IL18BP antibodies of the present disclosure may be produced using recombinant methods and compositions, e.g., as described in U.S. Pat. No. 4,816,567. In some embodiments, isolated nucleic acids having a nucleotide sequence encoding any of the anti-IL18BP antibodies of the present disclosure are provided. Such nucleic acids may encode an amino acid sequence comprising the V_{L} and/or an amino acid sequence comprising the V_{H} of the anti-IL18BP antibody (e.g., the light and/or heavy chains of the antibody). In some embodiments, one or more vectors (e.g., expression vectors) comprising such nucleic acids are provided. In some embodiments, a host cell comprising such nucleic acid is also provided. In some embodiments, the host cell comprises (e.g., has been transduced with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody.

In some embodiments, the host cell comprises (e.g., has been transduced with): (1) a nucleic acid that encodes an amino acid sequence comprising a light chain of an antibody, wherein the light chain comprises a V_{L} and (2) a nucleic acid that encodes an amino acid sequence comprising a heavy chain of an antibody, wherein the heavy chain comprises a V_{H}, wherein the VL and the VH form an antigen-binding domain that binds to IL18BP In some embodiments, the host cell comprises (e.g., has been transduced with): (1) a nucleic acid that encodes an amino acid sequence comprising a light chain of an antibody, wherein the light chain comprises a V_{L}, (2) a nucleic acid that encodes an amino acid sequence comprising a heavy chain of an antibody, wherein the heavy chain comprises a V_{H}, and (3) a nucleic acid that encodes a fragment of a heavy chain, wherein the heavy chain not comprise a V_{H} (e.g., a fragment of a heavy chain comprising a CH2 and a CH3 domain), wherein the V_{L} and the V_{H} form an antigen-binding domain that binds to IL18BP. The nucleic acids can be within the same vector or can be in different vectors.

In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-IL18BP antibody of the present disclosure are provided. In some embodiments, the method includes culturing a host cell of the present disclosure comprising a nucleic acid encoding the anti-IL18BP antibody, under conditions suitable for expression of the antibody. In some embodiments, the antibody is subsequently recovered from the host cell (or host cell culture medium).

For recombinant production of an anti-IL18BP antibody of the present disclosure, a nucleic acid encoding the anti-IL18BP antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors comprising a nucleic acid sequence encoding any of the anti-IL18BP antibodies of the present disclosure, or cell-surface expressed fragments or polypeptides thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones comprising the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad. Strategene, and Invitrogen.

Suitable host cells for cloning or expression of antibody-encoding vectors include prokaryotic or eukaryotic cells. For example, anti-IL18BP antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523. After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized.” resulting in the production of an antibody with a partially or fully human glycosylation pattern (e.g., Gerngross Nat. Biotech. 22:1409-1414 (2004); and Li et al. Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al. J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK: buffalo rat liver cells (BRL 3A); human lung cells (W138); human liver cells (Hep G2); mouse mammary tumor (MMT 060562); TRI cells, as described, e.g., in Mather et al. Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al. Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu. Methods in Molecular Biology, Vol. 248 (B.K.C. Lo, ed., Humana Press. Totowa, NJ), pp. 255-268 (2003).

### Pharmaceutical Compositions/Formulations

Provided herein are pharmaceutical compositions and/or pharmaceutical formulations comprising the anti-IL18BP antibodies of the present disclosure and a pharmaceutically acceptable carrier.

In some embodiments, the antibody or antigen-binding fragment thereof having the desired degree of purity is present in a formulation comprising, e.g., a physiologically acceptable carrier, excipient or stabilizer (Remington's Pharmaceutical Sciences (1990) Mack Publishing Co., Easton, PA). In some embodiments, pharmaceutically acceptable carriers preferably are nontoxic to recipients at the dosages and concentrations employed.

Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In some aspects, a pharmaceutical composition comprises an anti-IL18BP antibody or antigen-binding fragment thereof as described herein, and a pharmaceutically acceptable carrier (see, e.g., Gennaro. Remington: The Science and Practice of Pharmacy with Facts and Comparisons: Drugfacts Plus. 20th ed. (2003); Ansel et al., Pharmaceutical Dosage Forms and Drug Delivery Systems. 7th ed., Lippencott Williams and Wilkins (2004); Kibbe et al., Handbook of Pharmaceutical Excipients. 3rd ed., Pharmaceutical Press (2000)).

In some embodiments, a pharmaceutically acceptable carrier preferably is nontoxic to recipients at the dosages and concentrations employed. The pharmaceutical compositions and/or pharmaceutical formulations to be used for in vivo administration can be sterile. This is readily accomplished by filtration through, e.g., sterile filtration membranes

Pharmaceutical compositions and/or pharmaceutical formulations provided herein are useful as a medicament, e.g., for treating cancer or for treating a neurodegenerative disorder.

### Therapeutic Uses

As disclosed herein, anti-IL18BP antibodies of the present disclosure may be used for treating diseases, disorders, and conditions. In some embodiments, the present disclosure provides methods for treating an individual having a neurodegenerative disorder, such as, for example. Parkinson's disease, comprising administering to the individual a therapeutically effective amount of an anti-IL18BP antibody of the present disclosure. In other embodiments, the present disclosure provides methods for treating an individual with a lysosomal storage disease comprising administering to the individual a therapeutically effective amount of an anti-IL18BP antibody of the present disclosure. In some embodiments, the lysosomal storage disease is Gaucher's disease. In other embodiments, the present disclosure provides methods for treating an individual having cancer comprising administering to the individual a therapeutically effective amount of an anti-IL18BP antibody of the present disclosure.

Ectopic or expression of IL18BP has been observed in various tumors; overexpression and activation of IL18BP has been implicated in lymphoid leukemia, lymphoma, adenoma, melanoma, gastric, prostate, and breast cancers; and IL18BP overexpression has been associated with metastasis. Accordingly, modulating the activity of IL18BP with an anti-IL18BP antibody of the present disclosure is an effective means of treating cancer.

In certain aspects, provided herein are methods for treating cancer in a subject in need thereof, the method comprising administering to the subject an anti-IL18BP antibody of the present disclosure, or a pharmaceutical composition comprising an anti-IL18BP antibody of the present disclosure. In some embodiments, a method is provided for treating cancer in a subject in need thereof, the method comprising administering to the subject an anti-IL18BP antibody of the present disclosure.

In some embodiments, the cancer is selected from cholangiocarcinoma, diffuse large B cell lymphoma, glioblastoma multiform, head and neck squamous carcinoma, kidney renal clear cell carcinoma, sarcoma, bladder cancer, breast cancer, colon cancer, endometrial cancer, kidney cancer, renal cancer, pancreatic adenocarcinoma, skin cutaneous melanoma, stomach adenocarcinoma, lung cancer, non-small cell lung cancer, melanoma, lymphoma, pancreatic cancer, prostate cancer, ovarian cancer, stomach cancer, thyroid cancer, cancer of the uterus, liver cancer, cervical cancer, testicular cancer, squamous cell carcinoma, glioma, glioblastoma, adenoma, and neuroblastoma. In some embodiments, the cancer is selected from glioblastoma multiforme, bladder carcinoma, and esophageal carcinoma. In some embodiments, the cancer is triple-negative breast carcinoma. In some embodiments, the cancer may be a primary tumor. In some embodiments, the cancer may be a metastatic tumor at a second site derived from any of the above types of cancer.

In some embodiments, an anti-IL18BP antibody of the present disclosure may be administered in conjunction with one or more therapeutic agents that act as a checkpoint inhibitor. In some embodiments, the method further includes administering to the individual at least one antibody that specifically binds to an inhibitory immune checkpoint molecule, and/or another standard or investigational anti-cancer therapy. In some embodiments, the inhibitory checkpoint molecule is selected from PD1, PD-L1, and PD-L2. In some embodiments, the at least one antibody that specifically binds to an inhibitory checkpoint molecule is administered in combination with an anti-IL18BP antibody of the present disclosure.

In some embodiments, the at least one antibody that specifically binds to an inhibitory checkpoint molecule is selected from an anti-PD-L1 antibody, an anti-PD-L2 antibody, and an anti-PD-1 antibody.

In some embodiments, a subject or individual is a mammal. Mammals include, without limitation, domesticated animals (e.g., cows, sheep, cats, dogs, and horses), primates (e.g., humans and non-human primates such as monkeys), rabbits, and rodents (e.g., mice and rats). In some embodiments, the subject or individual is a human.

### Articles of Manufacture

Provided herein are articles of manufacture (e.g., kit) comprising a monovalent anti-IL18BP antibody described herein. Article of manufacture may include one or more containers comprising an antibody described herein. Containers may be any suitable packaging including, but is not limited to, vials, bottles, jars, flexible packaging (e.g., sealed Mylar or plastic bags), and the like. The containers may be unit doses, bulk packages (e.g., multi-dose packages) or sub-unit doses.

In some embodiments, the kits may further include a second agent. In some embodiments, the second agent is a pharmaceutically-acceptable buffer or diluting agent including. In some embodiments, the second agent is a pharmaceutically active agent.

In some embodiments of any of the articles of manufacture, the article of manufactures further include instructions for use in accordance with the methods of this disclosure. The instructions generally include information as to dosage, dosing schedule, and route of administration for the intended treatment. In some embodiments, these instructions comprise a description of administration of the isolated antibody of the present disclosure (e.g., an anti-IL18BP antibody described herein) to treat an individual having a disease, disorder, or injury, such as for example cancer or a neurodegenerative disorder (e.g., Parkinson's disease), according to any methods of this disclosure. In some embodiments, the instructions include instructions for use of the anti-IL18BP antibody and the second agent (e.g., second pharmaceutically active agent).

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Production of Avi- and Poly-His-Tagged IL-18BP Polypeptides

Recombinant fusion polypeptide constructs of human, cynomolgus, and murine IL-18BP polypeptides containing Avi and poly-His tags for use in the generation and characterization of anti-IL-18BP antibodies of the present disclosure were generated as follows. Nucleic acids encoding the human IL-18BPa (SEQ ID NO: 1), cynomolgus IL-18BP (SEQ ID NO: 4), and murine IL-18BP (SEQ ID NO: 3) were each cloned into a mammalian expression vector containing nucleic acid encoding a heterologous signal peptide as well as containing Avi and poly-His tags either at the N-terminus or C-terminus of the recombinant polypeptide constructs.

The amino acid sequences of human IL-18BP, cynomolgus IL-18BP, and murine IL-18BP are set forth below.

Human IL-18BP Isoform A amino acid sequence
(SEQ ID NO: 1)
MTMRHNWTPDLSPLWVLLLCAHVVTLLVRATPVSQTTTAATASVRSTKDP
CPSQPPVFPAAKQCPALEVTWPEVEVPLNGTLSLSCVACSRFPNFSILYW
LGNGSFIEHLPGRLWEGSTSRERGSTGTQLCKALVLEQLTPALHSTNFSC
VLVDPEQVVQRHVVLAQLWAGLRATLPPTQEALPSSHSSPQQQG
Human IL-18BP Isoform C amino acid sequence
(SEQ ID NO: 2)
MTMRHNWTPDLSPLWVLLLCAHVVTLLVRATPVSQTTTAATASVRSTKDP
CPSQPPVFPAAKQCPALEVTWPEVEVPLNGTLSLSCVACSRFPNFSILYW
LGNGSFIEHLPGRLWEGSTSRERGSTGTQLCKALVLEQLTPALHSTNFSC
VLVDPEQVVQRHVVLAQLWVRSPRRGLQEQEELCFHMWGGKGGLCQSSL
Mouse IL-18BP Isoform A amino acid sequence
(SEQ ID NO: 3)
MTMRHCWTAGPSSWWVLLLYVHVILARATSAPQTTATVLTGSSKDPCSSW
SPAVPTKQYPALDVIWPEKEVPLNGTLTLSCTACSRFPYFSILYWLGNGS
FIEHLPGRLKEGHTSREHRNTSTWLHRALVLEELSPTLRSTNFSCLFVDP
GQVAQYHIILAQLWDGLKTAPSPSQETLSSHSPVSRSAGPGVA
Cynomolgus IL-18BP amino acid sequence
(SEQ ID NO: 4)
MRHNWTPDLSFLWVLLCAHIITLLVRATPVSQTTTAATASSRSTKDPCPS
QPPVFPAAKQCPALEVTWPEVEMPLNGTLTLSCTACSRFPNFSMLYWLGN
GSFIEHLPGQLWEGSTSREHGSTGTRLYKALVLEQLTPALHSTNFSCVLM
DPEQVVQRHVILAQLWAGLRTTLPPTQEALPSSHSTGPQQPTAAGLRLST
GPAAARP
Avi/His-HuIL18BPa amino acid sequence
(SEQ ID NO: 5)
GLNDIFEAQKIEWHEGGHHHHHHHHGSGGTPVSQTTTAATASVRSTKDPC
PSQPPVFPAAKQCPALEVTWPEVEVPLNGTLSLSCVACSRFPNFSILYWL
GNGSFIEHLPGRLWEGSTSRERGSTGTQLCKALVLEQLTPALHSTNFSCV
LVDPEQVVQRHVVLAQLWAGLRATLPPTQEALPSSHSSPQQQG
Avi/HisMsIL18BPd amino acid sequence
(SEQ ID NO: 6)
GLNDIFEAQKIEWHEGGHHHHHHHHGSGGTSAPQTTATVLTGSSKDPCSS
WSPAVPTKQYPALDVIWPEKEVPLNGTLTLSCTACSRFPYFSILYWLGNG
SFIEHLPGRLKEGHTSREHRNTSTWLHRALVLEELSPTLRSTNFSCLFVD
PGQVAQYHIILAQLWDGLKTAPSPSQETLSSHSPVSRSAGPGVA
His/Avi-CynoIL18BP amino acid sequence
(SEQ ID NO: 7)
HHHHHHGLNDIFEAQKIEWHETPVSQTTTAATASSRSTKDPCPSQPPVFP
AAKQCPALEVTWPEVEMPLNGTLTLSCTACSRFPNFSMLYWLGNGSFIEH
LPGQLWEGSTSREHGSTGTRLYKALVLEQLTPALHSTNFSCVLMDPEQVV
QRHVILAQLWAGLRTTLPPTQEALPSSHSTGPQQPTAAGLRLSTG
PAAARP

The above recombinant nucleic acid constructs encoding poly-His- and Avi-tagged human, cynomolgus, and murine IL-18BP fusion polypeptides were transiently transfected into Expi293 cells. The recombinant fusion polypeptides were purified from the supernatants of the cells using HisPur™ Ni-NTA Resin (ThermoFisher, Cat #88221) following the manufacturer's instructions. To generate the biotinylated Avi-His-tagged polypeptides, purified human IL-18BP and mouse IL-18BP were labelled using a the BirA biotin-protein ligase standard reaction kit (Avidity, Cat #BirA500) according to the manufacturer's instructions.

Additionally, commercially available recombinant cynomolgus monkey IL-18BP-His (R&D Systems, Cat #2523-BP-050), mouse IL-18BPd-human IgG Fc (R&D Systems, Cat. #122-BP-100), human IL-18 (R&D Systems, Cat. #9124-IL-050), rhesus macaque IL-18 (R&D systems, Cat. #2548-RM-025), mouse IL-18 (R&D Systems, Cat. #9139-IL/CF), and recombinant human IL-12 (R&D Systems, Cat. #219-IL-005) were also used for anti-IL-18BP antibody generation, characterization, and functional assays as described below. His-tagged cynomolgus monkey IL-18BP and mouse IL-18 were biotinylated using EZ-Link Micro Sulfo-NHS-Biotinylation kit (Thermo Fisher, Cat. #21925) following standard procedure.

### Example 2: Generation of Anti-IL18BP Hybridoma Antibodies

BALB/c mice, CFW mice, or SJL mice (Charles River Laboratories. Wilmington. MA) were immunized twice a week by subcutaneous or intraperitoneal injections of purified C-terminal polypeptides of human, cynomolgus, and/or mouse IL-18BP (produced as described above in Example 1) with or without adjuvant. After a total of 12 injections and three days following the final boost, the lymph nodes and spleens were harvested from the mice for hybridoma cell line generation. Sera from the animals were analyzed for reactivity to IL-18BP by ELISA on human, cynomolgus, and/or mouse IL-18BP His-tagged or biotinylated Avi-His-tagged polypeptides (generated as described below).

Lymphocytes from animals whose sera demonstrated strong binding to IL-18BP polypeptides by ELISA assay were isolated and fused with SP2/mIL-6 (CRL-2016. American Type Culture Collection. Rockville. MD) mouse myeloma cells via electrofusion. The fused cells were mixed with anti-mouse IgG Fc-FITC and plated into methylcellulose-based ClonaCell-HY Medium D (Stemcell Technologies. Cat #03804) containing HAT components. Fluorescent IgG positive colonies were selected and transferred by a Clonepix 2 system (Molecular Devices. Sunnyvale. CA) into 96-well plates containing standard liquid hybridoma media. In total, 1989 hybridoma clones were isolated and screened for binding to IL-18BP as described below.

### Example 3: Screening of Anti-IL18BP Antibody Hybridoma Supernatants by ELISA

Supernatants from the hybridomas obtained as described above were screened for their ability to differentially bind human, cynomolgus, and/or mouse IL-18BP His-tagged or biotinylated Avi-His-tagged polypeptides (generated as described above) as compared to an irrelevant biotinylated Avi-His-tagged control polypeptide by ELISA. Briefly. 96-well polystyrene plates were coated with the recombinant IL-18BP polypeptides, washed, and blocked with ELISA diluent (PBS+0.5% BSA+0.05% Tween20). After primary incubation of hybridoma cell culture supernatants, the plates were washed and incubated with anti-mouse IgG Fc-HRP (Jackson Immunoresearch. Cat #115-035-071). After a final round of washing, the plates were developed with TMB substrate, quenched with sulfuric acid, and read on a SpectraMax M5 for absorbance at 450 nm. A total of 299 anti-IL18BP hybridoma clones were identified that displayed greater than 10-fold difference in binding to recombinant IL-18BP over background. Anti-IL18BP antibodies identified using this screen were further characterized as described below.

### Example 4: Generation of Anti-IL18BP Antibodies Using Phage Display

Antibody phage display was used to identify IL-18BP-specific antibodies from a semi-synthetic phage library. Three to four rounds of phage panning were performed against biotinylated human IL-18BP or mouse IL-18BP antigens. For each round of panning, phage library or amplified output from the previous round of panning was depleted with non-conjugated magnetic beads (Dynabeads M-280 Streptavidin). The depleted phage library was then incubated with biotinylated IL-18BP antigen conjugated to streptavidin magnetic beads. After binding and washing with PBST, bound phages were eluted and used to infect freshly grown ER2738 cells. Phage libraries for subsequent rounds of panning were produced by co-infection of infected ER2738 cells with helper phage (M13K07). Monoclonal phage-scFv (single-chain variable fragment) supernatants or bacterial periplasmic extract (PPE) obtained from the phage panning campaign were screened by ELISA as described above for binding to human, cynomolgus, and mouse IL-18BP. Twelve 96-well plates were screened, and 175 sequence-unique scFv binders were identified for further characterization.

### Example 5: Molecular Cloning of Anti-IL18BP Antibodies

Anti-IL18BP antibodies of the present disclosure derived from the hybridoma clones and phage clones described above were sequenced as follows. For the hybridoma clones, RNA was extracted, and cDNA synthesis was performed. The variable regions of IgG/IgM, IgK, and IgL were amplified using 5′ RACE strategy and sequenced on the Illumina MiSeq. For the phage clones, variable regions were sequenced directly from the phagemid. Unique amino acid sequences of the variable heavy chains and variable light chains of anti-IL18BP antibodies of the present disclosure are provided below. The Kabat heavy chain CDR sequences and light chain CDR sequences of the antibodies are set forth in Table 1 and Table 2 below, respectively. The heavy chain variable region and light chain variable region sequences of the antibodies are set forth in Table 3 below (in which the Kabat CDR sequences are underlined).

TABLE 1

| | | SEQ | | SEQ | | SEQ |
|---|---|---|---|---|---|---|
| Anti- | | ID | | ID | | ID |
| body | HVR-H1 | NO: | HVR-H2 | NO: | HVR-H3 | NO: |
| BP-01 | DFAMY | 60 | VISTDNGDTS | 71 | SRGPFDY | 87 |
| | | | YNQKFKG | | | |
| BP-02 | DYNMD | 61 | NINTNNGGTV | 72 | SDTTVVASR | 88 |
| | | | YNQKFKG | | YYAMDY | |
| BP-03 | DYNMD | 61 | NINTDNGGTV | 73 | SDTTVVASR | 88 |
| | | | YNQKFKG | | YYAMDY | |
| BP-04 | DYDVH | 62 | VISTYYGDVG | 74 | SGTYYYGTR | 89 |
| | | | YNQEFKG | | ESHALDY | |
| BP-05 | DYNMD | 61 | NINTNNGGTV | 75 | SDTTVVASR | 90 |
| | | | YNQKFKD | | YYALDY | |
| BP-06 | DYYLN | 63 | WIYPGSGNTK | 76 | RGVAWFAY | 91 |
| | | | YTEKFKD | | | |
| BP-07 | DYNMD | 61 | NINLNNGGSI | 77 | SDTTVVASR | 88 |
| | | | YNQKFKG | | YYAMDY | |
| BP-08 | SYWMH | 64 | MIDPNSDSSR | 78 | SRLGQDY | 92 |
| | | | YNEKFKN | | | |
| BP-09 | DYAMH | 65 | IIDPETGDTA | 79 | PRGAMDY | 93 |
| | | | FNQKFKG | | | |
| BP-10 | NYWMH | 66 | MIHPESGSTN | 80 | WDRGYFDY | 94 |
| | | | NNEKFKS | | | |
| BP-11 | NYWMH | 66 | HIDPSTGYTE | 81 | GLGGLFGY | 95 |
| | | | YNQKFKD | | | |
| BP-12 | EYTMH | 67 | GVNPNNGGTT | 82 | LWRVFDY | 96 |
| | | | YNQKERD | | | |
| BP-13 | DYEMH | 68 | PIDPETGDTA | 83 | GSRGVFDY | 97 |
| | | | YNQKFKG | | | |
| BP-14 | GYYMH | 69 | IINPRGGSTS | 84 | GLELHWFDP | 98 |
| | | | HAQKFQG | | | |
| BP-15 | TYDIN | 70 | IINPSGGSTS | 85 | GIGELDFDY | 99 |
| | | | YAQKFQG | | | |
| BP-16 | TYDIN | 70 | IIGSYGGATS | 86 | AEEELRFDY | 100 |
| | | | YAQKFQG | | | |

TABLE 2

| | | SEQ | | SEQ | | SEQ |
|---|---|---|---|---|---|---|
| Anti- | | ID | | ID | | ID |
| body | HVR-L1 | NO: | HVR-L2 | NO: | HVR-L3 | NO: |
| BP-01 | KASQDVG | 101 | WASTRHI | 112 | QRYSSYPWT | 122 |
| | TAVA | | | | | |
| BP-02 | RASESVS | 102 | HASNLET | 113 | LQSRKIPPT | 123 |
| | IIATNLI | | | | | |
| | H | | | | | |
| BP-03 | RASESVS | 103 | HASNLET | 113 | LQSRKIPPT | 123 |
| | IIGTNLI | | | | | |
| | H | | | | | |
| BP-04 | RTSDNVY | 104 | GAINLAD | 114 | QHFWGNPYT | 124 |
| | SNLA | | | | | |
| BP-05 | RASESVS | 105 | HASNLET | 113 | LQSRKIPPT | 123 |
| | IIGTDLI | | | | | |
| | H | | | | | |
| BP-06 | KASQDIN | 106 | RANRLVD | 115 | LQHDDFPPT | 125 |
| | SYLS | | | | | |
| BP-07 | RASESVS | 103 | HASNLET | 113 | LQSRKIPPT | 123 |
| | IIGTNLI | | | | | |
| | H | | | | | |
| BP-08 | KASQDVS | 107 | WASTRHT | 116 | QQHFSTPYT | 126 |
| | TAVA | | | | | |
| BP-09 | KASQDVG | 101 | WASTRHT | 116 | QQYSSHPYT | 127 |
| | TAVA | | | | | |
| BP-10 | KASQDVR | 108 | WTSTRHT | 117 | QQHYSTPYT | 128 |
| | STVG | | | | | |
| BP-11 | KASQDIN | 109 | HTSTLMP | 118 | LQYDNLLWT | 129 |
| | KFIA | | | | | |
| BP-12 | KASQDVG | 101 | WASTRHT | 116 | HQFSRYPLT | 130 |
| | TAVA | | | | | |
| BP-13 | KASQDVG | 101 | WASARHT | 119 | QHYFNFPRT | 131 |
| | TAVA | | | | | |
| BP-14 | RASQNIR | 110 | AASSLOS | 120 | QQANSFPYT | 132 |
| | SWLA | | | | | |
| BP-15 | RASQSIS | 111 | KASTLHN | 121 | QQAISFPLT | 133 |
| | SYLN | | | | | |
| BP-16 | RASQSIS | 111 | KASTLHN | 121 | QQAISFPLT | 133 |
| | SYLN | | | | | |

TABLE 3

| | | SEQ | | SEQ |
|---|---|---|---|---|
| Anti- | | ID | | ID |
| body | Heavy Chain Variable | NO: | Light Chain Variable | NO: |
| BP-01 | QVQLQQSGPEVVRPGVSVKISCK | 29 | DIVMTQSHKFKSTSVGDRVNI | 45 |
| | GSGYTFTDFAMYWVKQSHAKSLE | | TCKASQDVGTAVAWYQQKPGQ | |
| | WIGVISTDNGDTSYNQKFKGKAT | | SPKLLIYWASTRHIGVPDRFT | |
| | MTVDKSSSTAYMELDRLTSEDSA | | GSGSGTDFTLTIRNVQSEDLA | |
| | IYYCARSRGPFDYWGQGTALTVS | | DYFCQRYSSYPWTFGGGTKLE | |
| | S | | IK | |
| BP-02 | EVQLQQSGPELVKPGASVKIPCK | 30 | DIVLTQSPASLAMSLGKRATI | 46 |
| | ASGYTFTDYNMDWVKQSHGKSLE | | SCRASESVSIIATNLIHWYQQ | |
| | WIGNINTNNGGTVYNQKFKGKAT | | KPGQPPKLLIYHASNLETGVP | |
| | LTVDKSSSTAYMELRSLTSEDTA | | ARFSGSGSRTDETLTIDPVEK | |
| | VYYCARSDTTVVASRYYAMDYWG | | DDVAIYYCLQSRKIPPTFGGG | |
| | QGTSVTVSL | | TKLEIK | |
| BP-03 | EVQLQQSGPELVKPGASVKIPCK | 31 | DIVLTQSPASLAMSLGKRATI | 47 |
| | ASGYTFTDYNMDWVKQSHGKSLE | | SCRASESVSIIGTNLIHWYQQ | |
| | WIGNINTDNGGTVYNQKFKGKAT | | KPGQPPKLLIYHASNLETGVP | |
| | LTVDQSSSTAYMELRSLTSEDTA | | ARFSGSGSRTDFTLTIDPVEE | |
| | VYYCARSDTTVVASRYYAMDYWG | | DDVAIYYCLQSRKIPPTFGGG | |
| | QGTSVTVSS | | TKLEIK | |
| BP-04 | HVHLHQSGAELVRPGVSVKISCR | 32 | DIQMTQSPASLSVSVGETVTI | 48 |
| | GSGYTFTDYDVHWVKQSHAKSLE | | TCRTSDNVYSNLAWYQQKOGK | |
| | WIGVISTYYGDVGYNQEFKGKAT | | SPQLLVYGAINLADGVPSRFS | |
| | MTLDKSSSTSYLELARLTSEDSA | | GSGSDTQYSLKINNLQSEDFG | |
| | IYYCARSGTYYYGTRESHALDYW | | SYYCQHFWGNPYTFGGGTKLE | |
| | GQGTSVTVSS | | IK | |
| BP-05 | EVQLQQSGPELVKPGASVKIPCK | 33 | DIVLTQSPASLAMSLGKRATI | 49 |
| | ASGYTFTDYNMDWVKQSHGKSLE | | SCRASESVSIIGTDLIHWYQQ | |
| | WIGNINTNNGGTVYNQKFKDKAT | | KPGQPPKLLIYHASNLETGVP | |
| | LTVDKSSSTAYMELRSLTSEDTA | | ARFSGSGSRTDFTLTIDPVEE | |
| | VYYCARSDTTVVASRYYALDYWG | | DDVAIYYCLQSRKIPPTFGGG | |
| | QGTSVTVSS | | TKLEIK | |
| BP-06 | QIQLQQSGPELVKPGASVKISCK | 34 | DIKMTQSPSSIYASLGERVTI | 50 |
| | ASGYTFTDYYLNWVKQTPGOGLE | | TCKASQDINSYLSWFQQKPGK | |
| | WIGWIYPGSGNTKYTEKFKDKAS | | SPKTLIHRANRLVDGVPSRFS | |
| | LTVDTSSNTAYMQLSSLTSDDTA | | GSGSGQDYSLTISSLEYEDMG | |
| | VYFCARRGVAWFAYWGQGTLVTV | | IYYCLQHDDFPPTFGAGTNLE | |
| | SA | | LK | |
| BP-07 | EVQLQQSGPELVKPGASVKISCR | 35 | DIVLTQSPASLAMSLGKRATI | 51 |
| | ASGFTITDYNMDWVRQSHGKSLE | | SCRASESVSIIGTNLIHWYQQ | |
| | WIGNINLNNGGSIYNQKFKGKAT | | KPGQPPKLLIYHASNLETGVP | |
| | LTIDKSSSTAYMELRSLTSEDTA | | VRESGSGSRTDETLTIDPVEE | |
| | VYYCARSDTTVVASRYYAMDYWG | | DDVAIYYCLQSRKIPPTFGGG | |
| | QGTSVTVSS | | TKLEIK | |
| BP-08 | QVQLQQPGAELVKPGASVKLSCK | 36 | DIVMTQSHKFMSTSVGDRVSI | 52 |
| | VSGYTFTSYWMHWVKQRPGQGLE | | TCKASQDVSTAVAWYQQKPGQ | |
| | WIGMIDPNSDSSRYNEKFKNRAT | | SPKLLIYWASTRHTGVPDRFT | |
| | LTADKSSSTAFMQFSSLTSEDSA | | GSESGTDYTLIISSVQAEDLA | |
| | VYYCARSRLGQDYWGQGTTLTVS | | LYYCQQHFSTPYTFGGGTKLE | |
| | S | | IK | |
| BP-09 | QVQLQQSGAELVRPGASVTLSCK | 37 | DIVMTQSHKFMSTSGGDRVSI | 53 |
| | ASGYRFTDYAMHWVKQTPVHGLQ | | TCKASQDVGTAVAWYQQKPGI | |
| | WIGIIDPETGDTAFNQKFKGKAT | | SPKLLIYWASTRHTGVPDRFT | |
| | LTADKSSSTAYMELSSLTSEDSA | | GSGSGTDETLAISNVQSEDLA | |
| | VYYCTRPRGAMDYWGQGTSVTVS | | DYFCQQYSSHPYTFGGGTKLE | |
| | S | | IK | |
| BP-10 | QVQLQQPGAERVEPGASVRLSCK | 38 | DIVMTQSHKFMSTSVGDRVSI | 54 |
| | TSGYTFTNYWMHWVKORPGOGLE | | TCKASQDVRSTVGWYQQRPGQ | |
| | WIGMIHPESGSTNNNEKFKSKAT | | SPKLLIYWTSTRHTGVPDRFT | |
| | LTVDKSSSTAYMQLSSLTSEDSA | | GSGSGTDYTLTISSVQAEDLA | |
| | VYYCARWDRGYFDYWGQGTTLTV | | LYYCQQHYSTPYTFGGGTTLE | |
| | SS | | IK | |
| BP-11 | QVQLQQSGAELAKPGASVKMSCK | 39 | DIQMTQSPSSLSASLGGKVTI | 55 |
| | ASGYTFTNYWMHWVKQRPGQGLE | | TCKASQDINKFIAWYQHKPGK | |
| | WIGHIDPSTGYTEYNQKFKDKAT | | GPRLLIHHTSTLMPGIPSRFS | |
| | LTADKSSSTAYMQLSSLTSEDSA | | GSGSGREYSFSISNLEPEDIA | |
| | VYYCARGLGGLFGYWGQGTLVTV | | TYYCLQYDNLLWTFGGGTKLE | |
| | SA | | IR | |
| BP-12 | EVQLQQSGPELVKPGASVKMSCK | 40 | DIVMTQSHKFMSSSVGDRVSI | 56 |
| | TSGYTFTEYTMHWVKQSHGKSLE | | TCKASQDVGTAVAWYQQKPGQ | |
| | WIGGVNPNNGGTTYNQKFRDKAT | | SPKLLIYWASTRHTGVPDRFT | |
| | LTVDKSSSTTYMEVRTLTSDDSA | | GSGSGTDFTLTISNVQSEDLA | |
| | VYYCAPLWRVFDYWGQGTTLTVS | | DYFCHQFSRYPLTFGAGTKLE | |
| | S | | LK | |
| BP-13 | QVQLQQSGAELVRPGASVTLSCK | 41 | DIVMTQSHKFMSTSVGDRVSI | 57 |
| | ASGYTFTDYEMHWVKQTPVHGLE | | TCKASQDVGTAVAWYQQKPGQ | |
| | WIGPIDPETGDTAYNQKFKGQAT | | SPKLLIYWASARHTGVPDRFT | |
| | LTADKSSSTAYMELSSLTSEDSA | | GSGSGTDFTLTISNVQSEDLA | |
| | VYYCTRGSRGVFDYWGQGTALTV | | DYFCQHYFNFPRTFGGGTKLE | |
| | SS | | IT | |
| BP-14 | QVQLVQSGAEVKKPGASVKVSCK | 42 | DIQMTQSPSSLSASVGDRVTI | 58 |
| | ASGYTFTGYYMHWVRQAPGOGLE | | TCRASQNIRSWLAWYQQKPGK | |
| | WMGIINPRGGSTSHAQKFQGRVT | | APKLLIYAASSLQSGVPSRFS | |
| | MTRDTSTSTVYMELSSLRSEDTA | | GSGSGTDFTLTISSLQPEDFA | |
| | VYYCARGLELHWFDPWGQGTLVT | | TYYCQQANSFPYTFGQGTKLE | |
| | VSS | | IK | |
| BP-15 | QVQLVQSGAEVKKPGASVKVSCK | 43 | DIQMTQSPSSLSASVGDRVTI | 59 |
| | ASGYTFTTYDINWVRQAPGQGLE | | TCRASQSISSYLNWYQQKPGK | |
| | WMGIINPSGGSTSYAQKFQGRVT | | APKLLIYKASTLHNGVPSRFS | |
| | MTRDTSTSTVYMELSSLRSEDTA | | GSGSGTDFTLTISNLQPEDFA | |
| | VYYCATGIGELDFDYWGQGTLVT | | TYYCQQAISFPLTFGGGTKVE | |
| | VSS | | IK | |
| BP-16 | QVQLVQSGAEVKKPGASVKVSCK | 44 | DIQMTQSPSSLSASVGDRVTI | 59 |
| | ASGYTFTTYDINWVRQAPGQGLE | | TCRASQSISSYLNWYQQKPGK | |
| | WMGIIGSYGGATSYAQKFQGRVT | | APKLLIYKASTLHNGVPSRFS | |
| | MTRDTSTSTVYMELSSLRSEDTA | | GSGSGTDFTLTISNLQPEDFA | |
| | VYYCATAEEELRFDYWGQGTLVT | | TYYCQQAISFPLTFGGGTKVE | |
| | VSS | | IK | |

### Example 6: Production of Recombinant Anti-IL18BP Antibodies

Anti-IL18BP antibody variable gene regions were synthesized and subcloned into mammalian expression vectors encoding human IgG1-Fc-LALAPS (human IgG1 Fc with the following amino acid substitutions according to EU numbering: L234A, L235A, P331S) and IgK to express recombinant antibodies in Expi293 or ExpiCHO cells (Invitrogen) using standard procedures. Anti-IL18BP antibodies from the supernatants were purified using protein A chromatography, and the antibody concentrations were determined by absorbance at 280 nm measurements.

### Example 7: Screening of Purified Anti-IL18BP Antibodies by ELISA

Purified recombinant anti-IL18BP antibodies (huIgG-Fc-LALAPS) of the present disclosure were screened by ELISA as described above for their ability to bind human, cynomolgus, and/or mouse IL-18BP His-tagged or biotinylated Avi-His-tagged polypeptides. The absorbance values as a ratio of IL-18BP binding over background was calculated and summarized in Table 4 below. Out of the 16 recombinant antibodies tested in this assay, 15 antibodies were cross-reactive to human, cynomolgus, and mouse IL-18BP. Of these, anti-IL18BP antibodies BP-05 and BP-14 had weaker binding to cynomolgus IL-18BP compared to that observed for binding to human IL-18BP and mouse IL-18BP (i.e., these anti-IL18BP antibodies showed less than 10-fold binding to cynomolgus IL-18BP compared to that observed with control antibody). Anti-IL18BP antibody BP-13 showed weaker binding to mouse IL-18BP than to human IL-18BP and cynomolgus IL-18BP. Anti-IL18BP antibody BP-06 only showed binding to human IL-18BP and cynomolgus IL-18BP and did not bind mouse IL-18BP.

TABLE 4

| | ELISA binding ratio (fold-change over background) | | |
|---|---|---|---|
| Antibody | Human IL-18BP | Mouse IL-18BP | Cynomolgus IL-18BP |
| BP-01 | 40.7 | 36.7 | 47.4 |
| BP-02 | 35.1 | 39.2 | 29.7 |
| BP-03 | 41.9 | 41.6 | 36.5 |
| BP-04 | 39.7 | 39.1 | 45.2 |
| BP-05 | 38.3 | 32.1 | 9.7 |
| BP-06 | 42.1 | 1.2 | 44.3 |
| BP-07 | 38.5 | 39.3 | 31.1 |
| BP-08 | 38.8 | 35.0 | 41.5 |
| BP-09 | 44.4 | 32.7 | 44.9 |
| BP-10 | 42.1 | 40.8 | 48.1 |
| BP-11 | 44.4 | 43.6 | 47.6 |
| BP-12 | 43.2 | 34.2 | 44.0 |
| BP-13 | 44.7 | 16.9 | 49.0 |
| BP-14 | 36.3 | 45.1 | 7.3 |
| BP-15 | 32.7 | 30.1 | 34.5 |
| BP-16 | 23.6 | 22.1 | 25.1 |

Taken together, these binding data showed that, in general, anti-IL18BP antibodies of the present disclosure bound human, mouse, and cynomolgus IL-18BP; a small number of anti-IL18BP antibodies of the present disclosure were less effective at binding mouse or cynomolgus IL-18BP.

### Example 8: Affinity Maturation of Anti-IL18BP Antibodies

Certain anti-IL18BP antibodies of the present disclosure were further engineered using affinity maturation methodologies. Briefly, amino acid residues in heavy chain CDRs and light chain CDRs were randomized to generate CDR mutagenesis libraries. The CDR mutagenesis libraries were subjected to three to four rounds of selection to enrich for antibody variants showing improved binding to human IL-18BP. Individual anti-IL18BP antibody variants obtained from these methods were screened as described above; those antibodies showing improved binding to human IL-18BP and cross-reactivity to cynomolgus IL-18BP were further characterized as described below.

### Example 9: Characterization of Binding Kinetics and Cross-Reactivity of Anti-IL18BP Antibodies

Binding kinetics of the mouse/human chimeric anti-IL18BP-IgG1-Fc-LALAPS antibodies of the present disclosure were evaluated using a Carterra® LSA instrument (Carterra, Salt Lake City, UT). Briefly, anti-IL18BP antibodies were immobilized onto an HC30M sensor chip (Carterra) by amine coupling according to the manufacturer's recommended protocol. After priming with HBS-EP+ (Teknova, Hollister, CA, #8022) running buffer with 0.5 mg/mL BSA (MP Biomedicals LLC, #820451), the immobilized antibodies were tested for their ability to bind to several forms of recombinant IL-18BP orthologs produced as described above. IL-18BP analytes were diluted to 1 μM in running buffer, diluted serially to 40 μM, and injected for five minutes over the entire chip using the single channel flow cell. Dissociation was measured for 15 min, followed by regeneration of the chip with two 30-second injections of 10 mM glycine pH 2.0. The data were analyzed using the NextGenKIT high-throughput kinetics analysis software from Carterra.

Several anti-IL18BP antibodies showing dissociation rates too slow to accurately measure binding kinetics by this method were further evaluated using the Biacore® T200 (Cytiva) with a capture protocol with extended dissociation times. Briefly, anti-IL18BP antibodies were diluted to 2 μg/mL and captured using a Protein A/G (Thermo Fisher, #21186) surface on a CM4 chip, that was prepared by amine coupling according to the manufacturer's recommendations. IL-18BP analytes were diluted to 1 μM in running buffer, diluted serially to 1 pM (human), and 1 nM (mouse and cynomolgus), and injected in triplicate for 2 minutes, followed by dissociation for 3 minutes. The highest concentration injections for anti-IL18BP antibody BP-16 were allowed to dissociate for 120 minutes to allow sufficient dissociation for curve fitting. Data were analyzed using Biacore® evaluation software to generate kinetic constants. The equilibrium dissociation constants (K_{D}) were calculated from the fitted association and dissociation rate constants (k-on and k-off) for each of the anti-IL18BP antibodies. These K_{D} values are summarized in Table 5 below.

TABLE 5

| Antibody | K_{D} (nM) human | K_{D} (nM) cyno |
|---|---|---|
| BP-01 | HB | 12.1 |
| BP-02 | 1.6 ± 0.8 | WB |
| BP-03 | 4.1 ± 1.0 | 2.34 |
| BP-04 | 0.8 ± 0.4 | 3.0 ± 1.1 |
| BP-05 | 1.67 ± 1.5 | WB |
| BP-06 | HB | HB |
| BP-07 | 2.7 ± 0.6 | HB |
| BP-08 | 6.9 ± 1.6 | 41.4 ± 53.3 |
| BP-09 | 2.01 ± 1.3 | 1.4 ± 1.0 |
| BP-10 | 1.0 | 0.02 |
| BP-11 | 2.9 ± 0.1* | 1.0* |
| BP-12 | 7.6 ± 3.2 | 0.5 ± 0.6 |
| BP-13 | 7.2 ± 4.5 | 0.3 ± 0.1 |
| BP-14 | 3.2 ± 0.3 | WB |
| BP-15 | 18.8 ± 2.9 | WB |
| BP-16 | <0.003* | 11.8 ± 3.1* |

HB= Heterogeneous binding, doesn't fit to 1:1 binding model
WB= Weak binding with K_{D} > 1 μM
*= values measured using Biacore ® T200; all others were measured using Carterra LSA

### Example 10: Species Specificity of Anti-IL18BP Antibodies of the Present Disclosure

Evaluation by SPR of binding of purified IgGs to human, mouse, and cynomolgus IL-18BP qualitatively showed that fourteen anti-IL18BP antibodies of the present disclosure (BP-01, BP-02, BP-03, BP-04, BP-05, BP-07, BP-09, BP-10, BP-11, BP-12, BP-13, BP-14, BP-15, and BP-16) were cross-reactive to IL-18BP from all three species. Two anti-IL18BP antibodies of the present disclosure (BP-06 and BP-08) bound only to human IL-18BP and cynomolgus IL-18BP but did not bind mouse IL-18BP. Five antibodies, BP-01, BP-09, BP-10, BP-12, and BP-13, bound more strongly to human and cyno IL-18BP than to mouse IL-18BP, while five antibodies, BP-02, BP-05, BP-07, BP-14, and BP-15 bound more strongly to human and mouse IL-18BP compared to the cynomolgus ortholog. These results of these studies are summarized in Table 6 below.

TABLE 6

| Human, cynomolgus, and | | | Human and cynomolgus only cross- |
|---|---|---|---|
| mouse cross-reactive | | | reactive |
| BP-03 | BP-01* | BP-02** | BP-06 |
| BP-04 | BP-09* | BP-05** | BP-08 |
| BP-11 | BP-10* | BP-07** | |
| BP-16† | BP-12* | BP-14** | |
| | BP-13* | BP-15** | |

*Mouse IL-18BP binding < human and cynomolgus
**Cynomolgus IL-18BP binding < human and mouse
†Human IL-18BP binding > mouse and cynomolgus

### Example 11: Epitope Binding Competition Between IgGs Binding to Human IL-18BP

Epitope binding competition analysis between the anti-IL18BP antibodies of the present disclosure was carried out using a standard sandwich binning approach on the Carterra® LSA instrument following the manufacturer's recommended protocol. Briefly, antibodies were immobilized as ligands in an array on an HC200M sensor chip and binding level of human IL-18BP was tested. In each cycle, human IL-18BP was injected over the array of immobilized antibodies, followed by an individual antibody, or running buffer as analyte. At regular intervals during the runs, buffer injection was repeated to monitor surface activity and allow comparison of binding by the second antibody. The data were analyzed using the Epitope high-throughput analysis software provided by Carterra, which allows stratification of different antibodies into bins based on their competition binding profiles. By this method, anti-IL18BP antibodies of the present disclosure were placed into the bins as shown in Table 7 below.

TABLE 7

| | | | | |
|---|---|---|---|---|
| Bin 1 | Bin 2 | Bin 3 | Bin 4 | Bin 5 |

| | | | | | |
|---|---|---|---|---|---|
| BP-04 | BP-02 | BP-08 | BP-01 | BP-10 | BP-06 |
| BP-15 | | | BP-03 | BP-11 | |
| BP-16 | | | BP-05 | BP-12 | |
| | | | BP-07 | BP-13 | |
| | | | BP-09 | BP-14 | |

These results indicated that anti-IL18BP antibodies of the present disclosure showed binding to human IL-18BP in five distinct bins as determined by this epitope binding competition assay.

### Example 12: Anti-IL18BP Antibodies Block IL-18 Binding to IL-18BP

The ability of anti-IL18BP antibody IgGs of the present disclosure to block IL-18 binding to IL-18BP was evaluated by SPR using the Carterra® LSA with two approaches using an immobilized IgG array as described for epitope binning above. In one approach, similar to epitope sandwich binning described above, IL-18BP was injected first to allow binding to the IgGs, followed immediately by injection of IL-18. None of the anti-IL18BP antibodies described herein allowed concurrent binding of IL-18 to the IgG-bound IL-18BP, while the control anti-His antibody (R&D Systems, MAB050) did allow such binding. In the second approach, IL-18BP was injected over the immobilized IgGs alone or in complex with IL-18. For all the antibodies described herein, pre-complexing with IL-18 blocked binding of IL-18BP to the IgGs, while IL-18BP alone bound robustly. The observed reciprocal blocking is diagnostic for an anti0IL18BP antibody blocking of IL-18BP binding to IL-18.

### Example 13: Antibody Blockade of IL-18BP from Binding to IL-18

Anti-IL18BP hybridoma and phage antibodies obtained as described above were assessed for their ability to block IL-18BP from binding to IL-18. For human and cynomolgus IL-18BP blocking assays, plates were coated with human or cynomolgus IL-18BP-Avi-His overnight at 4° C. After washing, the plates were blocked at room temperature for 1 hour. Anti-IL18BP antibodies of the present disclosure were then added to the plates and the plates were incubated at room temperature for 2 hours. Recombinant human IL-18 (R&D Systems, Cat. #9124-IL-050) or recombinant rhesus macaque IL-18 (R&D systems, Cat. #2548-RM-025) were added to the plates and the plates were then incubated at room temperature for 2 hours. The plates were then washed followed by the addition of anti-human/rhesus macaque IL-18 antibody (R&D Systems, Cat. #AF2548) and further incubated at room temperature for 1 hour. HRP-donkey anti-goat IgG (Jackson Immuno Research, Cat. #705-035-003) as was used for detection. ELISA signal was measured using SuperSignal ELISA Pico chemluminescent substrate (Thermo Fisher, Cat. #37069) on the Envision.

For mouse IL-18BP blocking assays, recombinant mouse IL-18 (R&D Systems, Cat. #9139-IL/CF) was biotinylated using EZ-Link Micro Sulfo-NHS-Biotinylation kit (Thermo Fisher, Cat. #21925). Rabbit anti-human Fc (Jackson Immuno Research, Cat. #309-005-008) was used to capture mouse IL-18BPd-Fc Chimera (R&D Systems, Cat. #122-BP-100). Anti-IL18BP antibodies of the present disclosure were then added, followed by biotinylated mouse IL-18. Assay signal was detected by Streptavidin-HRP.

Blocking signals were normalized to the isotype control signal, which was defined with a value of 1 in these studies. Any anti-IL18BP antibody showing a normalized signal less than 1 (e.g., less than the value obtained with isotype control antibody) was considered a blocking antibody. The results of these studies are shown in Table 8.

TABLE 8

| | Blocking ELISA | | |
|---|---|---|---|
| | Blocking: | Blocking: | Blocking: |
| Antibody | Human | Mouse | Cyno |
| BP-01 | 0.076 | 0.316 | 0.795 |
| BP-02 | 0.167 | 0.132 | 0.973 |
| BP-03 | 0.151 | 0.157 | 0.996 |
| BP-04 | 0.165 | 0.122 | 0.943 |
| BP-05 | 0.191 | 0.138 | 0.989 |
| BP-06 | 0.174 | 0.971 | 0.378 |
| BP-07 | 0.147 | 0.156 | 0.999 |
| BP-08 | 0.133 | 0.356 | 0.478 |
| BP-09 | | | 0.315 |
| BP-10 | 0.067 | 0.326 | 0.280 |
| BP-11 | 0.089 | 0.140 | 0.490 |
| BP-12 | 0.117 | 0.351 | 0.360 |
| BP-13 | 0.128 | 0.530 | 0.331 |
| BP-14 | 0.069 | 0.202 | 1.01 |
| BP-15 | | 0.211 | |

As shown in Table 8, anti-IL18BP antibodies of the present disclosure were effective at blocking the interaction (i.e., binding) of IL-18 to IL18BP. Specifically, anti-IL18BP antibodies BP-01, BP-02, BP-03, BP-04, BP-05, BP-06, BP-07, BP-08, BP-10, BP-11, BP-12, BP-13, and BP-14 were very effective at blocking binding of human IL-18 to human IL18BP; anti-IL18BP antibodies BP-01, BP-02, BP-03, BP-04, BP-05, BP-07, BP-08, BP-10, BP-11, BP-12, BP-13, BP-14, and BP-15 were very effective at blocking binding of mouse IL-18 to mouse IL18BP (but not BP-06); and BP-01, BP06, BP-08, BP-09-BP-10, BP-11, BP-12, and BP-13 were very effective at blocking binding of cynomolgus IL-18 to cynomolgus IL18BP (but not BP-02, BP-03, BP-04, BP-05, BP07, and BP-14). Additionally, these results showed that certain anti-IL18BP antibodies of the present disclosure that were effective at blocking binding of human IL-18 to human IL18BP were also effective at blocking binding of mouse and/or cynomolgus IL-18 to mouse and/or cynomolgus IL18BP, respectively.

### Example 14: Effect of Anti-IL18BP Antibodies on Interferon-Gamma (IFNγ) Release In Vitro

The effect of anti-IL18BP antibodies of the present disclosure on IFNγ release in human peripheral blood mononuclear cells (PBMC) and mouse splenocytes was examined as follows. Human PBMCs were obtained from healthy human subjects and isolated using a standard Ficoll protocol. The isolated PBMCs were seeded in 96-well plates in RPMI medium containing FBS. The cells were then incubated with recombinant human IL-12 (R&D Systems, Cat. #219-IL-005) and IL-18 (R&D Systems, Cat. #9124-IL-050) to activate the IL-18 pathway; with human IL-18BP-Avi-His protein was then added to the wells block the stimulatory response. Cells were incubated with a mixture of IL-12, IL-18, IL-18BP and anti-IL-18BP antibodies overnight at 37° C., and supernatant was collected for IFNγ analysis using the MSD human IFNγ Tissue Culture kit (MSD, Cat. #K211AEB).

This IFNγ release assay was also performed in mouse splenocytes as follows. Anti-IL18BP antibodies of the present disclosure were tested in the mouse splenocyte culture system to evaluate antibody activity in mouse primary splenocytes. Mouse splenocytes were isolated by dissociating spleens from mice using the GentleMacs (Miltenyi Biotec) per manufacturer's protocol. Mouse splenocytes were incubated with a mixture of murine IL-18 (R&D Systems, Cat. #9139-IL/CF), IL-12 (R&D Systems, Cat. #419-ML-050), IL-18BP-Avi-His protein, and anti-IL18BP antibodies overnight at 37° C. Supernatant was collected and analyzed for IFNγ levels using MSD mouse IFNγ kit (MSD, Cat. #B22TT).

EC50 values of the anti-IL18BP antibodies in these assays was calculated using GraphPad Prism. Fold-change of IFNγ production at 10 μg/ml antibody concentration vs baseline was calculated to reflect antibody efficacy in this assay. The results of these studies are shown below in Table 9.

TABLE 9

| | |
|---|---|
| Human PBMC | Mouse splenocytes |

| | EC50 | | EC50 | |
|---|---|---|---|---|
| Antibody | (nM) | Fold change | (nM) | Fold change |
| BP-01 | 0.75 | 14.91 | 8.36 | 1.65 |
| BP-02 | 0.75 | 16.68 | 4.30 | 9.34 |
| BP-03 | 1.14 | 11.27 | 4.49 | 5.93 |
| BP-04 | 0.67 | 18.99 | 4.29 | 2.74 |
| BP-05 | 1.33 | 16.44 | 2.94 | 3.15 |
| BP-06 | 1.23 | 10.90 | Inactive | 1.11 |
| BP-07 | 1.66 | 16.61 | 8.16 | 2.78 |
| BP-08 | 2.01 | 5.19 | Inactive | 0.75 |
| BP-09 | 1.41 | 15.41 | 425.90 | 2.88 |
| BP-10 | 2.05 | 15.53 | 538.40 | 2.85 |
| BP-11 | 4.25 | 9.72 | 7.00 | 2.75 |
| BP-12 | 6.09 | 10.29 | 15.74 | 1.88 |
| BP-13 | 3.89 | 11.84 | 640.50 | 1.74 |
| BP-14 | 3.47 | 8.86 | 2.93 | 3.49 |
| BP-15 | 100.50 | 35.96 | 5.85 | 7.94 |
| BP-16 | 1.663 | 237.73 | 3.075 | 11.93 |

As shown in Table 9, anti-IL18BP antibodies of the present disclosure were effective at increasing IFNγ expression in human PBMCs and in mouse splenocytes. Anti-IL18BP antibodies of the present disclosure displayed a range of EC50 values for increased IFNγ levels in the range of approximately 0.75-100 nM in PBMCs and in the range of approximately 3-630 nM in mouse splenocytes. Additionally, anti-IL18BP antibodies of the present disclosure increased INFγ expression levels by approximately 5-237-fold above control in PBMCs, and by approximately 1.65-12-fold above control in mouse splenocytes.

This IFNγ release assay was also performed in human NK cells as follows. Anti-IL18BP antibodies of the present disclosure were tested for their ability to block the inhibitory effects of IL18BP on IL-18-mediated activation. Human peripheral blood NK cells (cat no. 70036, STEMCELL) were assessed for their ability to respond to recombinant rhesus macaque IL-18/IL-1F4 protein (cat no. 2548-RM, R&D), and showed similar levels of IFNγ release in comparison to stimulation with recombinant human IL-18/IL-1F4 protein (cat no. 9124-IL, R&D), data not shown. In subsequent experiments, human NK cells were activated using huIL-12 and rhesus IL-18 and cultured overnight at 37° C. in the presence of anti-IL18BP antibodies in addition to cynomolgus IL-18BP protein. Supernatant was collected form the cells, and the ability of anti-IL18BP antibodies to block cynomolgus IL18BP was measured via NK activation and downstream IFNγ release as measured by MSD (cat no. K156TTK-4). Antibody EC50 values were calculated using GraphPad Prism. Percent (%) of Max Gamma was calculated based on the anti-IL18BP antibody that elicited the maximal IFNγ response, which was set to 100%, while converting supernatant concentrations of IFNγ (pg/ml) secreted to a percentage of the maximal response. The results of these studies are shown in Table 10.

TABLE 10

Human NK

| Antibody | EC50 (nM) | % of Max Gamma |
|---|---|---|
| BP-01 | 12.54 | 52.18 |
| BP-02 | 1.986 | 10.44 |
| BP-04 | 2.412 | 25.94 |
| BP-06 | 8.067 | 75.75 |
| BP-08 | 19.16 | 58.38 |
| BP-09 | 51.68 | 46.64 |
| BP-10 | 6.002 | 55.23 |
| BP-11 | 37.06 | 100.00 |
| BP-12 | 70 | 76.45 |
| BP-13 | 39.73 | 54.06 |
| BP-14 | 0.1 | 9.4 |
| BP-15 | 0.0001061 | 10 |
| BP-16 | 8.4 | 51.60 |

As shown in Table 10, anti-IL18BP antibodies of the present disclosure were effective at increasing IFNγ expression in NK cells. Anti-IL18BP antibodies of the present disclosure displayed a range of EC50 values for increased IFNγ levels in the range of approximately 0.1-51 nM in NK cells.

### Example 15: Anti-IL-18BP Antibodies in Macrophage Activation Syndrome

The effect of anti-IL18BP antibodies of the present disclosure on macrophage activation syndrome (MAS) as follows. Wild type B6 mice were treated with cytosine guanine 1826 oligonucleotide (CpG) to induce MAS using a protocol previously described (McClain and Allen, 2018, Blood, 131:1393-1394). CpG injections result in clinical signs of MAS with elevation of IFNγ and IFNγ-related genes. On alternate days, animals were dosed with 10 mg/kg of anti-IL18BP antibodies of the present disclosure (BP-14, BP-11, BP-04, and BP-16), and major organs and serum were harvested on day 10 post study initiation. Organs were weighed, serum was analyzed for cytokine levels using mouse proinflammatory panel 1 (MesoScale Diagnostics), and FACS analysis performed using splenocytes (NK1.1, CD11b, CD19, Ly6G, Ly6C, CD3, F4/80, CD11c, Biolegend). Evidence of splenomegaly, alterations of immune cell frequencies in the spleen, and increased serum cytokines were observed, consistent with previous reports describing this model. Spleen weights were normalized to animal whole body weight. Data analysis was performed using FlowJO® and GraphPad® Prism software. Significance was determined via one-way ANOVA.

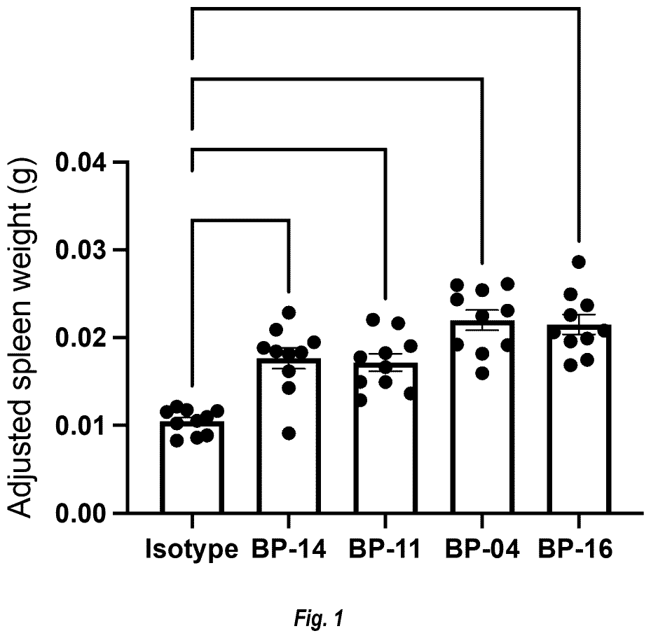

FIG. 1 shows changes in spleen weight in mice administered anti-IL18BP antibodies of the present disclosure compared to that observed in isotype control treated mice. As shown in FIG. 1, mice administered anti-IL18BP antibody BP-14, BP-11, BP-04, and BP-16 showed increases in adjusted spleen weight compared to that of isotype control animals. This data indicated that by blocking IL-18 binding to IL-18BP, anti-IL18BP antibodies of the present disclosure exacerbated the pathological symptoms observed in the spleen due to free (i.e., unbound) IL-18-induced inflammation in the MAS rodent model.

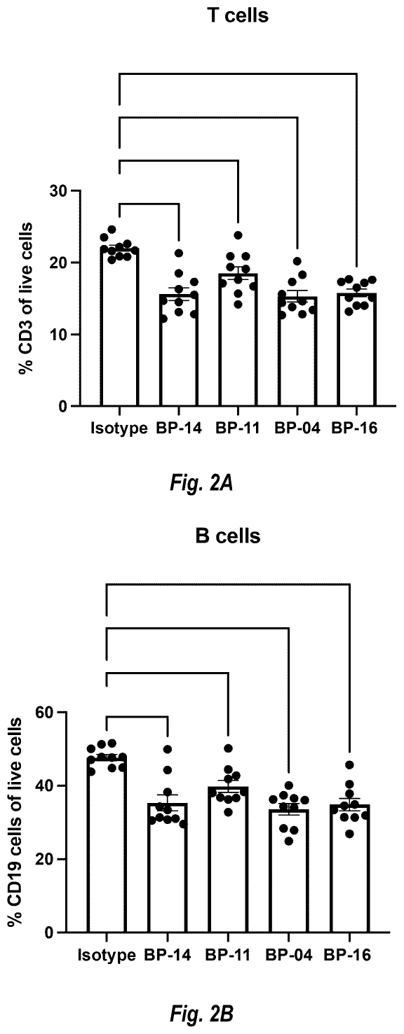

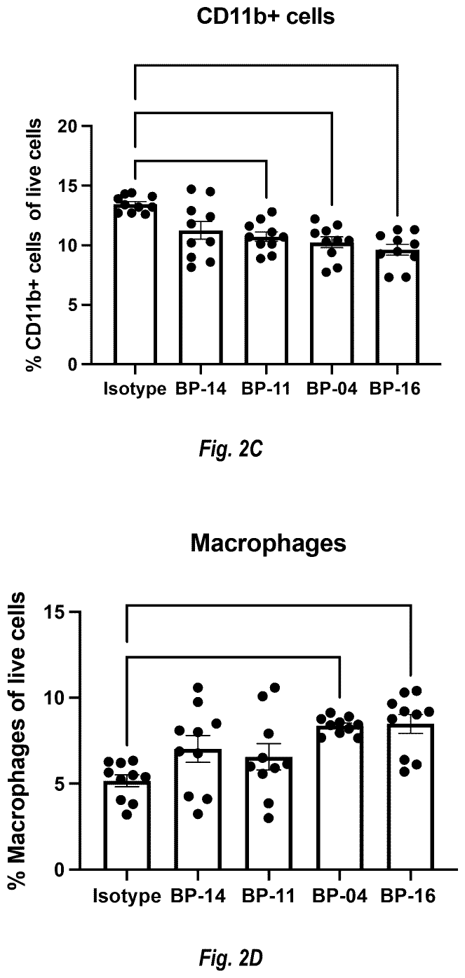

FIGS. 2A-2D show changes in the percentage of various cells types from splenocytes (as measured by FACS analysis) derived from mice administered certain anti-IL18BP antibodies of the present disclosure. As shown in FIGS. 2A-2D, the percentage of T cells (CD3+ cells) in the splenocyte population of cells decreased in response to anti-IL18BP antibody administration (2A); the percentage of B cells (CD19+ cells) in the splenocyte population of cells decreased in response to anti-IL18BP antibody administration (2B); the percentage of CD11b+ cells in the splenocyte population of cells decreased in response to anti-IL18BP antibody administration (2C); and the percentage of macrophages in the splenocyte population of cells increased in response to anti-IL18BP antibody administration (2D).

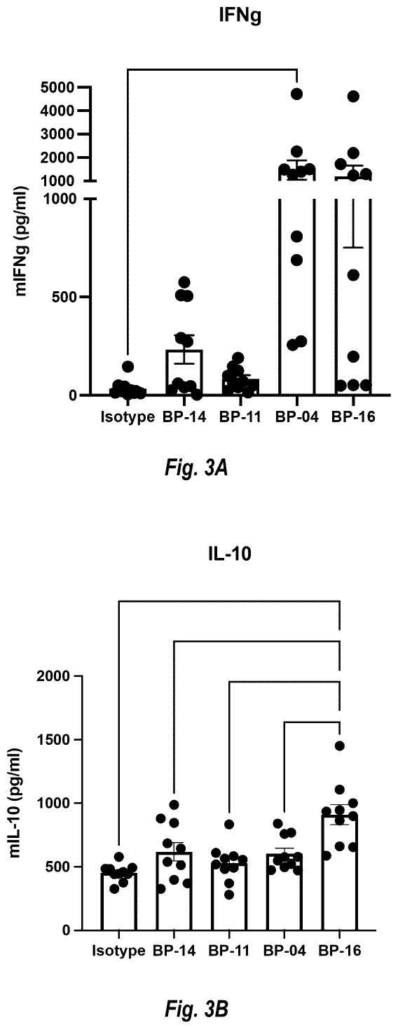

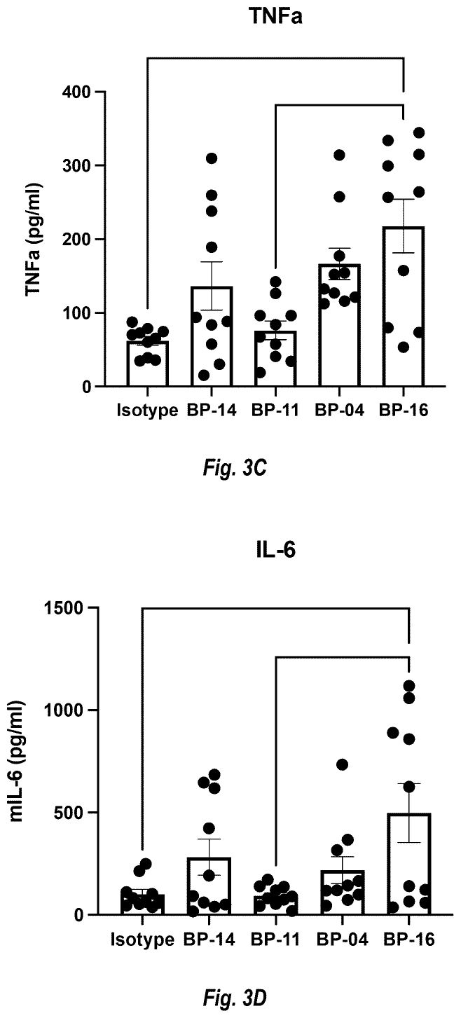

FIGS. 3A-3D show changes in various cytokine levels in serum of mice administered anti-IL18BP antibodies of the present disclosure in this MAS assay. As shown in FIGS. 3A-3D, anti-IL18BP antibodies of the present disclosure, in general, increased serum levels of the inflammatory cytokines IFNγ (3A), IL-10 (3B), TNFα (3C), and IL-6 (3D) compared to that observed in mice administered an isotype control antibody. These results indicated that anti-IL18BP antibody treatment in the rodent MAS model in inflammation lead to a further increase in the inflammatory response due to the blocking of Il-18 binding to IL-18BP.

### Example 16: Anti-IL18BP Antibodies Demonstrate Efficacy in E0771 Syngeneic Tumor Model

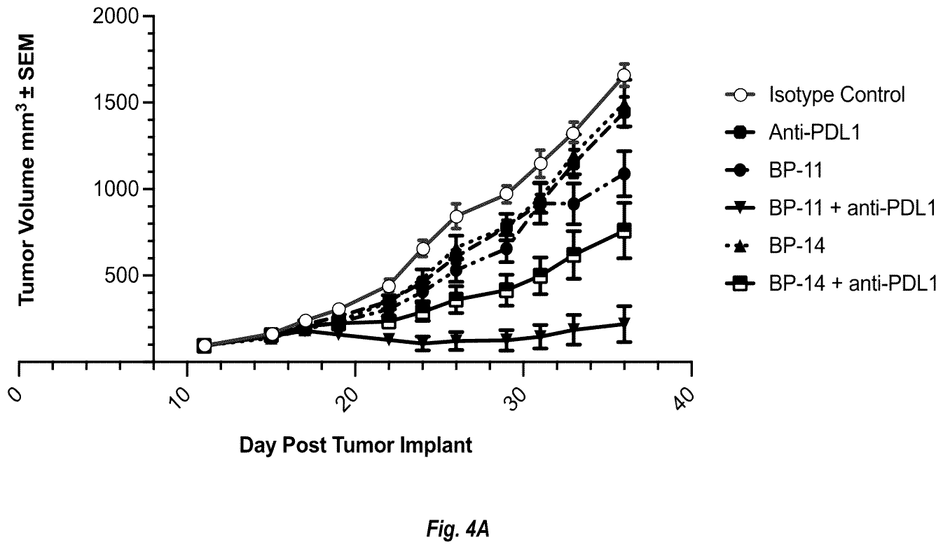

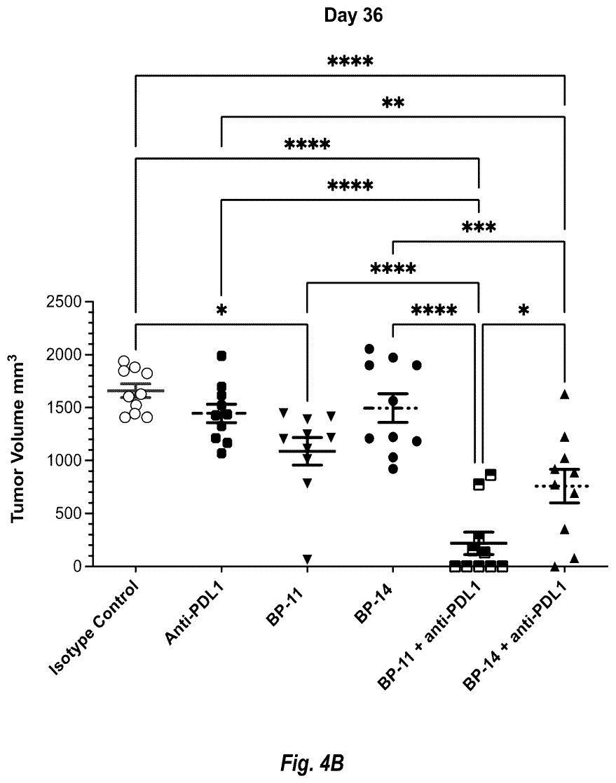

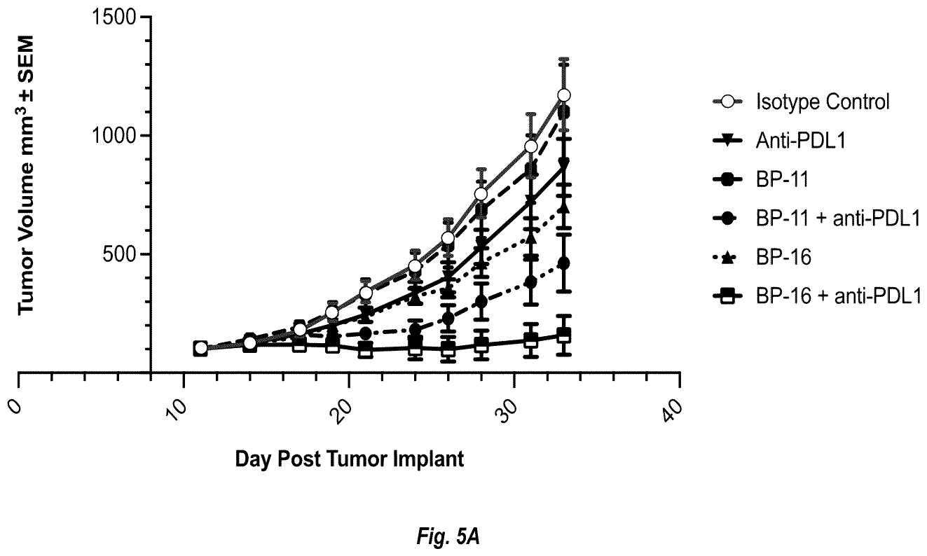

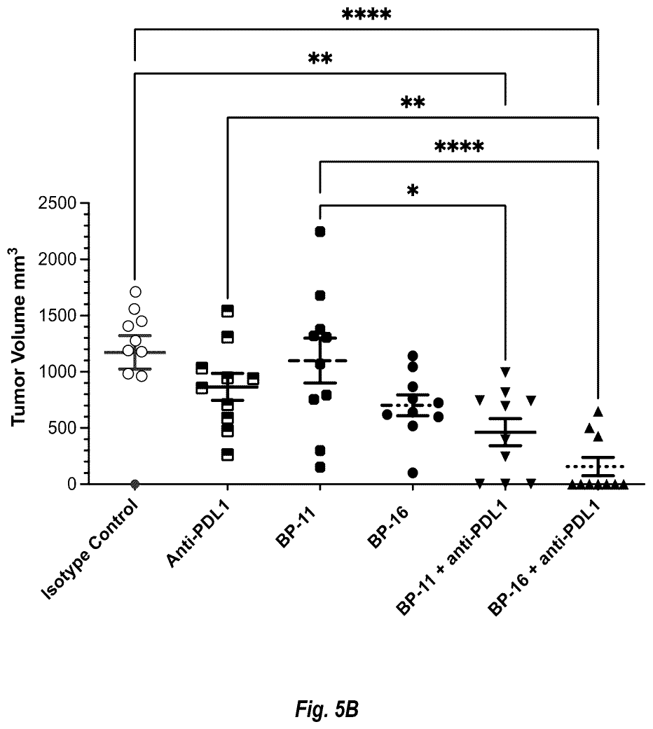

To examine the effects of anti-IL18BP antibodies on tumor grown in an animal model, the following studies were performed. WT B6 mice were inoculated with 400,000 E0771 cells in the flank. On day 12 post inoculation, mice were randomized and dosed with 10 mg/kg of anti-IL18Bp antibodies of the present disclosure with or without coadministration of 3 mg/kg of anti-PDL1 antibody or isotype antibody control. Animals were administered a total of 6 antibody doses (dosed biweekly) with tumor caliper measurements taken triweekly. Inhibition of tumor growth was observed across two independent experiments. Data analysis was performed using GraphPad R Prism software. Significance was determined via one-way ANOVA. Tumor volume mean±SEM and individual tumor volumes on indicated days are shown in FIGS. 4A and 4B and FIGS. 5A and 5B.

As shown in FIGS. 4A and 4B, monotherapy administration of anti-IL18BP antibody BP-11 or anti-IL18BP antibody BP-14 showed reduced tumor volume increases similar to (for BP-14) or better than (for BP-11) that observed with monotherapy administration of anti-PDL1 over time. Combination therapy using anti-IL18BP antibody BP-11 or anti-IL18BP antibody BP-14 together with anti-PDL1 administration showed a significant reduction in tumor volume growth over time compared to that observed with monotherapy treatment with anti-IL18BP antibody BP-11, anti-IL18BP antibody BP-14, or anti-PDL1 antibody.

Similarly, as shown in FIGS. 5A and 5B, monotherapy administration of anti-IL18BP antibody BP-11 or anti-IL18BP antibody BP-16 showed reduced tumor volume increases similar to (for BP-11) or better than (for BP-16) that observed with monotherapy administration of anti-PDL1 over time. Combination therapy using anti-IL18BP antibody BP-11 or anti-IL18BP antibody BP-14 together with anti-PDL1 administration showed a significant reduction in tumor volume growth over time compared to that observed with monotherapy treatment with anti-IL18BP antibody BP-11, anti-IL18BP antibody BP-16, or anti-PDL1 antibody.

Taken together, these results showed that anti-IL18BP antibodies of the present disclosure were effective at reducing tumor volume as monotherapy and in combination therapy with anti-PDL1 antibody, a check-point inhibitor. These results also showed that anti-IL18BP antibodies of the present disclosure were effective at reducing rate of tumor growth both as a monotherapy and in combination with anti-PDL1 antibody treatment.

### Example 17: Anti-IL-18BP Antibodies Increase Free IL-18 in the Serum

A mouse-free IL-18 ELISA assay was developed to measure the amount of free IL-18 in serum as follows. Human IL-18BP-Avi-His protein was biotinylated using EZ-Link Sulfo-NHS-Biotin labeling kit (Life Technologies. Cat. #A39256). Biotinylated human IL-18BP was coated on MSD streptavidin single spot plate (MSD. Cat. #L21SA-1) overnight at 4° C. After washing, mouse IL-18 standards or mouse serum samples were added to the coated plates and incubated overnight at 4° C. The anti-mouse IL-18 antibody (Novus, Cat. #NBP2-90394) diluted in MSD buffer 45 was added to the plates after washing. Plates were incubated at room temperature for 2 hours. The plates were then washed followed by the addition of secondary antibody (MSD goat anti-rabbit antibody. Cat. #R32AB-1) diluted in MSD buffer 45. Plates were incubated for another hour at room temperature. Assay signal was measured on an MSD plate reader with MSD GOLD read buffer.

In order to test the ability of the anti-IL-18BP antibodies of the present disclosure to increase free IL-18 in the serum, the MAS model, described above in Example 15, was utilized. Serum was harvested on day 10 post study initiation. In this experiment, mouse IL-18BP was measured using a mouse IL-18BP ELISA kit (Abcam. Cat. # ab254509) and total mouse IL-18 levels were measured using a mouse IL-18 ELISA kit (Abcam, Cat. # ab216165).

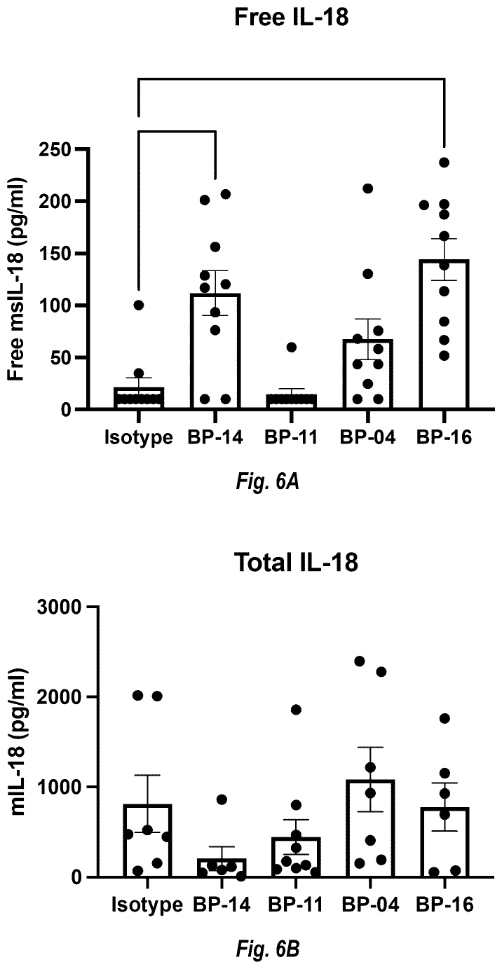

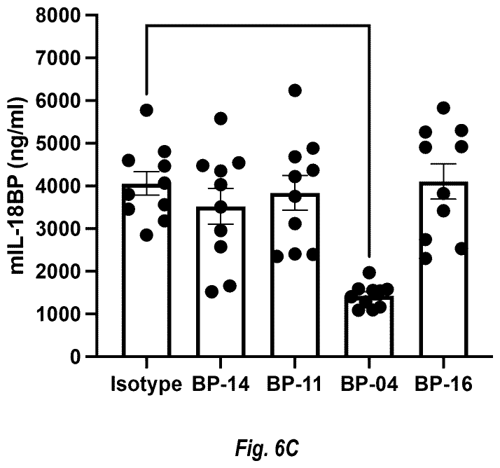

As shown in FIG. 6A, anti-IL-18BP antibodies BP-04, BP-14, and BP-16 increased free IL-18 in serum in this model. Anti-IL-18BP antibody BP-11 had no effect on free IL-18 as measured in this assay: total serum IL-18 levels were not significantly different after treatment with IL-18BP antibodies (FIG. 6B). Serum IL-18BP levels were unchanged for all antibodies except BP-04, which showed a decrease in serum Il-18BP levels (FIG. 6C). These results demonstrated that anti-IL-18BP antibodies of the present disclosure are effective at increasing the levels of free IL-18 in serum.

### Example 18: Anti-IL-18BP Antibody BP-04 Efficacy in a Mouse Tumor Model as Monotherapy or in Combination with Anti-PD-L1

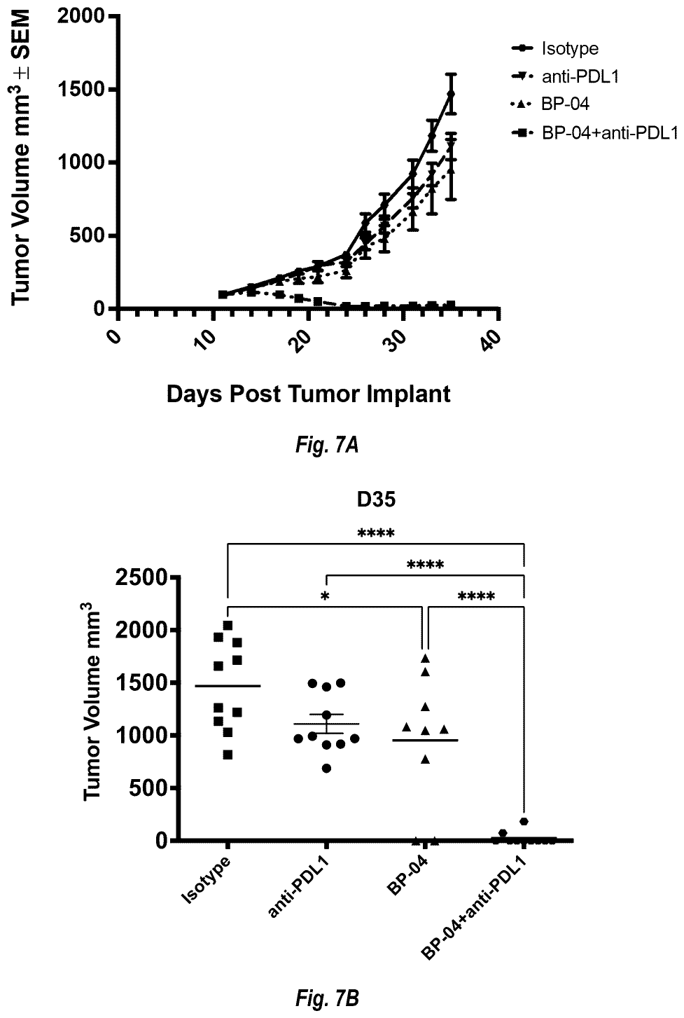

Anti-IL-18BP antibody BP-04 was tested as a monotherapy or in combination with anti-PD-L1 antibody in the E0771 mouse syngeneic tumor model, similar to that described above in Example 16. Monotherapy treatment with anti-IL-18BP antibody BP-04 resulted in significantly reduced tumor volume compared to that observed in the isotype treated group (FIG. 7A and FIG. 7B). Combination treatment of anti-IL-18BP antibody BP-04 together with anti-PD-L1 antibody led to a significant reduction in tumor volume relative to monotherapy treatment with either anti-IL-18BP antibody BP-04 or anti-PD-L1 antibody alone as monotherapy treatments (FIG. 7A and FIG. 7B). Of note, combination therapy with anti-IL-18BP antibody BP-04 and anti-PD-L1 antibody resulted in tumor cures in 8/10 mice in this model (FIG. 7B). These data provide further evidence of the anti-tumor effect of IL-18BP antibodies of the present disclosure in a mouse model of cancer.

### Example 19: Anti-IL-18BP Antibody BP-04 Decreased Serum IL-18BP Levels and Increased Levels of Proinflammatory Cytokines and Chemokines in Tumor Lysate in the E0771 Model

In order to examine the effect of anti-IL-18BP antibodies of the present disclosure on the tumor microenvironment, E0771 mouse tumor model was utilized. Wildtype B6 mice were inoculated with 400,000 E0771 cells in the flank. On day 11 post inoculation, mice were randomized and dosed with 10 mg/kg of anti-IL-18BP antibody BP-04 as monotherapy or in combination with 3 mg/kg anti-PD-L1 antibody. Animals were dosed twice weekly. 24 hours after the 3rd dose, serum and tumor samples were harvested from the animals. Tumors were lysed as described (Zhou et al. 2020, Immunity, 52, 357-373). Briefly, tumors were homogenized in PBS supplemented with Halt™ Protease and Phosphatase Inhibitor Cocktail (ThermoFisher Scientific. Cat. #78440) in gentle MACSMTubes (Miltenyi Biotec) using gentleMACS Dissociator (Miltenyi Biotec) following the manufacturer's protocol. For every 100 mg of tumor tissue. 500 μl buffer was used. Tumor homogenates were clarified by centrifugation. Homogenates were normalized based on total protein concentrations determined by BCA Protein Assay Kit (Pierce). Serum and tumor lysate were analyzed using V-plex mouse cytokine 19-Plex kit (MSD. Cat. #K15255D). IL-18BP levels were measured using mouse IL-18BP ELISA kit (Abcam. Cat. # ab254509). Total IL-18 levels were measured using mouse IL-18 ELISA kit (Abcam. Cat. # ab216165).

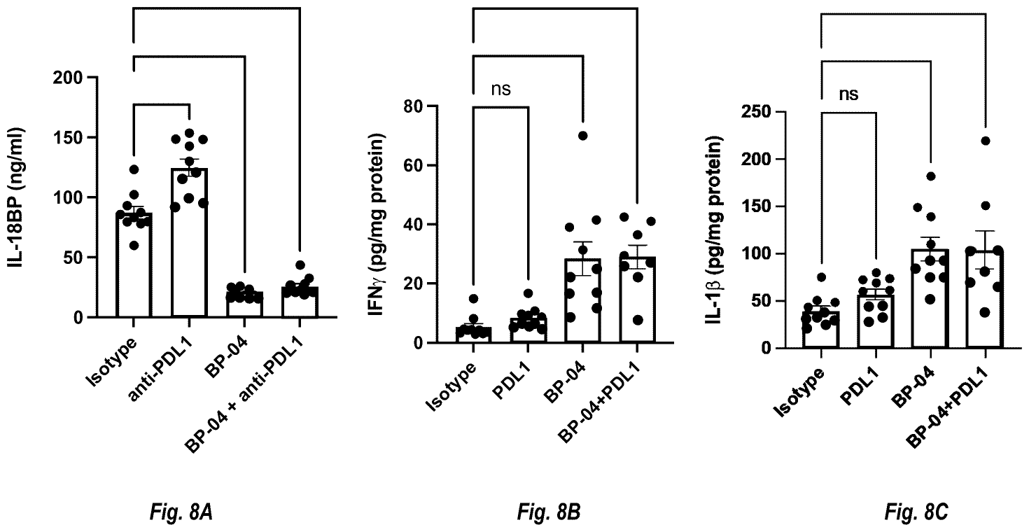

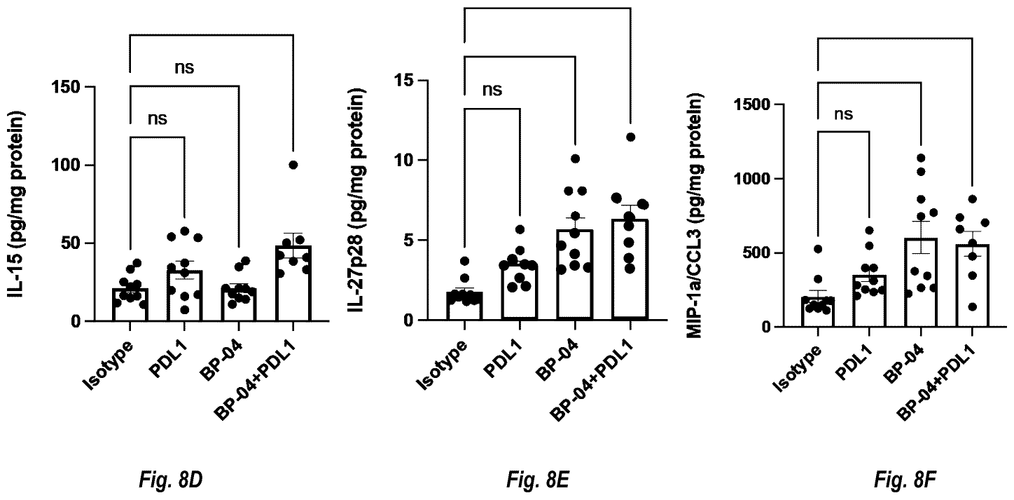

As shown in FIG. 8A, animals that received anti-IL-18BP antibody BP-04 had significantly reduced IL-18BP levels in the serum. These results were consistent with those provided herein using a MAS model (FIG. 6C). In tumor lysate, anti-IL-18BP antibody BP-04 treatment alone or in combination with anti-PD-L1 antibody significantly increased levels of proinflammatory cytokines and chemokines IFNγ, IL-1B, IL-15, IL-27, and MIP-1α (FIGS. 8B, 8C, 8D, 8E, and 8F). These results indicated that anti-IL-18BP antibody BP-04 treatment decreased IL-18BP levels in serum and increased proinflammatory cytokine and chemokine levels in the tumor microenvironment. Increased levels of proinflammatory cytokines and chemokines in the tumor microenvironment are consistent with the anti-tumor effect observed following administration of anti-IL-18BP antibodies of the present disclosure.

### Example 20: Humanization and Affinity Maturation of Anti-IL-18BP Antibody BP-04

Anti-IL-18BP antibody BP-04 of the present disclosure was humanized and affinity-matured using standard techniques known to one of skill in the art.

Unique amino acid sequences of the variable heavy chains and variable light chains of anti-IL18BP antibody variants of the present disclosure are provided below. The Kabat heavy chain CDR sequences and light chain CDR sequences of the humanized and affinity-matured antibodies are set forth in Table 12 and Table 13 below, respectively. The heavy chain variable region and light chain variable region sequences of the humanized and affinity-matured antibodies are set forth in Table 11 below (in which the Kabat CDR sequences are underlined in the variable heavy chain region sequences).

TABLE 11

| | | SEQ | | SEQ |
|---|---|---|---|---|
| | | ID | | ID |
| Antibody | Heavy Chain Variable | NO: | Light Chain Variable | NO: |
| BP-04.01 | QVQLVQSGAEVKKPGASVKVSC | 134 | DIQMTQSPSSVSASVGDRVTI | 166 |
| | KASGYTFTDYDMWVRQAPGQGL | | TCRASDNVYSNLAWYQQKPGK | |
| | EWMGIISTYYGDVSYAIKFQGR | | APKLLIYGAISLADGVPSRES | |
| | VTMTRDKSTSTVYMELSSLRSE | | GSGSGTDFTLTISSLQPEDFA | |
| | DTAVYYCARSGTYYYGTRESAL | | TYYCQHFWGNPYTFGQGTKLE | |
| | DYWGQGTLVTVSS | | IK | |
| BP-04.02 | QVQLVQSGAEVKKPGASVKVSC | 135 | DIQMTQSPSSVSASVGDRVTI | 166 |
| | KASGYTFTDYDLHWVRQAPGQG | | TCRASDNVYSNLAWYQQKPGK | |
| | LEWMGIISTYYGDVSYAQKFQG | | APKLLIYGAISLADGVPSRES | |
| | RVTMTRDKSTSTVYMELSSLRS | | GSGSGTDFTLTISSLQPEDFA | |
| | EDTAVYYCARSGMYYYGTRESH | | TYYCQHFWGNPYTFGQGTKLE | |
| | ALDYWGQGTLVTVSS | | IK | |
| BP-04.03 | QVQLVQSGAEVKKPGASVKVSC | 136 | DIQMTQSPSSVSASVGDRVTI | 166 |
| | KASGYTFTDYDMHWVRQAPGQG | | TCRASDNVYSNLAWYQQKPGK | |
| | LEWMGIISTYYGDVSYAQKFQG | | APKLLIYGAISLADGVPSRES | |
| | RVTMTRDKSTSTVYMELSSLRS | | GSGSGTDFTLTISSLQPEDFA | |
| | ENTAVYYCARSGTYYYGTREQH | | TYYCQHFWGNPYTFGQGTKLE | |
| | ALDYWGQGTLVTVSS | | IK | |
| BP-04.04 | QVQLVQSGAEVKKPGASVKVSC | 137 | DIQMTQSPSSVSASVGDRVTI | 166 |
| | KASGYTFTDYDMHWVRQAPGQG | | TCRASDNVYSNLAWYQQKPGK | |
| | LEWMGIISTYYGDVSYAQKKQG | | APKLLIYGAISLADGVPSRES | |
| | RVTMTRDKSTSTVYMELSSLRS | | GSGSGTDFTLTISSLQPEDFA | |
| | EDTAVYYCARSGTYNYGTRESH | | TYYCQHFWGNPYTFGQGTKLE | |
| | PLDYWGQGTLVTVSS | | IK | |
| BP-04.05 | QVQLVQSGAEVKKPGASVKVSC | 138 | DIQMTQSPSSVSASVGDRVTI | 166 |
| | KASGYTFTDYDMHWVRQAPGQG | | TCRASDNVYSNLAWYQQKPGK | |
| | LEWMGIISTYYGVVSYAQKKQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | AFDYWGQGTLVTVSS | | IK | |
| BP-04.06 | QVQLVQSGAEVKKPGASVKVSC | 139 | DIQMTQSPSSVSASVGDRVTI | 166 |
| | KASGYTFTDYDMHWVRQAPGQG | | TCRASDNVYSNLAWYQQKPGK | |
| | LEWMGIISTYYGVVSYAQKKQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | PLDYWGQGTLVTVSS | | IK | |
| BP-04.07 | QVQLVQSGAEVKKPGASVKVSC | 140 | DIQMTQSPSSVSASVGDRVTI | 166 |
| | KASGYTFTDYDMHWVRQAPGQG | | TCRASDNVYSNLAWYQQKPGK | |
| | LEWMGIISTYYGVVSYAQKKQG | | APKLLIYGAISLADGVPSRES | |
| | RVTMTRDKSTSTVYMELSSLRS | | GSGSGTDFTLTISSLQPEDFA | |
| | EDTAVYYCARSGTYNYGTRESH | | TYYCQHFWGNPYTFGQGTKLE | |
| | ALDYWGQGTLVTVSS | | IK | |
| BP-04.08 | QVQLVQSGAEVKKPGASVKVSC | 141 | DIQMTQSPSSVSASVGDRVTI | 166 |
| | KASGYTFTDYDMHWVRQAPGQG | | TCRASDNVYSNLAWYQQKPGK | |
| | LEWMGIYSTYYGDVSYAQKKQG | | APKLLIYGAISLADGVPSRES | |
| | RVTMTRDKSTSTVYMELSSLRS | | GSGSGTDFTLTISSLQPEDFA | |
| | EDTAVYYCARSGTYNYGTRESH | | TYYCQHFWGNPYTFGQGTKLE | |
| | ALDYWGQGTLVTVSS | | IK | |
| BP-04.09 | QVQLVQSGAEVKKPGASVKVSC | 142 | DIQMTQSPSSVSASVGDRVTI | 166 |
| | KASGYTFTDYDMHWVRQAPGQG | | TCRASDNVYSNLAWYQQKPGK | |
| | LEWMGIYSTYYGVVSYAQKFQG | | APKLLIYGAISLADGVPSRES | |
| | RVTMTRDKSTSTVYMELSSLRS | | GSGSGTDETLTISSLQPEDFA | |
| | EDTAVYYCARSGTYYYGTRESH | | TYYCQHFWGNPYTFGQGTKLE | |
| | AFDYWGQGTLVTVSS | | IK | |
| BP-04.10 | QVQLVQSGAEVKKPGASVKVSC | 143 | DIQMTQSPSSVSASVGDRVTI | 166 |
| | KASGYTFTDYDMHWVRQAPGQG | | TCRASDNVYSNLAWYQQKPGK | |
| | LEWMGIYSTYYGVVSYAQKFQG | | APKLLIYGAISLADGVPSRES | |
| | RVTMTRDKSTSTVYMELSSLRS | | GSGSGTDETLTISSLQPEDFA | |
| | EDTAVYYCARSGTYYYGTRESH | | TYYCQHFWGNPYTFGQGTKLE | |
| | PLDYWGQGTLVTVSS | | IK | |
| BP-04.11 | QVQLVQSGAEVKKPGASVKVSC | 144 | DIQMTQSPSSVSASVGDRVTI | 166 |
| | KASGYTFTDYDMHWVRQAPGQG | | TCRASDNVYSNLAWYQQKPGK | |
| | LEWMGIYSTYYGVVSYAQKFQG | | APKLLIYGAISLADGVPSRES | |
| | RVTMTRDKSTSTVYMELSSLRS | | GSGSGTDETLTISSLQPEDFA | |
| | EDTAVYYCARSGTYNYGTRESH | | TYYCQHFWGNPYTFGQGTKLE | |
| | ALDYWGQGTLVTVSS | | IK | |
| BP-04.12 | QVQLVQSGAEVKKPGASVKVSC | 145 | DIQMTQSPSSVSASVGDRVTI | 166 |
| | KASGYTFTDYDMHWVRQAPGQG | | TCRASDNVYSNLAWYQQKPGK | |
| | LEWMGIYSTYYGVVSYAQKKQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | ALDYWGQGTLVTVSS | | IK | |
| BP-04.13 | QVQLVQSGAEVKKPGASVKVSC | 146 | DIQMTQSPSSVSASVGDRVTI | 166 |
| | KASGYTFTDYDMHWVRQAPGQG | | TCRASDNVYSNLAWYQQKPGK | |
| | LEWMGIISTYYGDVSYAQKFQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | PFDYWGQGTLVTVSS | | IK | |
| BP-04.14 | QVQLVQSGAEVKKPGASVKVSC | 147 | DIQMTQSPSSVSASVGDRVTI | 166 |
| | KASGYTFTDYDMHWVRQAPGQG | | TCRASDNVYSNLAWYQQKPGK | |
| | LEWMGIISTYYGDVSYAQKFQG | | APKLLIYGAISLADGVPSRFS | |
| | RVTMTRDKSTSTVYMELSSLRS | | GSGSGTDFTLTISSLQPEDFA | |
| | EDTAVYYCARSGTYNYGTRESH | | TYYCQHFWGNPYTFGQGTKLE | |
| | AFDYWGQGTLVTVSS | | IK | |
| BP-04.15 | QVQLVQSGAEVKKPGASVKVSC | 148 | DIQMTQSPSSVSASVGDRVTI | 166 |
| | KASGYTFTDYDMHWVRQAPGQG | | TCRASDNVYSNLAWYQQKPGK | |
| | LEWMGIISTYYGDVSYAQKFQG | | APKLLIYGAISLADGVPSRFS | |
| | RVTMTRDKSTSTVYMELSSLRS | | GSGSGTDFTLTISSLQPEDFA | |
| | EDTAVYYCARSGTYNYGTRESH | | TYYCQHFWGNPYTFGQGTKLE | |
| | PLDYWGQGTLVTVSS | | IK | |
| BP-04.16 | QVQLVQSGAEVKKPGASVKVSC | 149 | DIQMTQSPSSVSASVGDRVTI | 166 |
| | KASGYTFTDYDMHWVRQAPGQG | | TCRASDNVYSNLAWYQQKPGK | |
| | LEWMGIISTYYGDVSYAQKKQG | | APKLLIYGAISLADGVPSRFS | |
| | RVTMTRDKSTSTVYMELSSLRS | | GSGSGTDETLTISSLQPEDFA | |
| | EDTAVYYCARSGTYYYGTRESH | | TYYCQHFWGNPYTFGQGTKLE | |
| | AFDYWGQGTLVTVSS | | IK | |
| BP-04.17 | QVQLVQSGAEVKKPGASVKVSC | 150 | DIQMTQSPSSVSASVGDRVTI | 166 |
| | KASGYTFTDYDMHWVRQAPGQG | | TCRASDNVYSNLAWYQQKPGK | |
| | LEWMGIISTYYGDVSYAQKKQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | PLDYWGQGTLVTVSS | | IK | |
| BP-04.18 | QVQLVQSGAEVKKPGASVKVSC | 151 | DIQMTQSPSSVSASVGDRVTI | 166 |
| | KASGYTFTDYDMHWVRQAPGQG | | TCRASDNVYSNLAWYQQKPGK | |
| | LEWMGIISTYYGDVSYAQKKQG | | APKLLIYGAISLADGVPSRES | |
| | RVTMTRDKSTSTVYMELSSLRS | | GSGSGTDFTLTISSLQPEDFA | |
| | EDTAVYYCARSGTYNYGTRESH | | TYYCQHFWGNPYTFGQGTKLE | |
| | ALDYWGQGTLVTVSS | | IK | |
| BP-04.19 | QVQLVQSGAEVKKPGASVKVSC | 152 | DIQMTQSPSSVSASVGDRVTI | 166 |
| | KASGYTFTDYDMHWVRQAPGQG | | TCRASDNVYSNLAWYQQKPGK | |
| | LEWMGIISTYYGVVSYAQKFQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | AFDYWGQGTLVTVSS | | IK | |
| BP-04.20 | QVQLVQSGAEVKKPGASVKVSC | 153 | DIQMTQSPSSVSASVGDRVTI | 166 |
| | KASGYTFTDYDMHWVRQAPGQG | | TCRASDNVYSNLAWYQQKPGK | |
| | LEWMGIISTYYGVVSYAQKFQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | PLDYWGQGTLVTVSS | | IK | |
| BP-04.21 | QVQLVQSGAEVKKPGASVKVSC | 154 | DIQMTQSPSSVSASVGDRVTI | 166 |
| | KASGYTFTDYDMHWVRQAPGQG | | TCRASDNVYSNLAWYQQKPGK | |
| | LEWMGIISTYYGVVSYAQKFQG | | APKLLIYGAISLADGVPSRES | |
| | RVTMTRDKSTSTVYMELSSLRS | | GSGSGTDFTLTISSLQPEDFA | |
| | EDTAVYYCARSGTYNYGTRESH | | TYYCQHFWGNPYTFGQGTKLE | |
| | ALDYWGQGTLVTVSS | | IK | |
| BP-04.22 | QVQLVQSGAEVKKPGASVKVSC | 155 | DIQMTQSPSSVSASVGDRVTI | 166 |
| | KASGYTFTDYDMHWVRQAPGQG | | TCRASDNVYSNLAWYQQKPGK | |
| | LEWMGIISTYYGVVSYAQKKQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | ALDYWGQGTLVTVSS | | IK | |
| BP-04.23 | QVQLVQSGAEVKKPGASVKVSC | 156 | DIQMTQSPSSVSASVGDRVTI | 166 |
| | KASGYTFTDYDMHWVRQAPGQG | | TCRASDNVYSNLAWYQQKPGK | |
| | LEWMGIYSTYYGDVSYAQKFQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | AFDYWGQGTLVTVSS | | IK | |
| BP-04.24 | QVQLVQSGAEVKKPGASVKVSC | 157 | DIQMTQSPSSVSASVGDRVTI | 166 |
| | KASGYTFTDYDMHWVRQAPGQG | | TCRASDNVYSNLAWYQQKPGK | |
| | LEWMGIYSTYYGDVSYAQKFQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | PLDYWGQGTLVTVSS | | IK | |
| BP-04.25 | QVQLVQSGAEVKKPGASVKVSC | 158 | DIQMTQSPSSVSASVGDRVTI | 166 |
| | KASGYTFTDYDMHWVRQAPGQG | | TCRASDNVYSNLAWYQQKPGK | |
| | LEWMGIYSTYYGDVSYAQKFQG | | APKLLIYGAISLADGVPSRES | |
| | RVTMTRDKSTSTVYMELSSLRS | | GSGSGTDFTLTISSLQPEDFA | |
| | EDTAVYYCARSGTYNYGTRESH | | TYYCQHFWGNPYTFGQGTKLE | |
| | ALDYWGQGTLVTVSS | | IK | |
| BP-04.26 | QVQLVQSGAEVKKPGASVKVSC | 159 | DIQMTQSPSSVSASVGDRVTI | 166 |
| | KASGYTFTDYDMHWVRQAPGQG | | TCRASDNVYSNLAWYQQKPGK | |
| | LEWMGIYSTYYGVVSYAQKFQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | ALDYWGQGTLVTVSS | | IK | |
| BP-04.27 | QVQLVQSGAEVKKPGASVKVSC | 160 | DIQMTQSPSSVSASVGDRVTI | 166 |
| | KASGYTFTDYDMHWVRQAPGQG | | TCRASDNVYSNLAWYQQKPGK | |
| | LEWMGIISTYYGDVSYAQKFQG | | APKLLIYGAISLADGVPSRFS | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | AFDYWGQGTLVTVSS | | IK | |
| BP-04.28 | QVQLVQSGAEVKKPGASVKVSC | 161 | DIQMTQSPSSVSASVGDRVTI | 166 |
| | KASGYTFTDYDMHWVRQAPGQG | | TCRASDNVYSNLAWYQQKPGK | |
| | LEWMGIISTYYGDVSYAQKFQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | PLDYWGQGTLVTVSS | | IK | |
| BP-04.29 | QVQLVQSGAEVKKPGASVKVSC | 162 | DIQMTQSPSSVSASVGDRVTI | 166 |
| | KASGYTFTDYDMHWVRQAPGQG | | TCRASDNVYSNLAWYQQKPGK | |
| | LEWMGIISTYYGDVSYAQKFQG | | APKLLIYGAISLADGVPSRES | |
| | RVTMTRDKSTSTVYMELSSLRS | | GSGSGTDFTLTISSLQPEDFA | |
| | EDTAVYYCARSGTYNYGTRESH | | TYYCQHFWGNPYTFGQGTKLE | |
| | ALDYWGQGTLVTVSS | | IK | |
| BP-04.30 | QVQLVQSGAEVKKPGASVKVSC | 163 | DIQMTQSPSSVSASVGDRVTI | 166 |
| | KASGYTFTDYDMHWVRQAPGQG | | TCRASDNVYSNLAWYQQKPGK | |
| | LEWMGIISTYYGDVSYAQKKQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | ALDYWGQGTLVTVSS | | IK | |
| BP-04.31 | QVQLVQSGAEVKKPGASVKVSC | 164 | DIQMTQSPSSVSASVGDRVTI | 166 |
| | KASGYTFTDYDMHWVRQAPGQG | | TCRASDNVYSNLAWYQQKPGK | |
| | LEWMGIISTYYGVVSYAQKFQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | ALDYWGQGTLVTVSS | | IK | |
| BP-04.32 | QVQLVQSGAEVKKPGASVKVSC | 165 | DIQMTQSPSSVSASVGDRVTI | 166 |
| | KASGYTFTDYDMHWVRQAPGQG | | TCRASDNVYSNLAWYQQKPGK | |
| | LEWMGIYSTYYGDVSYAQKFQG | | APKLLIYGAISLADGVPSRES | |
| | RVTMTRDKSTSTVYMELSSLRS | | GSGSGTDFTLTISSLQPEDFA | |
| | EDTAVYYCARSGTYYYGTRESH | | TYYCQHFWGNPYTFGQGTKLE | |
| | ALDYWGQGTLVTVSS | | IK | |

TABLE 12

| | | SEQ | | SEQ | | SEQ |
|---|---|---|---|---|---|---|
| | | ID | | ID | | ID |
| Antibody | HVR-H1 | NO: | HVR-H2 | NO: | HVR-H3 | NO: |
| BP-04.01 | DYDM | 167 | IISTYYGDVS | 170 | SGTYYYGT | 179 |
| | | | YAIKFQG | | RESALDY | |
| BP-04.02 | DYDLH | 168 | IISTYYGDVS | 171 | SGMYYYGT | 180 |
| | | | YAQKFQG | | RESHALDY | |
| BP-04.03 | DYDMH | 169 | IISTYYGDVS | 171 | SGTYYYGT | 181 |
| | | | YAQKFQG | | REQHALDY | |
| BP-04.04 | DYDMH | 169 | IISTYYGDVS | 172 | SGTYNYGT | 182 |
| | | | YAQKKQG | | RESHPLDY | |
| BP-04.05 | DYDMH | 169 | IISTYYGVVS | 173 | SGTYYYGT | 183 |
| | | | YAQKKQG | | RESHAFDY | |
| BP-04.06 | DYDMH | 169 | IISTYYGVVS | 173 | SGTYYYGT | 184 |
| | | | YAQKKQG | | RESHPLDY | |
| BP-04.07 | DYDMH | 169 | IISTYYGVVS | 173 | SGTYNYGT | 185 |
| | | | YAQKKQG | | RESHALDY | |
| BP-04.08 | DYDMH | 169 | IYSTYYGDV | 174 | SGTYNYGT | 185 |
| | | | SYAQKKQG | | RESHALDY | |
| BP-04.09 | DYDMH | 169 | IYSTYYGVV | 175 | SGTYYYGT | 183 |
| | | | SYAQKFQG | | RESHAFDY | |
| BP-04.10 | DYDMH | 169 | IYSTYYGVV | 175 | SGTYYYGT | 184 |
| | | | SYAQKFQG | | RESHPLDY | |
| BP-04.11 | DYDMH | 169 | IYSTYYGVV | 175 | SGTYNYGT | 185 |
| | | | SYAQKFQG | | RESHALDY | |
| BP-04.12 | DYDMH | 169 | IYSTYYGVV | 176 | SGTYYYGT | 186 |
| | | | SYAQKKQG | | RESHALDY | |
| BP-04.13 | DYDMH | 169 | IISTYYGDVS | 171 | SGTYYYGT | 187 |
| | | | YAQKFQG | | RESHPFDY | |
| BP-04.14 | DYDMH | 169 | IISTYYGDVS | 171 | SGTYNYGT | 188 |
| | | | YAQKFQG | | RESHAFDY | |
| BP-04.15 | DYDMH | 169 | IISTYYGDVS | 171 | SGTYNYGT | 182 |
| | | | YAQKFQG | | RESHPLDY | |
| BP-04.16 | DYDMH | 169 | IISTYYGDVS | 172 | SGTYYYGT | 183 |
| | | | YAQKKQG | | RESHAFDY | |
| BP-04.17 | DYDMH | 169 | IISTYYGDVS | 172 | SGTYYYGT | 184 |
| | | | YAQKKQG | | RESHPLDY | |
| BP-04.18 | DYDMH | 169 | IISTYYGDVS | 172 | SGTYNYGT | 185 |
| | | | YAQKKQG | | RESHALDY | |
| BP-04.19 | DYDMH | 169 | IISTYYGVVS | 177 | SGTYYYGT | 183 |
| | | | YAQKFQG | | RESHAFDY | |
| BP-04.20 | DYDMH | 169 | IISTYYGVVS | 177 | SGTYYYGT | 184 |
| | | | YAQKFQG | | RESHPLDY | |
| BP-04.21 | DYDMH | 169 | IISTYYGVVS | 177 | SGTYNYGT | 185 |
| | | | YAQKFQG | | RESHALDY | |
| BP-04.22 | DYDMH | 169 | IISTYYGVVS | 173 | SGTYYYGT | 186 |
| | | | YAQKKQG | | RESHALDY | |
| BP-04.23 | DYDMH | 169 | IYSTYYGDV | 178 | SGTYYYGT | 183 |
| | | | SYAQKFQG | | RESHAFDY | |
| BP-04.24 | DYDMH | 169 | IYSTYYGDV | 178 | SGTYYYGT | 184 |
| | | | SYAQKFQG | | RESHPLDY | |
| BP-04.25 | DYDMH | 169 | IYSTYYGDV | 178 | SGTYNYGT | 185 |
| | | | SYAQKFQG | | RESHALDY | |
| BP-04.26 | DYDMH | 169 | IYSTYYGVV | 175 | SGTYYYGT | 186 |
| | | | SYAQKFQG | | RESHALDY | |
| BP-04.27 | DYDMH | 169 | IISTYYGDVS | 171 | SGTYYYGT | 183 |
| | | | YAQKFQG | | RESHAFDY | |
| BP-04.28 | DYDMH | 169 | IISTYYGDVS | 171 | SGTYYYGT | 184 |
| | | | YAQKFQG | | RESHPLDY | |
| BP-04.29 | DYDMH | 169 | IISTYYGDVS | 171 | SGTYNYGT | 185 |
| | | | YAQKFQG | | RESHALDY | |
| BP-04.30 | DYDMH | 169 | IISTYYGDVS | 172 | SGTYYYGT | 186 |
| | | | YAQKKQG | | RESHALDY | |
| BP-04.31 | DYDMH | 169 | IISTYYGVVS | 177 | SGTYYYGT | 186 |
| | | | YAQKFQG | | RESHALDY | |
| BP-04.32 | DYDMH | 169 | IYSTYYGDV | 178 | SGTYYYGT | 186 |
| | | | SYAQKFQG | | RESHALDY | |

TABLE 13

| | | SEQ | | SEQ | | SEQ |
|---|---|---|---|---|---|---|
| | | ID | | ID | | ID |
| Antibody | HVR-L1 | NO: | HVR-L2 | NO: | HVR-L3 | NO: |
| BP-04.LC | RASDNV | 247 | GAIS | 248 | QHFWG | 249 |
| | YSNLA | | LAD | | NPYT | |

### Example 21: Cross-Reactivity and Binding Kinetics of Anti-IL-18BP Antibodies

Binding kinetics of humanized and affinity-matured anti-IL-18BP antibody variants of the present disclosure were evaluated using Biacore T200 (Cytiva). Briefly, anti-IL-18BP antibody variants were diluted to 5 μg/mL and captured using a Protein A/G (ThermoFisher, #21186) surface on a CM5 chip (Cytiva) that was prepared by amine coupling according to the instrument manufacturer's recommendations.

The captured anti-IL-18BP antibody variants were tested for their ability to bind human and cynomolgus monkey IL18BP recombinant proteins (described above) as follows. Human and cynomolgus IL18BP analytes were diluted in running buffer (HBS-EP+, Teknova, #8022, with 0.5 mg/mL BSA, MP Biomedicals LLC, #820451), to a concentration of 300 nM, then diluted 3-fold serially to 100, 33.3, 11.1, 3.7, and 1.2 nM. Each analyte sample was injected for 3 minutes to allow association, followed by dissociation in buffer alone for 5 minutes. Each sample injection was followed by the regeneration of the chip with one 180-second injection of 10 mM glycine pH1.7. Fresh antibody was captured at the beginning of each cycle. Data were analyzed using Biacore evaluation software to generate kinetic constants. The equilibrium dissociation constants (K_{D}) were calculated from the fitted association and dissociation rate constants (k-on and k-off) for each of anti-IL-18BP antibody variants.

As shown in Table 14 below, about half of the affinity-matured clones showed higher affinity than the starting humanization template (i.e., BP-04 humanized mouse parental). Notably, humanized variants BP-04.03, BP-04.05, and PB-04.07 bound to both human and cynomolgus IL18BP with binding affinities matching the original hybridoma clone (BP-04).

TABLE 14

| | Human IL-18BP | | | CynoIL-18BP | | |
|---|---|---|---|---|---|---|
| | K-on | K-off | K_{D} | K-on | K-off | K_{D} |
| Antibody | (1/Ms) | (1/s) | (M) | (1/Ms) | (1/s) | (M) |
| BP-04 | 4.3E+05 | 7.1E−03 | 1.7E−08 | 3.5E+05 | 7.9E−03 | 2.3E−08 |
| (humanized | | | | | | |
| mouse | | | | | | |
| parental) | | | | | | |
| BP-04 | 5.8E+05 | 5.9E−04 | 1.0E−09 | 4.4E+05 | 3.2E−03 | 7.3E−09 |
| (hybridoma/ | 5.8E+05 | 6.2E−04 | 1.1E−09 | 3.9E+05 | 3.3E−03 | 8.5E−09 |
| in duplicate) | | | | | | |
| BP-04.25 | 3.0E+05 | 6.1E−03 | 2.1E−08 | 2.2E+05 | 2.2E−02 | 1.0E−07 |
| BP-04.24 | 2.9E+05 | 4.7E−03 | 1.6E−08 | 2.0E+05 | 5.9E−03 | 2.9E−08 |
| BP-04.15 | 2.4E+05 | 2.8E−03 | 1.2E−08 | 2.0E+05 | 4.0E−03 | 2.0E−08 |
| BP-04.09 | 5.3E+05 | 2.8E−04 | 5.2E−10 | 4.5E+05 | 6.0E−04 | 1.3E−09 |
| BP-04.08 | 2.1E+05 | 3.5E−03 | 1.6E−08 | 2.6E+05 | 1.4E−02 | 5.6E−08 |
| BP-04.32 | 3.7E+05 | 7.4E−03 | 2.0E−08 | 3.4E+05 | 2.0E−02 | 5.9E−08 |
| BP-04.07 | 3.5E+05 | 6.8E−04 | 1.9E−09 | 3.2E+05 | 7.6E−04 | 2.4E−09 |
| BP-04.05 | 5.1E+05 | 3.4E−04 | 6.7E−10 | 4.6E+05 | 3.8E−04 | 8.3E−10 |
| BP-04.03 | 4.7E+05 | 9.1E−04 | 1.9E−09 | 4.2E+05 | 1.3E−03 | 3.1E−09 |
| BP-04.29 | 3.9E+05 | 6.2E−03 | 1.6E−08 | 3.4E+05 | 1.1E−02 | 3.2E−08 |
| BP-04.28 | 4.1E+05 | 1.9E−03 | 4.7E−09 | 3.3E+05 | 2.3E−03 | 7.2E−09 |

### Example 22: Humanized and Affinity-Matured Anti-IL-18BP Antibody Variants Display Improved Potency in Human and Mouse Cell-Based Binding Assays

The functional effects of the humanized and affinity matured anti-IL-18BP antibody variants of the present disclosure were tested in cell-based binding assays using human PBMCs and mouse splenocytes, as described above. IFNγ release induced by each anti-IL-18BP antibody variant compared to the parental mouse chimera reference antibody is shown Table 15 below. These results showed that the EC50 of many of the anti-IL-18BP antibody variants of the present disclosure were more potent at cell binding than that observed with the anti-IL-18BP parental clone (BP-04), consistent with the enhanced binding capacity of these variants observed above.

TABLE 15

| | Human | Mouse |
|---|---|---|
| | PBMCs EC50 | splenocytes |
| Antibody | (nM) | EC50 (nM) |
| BP-04 | 15.70 | 14.86 |
| (parental) | | |
| BP-04.01 | undefined | 1.670 |
| BP-04.02 | 41.53 | 12.32 |
| BP-04.03 | 10.51 | 0.8813 |
| BP-04.04 | 18.55 | 31.78 |
| BP-04.05 | 3.178 | 4.915 |
| BP-04.06 | 1.318 | 67.59 |
| BP-04.07 | 4.301 | 51.94 |
| BP-04.08 | 173.7 | 94.10 |
| BP-04.09 | 3.743 | 2.040 |
| BP-04.10 | 10.51 | 501.1 |
| BP-04.11 | 3.943 | 3.606 |
| BP-04.12 | 31.87 | 0.8654 |
| BP-04.13 | 16.60 | 41.44 |
| BP-04.14 | 543.1 | 21.17 |
| BP-04.15 | 12.30 | 43.12 |
| BP-04.16 | 3.337 | 219.8 |
| BP-04.17 | 8.886 | 14.60 |
| BP-04.18 | 11.27 | 28.35 |
| BP-04.19 | 3.098 | 21.56 |
| BP-04.20 | 4.056 | 28.70 |
| BP-04.21 | 4.483 | 595.2 |
| BP-04.22 | 7.713 | 367.9 |
| BP-04.23 | 146.0 | 3.505 |
| BP-04.24 | 4.836 | undefined |
| BP-04.25 | 5.049 | 28.86 |
| BP-04.26 | 36.24 | 6.016 |
| BP-04.27 | 1.285 | 39.63 |
| BP-04.28 | 71.22 | 133.5 |
| BP-04.29 | 3.528 | 45.51 |
| BP-04.30 | undefined | 7.432 |
| BP-04.31 | 6.299 | undefined |
| BP-04.32 | 0.8381 | 9.613 |

### Example 23: Anti-IL-18BP Antibodies of the Present Disclosure are Effective in an EMT6 Animal Tumor Model as Monotherapy and in Combination with Anti-PD-L1

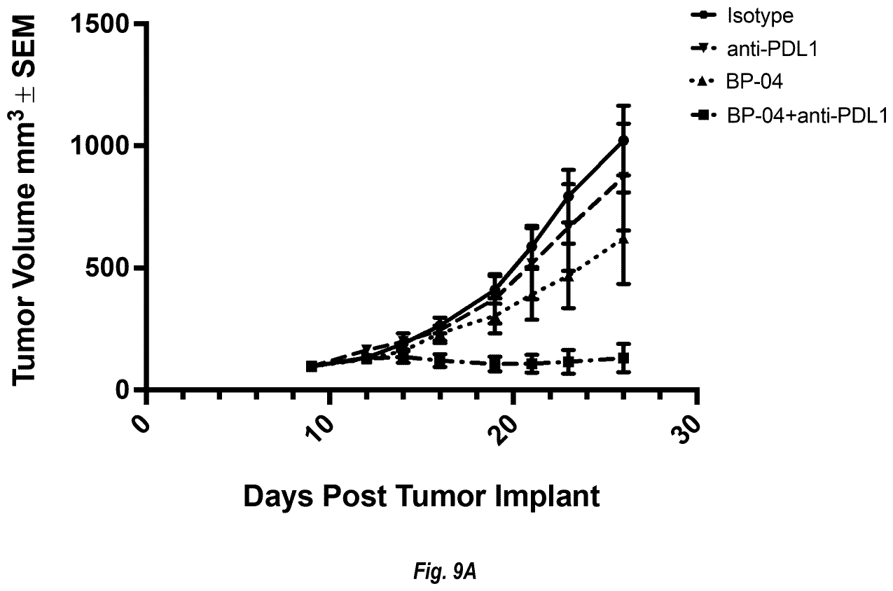

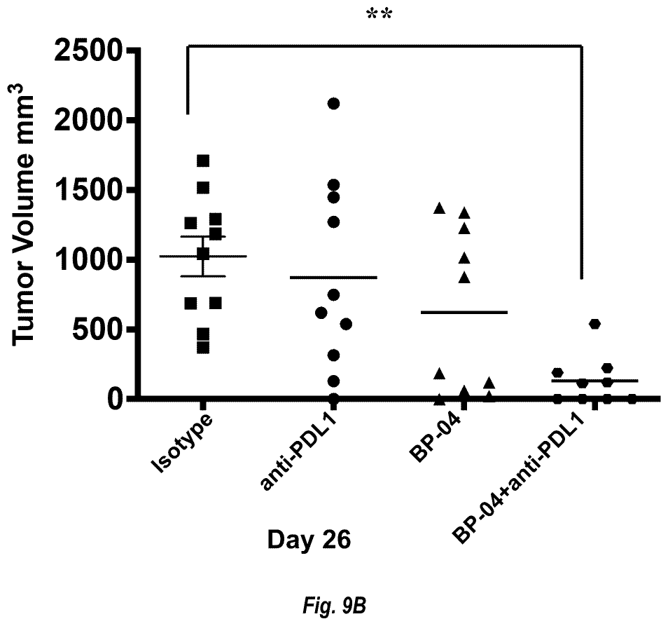

In addition to testing in the E0771 model, anti-IL-18BP antibody BP-04 was also tested as monotherapy or in combination with anti-PD-L1 in an EMT6 mouse syngeneic tumor model. Monotherapy treatment with anti-IL-18BP antibody BP-04 resulted in reduced tumor volume compared to that observed with the isotype control antibody treated group or in animals treated with anti-PD-L1 alone (FIG. 9A and FIG. 9B). Anti-IL-18BP antibody BP-04 in combination with anti-PD-L1 resulted in a significant reduction in tumor volume, with 4 tumor cures (FIGS. 9A and 9B). Taken together with the results seen in the E0771 animal tumor model, these data demonstrated that treatment with anti-IL-18BP antibodies effectively generates an anti-tumor response in multiple mouse models of cancer.

Provided below are amino acid sequences of various anti-IL-18BP antibodies of the present disclosure:

BP-04 Light Chain:
(SEQ ID NO: 189)
DIQMTQSPSSVSASVGDRVTITCRASDNVYSNLAWYQQKPGKAPK
LLIYGAISLADGVPSRFSGSGSGTDFTLTISSLQPEDFATYYCQH
FWGNPYTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC

TABLE 16

| Antibody | | SEQ ID |
|---|---|---|
| BP-04.03 | Heavy Chain | NO: |
| huIgG1 | QVQLVQSGAEVKKPGASVKVSCKASGYTFTDYDMHWVRQAPGQGLEWMGI | 190 |
| wild type | ISTYYGDVSYAQKFQGRVTMTRDKSTSTVYMELSSLRSENTAVYYCARSG | |
| | TYYYGTREQHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 191 |
| K322A | ISTYYGDVSYAQKFQGRVTMTRDKSTSTVYMELSSLRSENTAVYYCARSG | |
| | TYYYGTREQHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCAVSNKALPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 192 |
| L234A; | ISTYYGDVSYAQKFQGRVTMTRDKSTSTVYMELSSLRSENTAVYYCARSG | |
| L235A; | TYYYGTREQHALDYWGQGTLVTVSSASTKGPSVFPLAPSSKSTSGGTAAL | |
| P331S | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPASIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 193 |
| P331S | ISTYYGDVSYAQKFQGRVTMTRDKSTSTVYMELSSLRSENTAVYYCARSG | |
| | TYYYGTREQHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPASIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 194 |
| P331S; | ISTYYGDVSYAQKFQGRVTMTRDKSTSTVYMELSSLRSENTAVYYCARSG | |
| E430G | TYYYGTREQHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPASIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHGALHNHYTQKSLS | |
| | LSPGK | |
| huIgG1 | QVQLVQSGAEVKKPGASVKVSCKASGYTFTDYDMHWVRQAPGQGLEWMGI | 195 |
| N325S; | ISTYYGDVSYAQKFQGRVTMTRDKSTSTVYMELSSLRSENTAVYYCARSG | |
| L328F | TYYYGTREQHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSSKAFPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 196 |
| S267E; | ISTYYGDVSYAQKFQGRVTMTRDKSTSTVYMELSSLRSENTAVYYCARSG | |
| L328F | TYYYGTREQHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVEHEDPEVKENWYVDGVEVHNAKTKPR | |
| | EEQYNSTYRVVSVLTVLHQDWLNGKEYKCKVSNKAFPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVESCSVMHEALHNHYTQKSLS | |
| | LSPGK | |
| huIgG1 | QVQLVQSGAEVKKPGASVKVSCKASGYTFTDYDMHWVRQAPGQGLEWMGI | 197 |
| LS | ISTYYGDVSYAQKFQGRVTMTRDKSTSTVYMELSSLRSENTAVYYCARSG | |
| | TYYYGTREQHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPASIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVESCSVLHEALHSHYTQKSLS | |
| | LSPGK | |
| huIgG1 | QVQLVQSGAEVKKPGASVKVSCKASGYTFTDYDMHWVRQAPGQGLEWMGI | 198 |
| L234A; | ISTYYGDVSYAQKFQGRVTMTRDKSTSTVYMELSSLRSENTAVYYCARSG | |
| L235A; | TYYYGTREQHALDYWGQGTLVTVSSASTKGPSVFPLAPSSKSTSGGTAAL | |
| P329S | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALSAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 199 |
| L234A; | ISTYYGDVSYAQKFQGRVTMTRDKSTSTVYMELSSLRSENTAVYYCARSG | |
| L235A1 | TYYYGTREQHALDYWGQGTLVTVSSASTKGPSVFPLAPSSKSTSGGTAAL | |
| P331G | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPAGIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVESCSVMHEALHNHYTQKSLS | |
| | LSPGK | |
| huIgG1 | QVQLVQSGAEVKKPGASVKVSCKASGYTFTDYDMHWVRQAPGQGLEWMGI | 200 |
| L234A; | ISTYYGDVSYAQKFQGRVTMTRDKSTSTVYMELSSLRSENTAVYYCARSG | |
| L235A; | TYYYGTREQHALDYWGQGTLVTVSSASTKGPSVFPLAPSSKSTSGGTAAL | |
| P329G | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALGAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 201 |
| L234A; | ISTYYGDVSYAQKFQGRVTMTRDKSTSTVYMELSSLRSENTAVYYCARSG | |
| L235A | TYYYGTREQHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 202 |
| P329S | ISTYYGDVSYAQKFQGRVTMTRDKSTSTVYMELSSLRSENTAVYYCARSG | |
| | TYYYGTREQHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALSAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG4 | QVQLVQSGAEVKKPGASVKVSCKASGYTFTDYDMHWVRQAPGQGLEWMGI | 203 |
| | ISTYYGDVSYAQKFQGRVTMTRDKSTSTVYMELSSLRSENTAVYYCARSG | |
| | TYYYGTREQHALDYWGQGTLVTVSSASTKGPSVFPLAPCSRSTSESTAAL | |
| | GCLVKDYFPEPVTVSWNSGALTSGVHTFPAVLQSSGLYSLSSVVTVPSSS | |
| | LGTKTYTCNVDHKPSNTKVDKRVESKYGPPCPSCPAPEFLGGPSVELEPP | |
| | KPKDTLMISRTPEVTCVVVDVSQEDPEVQFNWYVDGVEVHNAKTKPREEQ | |
| | FNSTYRVVSVLTVLHQDWLNGKEYKCKVSNKGLPSSIEKTISKAKGQPRE | |
| | PQVYTLPPSQEEMTKNQVSLTCLVKGFYPSDIAVEWESNGQPENNYKTTP | |
| | PVLDSDGSFFLYSRLTVDKSRWQEGNVESCSVMHEALHNHYTQKSLSLSL | |
| | GK | |
| huIgG4 | QVQLVQSGAEVKKPGASVKVSCKASGYTFTDYDMHWVRQAPGQGLEWMGI | 204 |
| S228P | ISTYYGDVSYAQKFQGRVTMTRDKSTSTVYMELSSLRSENTAVYYCARSG | |
| | TYYYGTREQHALDYWGQGTLVTVSSASTKGPSVFPLAPCSRSTSESTAAL | |
| | GCLVKDYFPEPVTVSWNSGALTSGVHTFPAVLQSSGLYSLSSVVTVPSSS | |
| | LGTKTYTCNVDHKPSNTKVDKRVESKYGPPCPPCPAPEFLGGPSVELFPP | |
| | KPKDTLMISRTPEVTCVVVDVSQEDPEVQFNWYVDGVEVHNAKTKPREEQ | |
| | FNSTYRVVSVLTVLHQDWLNGKEYKCKVSNKGLPSSIEKTISKAKGQPRE | |
| | PQVYTLPPSQEEMTKNQVSLTCLVKGFYPSDIAVEWESNGQPENNYKTTP | |
| | PVLDSDGSFFLYSRLTVDKSRWQEGNVESCSVMHEALHNHYTQKSLSLSL | |
| | GK | |

TABLE 17

| Antibody | | SEQ ID |
|---|---|---|
| BP-04.05 | Heavy Chain | NO: |
| huIgG1 | QVQLVQSGAEVKKPGASVKVSCKASGYTFTDYDMHWVRQAPGQGLEWMGI | 205 |
| wild type | ISTYYGVVSYAQKKQGRVTMTRDKSTSTVYMELSSLRSEDTAVYYCARSG | |
| | TYYYGTRESHAFDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 206 |
| K322A | ISTYYGVVSYAQKKQGRVTMTRDKSTSTVYMELSSLRSEDTAVYYCARSG | |
| | TYYYGTRESHAFDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCAVSNKALPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 207 |
| L234A; | ISTYYGVVSYAQKKQGRVTMTRDKSTSTVYMELSSLRSEDTAVYYCARSG | |
| L235A; | TYYYGTRESHAFDYWGQGTLVTVSSASTKGPSVFPLAPSSKSTSGGTAAL | |
| P331S | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPASIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 208 |
| P331S | ISTYYGVVSYAQKKQGRVTMTRDKSTSTVYMELSSLRSEDTAVYYCARSG | |
| | TYYYGTRESHAFDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPASIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 209 |
| P331S; | ISTYYGVVSYAQKKQGRVTMTRDKSTSTVYMELSSLRSEDTAVYYCARSG | |
| E430G | TYYYGTRESHAFDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPASIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHGALHNHYTQKSLS | |
| | LSPGK | |
| huIgG1 | QVQLVQSGAEVKKPGASVKVSCKASGYTFTDYDMHWVRQAPGQGLEWMGI | 210 |
| N325S; | ISTYYGVVSYAQKKQGRVTMTRDKSTSTVYMELSSLRSEDTAVYYCARSG | |
| L328F | TYYYGTRESHAFDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSSKAFPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 211 |
| S267E; | ISTYYGVVSYAQKKQGRVTMTRDKSTSTVYMELSSLRSEDTAVYYCARSG | |
| L328F | TYYYGTRESHAFDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVEHEDPEVKENWYVDGVEVHNAKTKPR | |
| | EEQYNSTYRVVSVLTVLHQDWLNGKEYKCKVSNKAFPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 212 |
| LS | ISTYYGVVSYAQKKQGRVTMTRDKSTSTVYMELSSLRSEDTAVYYCARSG | |
| | TYYYGTRESHAFDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPASIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVLHEALHSHYTQKSLS | |
| | LSPGK | |
| huIgG1 | QVQLVQSGAEVKKPGASVKVSCKASGYTFTDYDMHWVRQAPGQGLEWMGI | 213 |
| L234A; | ISTYYGVVSYAQKKQGRVTMTRDKSTSTVYMELSSLRSEDTAVYYCARSG | |
| L235A; | TYYYGTRESHAFDYWGQGTLVTVSSASTKGPSVFPLAPSSKSTSGGTAAL | |
| P329S | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKFNWYVDGVEVHNAKTKPR | |
| | EEQYNSTYRVVSVLTVLHQDWLNGKEYKCKVSNKALSAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 214 |
| L234A; | ISTYYGVVSYAQKKQGRVTMTRDKSTSTVYMELSSLRSEDTAVYYCARSG | |
| L235A1 | TYYYGTRESHAFDYWGQGTLVTVSSASTKGPSVFPLAPSSKSTSGGTAAL | |
| P331G | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPAGIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 215 |
| L234A; | ISTYYGVVSYAQKKQGRVTMTRDKSTSTVYMELSSLRSEDTAVYYCARSG | |
| L235A; | TYYYGTRESHAFDYWGQGTLVTVSSASTKGPSVFPLAPSSKSTSGGTAAL | |
| P329G | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALGAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 216 |
| L234A; | ISTYYGVVSYAQKKQGRVTMTRDKSTSTVYMELSSLRSEDTAVYYCARSG | |
| L235A | TYYYGTRESHAFDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 217 |
| P329S | ISTYYGVVSYAQKKQGRVTMTRDKSTSTVYMELSSLRSEDTAVYYCARSG | |
| | TYYYGTRESHAFDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALSAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG4 | QVQLVQSGAEVKKPGASVKVSCKASGYTFTDYDMHWVRQAPGQGLEWMGI | 218 |
| | ISTYYGVVSYAQKKQGRVTMTRDKSTSTVYMELSSLRSEDTAVYYCARSG | |
| | TYYYGTRESHAFDYWGQGTLVTVSSASTKGPSVEPLAPCSRSTSESTAAL | |
| | GCLVKDYFPEPVTVSWNSGALTSGVHTFPAVLQSSGLYSLSSVVTVPSSS | |
| | LGTKTYTCNVDHKPSNTKVDKRVESKYGPPCPSCPAPEFLGGPSVELFPP | |
| | KPKDTLMISRTPEVTCVVVDVSQEDPEVQFNWYVDGVEVHNAKTKPREEQ | |
| | FNSTYRVVSVLTVLHQDWLNGKEYKCKVSNKGLPSSIEKTISKAKGQPRE | |
| | PQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSL | |
| | GK | |
| huIgG4 | QVQLVQSGAEVKKPGASVKVSCKASGYTFTDYDMHWVRQAPGQGLEWMGI | 219 |
| S228P | ISTYYGVVSYAQKKQGRVTMTRDKSTSTVYMELSSLRSEDTAVYYCARSG | |
| | TYYYGTRESHAFDYWGQGTLVTVSSASTKGPSVEPLAPCSRSTSESTAAL | |
| | GCLVKDYFPEPVTVSWNSGALTSGVHTFPAVLQSSGLYSLSSVVTVPSSS | |
| | LGTKTYTCNVDHKPSNTKVDKRVESKYGPPCPPCPAPEFLGGPSVELFPP | |
| | KPKDTLMISRTPEVTCVVVDVSQEDPEVQFNWYVDGVEVHNAKTKPREEQ | |
| | FNSTYRVVSVLTVLHQDWLNGKEYKCKVSNKGLPSSIEKTISKAKGQPRE | |
| | PQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSL | |
| | GK | |

TABLE 18

| Antibody | | SEQ ID |
|---|---|---|
| BP-04.07 | Heavy Chain | NO: |
| huIgG1 | QVQLVQSGAEVKKPGASVKVSCKASGYTFTDYDMHWVRQAPGQGLEWMGI | 220 |
| wild type | ISTYYGVVSYAQKKQGRVTMTRDKSTSTVYMELSSLRSEDTAVYYCARSG | |
| | TYNYGTRESHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVFL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 221 |
| K322A | ISTYYGVVSYAQKKQGRVTMTRDKSTSTVYMELSSLRSEDTAVYYCARSG | |
| | TYNYGTRESHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCAVSNKALPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 222 |
| L234A; | ISTYYGVVSYAQKKQGRVTMTRDKSTSTVYMELSSLRSEDTAVYYCARSG | |
| L235A; | TYNYGTRESHALDYWGQGTLVTVSSASTKGPSVFPLAPSSKSTSGGTAAL | |
| P331S | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPASIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 223 |
| P331S | ISTYYGVVSYAQKKQGRVTMTRDKSTSTVYMELSSLRSEDTAVYYCARSG | |
| | TYNYGTRESHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPASIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 224 |
| P331S; | ISTYYGVVSYAQKKQGRVTMTRDKSTSTVYMELSSLRSEDTAVYYCARSG | |
| E430G | TYNYGTRESHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPASIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHGALHNHYTQKSLS | |
| | LSPGK | |
| huIgG1 | QVQLVQSGAEVKKPGASVKVSCKASGYTFTDYDMHWVRQAPGQGLEWMGI | 225 |
| N325S; | ISTYYGVVSYAQKKQGRVTMTRDKSTSTVYMELSSLRSEDTAVYYCARSG | |
| L328F | TYNYGTRESHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSSKAFPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 226 |
| S267E; | ISTYYGVVSYAQKKQGRVTMTRDKSTSTVYMELSSLRSEDTAVYYCARSG | |
| L328F | TYNYGTRESHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVEHEDPEVKENWYVDGVEVHNAKTKPR | |
| | EEQYNSTYRVVSVLTVLHQDWLNGKEYKCKVSNKAFPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVESCSVMHEALHNHYTQKSLS | |
| | LSPGK | |
| huIgG1 | QVQLVQSGAEVKKPGASVKVSCKASGYTFTDYDMHWVRQAPGQGLEWMGI | 227 |
| LS | ISTYYGVVSYAQKKQGRVTMTRDKSTSTVYMELSSLRSEDTAVYYCARSG | |
| | TYNYGTRESHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPASIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVESCSVLHEALHSHYTQKSLS | |
| | LSPGK | |
| huIgG1 | QVQLVQSGAEVKKPGASVKVSCKASGYTFTDYDMHWVRQAPGQGLEWMGI | 228 |
| L234A; | ISTYYGVVSYAQKKQGRVTMTRDKSTSTVYMELSSLRSEDTAVYYCARSG | |
| L235A; | TYNYGTRESHALDYWGQGTLVTVSSASTKGPSVFPLAPSSKSTSGGTAAL | |
| P329S | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALSAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 229 |
| L234A; | ISTYYGVVSYAQKKQGRVTMTRDKSTSTVYMELSSLRSEDTAVYYCARSG | |
| L235A1 | TYNYGTRESHALDYWGQGTLVTVSSASTKGPSVFPLAPSSKSTSGGTAAL | |
| P331G | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPAGIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVESCSVMHEALHNHYTQKSLS | |
| | LSPGK | |
| huIgG1 | QVQLVQSGAEVKKPGASVKVSCKASGYTFTDYDMHWVRQAPGQGLEWMGI | 230 |
| L234A; | ISTYYGVVSYAQKKQGRVTMTRDKSTSTVYMELSSLRSEDTAVYYCARSG | |
| L235A; | TYNYGTRESHALDYWGQGTLVTVSSASTKGPSVFPLAPSSKSTSGGTAAL | |
| P329G | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALGAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVESCSVMHEALHNHYTQKSLS | |
| | LSPGK | |
| huIgG1 | QVQLVQSGAEVKKPGASVKVSCKASGYTFTDYDMHWVRQAPGQGLEWMGI | 231 |
| L234A; | ISTYYGVVSYAQKKQGRVTMTRDKSTSTVYMELSSLRSEDTAVYYCARSG | |
| L235A | TYNYGTRESHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPEAAGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALPAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLS | |
| | LSPGK | |
| huIgG1 | QVQLVQSGAEVKKPGASVKVSCKASGYTFTDYDMHWVRQAPGQGLEWMGI | 232 |
| P329S | ISTYYGVVSYAQKKQGRVTMTRDKSTSTVYMELSSLRSEDTAVYYCARSG | |
| | TYNYGTRESHALDYWGQGTLVTVSSASTKGPSVFPLAPSSKSTSGGTAAL | |
| | GCLVKDYFPEPVTVSWNSGALTSGVHTFPAVLQSSGLYSLSSVVTVPSSS | |
| | LGTQTYICNVNHKPSNTKVDKKVEPKSCDKTHTCPPCPAPELLGGPSVEL | |
| | FPPKPKDTLMISRTPEVTCVVVDVSHEDPEVKENWYVDGVEVHNAKTKPR | |
| | EEQYNSTYRVVSVLTVLHQDWLNGKEYKCKVSNKALSAPIEKTISKAKGQ | |
| | PREPQVYTLPPSRDELTKNQVSLTCLVKGFYPSDIAVEWESNGQPENNYK | |
| | TTPPVLDSDGSFFLYSKLTVDKSRWQQGNVESCSVMHEALHNHYTQKSLS | |
| | LSPGK | |
| huIgG4 | QVQLVQSGAEVKKPGASVKVSCKASGYTFTDYDMHWVRQAPGQGLEWMGI | 233 |
| | ISTYYGVVSYAQKKQGRVTMTRDKSTSTVYMELSSLRSEDTAVYYCARSG | |
| | TYNYGTRESHALDYWGQGTLVTVSSASTKGPSVFPLAPCSRSTSESTAAL | |
| | GCLVKDYFPEPVTVSWNSGALTSGVHTFPAVLQSSGLYSLSSVVTVPSSS | |
| | LGTKTYTCNVDHKPSNTKVDKRVESKYGPPCPSCPAPEFLGGPSVFLFPP | |
| | KPKDTLMISRTPEVTCVVVDVSQEDPEVQFNWYVDGVEVHNAKTKPREEQ | |
| | FNSTYRVVSVLTVLHQDWLNGKEYKCKVSNKGLPSSIEKTISKAKGQPRE | |
| | PQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSL | |
| | GK | |
| huIgG4 | QVQLVQSGAEVKKPGASVKVSCKASGYTFTDYDMHWVRQAPGQGLEWMGI | 234 |
| S228P | ISTYYGVVSYAQKKQGRVTMTRDKSTSTVYMELSSLRSEDTAVYYCARSG | |
| | TYNYGTRESHALDYWGQGTLVTVSSASTKGPSVEPLAPCSRSTSESTAAL | |
| | GCLVKDYFPEPVTVSWNSGALTSGVHTFPAVLQSSGLYSLSSVVTVPSSS | |
| | LGTKTYTCNVDHKPSNTKVDKRVESKYGPPCPPCPAPEFLGGPSVELFPP | |
| | KPKDTLMISRTPEVTCVVVDVSQEDPEVQFNWYVDGVEVHNAKTKPREEQ | |
| | FNSTYRVVSVLTVLHQDWLNGKEYKCKVSNKGLPSSIEKTISKAKGQPRE | |
| | PQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSL | |
| | GK | |

Provided below are various IgG Fc amino acid sequences for use in the instant disclosure.

huIgG1-WT with C-terminal lysine
(SEQ ID NO: 8)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
huIgG1-WT without C-terminal lysine
(SEQ ID NO: 9)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPG
huIgG1-K322A with C-terminal lysine
(SEQ ID NO: 10)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCAVSNKALPAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
huIgG1-K322A without C-terminal lysine
(SEQ ID NO: 11)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCAVSNKALPAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPG
huIgG1-LALAPS (L234A; L235A, P331S) with C-terminal
lysine
(SEQ ID NO: 12)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPASIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
huIgG1-LALAPS (L234A; L235A, P331S) without C-terminal
lysine
(SEQ ID NO: 13)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPASIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPG
huIgG1-PS (P331S) with C-terminal lysine
(SEQ ID NO: 14)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPASIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
huIgG1-PS (P331S) without C-terminal lysine
(SEQ ID NO: 15)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPASIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPG
huIgG1-PSEG (P331S; E430G) with C-terminal lysine
(SEQ ID NO: 16)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPASIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHGALHNHYTQKSLSLSPGK
huIgG1-PSEG (P331S; E430G) without C-terminal lysine
(SEQ ID NO: 17)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPASIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHGALHNHYTQKSLSLSPG
huIgG1-NSLF (N325S; L328F) with C-terminal lysine
(SEQ ID NO: 18)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSSKAFPAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
huIgG1-NSLF (N325S; L328F) without C-terminal lysine
(SEQ ID NO: 19)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSSKAFPAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPG
huIgG1-SELF (S267E; L328F) with C-terminal lysine
(SEQ ID NO: 20)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVEHEDPEVKFNWYVDGV
EVHNAKTKPREEQYNSTYRVVSVLTVLHQDWLNGKEYKCKVSNKAFPAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
huIgG1-SELF (S267E; L328F) without C-terminal lysine
(SEQ ID NO: 21)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVEHEDPEVKFNWYVDGV
EVHNAKTKPREEQYNSTYRVVSVLTVLHQDWLNGKEYKCKVSNKAFPAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPG
huIgG1-LS with C-terminal lysine
(SEQ ID NO: 22)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPASIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVLHEALHSHYTQKSLSLSPGK
huIgG1-LS without C-terminal lysine
(SEQ ID NO: 23)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPASIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVLHEALHSHYTQKSLSLSPG
huIgG2-WT with C-terminal lysine
(SEQ ID NO: 24)
ASTKGPSVFPLAPCSRSTSESTAALGCLVKDYFPEPVTVSWNSGALTSGVHTFPA
VLQSSGLYSLSSVVTVPSSNFGTQTYTCNVDHKPSNTKVDKTVERKCCVECPPCP
APPVAGPSVFLFPPKPKDTLMISRTPEVTCVVVDVSHEDPEVQFNWYVDGVEVHN
AKTKPREEQFNSTFRVVSVLTVVHQDWLNGKEYKCKVSNKGLPAPIEKTISKTKG
QPREPQVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPGK
huIgG2-WT without C-terminal lysine
(SEQ ID NO: 25)
ASTKGPSVFPLAPCSRSTSESTAALGCLVKDYFPEPVTVSWNSGALTSGVHTFPA
VLQSSGLYSLSSVVTVPSSNFGTQTYTCNVDHKPSNTKVDKTVERKCCVECPPCP
APPVAGPSVFLFPPKPKDTLMISRTPEVTCVVVDVSHEDPEVQFNWYVDGVEVHN
AKTKPREEQFNSTFRVVSVLTVVHQDWLNGKEYKCKVSNKGLPAPIEKTISKTKG
QPREPQVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
huIgG4-WT with C-terminal lysine
(SEQ ID NO: 26)
ASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPSCP
APEFLGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLGK
huIgG4-WT without C-terminal lysine
(SEQ ID NO: 27)
ASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPSCP
APEFLGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLG
Human IgG light chain constant region amino acid
sequence
(SEQ ID NO: 28)
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
huIgG1-LALAPS (L234A; L235A, P329S) with C-terminal
lysine
(SEQ ID NO: 235)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALSAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
huIgG1-LALAPS (L234A; L235A, P329S) without C-terminal
lysine
(SEQ ID NO: 236)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALSAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPG
huIgG1-LALAPG (L234A; L235A; P331G) with C-terminal
lysine
(SEQ ID NO: 237)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAGIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
huIgG1-LALAPG (L234A; L235A; P331G) without C-terminal
lysine
(SEQ ID NO: 238)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAGIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPG
huIgG1-LALAPG (L234A; L235A, P329G) with C-terminal
lysine
(SEQ ID NO: 239)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALGAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
huIgG1-LALAPG (L234A; L235A, P329G) without C-terminal
lysine
(SEQ ID NO: 240)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALGAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPG
huIgG1-LALA (L234A; L235A) with C-terminal lysine
(SEQ ID NO: 241)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
huIgG1-LALA (L234A; L235A) without C-terminal lysine
(SEQ ID NO: 242)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPG
huIgG1-PS (P329S) with C-terminal lysine
(SEQ ID NO: 243)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALSAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
huIgG1-PS (P329S) without C-terminal lysine
(SEQ ID NO: 244)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALSAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPG
huIgG4 (S228P)-with C-terminal lysine
(SEQ ID NO: 245)
ASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCP
APEFLGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLGK
huIgG4 (S228P)-without C-terminal lysine
(SEQ ID NO: 246)
ASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCP
APEFLGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLG

What is claimed is:

1. An isolated anti-interleukin-18-binding protein (IL18BP) antibody, wherein the anti-IL18BP antibody blocks binding of IL-18 to IL18BP.

2. The anti-IL18BP antibody of claim 1, wherein the anti-IL18BP antibody increases expression levels of interferon-gamma (IFNγ) in peripheral blood mononuclear cells.

3. The anti-IL18BP antibody of claim 1 or 2, wherein the anti-IL18BP antibody increases expression of IFNγ in natural killer cells.

4. The anti-IL18BP antibody of any one of claims 1-3, wherein the anti-IL18BP antibody increases levels of inflammatory cytokines in serum.

5. The anti-IL18BP antibody of any one of claims 1-4, wherein the anti-IL18BP antibody increases IFNγ levels in serum.

6. The anti-IL18BP antibody of any one of claims 1-5, wherein the anti-IL18BP antibody increases IL-10 levels in serum.

7. The anti-IL18BP antibody of any one of claims 1-6, wherein the anti-IL18BP antibody increases IL-6 levels in serum.

8. The anti-IL18BP antibody of any one of claims 1-7, wherein the anti-IL18BP antibody increases tumor necrosis factor alpha (TNFα) levels in serum.

9. The anti-IL18BP antibody of any one of claims 1-8, wherein the anti-IL18BP antibody decreases the percentage of CD3+ T cells in vivo.

10. The anti-IL18BP antibody of any one of claims 1-9, wherein the anti-IL18BP antibody decreases the percentage of CD19+ B cells in vivo.

11. The anti-IL18BP antibody of any one of claims 1-10, wherein the anti-IL18BP antibody decreases the percentage of CD11b+ cells in vivo.

12. The anti-IL18BP antibody of any one of claims 1-11, wherein the anti-IL18BP antibody increases the percentage of macrophages in vivo.

13. The anti-IL18BP antibody of any one of claims 1-12, wherein the anti-IL18BP antibody competes with one or more antibodies selected from BP-10, BP-02, BP-03, BP-04, BP-05, BP-06, BP-07, BP-08, BP-09, BP-10, BP-11, BP-12, BP-13, BP-14, BP-15, BP-16, BP-04.01, BP-04.02, BP-04.03, BP-04.04, BP-04.05, BP-04.06, BP-04.07, BP-04.08, BP-04.09, BP-04.10, BP-04.11, BP-04.12, BP-04.13, BP-04.14, BP-04.15, BP-04.16, BP-04.17, BP-04.18, BP-04.19, BP-04.20, BP-04.21, BP-04.22, BP-04.23, BP-04.24, BP-04.25, BP-04.26, BP-04.27, BP-04.28, BP-04.29, BP-04.30, BP-04.31, and BP-04.32, and any combination thereof for binding to IL18BP.

14. The anti-IL18BP antibody of any one of claims 1-13, wherein the anti-IL18BP antibody comprises a light chain variable domain and a heavy chain variable domain, wherein the light chain variable domain, the heavy chain variable domain, or both comprise at least one, at least two, at least three, at least four, at least five, or six HVRs selected from HVR-L1, HVR-L2, HVR-L3, HVR-H1, HVR-H2, and HVR-H3 of an antibody selected from: BP-01, BP-02, BP-03, BP-04, BP-05, BP-06, BP-07, BP-08, BP-09, BP-10, BP-11, BP-12, BP-13, BP-14, BP-15, BP-16, BP-04.01, BP-04.02, BP-04.03, BP-04.04, BP-04.05, BP-04.06, BP-04.07, BP-04.08, BP-04.09, BP-04.10, BP-04.11, BP-04.12, BP-04.13, BP-04.14, BP-04.15, BP-04.16, BP-04.17, BP-04.18, BP-04.19, BP-04.20, BP-04.21, BP-04.22, BP-04.23, BP-04.24, BP-04.25, BP-04.26, BP-04.27, BP-04.28, BP-04.29, BP-04.30, BP-04.31, and BP-04.32.

15. The anti-IL18BP antibody of claim 14, wherein:
a. the HVR-H1 comprises an amino acid sequence selected from the group consisting of SEQ ID NOs: 60, 61, 62, 63, 64, 65, 66, 67, 68, 69, 70, 167, 168, and 169;
b. the HVR-H2 comprises an amino acid sequence selected from the group consisting of SEQ ID NOs: 71, 72, 73, 74, 75, 76, 77, 78, 79, 80, 81, 82, 83, 84, 85, 86, 170, 171, 172, 173, 174, 175, 176, 177, and 178;
c. the HVR-H3 comprises an amino acid sequence selected from the group consisting of SEQ ID NOs: 87, 88, 89, 90, 91, 92, 93, 94, 95, 96, 97, 98, 99, 100, 179, 180, 181, 182, 183, 184, 185, 186, 187, and 188;
d. the HVR-L1 comprises an amino acid sequence selected from the group consisting of SEQ ID NOs: 101, 102, 103, 104, 105, 106, 107, 108, 109, 110, 111, and 247;
e. the HVR-L2 comprises an amino acid sequence selected from the group consisting of SEQ ID NOs: 112, 113, 114, 115, 116, 117, 118, 119, 120, 121, and 248; and/or
f. the HVR-L3 comprises an amino acid sequence selected from the group consisting of SEQ ID NOs: 122, 123, 124, 125, 126, 127, 128, 129, 130, 131, 132, 133, and 249.

16. The anti-IL18BP antibody of claim 14 or 15, wherein:
a. the HVR-H1 comprises the amino acid sequence of SEQ ID NO:60; the HVR-H2 comprises the amino acid sequence of SEQ ID NO:71; the HVR-H3 comprises the amino acid sequence of SEQ ID NO:87; the HVR-L1 comprises the amino acid sequence of SEQ ID NO: 101; the HVR-L2 comprises the amino acid sequence of SEQ ID NO: 112; and the HVR-L3 comprises the amino acid sequence of SEQ ID NO: 122; or
b. the HVR-H1 comprises the amino acid sequence of SEQ ID NO:61; the HVR-H2 comprises the amino acid sequence of SEQ ID NO: 72; the HVR-H3 comprises the amino acid sequence of SEQ ID NO:88; the HVR-L1 comprises the amino acid sequence of SEQ ID NO: 102; the HVR-L2 comprises the amino acid sequence of SEQ ID NO: 113; and the HVR-L3 comprises the amino acid sequence of SEQ ID NO: 123; or
c. the HVR-H1 comprises the amino acid sequence of SEQ ID NO:61; the HVR-H2 comprises the amino acid sequence of SEQ ID NO: 73; the HVR-H3 comprises the amino acid sequence of SEQ ID NO:88; the HVR-L1 comprises the amino acid sequence of SEQ ID NO: 103; the HVR-L2 comprises the amino acid sequence of SEQ ID NO: 113; and the HVR-L3 comprises the amino acid sequence of SEQ ID NO: 123; or
d. the HVR-H1 comprises the amino acid sequence of SEQ ID NO:62; the HVR-H2 comprises the amino acid sequence of SEQ ID NO: 74; the HVR-H3 comprises the amino acid sequence of SEQ ID NO:89; the HVR-L1 comprises the amino acid sequence of SEQ ID NO: 104; the HVR-L2 comprises the amino acid sequence of SEQ ID NO: 114; and the HVR-L3 comprises the amino acid sequence of SEQ ID NO: 124; or
e. the HVR-H1 comprises the amino acid sequence of SEQ ID NO:61; the HVR-H2 comprises the amino acid sequence of SEQ ID NO: 75; the HVR-H3 comprises the amino acid sequence of SEQ ID NO:90; the HVR-L1 comprises the amino acid sequence of SEQ ID NO: 105; the HVR-L2 comprises the amino acid sequence of SEQ ID NO: 113; and the HVR-L3 comprises the amino acid sequence of SEQ ID NO: 123; or
f. the HVR-H1 comprises the amino acid sequence of SEQ ID NO:63; the HVR-H2 comprises the amino acid sequence of SEQ ID NO: 76; the HVR-H3 comprises the amino acid sequence of SEQ ID NO:91; the HVR-L1 comprises the amino acid sequence of SEQ ID NO: 106; the HVR-L2 comprises the amino acid sequence of SEQ ID NO: 115; and the HVR-L3 comprises the amino acid sequence of SEQ ID NO: 125; or
g. the HVR-H1 comprises the amino acid sequence of SEQ ID NO:61; the HVR-H2 comprises the amino acid sequence of SEQ ID NO: 77; the HVR-H3 comprises the amino acid sequence of SEQ ID NO:88; the HVR-L1 comprises the amino acid sequence of SEQ ID NO: 103; the HVR-L2 comprises the amino acid sequence of SEQ ID NO: 113; and the HVR-L3 comprises the amino acid sequence of SEQ ID NO: 123; or
h. the HVR-H1 comprises the amino acid sequence of SEQ ID NO:64; the HVR-H2 comprises the amino acid sequence of SEQ ID NO: 78; the HVR-H3 comprises the amino acid sequence of SEQ ID NO:92; the HVR-L1 comprises the amino acid sequence of SEQ ID NO: 107; the HVR-L2 comprises the amino acid sequence of SEQ ID NO: 116; and the HVR-L3 comprises the amino acid sequence of SEQ ID NO: 126; or
i. the HVR-H1 comprises the amino acid sequence of SEQ ID NO:65; the HVR-H2 comprises the amino acid sequence of SEQ ID NO: 79; the HVR-H3 comprises the amino acid sequence of SEQ ID NO:93; the HVR-L1 comprises the amino acid sequence of SEQ ID NO: 101; the HVR-L2 comprises the amino acid sequence of SEQ ID NO: 116; and the HVR-L3 comprises the amino acid sequence of SEQ ID NO: 127; or
j. the HVR-H1 comprises the amino acid sequence of SEQ ID NO:66; the HVR-H2 comprises the amino acid sequence of SEQ ID NO:80; the HVR-H3 comprises the amino acid sequence of SEQ ID NO:94; the HVR-L1 comprises the amino acid sequence of SEQ ID NO: 108; the HVR-L2 comprises the amino acid sequence of SEQ ID NO: 117; and the HVR-L3 comprises the amino acid sequence of SEQ ID NO: 128; or
k. the HVR-H1 comprises the amino acid sequence of SEQ ID NO:66; the HVR-H2 comprises the amino acid sequence of SEQ ID NO:81; the HVR-H3 comprises the amino acid sequence of SEQ ID NO:95; the HVR-L1 comprises the amino acid sequence of SEQ ID NO: 109; the HVR-L2 comprises the amino acid sequence of SEQ ID NO: 118; and the HVR-L3 comprises the amino acid sequence of SEQ ID NO: 129; or
l. the HVR-H1 comprises the amino acid sequence of SEQ ID NO:67; the HVR-H2 comprises the amino acid sequence of SEQ ID NO:82; the HVR-H3 comprises the amino acid sequence of SEQ ID NO:96; the HVR-L1 comprises the amino acid sequence of SEQ ID NO: 101; the HVR-L2 comprises the amino acid sequence of SEQ ID NO: 116; and the HVR-L3 comprises the amino acid sequence of SEQ ID NO: 130; or
m. the HVR-H1 comprises the amino acid sequence of SEQ ID NO:68; the HVR-H2 comprises the amino acid sequence of SEQ ID NO:83; the HVR-H3 comprises the amino acid sequence of SEQ ID NO:97; the HVR-L1 comprises the amino acid sequence of SEQ ID NO: 101; the HVR-L2 comprises the amino acid sequence of SEQ ID NO: 119; and the HVR-L3 comprises the amino acid sequence of SEQ ID NO: 131; or
n. the HVR-H1 comprises the amino acid sequence of SEQ ID NO:69; the HVR-H2 comprises the amino acid sequence of SEQ ID NO:84; the HVR-H3 comprises the amino acid sequence of SEQ ID NO:98; the HVR-L1 comprises the amino acid sequence of SEQ ID NO: 110; the HVR-L2 comprises the amino acid sequence of SEQ ID NO: 120; and the HVR-L3 comprises the amino acid sequence of SEQ ID NO: 132; or
o. the HVR-H1 comprises the amino acid sequence of SEQ ID NO: 70; the HVR-H2 comprises the amino acid sequence of SEQ ID NO:85; the HVR-H3 comprises the amino acid sequence of SEQ ID NO:99; the HVR-L1 comprises the amino acid sequence of SEQ ID NO: 111; the HVR-L2 comprises the amino acid sequence of SEQ ID NO: 121; and the HVR-L3 comprises the amino acid sequence of SEQ ID NO: 133; or
p. the HVR-H1 comprises the amino acid sequence of SEQ ID NO: 70; the HVR-H2 comprises the amino acid sequence of SEQ ID NO:86; the HVR-H3 comprises the amino acid sequence of SEQ ID NO: 100; the HVR-L1 comprises the amino acid sequence of SEQ ID NO:111; the HVR-L2 comprises the amino acid sequence of SEQ ID NO: 121; and the HVR-L3 comprises the amino acid sequence of SEQ ID NO: 133, or
q. the HVR-H1 comprises the amino acid sequence of SEQ ID NO: 167; the HVR-H2 comprises the amino acid sequence of SEQ ID NO: 170; the HVR-H3 comprises the amino acid sequence of SEQ ID NO: 179; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
r. the HVR-H1 comprises the amino acid sequence of SEQ ID NO: 168; the HVR-H2 comprises the amino acid sequence of SEQ ID NO: 171; the HVR-H3 comprises the amino acid sequence of SEQ ID NO: 180; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
s. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 181; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
t. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 182; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
u. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
v. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
w. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
x. the HVR-H1 comprises the amino acid sequence of SEQ ID NO: 169; the HVR-H2 comprises the amino acid sequence of SEQ ID NO: 1704; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
y. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
z. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
aa. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
bb. the HVR-H1 comprises the amino acid sequence of SEQ ID NO: 169; the HVR-H2 comprises the amino acid sequence of SEQ ID NO: 176; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
cc. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 187; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
dd. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 188; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
ee. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 182; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
ff. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 183; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
gg. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
hh. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
ii. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
jj. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
kk. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
ll. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
mm. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
nn. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
oo. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 185; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
pp. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
qq. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
rr. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 184; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
ss. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 185; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249, or
tt. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
uu. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249, or
vv. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249.

17. An isolated anti-IL18BP antibody, wherein the anti-IL18BP antibody comprises a heavy chain variable region (VH) and a light chain variable region (VL), wherein the heavy chain variable region (VH) comprises:
a. an HVR-H1 comprising an amino acid sequence chosen from any one of SEQ ID NOs: 60, 61, 62, 63, 64, 65, 66, 67, 68, 69, 70, 167, 168, and 169;
b. an HVR-H2 comprising an amino acid sequence chosen from any one of SEQ ID NOs: 71, 72, 73, 74, 75, 76, 77, 78, 79, 80, 81, 82, 83, 84, 85, 86, 170, 171, 172, 173, 174, 175, 176, 177, and 178; and
c. an HVR-H3 comprising an amino acid sequence chosen from any one of SEQ ID NOs: 87, 88, 89, 90, 91, 92, 93, 94, 95, 96, 97, 98, 99, 100, 179, 180, 181, 182, 183, 184, 185, 186, 187, and 188.

18. The antibody of claim 17, wherein the light chain variable region comprises:
a. an HVR-L1 comprising an amino acid sequence chosen from any one of SEQ ID NOs: 101, 102, 103, 104, 105, 106, 107, 108, 109, 110, 111, and 247;
b. an HVR-L2 comprising an amino acid sequence chosen from any one of SEQ ID NOs: 112, 113, 114, 115, 116, 117, 118, 119, 120, 121, and 248; and
c. an HVR-L3 comprising an amino acid sequence chosen from any one of SEQ ID NOs: 122, 123, 124, 125, 126, 127, 128, 129, 130, 131, 132, 133; and 249.

19. The antibody of claim 17 or 18, wherein the antibody comprises a VH that is at least 90%, at least 95%, at least 97%, or at least 99% identical to an amino acid sequence selected from any one of SEQ ID NOs: 29, 30, 31, 32, 33, 34, 35, 36, 37, 38, 39, 40, 41, 42, 43, 44, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, and 165.

20. The antibody of any one of claims 17-19, wherein the antibody comprises a VL that is at least 90%, at least 95%, at least 97%, or at least 99% identical to an amino acid sequence selected from any one of SEQ ID NOs: 45, 46, 47, 48, 49, 50, 51, 52, 53, 54, 55, 56, 57, 58, 59, and 166.

21. The antibody of any one of claims 17-20, wherein the VH comprises 1, 2, 3, 4, 5, 6, 7, 8, 9, or 10 amino acid substitutions, insertions, and/or deletions compared to an amino acid sequence selected from any one of SEQ ID NOs: 29, 30, 31, 32, 33, 34, 35, 36, 37, 38, 39, 40, 41, 42, 43, 44, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, and 165.

22. The antibody of any one of claims 17-21, wherein the VL comprises 1, 2, 3, 4, 5, 6, 7, 8, 9, or 10 amino acid substitutions, insertions, and/or deletions compared to an amino acid sequence selected from any one of SEQ ID NOs: 45, 46, 47, 48, 49, 50, 51, 52, 53, 54, 55, 56, 57, 58, 59, and 166.

23. The antibody of any one of claims 17-22, wherein the antibody comprises a VH comprising an amino acid sequence selected from any one of SEQ ID NOs: 29, 30, 31, 32, 33, 34, 35, 36, 37, 38, 39, 40, 41, 42, 43, 44, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, and 165.

24. The antibody of any one of claims 17-23, wherein the antibody comprises a VL comprising an amino acid sequence selected from any one of SEQ ID NOs: 45, 46, 47, 48, 49, 50, 51, 52, 53, 54, 55, 56, 57, 58, 59, and 166.

25. An isolated anti-IL18BP antibody, wherein the anti-IL18BP antibody comprises:
a. an HVR-H1 comprising the amino acid sequence of SEQ ID NO:60; an HVR-H2 comprising the amino acid sequence of SEQ ID NO: 71; an HVR-H3 comprising the amino acid sequence of SEQ ID NO:87; an HVR-L1 comprising the amino acid sequence of SEQ ID NO: 101; an HVR-L2 comprising the amino acid sequence of SEQ ID NO: 112; and an HVR-L3 comprising the amino acid sequence of SEQ ID NO: 122;
b. an HVR-H1 comprising the amino acid sequence of SEQ ID NO:61; an HVR-H2 comprising the amino acid sequence of SEQ ID NO: 72; an HVR-H3 comprising the amino acid sequence of SEQ ID NO:88; an HVR-L1 comprising the amino acid sequence of SEQ ID NO: 102; an HVR-L2 comprising the amino acid sequence of SEQ ID NO: 113; and an HVR-L3 comprising the amino acid sequence of SEQ ID NO: 123;
c. an HVR-H1 comprising the amino acid sequence of SEQ ID NO:61; an HVR-H2 comprising the amino acid sequence of SEQ ID NO:73; an HVR-H3 comprising the amino acid sequence of SEQ ID NO:88; an HVR-L1 comprising the amino acid sequence of SEQ ID NO: 103; an HVR-L2 comprising the amino acid sequence of SEQ ID NO: 113; and an HVR-L3 comprising the amino acid sequence of SEQ ID NO: 123;
d. an HVR-H1 comprising the amino acid sequence of SEQ ID NO:62; an HVR-H2 comprising the amino acid sequence of SEQ ID NO: 74; an HVR-H3 comprising the amino acid sequence of SEQ ID NO:89; an HVR-L1 comprising the amino acid sequence of SEQ ID NO: 104; an HVR-L2 comprising the amino acid sequence of SEQ ID NO: 114; and an HVR-L3 comprising the amino acid sequence of SEQ ID NO: 124;
e. an HVR-H1 comprising the amino acid sequence of SEQ ID NO:61; an HVR-H2 comprising the amino acid sequence of SEQ ID NO:75; an HVR-H3 comprising the amino acid sequence of SEQ ID NO:90; an HVR-L1 comprising the amino acid sequence of SEQ ID NO: 105; an HVR-L2 comprising the amino acid sequence of SEQ ID NO: 113; and an HVR-L3 comprising the amino acid sequence of SEQ ID NO: 123;
f. an HVR-H1 comprising the amino acid sequence of SEQ ID NO:63; an HVR-H2 comprising the amino acid sequence of SEQ ID NO: 76; an HVR-H3 comprising the amino acid sequence of SEQ ID NO:91; an HVR-L1 comprising the amino acid sequence of SEQ ID NO: 106; an HVR-L2 comprising the amino acid sequence of SEQ ID NO: 115; and an HVR-L3 comprising the amino acid sequence of SEQ ID NO: 125;
g. an HVR-H1 comprising the amino acid sequence of SEQ ID NO:61; an HVR-H2 comprising the amino acid sequence of SEQ ID NO: 77; an HVR-H3 comprising the amino acid sequence of SEQ ID NO:88; an HVR-L1 comprising the amino acid sequence of SEQ ID NO: 103; an HVR-L2 comprising the amino acid sequence of SEQ ID NO: 113; and an HVR-L3 comprising the amino acid sequence of SEQ ID NO:123;
h. an HVR-H1 comprising the amino acid sequence of SEQ ID NO:64; an HVR-H2 comprising the amino acid sequence of SEQ ID NO:78; an HVR-H3 comprising the amino acid sequence of SEQ ID NO:92; an HVR-L1 comprising the amino acid sequence of SEQ ID NO: 107; an HVR-L2 comprising the amino acid sequence of SEQ ID NO: 116; and an HVR-L3 comprising the amino acid sequence of SEQ ID NO: 126;
i. an HVR-H1 comprising the amino acid sequence of SEQ ID NO:65; an HVR-H2 comprising the amino acid sequence of SEQ ID NO: 79; an HVR-H3 comprising the amino acid sequence of SEQ ID NO:93; an HVR-L1 comprising the amino acid sequence of SEQ ID NO: 101; an HVR-L2 comprising the amino acid sequence of SEQ ID NO: 116; and an HVR-L3 comprising the amino acid sequence of SEQ ID NO: 127;
j. an HVR-H1 comprising the amino acid sequence of SEQ ID NO:66; an HVR-H2 comprising the amino acid sequence of SEQ ID NO:80; an HVR-H3 comprising the amino acid sequence of SEQ ID NO:94; an HVR-L1 comprising the amino acid sequence of SEQ ID NO: 108; an HVR-L2 comprising the amino acid sequence of SEQ ID NO: 117; and an HVR-L3 comprising the amino acid sequence of SEQ ID NO: 128;
k. an HVR-H1 comprising the amino acid sequence of SEQ ID NO:66; an HVR-H2 comprising the amino acid sequence of SEQ ID NO:81; an HVR-H3 comprising the amino acid sequence of SEQ ID NO:95; an HVR-L1 comprising the amino acid sequence of SEQ ID NO: 109; an HVR-L2 comprising the amino acid sequence of SEQ ID NO: 118; and an HVR-L3 comprising the amino acid sequence of SEQ ID NO: 129;
l. an HVR-H1 comprising the amino acid sequence of SEQ ID NO:67; an HVR-H2 comprising the amino acid sequence of SEQ ID NO:82; an HVR-H3 comprising the amino acid sequence of SEQ ID NO:96; an HVR-L1 comprising the amino acid sequence of SEQ ID NO: 101; an HVR-L2 comprising the amino acid sequence of SEQ ID NO: 116; and an HVR-L3 comprising the amino acid sequence of SEQ ID NO: 130;
m. an HVR-H1 comprising the amino acid sequence of SEQ ID NO:68; an HVR-H2 comprising the amino acid sequence of SEQ ID NO:83; an HVR-H3 comprising the amino acid sequence of SEQ ID NO:97; an HVR-L1 comprising the amino acid sequence of SEQ ID NO: 101; an HVR-L2 comprising the amino acid sequence of SEQ ID NO: 119; and an HVR-L3 comprising the amino acid sequence of SEQ ID NO: 131;
n. an HVR-H1 comprising the amino acid sequence of SEQ ID NO:69; an HVR-H2 comprising the amino acid sequence of SEQ ID NO:84; an HVR-H3 comprising the amino acid sequence of SEQ ID NO:98; an HVR-L1 comprising the amino acid sequence of SEQ ID NO:110; an HVR-L2 comprising the amino acid sequence of SEQ ID NO: 120; and an HVR-L3 comprising the amino acid sequence of SEQ ID NO: 132;
o. an HVR-H1 comprising the amino acid sequence of SEQ ID NO: 70; an HVR-H2 comprising the amino acid sequence of SEQ ID NO:85; an HVR-H3 comprising the amino acid sequence of SEQ ID NO:99; an HVR-L1 comprising the amino acid sequence of SEQ ID NO: 111; an HVR-L2 comprising the amino acid sequence of SEQ ID NO: 121; and an HVR-L3 comprising the amino acid sequence of SEQ ID NO: 133;
p. an HVR-H1 comprising the amino acid sequence of SEQ ID NO: 70; an HVR-H2 comprising the amino acid sequence of SEQ ID NO:86; an HVR-H3 comprising the amino acid sequence of SEQ ID NO: 100; an HVR-L1 comprising the amino acid sequence of SEQ ID NO: 111; an HVR-L2 comprising the amino acid sequence of SEQ ID NO: 121; and an HVR-L3 comprising the amino acid sequence of SEQ ID NO: 133;
q. the HVR-H1 comprises the amino acid sequence of SEQ ID NO: 167; the HVR-H2 comprises the amino acid sequence of SEQ ID NO: 170; the HVR-H3 comprises the amino acid sequence of SEQ ID NO: 179; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
r. the HVR-H1 comprises the amino acid sequence of SEQ ID NO: 168; the HVR-H2 comprises the amino acid sequence of SEQ ID NO: 171; the HVR-H3 comprises the amino acid sequence of SEQ ID NO: 180; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
s. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 181; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
t. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 182; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
u. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
v. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
w. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
x. the HVR-H1 comprises the amino acid sequence of SEQ ID NO: 169; the HVR-H2 comprises the amino acid sequence of SEQ ID NO: 1704; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
y. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
z. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
aa. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
bb. the HVR-H1 comprises the amino acid sequence of SEQ ID NO: 169; the HVR-H2 comprises the amino acid sequence of SEQ ID NO: 176; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
cc. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 187; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
dd. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 188; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
ee, the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 182; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
ff. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
gg. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
hh. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
ii. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
jj. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
kk. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
ll. the HVR-H1 comprises the amino acid sequence of SEQ ID NO: 169; the HVR-H2 comprises the amino acid sequence of SEQ ID NO: 173; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
mm. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 183; the HVR-L1 comprises the amino acid sequence of SEQ ID NO: 247; the HVR-L2 comprises the amino acid sequence of SEQ ID NO:248; and the HVR-L3 comprises the amino acid sequence of SEQ ID NO:249;
nn. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
oo. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
pp. the HVR-H1 comprises the amino acid sequence of SEQ ID NO: 169; the HVR-H2 comprises the amino acid sequence of SEQ ID NO: 175; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
qq. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 183; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
rr. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 184; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
ss. the HVR-H1 comprises the amino acid sequence of SEQ ID NO: 169; the HVR-H2 comprises the amino acid sequence of SEQ ID NO: 171; the HVR-H3 comprises the amino acid sequence of SEQ ID NO: 185; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
tt. the HVR-H1 comprises the amino acid sequence of SEQ ID NO: 169; the HVR-H2 comprises the amino acid sequence of SEQ ID NO: 172; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249;
uu. the HVR-H1 comprises the amino acid sequence of SEQ ID NO: 169; the HVR-H2 comprises the amino acid sequence of SEQ ID NO: 177; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO:248; and the HVR-L3 comprises the amino acid sequence of SEQ ID NO:249; or
vv. the HVR-H1 comprises the amino acid sequence of SEQ ID NO: 169; the HVR-H2 comprises the amino acid sequence of SEQ ID NO: 178; the HVR-H3 comprises the amino acid sequence of SEQ ID NO: 186; the HVR-L1 comprises the amino acid sequence of SEQ ID NO:247; the HVR-L2 comprises the amino acid sequence of SEQ ID NO: 248; and the HVR-L3 comprises the amino acid sequence of SEQ ID NO:249.

26. The antibody of claim 25, wherein the antibody comprises:
a. the HVRs of claim 25.a, and further comprises a VH that is at least 90%, at least 95%, at least 97%, or at least 99% identical to the amino acid sequence of SEQ ID NO:29;
b. the HVRs of claim 25.b, and further comprises a VH that is at least 90%, at least 95%, at least 97%, or at least 99% identical to the amino acid sequence of SEQ ID NO:30;
c. the HVRs of claim 25.c, and further comprises a VH that is at least 90%, at least 95%, at least 97%, or at least 99% identical to the amino acid sequence of SEQ ID NO:31;
d. the HVRs of claim 25.d, and further comprises a VH that is at least 90%, at least 95%, at least 97%, or at least 99% identical to the amino acid sequence of SEQ ID NO:32;
c. the HVRs of claim 25.e, and further comprises a VH that is at least 90%, at least 95%, at least 97%, or at least 99% identical to the amino acid sequence of SEQ ID NO:33;
f. the HVRs of claim 25.f, and further comprises a VH that is at least 90%, at least 95%, at least 97%, or at least 99% identical to the amino acid sequence of SEQ ID NO:34;
g. the HVRs of claim 25.g, and further comprises a VH that is at least 90%, at least 95%, at least 97%, or at least 99% identical to the amino acid sequence of SEQ ID NO:35;
h. the HVRs of claim 25.h, and further comprises a VH that is at least 90%, at least 95%, at least 97%, or at least 99% identical to the amino acid sequence of SEQ ID NO:36;
i. the HVRs of claim 25.i, and further comprises a VH that is at least 90%, at least 95%, at least 97%, or at least 99% identical to the amino acid sequence of SEQ ID NO:37;
j. the HVRs of claim 25.j, and further comprises a VH that is at least 90%, at least 95%, at least 97%, or at least 99% identical to the amino acid sequence of SEQ ID NO:38;
k. the HVRs of claim 25.k, and further comprises a VH that is at least 90%, at least 95%, at least 97%, or at least 99% identical to the amino acid sequence of SEQ ID NO:39;
l. the HVRs of claim 25.l, and further comprises a VH that is at least 90%, at least 95%, at least 97%, or at least 99% identical to the amino acid sequence of SEQ ID NO:40;
m. the HVRs of claim 25.m, and further comprises a VH that is at least 90%, at least 95%, at least 97%, or at least 99% identical to the amino acid sequence of SEQ ID NO:41;
n. the HVRs of claim 25.n, and further comprises a VH that is at least 90%, at least 95%, at least 97%, or at least 99% identical to the amino acid sequence of SEQ ID NO:42;
o. the HVRs of claim 25.o, and further comprises a VH that is at least 90%, at least 95%, at least 97%, or at least 99% identical to the amino acid sequence of SEQ ID NO:43;
p. the HVRs of claim 25.p, and further comprises a VH that is at least 90%, at least 95%, at least 97%, or at least 99% identical to the amino acid sequence of SEQ ID NO:44
q. the HVRs of claim 25.q, and further comprises a VH that is at least 90%, at least 95%, at least 97%, or at least 99% identical to the amino acid sequence of SEQ ID NO: 134;
r. the HVRs of claim 25.r, and further comprises a VH that is at least 90%, at least 95%, at least 97%, or at least 99% identical to the amino acid sequence of SEQ ID NO: 135;
s. the HVRs of claim 25.s, and further comprises a VH that is at least 90%, at least 95%, at least 97%, or at least 99% identical to the amino acid sequence of SEQ ID NO: 136;
t. the HVRs of claim 25.t, and further comprises a VH that is at least 90%, at least 95%, at least 97%, or at least 99% identical to the amino acid sequence of SEQ ID NO: 137;
u. the HVRs of claim 25.u, and further comprises a VH that is at least 90%, at least 95%, at least 97%, or at least 99% identical to the amino acid sequence of SEQ ID NO: 138;
v. the HVRs of claim 25.v, and further comprises a VH that is at least 90%, at least 95%, at least 97%, or at least 99% identical to the amino acid sequence of SEQ ID NO: 139;
w. the HVRs of claim 25.w, and further comprises a VH that is at least 90%, at least 95%, at least 97%, or at least 99% identical to the amino acid sequence of SEQ ID NO: 140;
x. the HVRs of claim 25.x, and further comprises a VH that is at least 90%, at least 95%, at least 97%, or at least 99% identical to the amino acid sequence of SEQ ID NO: 141;
y. the HVRs of claim 25.y, and further comprises a VH that is at least 90%, at least 95%, at least 97%, or at least 99% identical to the amino acid sequence of SEQ ID NO: 142;
z. the HVRs of claim 25.z, and further comprises a VH that is at least 90%, at least 95%, at least 97%, or at least 99% identical to the amino acid sequence of SEQ ID NO: 143;
aa. the HVRs of claim 25.aa, and further comprises a VH that is at least 90%, at least 95%, at least 97%, or at least 99% identical to the amino acid sequence of SEQ ID NO: 144;
bb. the HVRs of claim 25.bb, and further comprises a VH that is at least 90%, at least 95%, at least 97%, or at least 99% identical to the amino acid sequence of SEQ ID NO: 145;
cc. the HVRs of claim 25.cc, and further comprises a VH that is at least 90%, at least 95%, at least 97%, or at least 99% identical to the amino acid sequence of SEQ ID NO: 146;
dd. the HVRs of claim 25.dd, and further comprises a VH that is at least 90%, at least 95%, at least 97%, or at least 99% identical to the amino acid sequence of SEQ ID NO: 147;
ee, the HVRs of claim 25.ee, and further comprises a VH that is at least 90%, at least 95%, at least 97%, or at least 99% identical to the amino acid sequence of SEQ ID NO: 148;
ff. the HVRs of claim 25.ff, and further comprises a VH that is at least 90%, at least 95%, at least 97%, or at least 99% identical to the amino acid sequence of SEQ ID NO: 149;
gg. the HVRs of claim 25.gg, and further comprises a VH that is at least 90%, at least 95%, at least 97%, or at least 99% identical to the amino acid sequence of SEQ ID NO: 150;
hh. the HVRs of claim 25.hh, and further comprises a VH that is at least 90%, at least 95%, at least 97%, or at least 99% identical to the amino acid sequence of SEQ ID NO: 151;
ii. the HVRs of claim 25.ii, and further comprises a VH that is at least 90%, at least 95%, at least 97%, or at least 99% identical to the amino acid sequence of SEQ ID NO: 152;
jj. the HVRs of claim 25.jj, and further comprises a VH that is at least 90%, at least 95%, at least 97%, or at least 99% identical to the amino acid sequence of SEQ ID NO: 153;
kk. the HVRs of claim 25.kk, and further comprises a VH that is at least 90%, at least 95%, at least 97%, or at least 99% identical to the amino acid sequence of SEQ ID NO: 154;
ll. the HVRs of claim 25.ll, and further comprises a VH that is at least 90%, at least 95%, at least 97%, or at least 99% identical to the amino acid sequence of SEQ ID NO: 155;
mm. the HVRs of claim 25.mm, and further comprises a VH that is at least 90%, at least 95%, at least 97%, or at least 99% identical to the amino acid sequence of SEQ ID NO: 156;
nn. the HVRs of claim 25.nn, and further comprises a VH that is at least 90%, at least 95%, at least 97%, or at least 99% identical to the amino acid sequence of SEQ ID NO: 157;
oo. the HVRs of claim 25.oo, and further comprises a VH that is at least 90%, at least 95%, at least 97%, or at least 99% identical to the amino acid sequence of SEQ ID NO: 158;
pp. the HVRs of claim 25.pp, and further comprises a VH that is at least 90%, at least 95%, at least 97%, or at least 99% identical to the amino acid sequence of SEQ ID NO: 159;
qq. the HVRs of claim 25.qq, and further comprises a VH that is at least 90%, at least 95%, at least 97%, or at least 99% identical to the amino acid sequence of SEQ ID NO: 160;
rr. the HVRs of claim 25.rr, and further comprises a VH that is at least 90%, at least 95%, at least 97%, or at least 99% identical to the amino acid sequence of SEQ ID NO: 161;
ss. the HVRs of claim 25.ss, and further comprises a VH that is at least 90%, at least 95%, at least 97%, or at least 99% identical to the amino acid sequence of SEQ ID NO: 162;
tt. the HVRs of claim 25.tt, and further comprises a VH that is at least 90%, at least 95%, at least 97%, or at least 99% identical to the amino acid sequence of SEQ ID NO: 163;
uu. the HVRs of claim 25.uu, and further comprises a VH that is at least 90%, at least 95%, at least 97%, or at least 99% identical to the amino acid sequence of SEQ ID NO: 164;
vv. the HVRs of claim 25.vv, and further comprises a VH that is at least 90%, at least 95%, at least 97%, or at least 99% identical to the amino acid sequence of SEQ ID NO: 165.

27. The antibody of claim 25 or 26, wherein the antibody comprises:
a. the HVRs of claim 25.a, and further comprises a VL that is at least 90%, at least 95%, at least 97%, or at least 99% identical to the amino acid sequence of SEQ ID NO:45
b. the HVRs of claim 25.b, and further comprises a VL that is at least 90%, at least 95%, at least 97%, or at least 99% identical to the amino acid sequence of SEQ ID NO: 46;
c. the HVRs of claim 25.c, and further comprises a VL that is at least 90%, at least 95%, at least 97%, or at least 99% identical to the amino acid sequence of SEQ ID NO: 47;
d. the HVRs of claim 25.d, and further comprises a VL that is at least 90%, at least 95%, at least 97%, or at least 99% identical to the amino acid sequence of SEQ ID NO: 48;
e. the HVRs of claim 25.e, and further comprises a VL that is at least 90%, at least 95%, at least 97%, or at least 99% identical to the amino acid sequence of SEQ ID NO: 49;
f. the HVRs of claim 25.f, and further comprises a VL that is at least 90%, at least 95%, at least 97%, or at least 99% identical to the amino acid sequence of SEQ ID NO: 50;
g. the HVRs of claim 25.g, and further comprises a VL that is at least 90%, at least 95%, at least 97%, or at least 99% identical to the amino acid sequence of SEQ ID NO: 51;
h. the HVRs of claim 25.h, and further comprises a VL that is at least 90%, at least 95%, at least 97%, or at least 99% identical to the amino acid sequence of SEQ ID NO: 52;
l. the HVRs of claim 25.i, and further comprises a VL that is at least 90%, at least 95%, at least 97%, or at least 99% identical to the amino acid sequence of SEQ ID NO: 53;
j. the HVRs of claim 25.j, and further comprises a VL that is at least 90%, at least 95%, at least 97%, or at least 99% identical to the amino acid sequence of SEQ ID NO: 54;
k. the HVRs of claim 25.k, and further comprises a VL that is at least 90%, at least 95%, at least 97%, or at least 99% identical to the amino acid sequence of SEQ ID NO: 55;
l. the HVRs of claim 25.l, and further comprises a VL that is at least 90%, at least 95%, at least 97%, or at least 99% identical to the amino acid sequence of SEQ ID NO: 56;
m. the HVRs of claim 25.m, and further comprises a VL that is at least 90%, at least 95%, at least 97%, or at least 99% identical to the amino acid sequence of SEQ ID NO: 57
n. the HVRs of claim 25.n, and further comprises a VL that is at least 90%, at least 95%, at least 97%, or at least 99% identical to the amino acid sequence of SEQ ID NO: 58;
o. the HVRs of claim 25.o, and further comprises a VL that is at least 90%, at least 95%, at least 97%, or at least 99% identical to the amino acid sequence of SEQ ID NO: 59;
p. the HVRs of claim 25.p, and further comprises a VL that is at least 90%, at least 95%, at least 97%, or at least 99% identical to the amino acid sequence of SEQ ID NO: 59;
q. the HVRs of claim 25.q, and further comprises a VL that is at least 90%, at least 95%, at least 97%, or at least 99% identical to the amino acid sequence of SEQ ID NO: 166;
r. the HVRs of claim 25.r, and further comprises a VL that is at least 90%, at least 95%, at least 97%, or at least 99% identical to the amino acid sequence of SEQ ID NO: 166;
s. the HVRs of claim 25.s, and further comprises a VL that is at least 90%, at least 95%, at least 97%, or at least 99% identical to the amino acid sequence of SEQ ID NO: 166;
t. the HVRs of claim 25.t, and further comprises a VL that is at least 90%, at least 95%, at least 97%, or at least 99% identical to the amino acid sequence of SEQ ID NO: 166;
u. the HVRs of claim 25.u, and further comprises a VL that is at least 90%, at least 95%, at least 97%, or at least 99% identical to the amino acid sequence of SEQ ID NO: 166;
v. the HVRs of claim 25.v, and further comprises a VL that is at least 90%, at least 95%, at least 97%, or at least 99% identical to the amino acid sequence of SEQ ID NO: 166;
w. the HVRs of claim 25.w, and further comprises a VL that is at least 90%, at least 95%, at least 97%, or at least 99% identical to the amino acid sequence of SEQ ID NO: 166;
x. the HVRs of claim 25.x, and further comprises a VL that is at least 90%, at least 95%, at least 97%, or at least 99% identical to the amino acid sequence of SEQ ID NO: 166;
y. the HVRs of claim 25.y, and further comprises a VL that is at least 90%, at least 95%, at least 97%, or at least 99% identical to the amino acid sequence of SEQ ID NO: 166;
z. the HVRs of claim 25.z, and further comprises a VL that is at least 90%, at least 95%, at least 97%, or at least 99% identical to the amino acid sequence of SEQ ID NO: 166;
aa. the HVRs of claim 25.aa, and further comprises a VL that is at least 90%, at least 95%, at least 97%, or at least 99% identical to the amino acid sequence of SEQ ID NO: 166;
bb. the HVRs of claim 25.bb, and further comprises a VL that is at least 90%, at least 95%, at least 97%, or at least 99% identical to the amino acid sequence of SEQ ID NO: 166;
cc. the HVRs of claim 25.cc, and further comprises a VL that is at least 90%, at least 95%, at least 97%, or at least 99% identical to the amino acid sequence of SEQ ID NO: 166;
dd. the HVRs of claim 25.dd, and further comprises a VL that is at least 90%, at least 95%, at least 97%, or at least 99% identical to the amino acid sequence of SEQ ID NO: 166;
ee, the HVRs of claim 25.cc, and further comprises a VL that is at least 90%, at least 95%, at least 97%, or at least 99% identical to the amino acid sequence of SEQ ID NO: 166;
ff. the HVRs of claim 25.ff, and further comprises a VL that is at least 90%, at least 95%, at least 97%, or at least 99% identical to the amino acid sequence of SEQ ID NO: 166;
gg. the HVRs of claim 25.gg, and further comprises a VL that is at least 90%, at least 95%, at least 97%, or at least 99% identical to the amino acid sequence of SEQ ID NO: 166;
hh. the HVRs of claim 25.hh, and further comprises a VL that is at least 90%, at least 95%, at least 97%, or at least 99% identical to the amino acid sequence of SEQ ID NO: 166;
ii. the HVRs of claim 25.ii, and further comprises a VL that is at least 90%, at least 95%, at least 97%, or at least 99% identical to the amino acid sequence of SEQ ID NO: 166;
jj. the HVRs of claim 25.jj, and further comprises a VL that is at least 90%, at least 95%, at least 97%, or at least 99% identical to the amino acid sequence of SEQ ID NO: 166;
kk. the HVRs of claim 25.kk, and further comprises a VL that is at least 90%, at least 95%, at least 97%, or at least 99% identical to the amino acid sequence of SEQ ID NO: 166;
ll. the HVRs of claim 25.ll, and further comprises a VL that is at least 90%, at least 95%, at least 97%, or at least 99% identical to the amino acid sequence of SEQ ID NO: 166;
mm. the HVRs of claim 25.mm, and further comprises a VL that is at least 90%, at least 95%, at least 97%, or at least 99% identical to the amino acid sequence of SEQ ID NO: 166;
nn. the HVRs of claim 25.nn, and further comprises a VL that is at least 90%, at least 95%, at least 97%, or at least 99% identical to the amino acid sequence of SEQ ID NO: 166;
oo. the HVRs of claim 25.oo, and further comprises a VL that is at least 90%, at least 95%, at least 97%, or at least 99% identical to the amino acid sequence of SEQ ID NO: 166;
pp. the HVRs of claim 25.pp, and further comprises a VL that is at least 90%, at least 95%, at least 97%, or at least 99% identical to the amino acid sequence of SEQ ID NO: 166;
qq. the HVRs of claim 25.qq, and further comprises a VL that is at least 90%, at least 95%, at least 97%, or at least 99% identical to the amino acid sequence of SEQ ID NO: 166;
rr. the HVRs of claim 25.rr, and further comprises a VL that is at least 90%, at least 95%, at least 97%, or at least 99% identical to the amino acid sequence of SEQ ID NO: 166;
ss. the HVRs of claim 25.ss, and further comprises a VL that is at least 90%, at least 95%, at least 97%, or at least 99% identical to the amino acid sequence of SEQ ID NO: 166;
tt. the HVRs of claim 25.tt, and further comprises a VL that is at least 90%, at least 95%, at least 97%, or at least 99% identical to the amino acid sequence of SEQ ID NO: 166;
uu. the HVRs of claim 25.uu, and further comprises a VL that is at least 90%, at least 95%, at least 97%, or at least 99% identical to the amino acid sequence of SEQ ID NO: 166; or
vv. the HVRs of claim 25.vv, and further comprises a VL that is at least 90%, at least 95%, at least 97%, or at least 99% identical to the amino acid sequence of SEQ ID NO: 166.

28. The antibody of any one of claims 25-27, wherein the antibody comprises:
a. the HVRs of claim 25.a, and further comprises a VH comprising the amino acid sequence of SEQ ID NO:29;
b. the HVRs of claim 25.b, and further comprises a VH comprising the amino acid sequence of SEQ ID NO:30;
c. the HVRs of claim 25.c, and further comprises a VH comprising the amino acid sequence of SEQ ID NO:31;
d. the HVRs of claim 25.d, and further comprises a VH comprising the amino acid sequence of SEQ ID NO:32;
e. the HVRs of claim 25.e, and further comprises a VH comprising the amino acid sequence of SEQ ID NO:33;
f. the HVRs of claim 25.f, and further comprises a VH comprising the amino acid sequence of SEQ ID NO:34;
g. the HVRs of claim 25.g, and further comprises a VH comprising the amino acid sequence of SEQ ID NO:35;
h. the HVRs of claim 25.h, and further comprises a VH comprising the amino acid sequence of SEQ ID NO:36;
1, the HVRs of claim 25.i, and further comprises a VH comprising the amino acid sequence of SEQ ID NO:37;
j. the HVRs of claim 25.j, and further comprises a VH comprising the amino acid sequence of SEQ ID NO:38;
k. the HVRs of claim 25.k, and further comprises a VH comprising the amino acid sequence of SEQ ID NO:39;
l. the HVRs of claim 25.l, and further comprises a VH comprising the amino acid sequence of SEQ ID NO:40;
m. the HVRs of claim 25.m, and further comprises a comprising the amino acid sequence of SEQ ID NO:41;
n. the HVRs of claim 25.n, and further comprises a VH comprising the amino acid sequence of SEQ ID NO:42;
o. the HVRs of claim 25.o, and further comprises a VH comprising the amino acid sequence of SEQ ID NO:43;
p. the HVRs of claim 25.p, and further comprises a VH comprising the amino acid sequence of SEQ ID NO:44;
q. the HVRs of claim 25.q, and further comprises a VH comprising the amino acid sequence of SEQ ID NO: 134;
r. the HVRs of claim 25.r, and further comprises a VH comprising the amino acid sequence of SEQ ID NO: 135;
s. the HVRs of claim 25.s, and further comprises a VH comprising the amino acid sequence of SEQ ID NO:136;
t. the HVRs of claim 25.t, and further comprises a VH comprising the amino acid sequence of SEQ ID NO: 137;
u. the HVRs of claim 25.u, and further comprises a VH comprising the amino acid sequence of SEQ ID NO: 138;
v. the HVRs of claim 25.v, and further comprises a VH comprising the amino acid sequence of SEQ ID NO:139;
w. the HVRs of claim 25.w, and further comprises a VH comprising the amino acid sequence of SEQ ID NO: 140;
x. the HVRs of claim 25.x, and further comprises a VH comprising the amino acid sequence of SEQ ID NO: 141;
y. the HVRs of claim 25.y, and further comprises a VH comprising the amino acid sequence of SEQ ID NO:142;
z. the HVRs of claim 25.z, and further comprises a VH comprising the amino acid sequence of SEQ ID NO: 143;
aa. the HVRs of claim 25.aa, and further comprises a VH comprising the amino acid sequence of SEQ ID NO: 144;
bb. the HVRs of claim 25.bb, and further comprises a VH comprising the amino acid sequence of SEQ ID NO: 145;
cc. the HVRs of claim 25.cc, and further comprises a VH comprising the amino acid sequence of SEQ ID NO: 146;
dd. the HVRs of claim 25.dd, and further comprises a VH comprising the amino acid sequence of SEQ ID NO: 147;
ee, the HVRs of claim 25.ee, and further comprises a VH comprising the amino acid sequence of SEQ ID NO: 148;
ff. the HVRs of claim 25.ff, and further comprises a VH comprising the amino acid sequence of SEQ ID NO: 149;
gg. the HVRs of claim 25.gg, and further comprises a VH comprising the amino acid sequence of SEQ ID NO: 150;
hh. the HVRs of claim 25.hh, and further comprises a VH comprising the amino acid sequence of SEQ ID NO: 151;
ii. the HVRs of claim 25.ii, and further comprises a VH comprising the amino acid sequence of SEQ ID NO: 152;
jj. the HVRs of claim 25.jj, and further comprises a VH comprising the amino acid sequence of SEQ ID NO: 153;
kk. the HVRs of claim 25.kk, and further comprises a VH that is at least 90%, at least 95%, at least 97%, or at least 99% identical t VH comprising o the amino acid sequence of SEQ ID NO: 154;
ll. the HVRs of claim 25.ll, and further comprises a VH comprising the amino acid sequence of SEQ ID NO: 155;
mm. the HVRs of claim 25.mm, and further comprises a VH comprising the amino acid sequence of SEQ ID NO: 156;
nn. the HVRs of claim 25.nn, and further comprises a VH comprising the amino acid sequence of SEQ ID NO: 157;
oo. the HVRs of claim 25.oo, and further comprises a VH comprising amino acid sequence of SEQ ID NO: 158;
pp. the HVRs of claim 25.pp, and further comprises a VH comprising the amino acid sequence of SEQ ID NO: 159;
qq. the HVRs of claim 25.qq, and further comprises a VH comprising the amino acid sequence of SEQ ID NO: 160;
rr. the HVRs of claim 25.rr, and further comprises a VH comprising the amino acid sequence of SEQ ID NO: 161;
ss. the HVRs of claim 25.ss, and further comprises a VH comprising the amino acid sequence of SEQ ID NO: 162;
tt. the HVRs of claim 25.tt, and further comprises a VH comprising the amino acid sequence of SEQ ID NO: 163;
uu. the HVRs of claim 25.uu, and further comprises a VH comprising the amino acid sequence of SEQ ID NO: 164; or
vv. the HVRs of claim 25.vv, and further comprises a VH comprising the amino acid sequence of SEQ ID NO: 165.

29. The antibody of any one of claims 25-28, wherein the antibody comprises:
a. the HVRs of claim 25.a, and further comprises a VL comprising the amino acid sequence of SEQ ID NO:45 b, the HVRs of claim 25.b, and further comprises a VL comprising amino acid sequence of SEQ ID NO: 46;
c. the HVRs of claim 25.c, and further comprises a VL comprising the amino acid sequence of SEQ ID NO: 47;
d. the HVRs of claim 25.d, and further comprises a VL comprising the amino acid sequence of SEQ ID NO: 48;
e. the HVRs of claim 25.e, and further comprises a VL comprising the amino acid sequence of SEQ ID NO: 49;
f. the HVRs of claim 25.f, and further comprises a VL comprising the amino acid sequence of SEQ ID NO: 50;
g. the HVRs of claim 25.g, and further comprises a VL comprising the amino acid sequence of SEQ ID NO: 51;
h. the HVRs of claim 25.h, and further comprises a VL comprising the amino acid sequence of SEQ ID NO: 52;
1, the HVRs of claim 25.i, and further comprises a VL comprising the amino acid sequence of SEQ ID NO: 53;
j. the HVRs of claim 25.j, and further comprises a VL comprising the amino acid sequence of SEQ ID NO: 54;
k. the HVRs of claim 25.k, and further comprises a VL comprising the amino acid sequence of SEQ ID NO: 55;
l. the HVRs of claim 25.l, and further comprises a VL comprising the amino acid sequence of SEQ ID NO: 56;
m. the HVRs of claim 25.m, and further comprises a VL comprising the amino acid sequence of SEQ ID NO: 57
n. the HVRs of claim 25.n, and further comprises a VL comprising the amino acid sequence of SEQ ID NO: 58;
o. the HVRs of claim 25.o, and further comprises a VL comprising the amino acid sequence of SEQ ID NO: 59;
p. the HVRs of claim 25.p, and further comprises a VL comprising the amino acid sequence of SEQ ID NO: 59;
q. the HVRs of claim 25.q, and further comprises a VL comprising the amino acid sequence of SEQ ID NO: 166;
r. the HVRs of claim 25.r, and further comprises a VL comprising the amino acid sequence of SEQ ID NO: 166;
s. the HVRs of claim 25.s, and further comprises a VL comprising the amino acid sequence of SEQ ID NO: 166;
t. the HVRs of claim 25.t, and further comprises a VL comprising the amino acid sequence of SEQ ID NO: 166;
u. the HVRs of claim 25.u, and further comprises a VL comprising the amino acid sequence of SEQ ID NO: 166;
v. the HVRs of claim 25.v, and further comprises a VL comprising the amino acid sequence of SEQ ID NO: 166;
w. the HVRs of claim 25.w, and further comprises a VL comprising the amino acid sequence of SEQ ID NO: 166;
x. the HVRs of claim 25.x, and further comprises a VL comprising the amino acid sequence of SEQ ID NO: 166;
y. the HVRs of claim 25.y, and further comprises a VL comprising the amino acid sequence of SEQ ID NO: 166;
z. the HVRs of claim 25.z, and further comprises a VL comprising the amino acid sequence of SEQ ID NO: 166;
aa. the HVRs of claim 25.aa, and further comprises a VL comprising the amino acid sequence of SEQ ID NO: 166;
bb. the HVRs of claim 25.bb, and further comprises a VL comprising the amino acid sequence of SEQ ID NO: 166;
cc. the HVRs of claim 25.cc, and further comprises a VL comprising the amino acid sequence of SEQ ID NO: 166;
dd. the HVRs of claim 25.dd, and further comprises a VL comprising the amino acid sequence of SEQ ID NO: 166;
ee, the HVRs of claim 25.ee, and further comprises a VL comprising the amino acid sequence of SEQ ID NO: 166;
ff. the HVRs of claim 25.ff, and further comprises a VL comprising the amino acid sequence of SEQ ID NO: 166;
gg. the HVRs of claim 25.gg, and further comprises a VL comprising the amino acid sequence of SEQ ID NO: 166;
hh. the HVRs of claim 25.hh, and further comprises a VL comprising the amino acid sequence of SEQ ID NO: 166;
ii. the HVRs of claim 25.ii, and further comprises a VL comprising the amino acid sequence of SEQ ID NO: 166;
jj. the HVRs of claim 25.jj, and further comprises a VL comprising the amino acid sequence of SEQ ID NO: 166;
kk. the HVRs of claim 25.kk, and further comprises a VL comprising the amino acid sequence of SEQ ID NO: 166;
ll. the HVRs of claim 25.ll, and further comprises a VL comprising the amino acid sequence of SEQ ID NO: 166;
mm. the HVRs of claim 25.mm, and further comprises a VL comprising the amino acid sequence of SEQ ID NO: 166;
nn. the HVRs of claim 25.nn, and further comprises a VL comprising the amino acid sequence of SEQ ID NO: 166;
oo. the HVRs of claim 25.oo, and further comprises a VL comprising the amino acid sequence of SEQ ID NO: 166;
pp. the HVRs of claim 25.pp, and further comprises a VL comprising the amino acid sequence of SEQ ID NO: 166;
qq. the HVRs of claim 25.qq, and further comprises a VL comprising the amino acid sequence of SEQ ID NO: 166;
rr. the HVRs of claim 25.rr, and further comprises a VL comprising the amino acid sequence of SEQ ID NO: 166;
ss. the HVRs of claim 25.ss, and further comprises a VL comprising the amino acid sequence of SEQ ID NO: 166;
tt. the HVRs of claim 25.tt, and further comprises a VL comprising the amino acid sequence of SEQ ID NO: 166;
uu. the HVRs of claim 25.uu, and further comprises a VL comprising the amino acid sequence of SEQ ID NO: 166; or
vv. the HVRs of claim 25.vv, and further comprises a VL comprising the amino acid sequence of SEQ ID NO: 166.

30. The antibody of claim 25, wherein the antibody comprises:
a. a VH comprising the amino acid sequence of SEQ ID NO: 29 and a VL comprising the amino acid sequence of SEQ ID NO: 45;
b. a VH comprising the amino acid sequence of SEQ ID NO: 30 and a VL comprising the amino acid sequence of SEQ ID NO: 46;
c. a VH comprising the amino acid sequence of SEQ ID NO: 31 and a VL comprising the amino acid sequence of SEQ ID NO: 47;
d. a VH comprising the amino acid sequence of SEQ ID NO: 32 and a VL comprising the amino acid sequence of SEQ ID NO: 48;
e. a VH comprising the amino acid sequence of SEQ ID NO: 33 and a VL comprising the amino acid sequence of SEQ ID NO: 49;
f. a VH comprising the amino acid sequence of SEQ ID NO: 34 and a VL comprising the amino acid sequence of SEQ ID NO: 50;
g. a VH comprising the amino acid sequence of SEQ ID NO: 35 and a VL comprising the amino acid sequence of SEQ ID NO: 51;
h. a VH comprising the amino acid sequence of SEQ ID NO: 36 and a VL comprising the amino acid sequence of SEQ ID NO: 52;
i. a VH comprising the amino acid sequence of SEQ ID NO: 37 and a VL comprising the amino acid sequence of SEQ ID NO: 53;
j. a VH comprising the amino acid sequence of SEQ ID NO: 38 and a VL comprising the amino acid sequence of SEQ ID NO: 54;
k. a VH comprising the amino acid sequence of SEQ ID NO: 39 and a VL comprising the amino acid sequence of SEQ ID NO: 55;
l. a VH comprising the amino acid sequence of SEQ ID NO: 40 and a VL comprising the amino acid sequence of SEQ ID NO: 56;
m. a VH comprising the amino acid sequence of SEQ ID NO: 41 and a VL comprising the amino acid sequence of SEQ ID NO: 57;
n. a VH comprising the amino acid sequence of SEQ ID NO: 42 and a VL comprising the amino acid sequence of SEQ ID NO: 58;
o. a VH comprising the amino acid sequence of SEQ ID NO: 43 and a VL comprising the amino acid sequence of SEQ ID NO: 59;
p. a VH comprising the amino acid sequence of SEQ ID NO: 44 and a VL comprising the amino acid sequence of SEQ ID NO: 59;
q. a VH comprising the amino acid sequence of SEQ ID NO: 134 and a VL comprising the amino acid sequence of SEQ ID NO: 166;
r. a VH comprising the amino acid sequence of SEQ ID NO: 135 and a VL comprising the amino acid sequence of SEQ ID NO: 166;
s. a VH comprising the amino acid sequence of SEQ ID NO: 136 and a VL comprising the amino acid sequence of SEQ ID NO: 166;
t. a VH comprising the amino acid sequence of SEQ ID NO: 137 and a VL comprising the amino acid sequence of SEQ ID NO: 166;
u. a VH comprising the amino acid sequence of SEQ ID NO: 138 and a VL comprising the amino acid sequence of SEQ ID NO: 166;
v. a VH comprising the amino acid sequence of SEQ ID NO: 139 and a VL comprising the amino acid sequence of SEQ ID NO: 166;
w. a VH comprising the amino acid sequence of SEQ ID NO: 140 and a VL comprising the amino acid sequence of SEQ ID NO: 166;
x. a VH comprising the amino acid sequence of SEQ ID NO: 141 and a VL comprising the amino acid sequence of SEQ ID NO: 166;
y. a VH comprising the amino acid sequence of SEQ ID NO: 142 and a VL comprising the amino acid sequence of SEQ ID NO: 166;
z. a VH comprising the amino acid sequence of SEQ ID NO: 143 and a VL comprising the amino acid sequence of SEQ ID NO: 166;
aa. a VH comprising the amino acid sequence of SEQ ID NO: 144 and a VL comprising the amino acid sequence of SEQ ID NO: 166;
bb. a VH comprising the amino acid sequence of SEQ ID NO: 145 and a VL comprising the amino acid sequence of SEQ ID NO: 166;
cc. a VH comprising the amino acid sequence of SEQ ID NO: 146 and a VL comprising the amino acid sequence of SEQ ID NO: 166;
dd. a VH comprising the amino acid sequence of SEQ ID NO: 147 and a VL comprising the amino acid sequence of SEQ ID NO: 166;
ee. a VH comprising the amino acid sequence of SEQ ID NO: 148 and a VL comprising the amino acid sequence of SEQ ID NO: 166;
ff. a VH comprising the amino acid sequence of SEQ ID NO: 149 and a VL comprising the amino acid sequence of SEQ ID NO: 166;
gg. a VH comprising the amino acid sequence of SEQ ID NO: 150 and a VL comprising the amino acid sequence of SEQ ID NO: 166;
hh. a VH comprising the amino acid sequence of SEQ ID NO: 151 and a VL comprising the amino acid sequence of SEQ ID NO: 166;
ii. a VH comprising the amino acid sequence of SEQ ID NO: 152 and a VL comprising the amino acid sequence of SEQ ID NO: 166;
jj. a VH comprising the amino acid sequence of SEQ ID NO: 153 and a VL comprising the amino acid sequence of SEQ ID NO: 166;
kk. a VH comprising the amino acid sequence of SEQ ID NO: 154 and a VL comprising the amino acid sequence of SEQ ID NO: 166;
ll. a VH comprising the amino acid sequence of SEQ ID NO: 155 and a VL comprising the amino acid sequence of SEQ ID NO: 166;
mm. a VH comprising the amino acid sequence of SEQ ID NO: 156 and a VL comprising the amino acid sequence of SEQ ID NO: 166;
nn. a VH comprising the amino acid sequence of SEQ ID NO: 157 and a VL comprising the amino acid sequence of SEQ ID NO: 166;
oo. a VH comprising the amino acid sequence of SEQ ID NO: 158 and a VL comprising the amino acid sequence of SEQ ID NO: 166;
pp. a VH comprising the amino acid sequence of SEQ ID NO: 159 and a VL comprising the amino acid sequence of SEQ ID NO: 166;
qq. a VH comprising the amino acid sequence of SEQ ID NO: 160 and a VL comprising the amino acid sequence of SEQ ID NO: 166;
rr. a VH comprising the amino acid sequence of SEQ ID NO: 161 and a VL comprising the amino acid sequence of SEQ ID NO: 166;
ss. a VH comprising the amino acid sequence of SEQ ID NO: 162 and a VL comprising the amino acid sequence of SEQ ID NO: 166;
tt. a VH comprising the amino acid sequence of SEQ ID NO: 163 and a VL comprising the amino acid sequence of SEQ ID NO: 166;
uu. a VH comprising the amino acid sequence of SEQ ID NO: 164 and a VL comprising the amino acid sequence of SEQ ID NO: 166; or
vv. a VH comprising the amino acid sequence of SEQ ID NO: 165 and a VL comprising the amino acid sequence of SEQ ID NO: 166.

31. The antibody of claim 25, wherein the antibody comprises:
a. the HVRs of claim 25.s, and further comprises a heavy chain that is at least 90%, at least 95%, at least 97%, or at least 99% identical to the amino acid sequence of SEQ ID NO: 190;
b. the HVRs of claim 25.s, and further comprises a heavy chain that is at least 90%, at least 95%, at least 97%, or at least 99% identical to the amino acid sequence of SEQ ID NO: 191;
c. the HVRs of claim 25.s, and further comprises a heavy chain that is at least 90%, at least 95%, at least 97%, or at least 99% identical to the amino acid sequence of SEQ ID NO: 192;
d. the HVRs of claim 25.s, and further comprises a heavy chain that is at least 90%, at least 95%, at least 97%, or at least 99% identical to the amino acid sequence of SEQ ID NO: 193;
e. the HVRs of claim 25.s, and further comprises a heavy chain that is at least 90%, at least 95%, at least 97%, or at least 99% identical to the amino acid sequence of SEQ ID NO: 194;
f. the HVRs of claim 25.s, and further comprises a heavy chain that is at least 90%, at least 95%, at least 97%, or at least 99% identical to the amino acid sequence of SEQ ID NO: 195;
g. the HVRs of claim 25.s, and further comprises a heavy chain that is at least 90%, at least 95%, at least 97%, or at least 99% identical to the amino acid sequence of SEQ ID NO: 196;
h. the HVRs of claim 25.s, and further comprises a heavy chain that is at least 90%, at least 95%, at least 97%, or at least 99% identical to the amino acid sequence of SEQ ID NO: 197;
i. the HVRs of claim 25.s, and further comprises a heavy chain that is at least 90%, at least 95%, at least 97%, or at least 99% identical to the amino acid sequence of SEQ ID NO: 198;
j. the HVRs of claim 25.s, and further comprises a heavy chain that is at least 90%, at least 95%, at least 97%, or at least 99% identical to the amino acid sequence of SEQ ID NO: 199;
k. the HVRs of claim 25.s, and further comprises a heavy chain that is at least 90%, at least 95%, at least 97%, or at least 99% identical to the amino acid sequence of SEQ ID NO: 200;
l. the HVRs of claim 25.s, and further comprises a heavy chain that is at least 90%, at least 95%, at least 97%, or at least 99% identical to the amino acid sequence of SEQ ID NO: 201;
m. the HVRs of claim 25.s, and further comprises a heavy chain that is at least 90%, at least 95%, at least 97%, or at least 99% identical to the amino acid sequence of SEQ ID NO: 202;
n. the HVRs of claim 25.s, and further comprises a heavy chain that is at least 90%, at least 95%, at least 97%, or at least 99% identical to the amino acid sequence of SEQ ID NO: 203;
o. the HVRs of claim 25.s, and further comprises a heavy chain that is at least 90%, at least 95%, at least 97%, or at least 99% identical to the amino acid sequence of SEQ ID NO: 204;
p. the HVRs of claim 25.u, and further comprises a heavy chain that is at least 90%, at least 95%, at least 97%, or at least 99% identical to the amino acid sequence of SEQ ID NO: 205;
q. the HVRs of claim 25.u, and further comprises a heavy chain that is at least 90%, at least 95%, at least 97%, or at least 99% identical to the amino acid sequence of SEQ ID NO: 206;
r. the HVRs of claim 25.u, and further comprises a heavy chain that is at least 90%, at least 95%, at least 97%, or at least 99% identical to the amino acid sequence of SEQ ID NO: 207;
s. the HVRs of claim 25.u, and further comprises a heavy chain that is at least 90%, at least 95%, at least 97%, or at least 99% identical to the amino acid sequence of SEQ ID NO: 208;
t. the HVRs of claim 25.u, and further comprises a heavy chain that is at least 90%, at least 95%, at least 97%, or at least 99% identical to the amino acid sequence of SEQ ID NO: 209;
u. the HVRs of claim 25.u, and further comprises a heavy chain that is at least 90%, at least 95%, at least 97%, or at least 99% identical to the amino acid sequence of SEQ ID NO: 210;
v. the HVRs of claim 25.u, and further comprises a heavy chain that is at least 90%, at least 95%, at least 97%, or at least 99% identical to the amino acid sequence of SEQ ID NO: 211;
w. the HVRs of claim 25.u, and further comprises a heavy chain that is at least 90%, at least 95%, at least 97%, or at least 99% identical to the amino acid sequence of SEQ ID NO: 212;
x. the HVRs of claim 25.u, and further comprises a heavy chain that is at least 90%, at least 95%, at least 97%, or at least 99% identical to the amino acid sequence of SEQ ID NO: 213;
y. the HVRs of claim 25.u, and further comprises a heavy chain that is at least 90%, at least 95%, at least 97%, or at least 99% identical to the amino acid sequence of SEQ ID NO: 214;
z. the HVRs of claim 25.u, and further comprises a heavy chain that is at least 90%, at least 95%, at least 97%, or at least 99% identical to the amino acid sequence of SEQ ID NO: 215;
aa. the HVRs of claim 25.u, and further comprises a heavy chain that is at least 90%, at least 95%, at least 97%, or at least 99% identical to the amino acid sequence of SEQ ID NO: 216;
bb. the HVRs of claim 25.u, and further comprises a heavy chain that is at least 90%, at least 95%, at least 97%, or at least 99% identical to the amino acid sequence of SEQ ID NO: 217;
cc. the HVRs of claim 25.u, and further comprises a heavy chain that is at least 90%, at least 95%, at least 97%, or at least 99% identical to the amino acid sequence of SEQ ID NO: 218;
dd. the HVRs of claim 25.u, and further comprises a heavy chain that is at least 90%, at least 95%, at least 97%, or at least 99% identical to the amino acid sequence of SEQ ID NO: 219;
ee, the HVRs of claim 25.w, and further comprises a heavy chain that is at least 90%, at least 95%, at least 97%, or at least 99% identical to the amino acid sequence of SEQ ID NO: 220;
ff. the HVRs of claim 25.w, and further comprises a heavy chain that is at least 90%, at least 95%, at least 97%, or at least 99% identical to the amino acid sequence of SEQ ID NO: 221;
gg. the HVRs of claim 25.w, and further comprises a heavy chain that is at least 90%, at least 95%, at least 97%, or at least 99% identical to the amino acid sequence of SEQ ID NO: 222;
hh. the HVRs of claim 25.w, and further comprises a heavy chain that is at least 90%, at least 95%, at least 97%, or at least 99% identical to the amino acid sequence of SEQ ID NO: 223;
ii. the HVRs of claim 25.w, and further comprises a heavy chain that is at least 90%, at least 95%, at least 97%, or at least 99% identical to the amino acid sequence of SEQ ID NO: 224;
jj. the HVRs of claim 25.w, and further comprises a heavy chain that is at least 90%, at least 95%, at least 97%, or at least 99% identical to the amino acid sequence of SEQ ID NO: 225;
kk. the HVRs of claim 25.w, and further comprises a heavy chain that is at least 90%, at least 95%, at least 97%, or at least 99% identical to the amino acid sequence of SEQ ID NO: 226;
ll. the HVRs of claim 25.w, and further comprises a heavy chain that is at least 90%, at least 95%, at least 97%, or at least 99% identical to the amino acid sequence of SEQ ID NO: 227;
mm. the HVRs of claim 25.w, and further comprises a heavy chain that is at least 90%, at least 95%, at least 97%, or at least 99% identical to the amino acid sequence of SEQ ID NO: 228;
nn. the HVRs of claim 25.w, and further comprises a heavy chain that is at least 90%, at least 95%, at least 97%, or at least 99% identical to the amino acid sequence of SEQ ID NO: 229;
oo. the HVRs of claim 25.w, and further comprises a heavy chain that is at least 90%, at least 95%, at least 97%, or at least 99% identical to the amino acid sequence of SEQ ID NO: 230;
pp. the HVRs of claim 25.w, and further comprises a heavy chain that is at least 90%, at least 95%, at least 97%, or at least 99% identical to the amino acid sequence of SEQ ID NO: 231;
qq. the HVRs of claim 25.w, and further comprises a heavy chain that is at least 90%, at least 95%, at least 97%, or at least 99% identical to the amino acid sequence of SEQ ID NO: 232;
rr. the HVRs of claim 25.w, and further comprises a heavy chain that is at least 90%, at least 95%, at least 97%, or at least 99% identical to the amino acid sequence of SEQ ID NO: 233; or
ss. the HVRs of claim 25.w, and further comprises a heavy chain that is at least 90%, at least 95%, at least 97%, or at least 99% identical to the amino acid sequence of SEQ ID NO: 234.

32. The antibody of claim 25 or claim 31, wherein the antibody comprises:
a. the HVRs of claim 25.s, and further comprises a light chain that is at least 90%, at least 95%, at least 97%, or at least 99% identical to the amino acid sequence of SEQ ID NO: 189;
b. the HVRs of claim 25.u, and further comprises a light chain that is at least 90%, at least 95%, at least 97%, or at least 99% identical to the amino acid sequence of SEQ ID NO: 189; or
c. the HVRs of claim 25.w, and further comprises a light chain that is at least 90%, at least 95%, at least 97%, or at least 99% identical to the amino acid sequence of SEQ ID NO: 189.

33. The antibody of claim 25, wherein the antibody comprises:
a. a heavy chain that is at least 90%, at least 95%, at least 97%, or at least 99% identical to the amino acid sequence of SEQ ID NO: 190 and a light chain that is at least 90%, at least 95%, at least 97%, or at least 99% identical to the amino acid sequence of SEQ ID NO: 189;
b. a heavy chain that is at least 90%, at least 95%, at least 97%, or at least 99% identical to the amino acid sequence of SEQ ID NO: 191 and a light chain that is at least 90%, at least 95%, at least 97%, or at least 99% identical to the amino acid sequence of SEQ ID NO: 189;
c. a heavy chain that is at least 90%, at least 95%, at least 97%, or at least 99% identical to the amino acid sequence of SEQ ID NO: 192 and a light chain that is at least 90%, at least 95%, at least 97%, or at least 99% identical to the amino acid sequence of SEQ ID NO: 189;
d. a heavy chain that is at least 90%, at least 95%, at least 97%, or at least 99% identical to the amino acid sequence of SEQ ID NO: 193 and a light chain that is at least 90%, at least 95%, at least 97%, or at least 99% identical to the amino acid sequence of SEQ ID NO: 189;
e. a heavy chain that is at least 90%, at least 95%, at least 97%, or at least 99% identical to the amino acid sequence of SEQ ID NO: 194 and a light chain that is at least 90%, at least 95%, at least 97%, or at least 99% identical to the amino acid sequence of SEQ ID NO: 189;
f. a heavy chain that is at least 90%, at least 95%, at least 97%, or at least 99% identical to the amino acid sequence of SEQ ID NO: 195 and a light chain that is at least 90%, at least 95%, at least 97%, or at least 99% identical to the amino acid sequence of SEQ ID NO: 189;
g. a heavy chain that is at least 90%, at least 95%, at least 97%, or at least 99% identical to the amino acid sequence of SEQ ID NO: 196 and a light chain that is at least 90%, at least 95%, at least 97%, or at least 99% identical to the amino acid sequence of SEQ ID NO: 189;
h. a heavy chain that is at least 90%, at least 95%, at least 97%, or at least 99% identical to the amino acid sequence of SEQ ID NO: 197 and a light chain that is at least 90%, at least 95%, at least 97%, or at least 99% identical to the amino acid sequence of SEQ ID NO: 189;
i. a heavy chain that is at least 90%, at least 95%, at least 97%, or at least 99% identical to the amino acid sequence of SEQ ID NO: 198 and a light chain that is at least 90%, at least 95%, at least 97%, or at least 99% identical to the amino acid sequence of SEQ ID NO: 189;
j. a heavy chain that is at least 90%, at least 95%, at least 97%, or at least 99% identical to the amino acid sequence of SEQ ID NO: 199 and a light chain that is at least 90%, at least 95%, at least 97%, or at least 99% identical to the amino acid sequence of SEQ ID NO: 189;
k. a heavy chain that is at least 90%, at least 95%, at least 97%, or at least 99% identical to the amino acid sequence of SEQ ID NO:200 and a light chain that is at least 90%, at least 95%, at least 97%, or at least 99% identical to the amino acid sequence of SEQ ID NO: 189;
l. a heavy chain that is at least 90%, at least 95%, at least 97%, or at least 99% identical to the amino acid sequence of SEQ ID NO:201 and a light chain that is at least 90%, at least 95%, at least 97%, or at least 99% identical to the amino acid sequence of SEQ ID NO: 189;
m. a heavy chain that is at least 90%, at least 95%, at least 97%, or at least 99% identical to the amino acid sequence of SEQ ID NO:202 and a light chain that is at least 90%, at least 95%, at least 97%, or at least 99% identical to the amino acid sequence of SEQ ID NO: 189;
n. a heavy chain that is at least 90%, at least 95%, at least 97%, or at least 99% identical to the amino acid sequence of SEQ ID NO:203 and a light chain that is at least 90%, at least 95%, at least 97%, or at least 99% identical to the amino acid sequence of SEQ ID NO: 189;
o. a heavy chain that is at least 90%, at least 95%, at least 97%, or at least 99% identical to the amino acid sequence of SEQ ID NO:204 and a light chain that is at least 90%, at least 95%, at least 97%, or at least 99% identical to the amino acid sequence of SEQ ID NO: 189;
p. a heavy chain that is at least 90%, at least 95%, at least 97%, or at least 99% identical to the amino acid sequence of SEQ ID NO:205 and a light chain that is at least 90%, at least 95%, at least 97%, or at least 99% identical to the amino acid sequence of SEQ ID NO: 189;
q. a heavy chain that is at least 90%, at least 95%, at least 97%, or at least 99% identical to the amino acid sequence of SEQ ID NO:206 and a light chain that is at least 90%, at least 95%, at least 97%, or at least 99% identical to the amino acid sequence of SEQ ID NO: 189;
r. a heavy chain that is at least 90%, at least 95%, at least 97%, or at least 99% identical to the amino acid sequence of SEQ ID NO:207 and a light chain that is at least 90%, at least 95%, at least 97%, or at least 99% identical to the amino acid sequence of SEQ ID NO: 189;
s. a heavy chain that is at least 90%, at least 95%, at least 97%, or at least 99% identical to the amino acid sequence of SEQ ID NO:208 and a light chain that is at least 90%, at least 95%, at least 97%, or at least 99% identical to the amino acid sequence of SEQ ID NO: 189;
t. a heavy chain that is at least 90%, at least 95%, at least 97%, or at least 99% identical to the amino acid sequence of SEQ ID NO:209 and a light chain that is at least 90%, at least 95%, at least 97%, or at least 99% identical to the amino acid sequence of SEQ ID NO: 189;
u. a heavy chain that is at least 90%, at least 95%, at least 97%, or at least 99% identical to the amino acid sequence of SEQ ID NO:210 and a light chain that is at least 90%, at least 95%, at least 97%, or at least 99% identical to the amino acid sequence of SEQ ID NO: 189;
v. a heavy chain that is at least 90%, at least 95%, at least 97%, or at least 99% identical to the amino acid sequence of SEQ ID NO:211 and a light chain that is at least 90%, at least 95%, at least 97%, or at least 99% identical to the amino acid sequence of SEQ ID NO: 189;
w. a heavy chain that is at least 90%, at least 95%, at least 97%, or at least 99% identical to the amino acid sequence of SEQ ID NO:212 and a light chain that is at least 90%, at least 95%, at least 97%, or at least 99% identical to the amino acid sequence of SEQ ID NO: 189;
x. a heavy chain that is at least 90%, at least 95%, at least 97%, or at least 99% identical to the amino acid sequence of SEQ ID NO:213 and a light chain that is at least 90%, at least 95%, at least 97%, or at least 99% identical to the amino acid sequence of SEQ ID NO: 189;
y. a heavy chain that is at least 90%, at least 95%, at least 97%, or at least 99% identical to the amino acid sequence of SEQ ID NO:214 and a light chain that is at least 90%, at least 95%, at least 97%, or at least 99% identical to the amino acid sequence of SEQ ID NO: 189;
z. a heavy chain that is at least 90%, at least 95%, at least 97%, or at least 99% identical to the amino acid sequence of SEQ ID NO:215 and a light chain that is at least 90%, at least 95%, at least 97%, or at least 99% identical to the amino acid sequence of SEQ ID NO: 189;
aa. a heavy chain that is at least 90%, at least 95%, at least 97%, or at least 99% identical to the amino acid sequence of SEQ ID NO:216 and a light chain that is at least 90%, at least 95%, at least 97%, or at least 99% identical to the amino acid sequence of SEQ ID NO: 189;
bb. a heavy chain that is at least 90%, at least 95%, at least 97%, or at least 99% identical to the amino acid sequence of SEQ ID NO:217 and a light chain that is at least 90%, at least 95%, at least 97%, or at least 99% identical to the amino acid sequence of SEQ ID NO: 189;
cc. a heavy chain that is at least 90%, at least 95%, at least 97%, or at least 99% identical to the amino acid sequence of SEQ ID NO:218 and a light chain that is at least 90%, at least 95%, at least 97%, or at least 99% identical to the amino acid sequence of SEQ ID NO: 189;
dd. a heavy chain that is at least 90%, at least 95%, at least 97%, or at least 99% identical to the amino acid sequence of SEQ ID NO:219 and a light chain that is at least 90%, at least 95%, at least 97%, or at least 99% identical to the amino acid sequence of SEQ ID NO: 189;
ee. a heavy chain that is at least 90%, at least 95%, at least 97%, or at least 99% identical to the amino acid sequence of SEQ ID NO:220 and a light chain that is at least 90%, at least 95%, at least 97%, or at least 99% identical to the amino acid sequence of SEQ ID NO: 189;
ff. a heavy chain that is at least 90%, at least 95%, at least 97%, or at least 99% identical to the amino acid sequence of SEQ ID NO:221 and a light chain that is at least 90%, at least 95%, at least 97%, or at least 99% identical to the amino acid sequence of SEQ ID NO: 189;
gg. a heavy chain that is at least 90%, at least 95%, at least 97%, or at least 99% identical to the amino acid sequence of SEQ ID NO:222 and a light chain that is at least 90%, at least 95%, at least 97%, or at least 99% identical to the amino acid sequence of SEQ ID NO: 189;
hh. a heavy chain that is at least 90%, at least 95%, at least 97%, or at least 99% identical to the amino acid sequence of SEQ ID NO:223 and a light chain that is at least 90%, at least 95%, at least 97%, or at least 99% identical to the amino acid sequence of SEQ ID NO: 189;
ii. a heavy chain that is at least 90%, at least 95%, at least 97%, or at least 99% identical to the amino acid sequence of SEQ ID NO:224 and a light chain that is at least 90%, at least 95%, at least 97%, or at least 99% identical to the amino acid sequence of SEQ ID NO: 189;
jj. a heavy chain that is at least 90%, at least 95%, at least 97%, or at least 99% identical to the amino acid sequence of SEQ ID NO:225 and a light chain that is at least 90%, at least 95%, at least 97%, or at least 99% identical to the amino acid sequence of SEQ ID NO: 189;
kk. a heavy chain that is at least 90%, at least 95%, at least 97%, or at least 99% identical to the amino acid sequence of SEQ ID NO:226 and a light chain that is at least 90%, at least 95%, at least 97%, or at least 99% identical to the amino acid sequence of SEQ ID NO: 189;
ll. a heavy chain that is at least 90%, at least 95%, at least 97%, or at least 99% identical to the amino acid sequence of SEQ ID NO:227 and a light chain that is at least 90%, at least 95%, at least 97%, or at least 99% identical to the amino acid sequence of SEQ ID NO: 189;
mm. a heavy chain that is at least 90%, at least 95%, at least 97%, or at least 99% identical to the amino acid sequence of SEQ ID NO:228 and a light chain that is at least 90%, at least 95%, at least 97%, or at least 99% identical to the amino acid sequence of SEQ ID NO: 189;
nn. a heavy chain that is at least 90%, at least 95%, at least 97%, or at least 99% identical to the amino acid sequence of SEQ ID NO:229 and a light chain that is at least 90%, at least 95%, at least 97%, or at least 99% identical to the amino acid sequence of SEQ ID NO: 189;
oo. a heavy chain that is at least 90%, at least 95%, at least 97%, or at least 99% identical to the amino acid sequence of SEQ ID NO:230 and a light chain that is at least 90%, at least 95%, at least 97%, or at least 99% identical to the amino acid sequence of SEQ ID NO: 189;
pp. a heavy chain that is at least 90%, at least 95%, at least 97%, or at least 99% identical to the amino acid sequence of SEQ ID NO:231 and a light chain that is at least 90%, at least 95%, at least 97%, or at least 99% identical to the amino acid sequence of SEQ ID NO: 189;
qq. a heavy chain that is at least 90%, at least 95%, at least 97%, or at least 99% identical to the amino acid sequence of SEQ ID NO:232 and a light chain that is at least 90%, at least 95%, at least 97%, or at least 99% identical to the amino acid sequence of SEQ ID NO: 189;
rr. a heavy chain that is at least 90%, at least 95%, at least 97%, or at least 99% identical to the amino acid sequence of SEQ ID NO:233 and a light chain that is at least 90%, at least 95%, at least 97%, or at least 99% identical to the amino acid sequence of SEQ ID NO: 189; or
ss. a heavy chain that is at least 90%, at least 95%, at least 97%, or at least 99% identical to the amino acid sequence of SEQ ID NO:234 and a light chain that is at least 90%, at least 95%, at least 97%, or at least 99% identical to the amino acid sequence of SEQ ID NO: 189.

34. An isolated anti-IL18BP antibody, wherein the anti-IL18BP antibody comprises:
a. a heavy chain comprising the amino acid sequence of SEQ ID NO: 190 and a light chain comprising the amino acid sequence of SEQ ID NO:189;
b. a heavy chain comprising the amino acid sequence of SEQ ID NO: 191 and a light chain comprising the amino acid sequence of SEQ ID NO: 189;
c. a heavy chain comprising the amino acid sequence of SEQ ID NO: 192 and a light chain comprising the amino acid sequence of SEQ ID NO:189;
d. a heavy chain comprising the amino acid sequence of SEQ ID NO: 193 and a light chain comprising the amino acid sequence of SEQ ID NO: 189;
e. a heavy chain comprising the amino acid sequence of SEQ ID NO: 194 and a light chain comprising the amino acid sequence of SEQ ID NO:189;
f. a heavy chain comprising the amino acid sequence of SEQ ID NO: 195 and a light chain comprising the amino acid sequence of SEQ ID NO: 189;
g. a heavy chain comprising the amino acid sequence of SEQ ID NO: 196 and a light chain comprising the amino acid sequence of SEQ ID NO: 189;
h. a heavy chain comprising the amino acid sequence of SEQ ID NO: 197 and a light chain comprising the amino acid sequence of SEQ ID NO:189;
i. a heavy chain comprising the amino acid sequence of SEQ ID NO: 198 and a light chain comprising the amino acid sequence of SEQ ID NO: 189;
j. a heavy chain comprising the amino acid sequence of SEQ ID NO: 199 and a light chain comprising the amino acid sequence of SEQ ID NO: 189;
k. a heavy chain comprising the amino acid sequence of SEQ ID NO:200 and a light chain comprising the amino acid sequence of SEQ ID NO: 189;
l. a heavy chain comprising the amino acid sequence of SEQ ID NO:201 and a light chain comprising the amino acid sequence of SEQ ID NO: 189;
m. a heavy chain comprising the amino acid sequence of SEQ ID NO:202 and a light chain comprising the amino acid sequence of SEQ ID NO:189;
n. a heavy chain comprising the amino acid sequence of SEQ ID NO:203 and a light chain comprising the amino acid sequence of SEQ ID NO:189;
o. a heavy chain comprising the amino acid sequence of SEQ ID NO:204 and a light chain comprising the amino acid sequence of SEQ ID NO:189;
p. a heavy chain comprising the amino acid sequence of SEQ ID NO:205 and a light chain comprising the amino acid sequence of SEQ ID NO: 189;
q. a heavy chain comprising the amino acid sequence of SEQ ID NO:206 and a light chain comprising the amino acid sequence of SEQ ID NO: 189;
r. a heavy chain comprising the amino acid sequence of SEQ ID NO:207 and a light chain comprising the amino acid sequence of SEQ ID NO: 189;
s. a heavy chain comprising the amino acid sequence of SEQ ID NO:208 and a light chain comprising the amino acid sequence of SEQ ID NO:189;
t. a heavy chain comprising the amino acid sequence of SEQ ID NO:209 and a light chain comprising the amino acid sequence of SEQ ID NO:189;
u. a heavy chain comprising the amino acid sequence of SEQ ID NO:210 and a light chain comprising the amino acid sequence of SEQ ID NO: 189;
v. a heavy chain comprising the amino acid sequence of SEQ ID NO:211 and a light chain comprising the amino acid sequence of SEQ ID NO: 189;
w. a heavy chain comprising the amino acid sequence of SEQ ID NO:212 and a light chain comprising the amino acid sequence of SEQ ID NO:189;
x. a heavy chain comprising the amino acid sequence of SEQ ID NO:213 and a light chain comprising the amino acid sequence of SEQ ID NO: 189;
y. a heavy chain comprising the amino acid sequence of SEQ ID NO:214 and a light chain comprising the amino acid sequence of SEQ ID NO:189;
z. a heavy chain comprising the amino acid sequence of SEQ ID NO:215 and a light chain comprising the amino acid sequence of SEQ ID NO:189;
aa. a heavy chain comprising the amino acid sequence of SEQ ID NO:216 and a light chain comprising the amino acid sequence of SEQ ID NO:189;
bb. a heavy chain comprising the amino acid sequence of SEQ ID NO:217 and a light chain comprising the amino acid sequence of SEQ ID NO: 189;
cc. a heavy chain comprising the amino acid sequence of SEQ ID NO:218 and a light chain comprising the amino acid sequence of SEQ ID NO: 189;
dd. a heavy chain comprising the amino acid sequence of SEQ ID NO:219 and a light chain comprising the amino acid sequence of SEQ ID NO: 189;
ee. a heavy chain comprising the amino acid sequence of SEQ ID NO:220 and a light chain comprising the amino acid sequence of SEQ ID NO:189;
ff. a heavy chain comprising the amino acid sequence of SEQ ID NO:221 and a light chain comprising the amino acid sequence of SEQ ID NO:189;
gg. a heavy chain comprising the amino acid sequence of SEQ ID NO:222 and a light chain comprising the amino acid sequence of SEQ ID NO:189;
hh. a heavy chain comprising the amino acid sequence of SEQ ID NO:223 and a light chain comprising the amino acid sequence of SEQ ID NO: 189;
ii. a heavy chain comprising the amino acid sequence of SEQ ID NO:224 and a light chain comprising the amino acid sequence of SEQ ID NO:189;
jj. a heavy chain comprising the amino acid sequence of SEQ ID NO:225 and a light chain comprising the amino acid sequence of SEQ ID NO: 189;
kk. a heavy chain comprising the amino acid sequence of SEQ ID NO:226 and a light chain comprising the amino acid sequence of SEQ ID NO:189;
ll. a heavy chain comprising the amino acid sequence of SEQ ID NO:227 and a light chain comprising the amino acid sequence of SEQ ID NO:189;
mm. a heavy chain comprising the amino acid sequence of SEQ ID NO:228 and a light chain comprising the amino acid sequence of SEQ ID NO: 189;
nn. a heavy chain comprising the amino acid sequence of SEQ ID NO:229 and a light chain comprising the amino acid sequence of SEQ ID NO: 189;
oo. a heavy chain comprising the amino acid sequence of SEQ ID NO:230 and a light chain comprising the amino acid sequence of SEQ ID NO: 189;
pp. a heavy chain comprising the amino acid sequence of SEQ ID NO:321 and a light chain comprising the amino acid sequence of SEQ ID NO:189;
qq. a heavy chain comprising the amino acid sequence of SEQ ID NO:232 and a light chain comprising the amino acid sequence of SEQ ID NO: 189;
rr. a heavy chain comprising the amino acid sequence of SEQ ID NO:233 and a light chain comprising the amino acid sequence of SEQ ID NO: 189; or
ss. a heavy chain comprising the amino acid sequence of SEQ ID NO:234 and a light chain comprising the amino acid sequence of SEQ ID NO: 189.

35. An isolated anti-IL18BP antibody, wherein the anti-IL18BP antibody comprises a VH comprising HVR-H1, HVR-H2, and HVR-H3 and a VL comprising HVR-L1, HVR-L2, and HVR-L3 of any one of antibodies BP-01, BP-02, BP-03, BP-04, BP-05, BP-06, BP-07, BP-08, BP-09, BP-10, BP-11, BP-12, BP-13, BP-14, BP-15, BP-16, BP-04.01, BP-04.02, BP-04.03, BP-04.04, BP-04.05, BP-04.06, BP-04.07, BP-04.08, BP-04.09, BP-04.10, BP-04.11, BP-04.12, BP-04.13, BP-04.14, BP-04.15, BP-04.16, BP-04.17, BP-04.18, BP-04.19, BP-04.20, BP-04.21, BP-04.22, BP-04.23, BP-04.24, BP-04.25, BP-04.26, BP-04.27, BP-04.28, BP-04.29, BP-04.30, BP-04.31, and BP-04.32.

36. The isolated antibody of claim 35, wherein the antibody comprises a VH and/or a VL at least 90%, at least 95%, at least 97%, or at least 99% identical to those of any one of antibodies BP-01, BP-02, BP-03, BP-04, BP-05, BP-06, BP-07, BP-08, BP-09, BP-10, BP-11, BP-12, BP-13, BP-14, BP-15, BP-16, BP-04.01, BP-04.02, BP-04.03, BP-04.04, BP-04.05, BP-04.06, BP-04.07, BP-04.08, BP-04.09, BP-04.10, BP-04.11, BP-04.12, BP-04.13, BP-04.14, BP-04.15, BP-04.16, BP-04.17, BP-04.18, BP-04.19, BP-04.20, BP-04.21, BP-04.22, BP-04.23, BP-04.24, BP-04.25, BP-04.26, BP-04.27, BP-04.28, BP-04.29, BP-04.30, BP-04.31, and BP-04.32.

37. The isolated antibody of claim 35 or claim 36, wherein the antibody comprises the VH and/or the VL of any one of antibodies BP-01, BP-02, BP-03, BP-04, BP-05, BP-06, BP-07, BP-08, BP-09, BP-10, BP-11, BP-12, BP-13, BP-14, BP-15, BP-16, BP-04.01, BP-04.02, BP-04.03, BP-04.04, BP-04.05, BP-04.06, BP-04.07, BP-04.08, BP-04.09, BP-04.10, BP-04.11, BP-04.12, BP-04.13, BP-04.14, BP-04.15, BP-04.16, BP-04.17, BP-04.18, BP-04.19, BP-04.20, BP-04.21, BP-04.22, BP-04.23, BP-04.24, BP-04.25, BP-04.26, BP-04.27, BP-04.28, BP-04.29, BP-04.30, BP-04.31, and BP-04.32.

38. An isolated anti-IL18BP antibody, wherein the anti-IL18BP antibody comprises:
a. a VH and VL, wherein the VH comprises the HVR-H1, HVR-H2, and HVR-H3 of the antibody BP-01 (as shown in Table 1) and the VL comprises the HVR-L1, HVR-L2, and HVR-L3 of the antibody BP-01 (as shown in Table 2);
b. VH and VL, wherein the VH comprises the HVR-H1, HVR-H2, and HVR-H3 of the antibody BP-02 (as shown in Table 1) and the VL comprises the HVR-L1, HVR-L2, and HVR-L3 of the antibody BP-02 (as shown in Table 2);
c. VH and VL, wherein the VH comprises the HVR-H1, HVR-H2, and HVR-H3 of the antibody BP-03 (as shown in Table 1) and the VL comprises the HVR-L1, HVR-L2, and HVR-L3 of the antibody BP-03 (as shown in Table 2);
d. VH and VL, wherein the VH comprises the HVR-H1, HVR-H2, and HVR-H3 of the antibody BP-04 (as shown in Table 1) and the VL comprises the HVR-L1, HVR-L2, and HVR-L3 of the antibody BP-04 (as shown in Table 2);
e. VH and VL, wherein the VH comprises the HVR-H1, HVR-H2, and HVR-H3 of the antibody BP-05 (as shown in Table 1) and the VL comprises the HVR-L1, HVR-L2, and HVR-L3 of the antibody BP-05 (as shown in Table 2);
f. VH and VL, wherein the VH comprises the HVR-H1, HVR-H2, and HVR-H3 of the antibody BP-06 (as shown in Table 1) and the VL comprises the HVR-L1, HVR-L2, and HVR-L3 of the antibody BP-06 (as shown in Table 2);
g. VH and VL, wherein the VH comprises the HVR-H1, HVR-H2, and HVR-H3 of the antibody BP-07 (as shown in Table 1) and the VL comprises the HVR-L1, HVR-L2, and HVR-L3 of the antibody BP-07 (as shown in Table 2);
h. VH and VL, wherein the VH comprises the HVR-H1, HVR-H2, and HVR-H3 of the antibody BP-08 (as shown in Table 1) and the VL comprises the HVR-L1, HVR-L2, and HVR-L3 of the antibody BP-08 (as shown in Table 2);
i. VH and VL, wherein the VH comprises the HVR-H1, HVR-H2, and HVR-H3 of the antibody BP-09 (as shown in Table 1) and the VL comprises the HVR-L1, HVR-L2, and HVR-L3 of the antibody BP-09 (as shown in Table 2);
j. VH and VL, wherein the VH comprises the HVR-H1, HVR-H2, and HVR-H3 of the antibody BP-10 (as shown in Table 1) and the VL comprises the HVR-L1, HVR-L2, and HVR-L3 of the antibody BP-10 (as shown in Table 2);
k. VH and VL, wherein the VH comprises the HVR-H1, HVR-H2, and HVR-H3 of the antibody BP-11 (as shown in Table 1) and the VL comprises the HVR-L1, HVR-L2, and HVR-L3 of the antibody BP-11 (as shown in Table 2);
l. VH and VL, wherein the VH comprises the HVR-H1, HVR-H2, and HVR-H3 of the antibody BP-12 (as shown in Table 1) and the VL comprises the HVR-L1, HVR-L2, and HVR-L3 of the antibody BP-12 (as shown in Table 2);
m. VH and VL, wherein the VH comprises the HVR-H1, HVR-H2, and HVR-H3 of the antibody BP-13 (as shown in Table 1) and the VL comprises the HVR-L1, HVR-L2, and HVR-L3 of the antibody BP-13 (as shown in Table 2);
n. VH and VL, wherein the VH comprises the HVR-H1, HVR-H2, and HVR-H3 of the antibody BP-14 (as shown in Table 1) and the VL comprises the HVR-L1, HVR-L2, and HVR-L3 of the antibody BP-14 (as shown in Table 2);
o. VH and VL, wherein the VH comprises the HVR-H1, HVR-H2, and HVR-H3 of the antibody BP-15 (as shown in Table 1) and the VL comprises the HVR-L1, HVR-L2, and HVR-L3 of the antibody BP-15 (as shown in Table 2);
p. VH and VL, wherein the VH comprises the HVR-H1, HVR-H2, and HVR-H3 of the antibody BP-16 (as shown in Table 1) and the VL comprises the HVR-L1, HVR-L2, and HVR-L3 of the antibody BP-16 (as shown in Table 2);
q. VH and VL, wherein the VH comprises the HVR-H1, HVR-H2, and HVR-H3 of the antibody BP-04.01 (as shown in Table 12) and the VL comprises the HVR-L1, HVR-L2, and HVR-L3 of the antibody BP-04.LC (as shown in Table 13);
r. VH and VL, wherein the VH comprises the HVR-H1, HVR-H2, and HVR-H3 of the antibody BP-04.02 (as shown in Table 12) and the VL comprises the HVR-L1, HVR-L2, and HVR-L3 of the antibody BP-04.LC (as shown in Table 13);
s. VH and VL, wherein the VH comprises the HVR-H1, HVR-H2, and HVR-H3 of the antibody BP-04.03 (as shown in Table 12) and the VL comprises the HVR-L1, HVR-L2, and HVR-L3 of the antibody BP-04.LC (as shown in Table 13);
t. VH and VL, wherein the VH comprises the HVR-H1, HVR-H2, and HVR-H3 of the antibody BP-04.04 (as shown in Table 12) and the VL comprises the HVR-L1, HVR-L2, and HVR-L3 of the antibody BP-04.LC (as shown in Table 13);
u. VH and VL, wherein the VH comprises the HVR-H1, HVR-H2, and HVR-H3 of the antibody BP-04.05 (as shown in Table 12) and the VL comprises the HVR-L1, HVR-L2, and HVR-L3 of the antibody BP-04.LC (as shown in Table 13);
v. VH and VL, wherein the VH comprises the HVR-H1, HVR-H2, and HVR-H3 of the antibody BP-04.06 (as shown in Table 12) and the VL comprises the HVR-L1, HVR-L2, and HVR-L3 of the antibody BP-04.LC (as shown in Table 13);
w. VH and VL, wherein the VH comprises the HVR-H1, HVR-H2, and HVR-H3 of the antibody BP-04.07 (as shown in Table 12) and the VL comprises the HVR-L1, HVR-L2, and HVR-L3 of the antibody BP-04.LC (as shown in Table 13);
x. VH and VL, wherein the VH comprises the HVR-H1, HVR-H2, and HVR-H3 of the antibody BP-04.08 (as shown in Table 12) and the VL comprises the HVR-L1, HVR-L2, and HVR-L3 of the antibody BP-04.LC (as shown in Table 13);
y. VH and VL, wherein the VH comprises the HVR-H1, HVR-H2, and HVR-H3 of the antibody BP-04.09 (as shown in Table 12) and the VL comprises the HVR-L1, HVR-L2, and HVR-L3 of the antibody BP-04.LC (as shown in Table 13);
z. VH and VL, wherein the VH comprises the HVR-H1, HVR-H2, and HVR-H3 of the antibody BP-04.10 (as shown in Table 12) and the VL comprises the HVR-L1, HVR-L2, and HVR-L3 of the antibody BP-04.LC (as shown in Table 13);
aa. VH and VL, wherein the VH comprises the HVR-H1, HVR-H2, and HVR-H3 of the antibody BP-04.11 (as shown in Table 12) and the VL comprises the HVR-L1, HVR-L2, and HVR-L3 of the antibody BP-04.LC (as shown in Table 13);
bb. VH and VL, wherein the VH comprises the HVR-H1, HVR-H2, and HVR-H3 of the antibody BP-04.12 (as shown in Table 12) and the VL comprises the HVR-L1, HVR-L2, and HVR-L3 of the antibody BP-04.LC (as shown in Table 13);
cc. VH and VL, wherein the VH comprises the HVR-H1, HVR-H2, and HVR-H3 of the antibody BP-04.13 (as shown in Table 12) and the VL comprises the HVR-L1, HVR-L2, and HVR-L3 of the antibody BP-04.LC (as shown in Table 13);
dd. VH and VL, wherein the VH comprises the HVR-H1, HVR-H2, and HVR-H3 of the antibody BP-04.14 (as shown in Table 12) and the VL comprises the HVR-L1, HVR-L2, and HVR-L3 of the antibody BP-04.LC (as shown in Table 13);
ec. VH and VL, wherein the VH comprises the HVR-H1, HVR-H2, and HVR-H3 of the antibody BP-04.15 (as shown in Table 12) and the VL comprises the HVR-L1, HVR-L2, and HVR-L3 of the antibody BP-04.LC (as shown in Table 13);
ff. VH and VL, wherein the VH comprises the HVR-H1, HVR-H2, and HVR-H3 of the antibody BP-04.16 (as shown in Table 12) and the VL comprises the HVR-L1, HVR-L2, and HVR-L3 of the antibody BP-04.LC (as shown in Table 13);
gg. VH and VL, wherein the VH comprises the HVR-H1, HVR-H2, and HVR-H3 of the antibody BP-04.17 (as shown in Table 12) and the VL comprises the HVR-L1, HVR-L2, and HVR-L3 of the antibody BP-04.LC (as shown in Table 13);
hh. VH and VL, wherein the VH comprises the HVR-H1, HVR-H2, and HVR-H3 of the antibody BP-04.18 (as shown in Table 12) and the VL comprises the HVR-L1, HVR-L2, and HVR-L3 of the antibody BP-04.LC (as shown in Table 13);
ii. VH and VL, wherein the VH comprises the HVR-H1, HVR-H2, and HVR-H3 of the antibody BP-04.19 (as shown in Table 12) and the VL comprises the HVR-L1, HVR-L2, and HVR-L3 of the antibody BP-04.LC (as shown in Table 13);
jj. VH and VL, wherein the VH comprises the HVR-H1, HVR-H2, and HVR-H3 of the antibody BP-04.20 (as shown in Table 12) and the VL comprises the HVR-L1, HVR-L2, and HVR-L3 of the antibody BP-04.LC (as shown in Table 13);
kk. VH and VL, wherein the VH comprises the HVR-H1, HVR-H2, and HVR-H3 of the antibody BP-04.21 (as shown in Table 12) and the VL comprises the HVR-L1, HVR-L2, and HVR-L3 of the antibody BP-04.LC (as shown in Table 13);
ll. VH and VL, wherein the VH comprises the HVR-H1, HVR-H2, and HVR-H3 of the antibody BP-04.22 (as shown in Table 12) and the VL comprises the HVR-L1, HVR-L2, and HVR-L3 of the antibody BP-04.LC (as shown in Table 13);
mm. VH and VL, wherein the VH comprises the HVR-H1, HVR-H2, and HVR-H3 of the antibody BP-04.23 (as shown in Table 12) and the VL comprises the HVR-L1, HVR-L2, and HVR-L3 of the antibody BP-04.LC (as shown in Table 13);
nn. VH and VL, wherein the VH comprises the HVR-H1, HVR-H2, and HVR-H3 of the antibody BP-04.24 (as shown in Table 12) and the VL comprises the HVR-L1, HVR-L2, and HVR-L3 of the antibody BP-04.LC (as shown in Table 13);
oo. VH and VL, wherein the VH comprises the HVR-H1, HVR-H2, and HVR-H3 of the antibody BP-04.25 (as shown in Table 12) and the VL comprises the HVR-L1, HVR-L2, and HVR-L3 of the antibody BP-04.LC (as shown in Table 13);
pp. VH and VL, wherein the VH comprises the HVR-H1, HVR-H2, and HVR-H3 of the antibody BP-04.26 (as shown in Table 12) and the VL comprises the HVR-L1, HVR-L2, and HVR-L3 of the antibody BP-04.LC (as shown in Table 13);
qq. VH and VL, wherein the VH comprises the HVR-H1, HVR-H2, and HVR-H3 of the antibody BP-04.27 (as shown in Table 12) and the VL comprises the HVR-L1, HVR-L2, and HVR-L3 of the antibody BP-04.LC (as shown in Table 13);
rr. VH and VL, wherein the VH comprises the HVR-H1, HVR-H2, and HVR-H3 of the antibody BP-04.28 (as shown in Table 12) and the VL comprises the HVR-L1, HVR-L2, and HVR-L3 of the antibody BP-04.LC (as shown in Table 13);
ss. VH and VL, wherein the VH comprises the HVR-H1, HVR-H2, and HVR-H3 of the antibody BP-04.29 (as shown in Table 12) and the VL comprises the HVR-L1, HVR-L2, and HVR-L3 of the antibody BP-04.LC (as shown in Table 13);
tt. VH and VL, wherein the VH comprises the HVR-H1, HVR-H2, and HVR-H3 of the antibody BP-04.30 (as shown in Table 12) and the VL comprises the HVR-L1, HVR-L2, and HVR-L3 of the antibody BP-04.LC (as shown in Table 13);
uu. VH and VL, wherein the VH comprises the HVR-H1, HVR-H2, and HVR-H3 of the antibody BP-04.31 (as shown in Table 12) and the VL comprises the HVR-L1, HVR-L2, and HVR-L3 of the antibody BP-04.LC (as shown in Table 13); or
vv. VH and VL, wherein the VH comprises the HVR-H1, HVR-H2, and HVR-H3 of the antibody BP-04.32 (as shown in Table 12) and the VL comprises the HVR-L1, HVR-L2, and HVR-L3 of the antibody BP-04.LC (as shown in Table 13).

39. The antibody of any one of claims 25-38, wherein the anti-IL18BP antibody blocks binding of IL-18 to IL18BP.

40. The antibody of any one of claims 25-39, wherein the antibody has one or more of the following properties:
a. the anti-IL18BP antibody increases expression levels of interferon-gamma (IFNγ) in peripheral blood mononuclear cells;
b. the anti-IL18BP antibody increases expression of IFNγ in natural killer cells;
c. the anti-IL18BP antibody increases levels of inflammatory cytokines in serum;
d. the anti-IL18BP antibody increases IFNγ levels in serum;
e. the anti-IL18BP antibody increases IL-10 levels in serum;
f. the anti-IL18BP antibody increases IL-6 levels in serum;
g. the anti-IL18BP antibody increases tumor necrosis factor alpha (TNFα) levels in serum;
h. the anti-IL18BP antibody decreases the percentage of CD3+ T cells in vivo;
i. the anti-IL18BP antibody decreases the percentage of CD19+ B cells in vivo;
j. the anti-IL18BP antibody decreases the percentage of CD11b+ cells in vivo;
k. the anti-IL18BP antibody increases the percentage of macrophages in vivo;
l. the anti-IL18BP antibody increases free IL-18 serum levels;
m. the anti-IL18BP antibody decreases IL-18BP serum levels;
n. the anti-IL18BP antibody increases IFNγ levels in tumor lysates;
o. the anti-IL18BP antibody increases IL-1α levels in tumor lysates;
p. the anti-IL18BP antibody increases IL-15 levels in tumor lysates;
q. the anti-IL18BP antibody increases IL-27 levels in tumor lysates;
r. the anti-IL18BP antibody increases MIP-1α levels in tumor lysates; and
s. the anti-IL18BP antibody decreases tumor volume.

41. The antibody of any one of claims 1-30 and 35-40, wherein the antibody is a monoclonal antibody.

42. The antibody of any one of claims 1-41, wherein the antibody is a humanized antibody.

43. The antibody of any one of claims 1-42, wherein the antibody is an antigen binding fragment, such as an Fab, Fab′, Fab′-SH, F(ab′)2, Fv, or scFv fragment.

44. The antibody of any one of claims 1-43, wherein the antibody is a bispecific or multispecific antibody.

45. The antibody of any one of claims 1-44, wherein the antibody is of the IgG class, the IgM class, or the IgA class.

46. The antibody of claim 45, wherein the antibody is of the IgG class and is of a human IgG1, IgG2, IgG3, or IgG4 isotype or of a mouse IgG1 or IgG2 isotype.

47. The antibody of any one of claims 1-46, wherein the antibody has a human or mouse IgG1 isotype and comprises one or more amino acid substitutions in the Fc region at an amino acid residue selected from the group consisting of N297A, D265A, D270A, L234A, L235A, G237A, P238D, L328E, E233D, G237D, H268D, P271G, A330R, C226S, C229S, E233P, L234V, L234F, L235E, P331S, P331G, S267E, L328F, A330L, M252Y, S254T, T256E, N297Q, P238S, P238A, A327Q, A327G, P329A, P329S, P329G, K322A, N325S, T394D, A330S, E430G, E430S, E430F, E430T, E345K, E345Q, E345R, E345Y, S440Y, S440W, and any combination thereof, wherein the numbering of the residues is according to EU numbering.

48. The antibody of any one of claim 1-30 or 35-46, wherein the antibody has a human or mouse IgG2 isotype and comprises one or more amino acid substitutions in the Fc region at an amino acid residue selected from the group consisting of A330S, C127S, C214S, C219S, C220S, E345K, E345Q, E345R, E345Y, E430F, E430G, E430S, E430T, G237A, H268Q, L328F, M252Y, P331S, S254T, S267E, S440W, S440Y, T256E, V234A, V309L, and any combination thereof, wherein the numbering of the residues is according to EU numbering.

49. The antibody of any one of claims 1-46, wherein the antibody has a human or mouse IgG4 isotype and comprises one or more amino acid substitutions in the Fc region at an amino acid residue selected from the group consisting of C127S, E318A, E345R, E430G, F234A, G237A, K322A, L235A, L235E, L236E, L243A, L328F, M252Y, P331S, S228P, S229P, S254T, S267E, S440Y, T256E, and any combination thereof, wherein the numbering of the residues is according to EU numbering.

50. The antibody of any one of claims 1-49, wherein the antibody comprises one or more amino acid substitutions in the Fc region at a residue position selected from the group consisting of A330L, A330S, C127S, E345R, E430G, K322A, L234A, L234F, L235A, L235E, L243A, L328F, P331S, P331G, S267E, S440Y, and any combination thereof, wherein the numbering of the amino acid residues is according to EU or Kabat numbering.

51. The antibody of any one of claim 1-50, where in the IgG Fc amino acid sequences is selected from the group consisting of SEQ ID NOs: 8-27 and 235-246.

52. A pharmaceutical composition comprising the anti-IL18BP antibody of any one of claims 1-51 and a pharmaceutically acceptable carrier.

53. An isolated nucleic acid comprising a nucleic acid sequence encoding the anti-IL18BP antibody of any one of claims 1-51.

54. An isolated vector comprising the nucleic acid of claim 53.

55. An isolated host cell comprising the nucleic acid of claim 53 or the vector of claim 54.

56. A method of producing an anti-IL18BP antibody, comprising culturing the cell of claim 55 so that the antibody is produced.

57. The method of claim 56, further comprising recovering the antibody produced by the cell.

58. A method of treating a cancer in a subject in need thereof, the method comprising administering to an individual in need thereof a therapeutically effective amount of an anti-IL18BP antibody of any one of claims 1-51, thereby treating the cancer.

59. The method of claim 58, further comprising administering one or more therapeutic agents.

60. The method of claim 59, wherein at least one therapeutic agent is a checkpoint inhibitor.

61. The method of claim 60, where the checkpoint inhibitor is selected from a PD1, PD-L1, and PD-L2 inhibitor.

62. The method of claim 60, where in the checkpoint inhibitor is selected from an anti-PD-L1 antibody, an anti-PD-L2 antibody, and an anti-PD-1 antibody.

63. The method of any one of claims 58-62, wherein the cancer is cancer is cholangiocarcinoma, diffuse large B cell lymphoma, glioblastoma multiform, head and neck squamous carcinoma, kidney renal clear cell carcinoma, sarcoma, bladder cancer, breast cancer, colon cancer, endometrial cancer, kidney cancer, renal cancer, pancreatic adenocarcinoma, skin cutaneous melanoma, stomach adenocarcinoma, lung cancer, non-small cell lung cancer, melanoma, lymphoma, pancreatic cancer, prostate cancer, ovarian cancer, stomach cancer, thyroid cancer, cancer of the uterus, liver cancer, cervical cancer, testicular cancer, squamous cell carcinoma, glioma, glioblastoma, adenoma, neuroblastoma, bladder carcinoma, esophageal carcinoma, or triple-negative breast carcinoma.

64. A method of detecting the presence of IL18BP in a sample in vitro or an individual, the method comprising an anti-IL18BP antibody of any one of claims 1-51.

65. The method of claim 64, further comprising quantification of antigen-bound anti-IL18BP antibody.


SEQUENCE LISTING (249 sequences)

SEQ ID NO: 1 - protein, 194 aa, Homo sapiens
MTMRHNWTPDLSPLWVLLLCAHVVTLLVRATPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQG

SEQ ID NO: 2 - protein, 199 aa, Homo sapiens
MTMRHNWTPDLSPLWVLLLCAHVVTLLVRATPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWVRSPRRGLQEQEELCFHMWGGKGGLCQSSL

SEQ ID NO: 3 - protein, 193 aa, Mus sp.
MTMRHCWTAGPSSWWVLLLYVHVILARATSAPQTTATVLTGSSKDPCSSWSPAVPTKQYPALDVIWPEKEVPLNGTLTLSCTACSRFPYFSILYWLGNGSFIEHLPGRLKEGHTSREHRNTSTWLHRALVLEELSPTLRSTNFSCLFVDPGQVAQYHIILAQLWDGLKTAPSPSQETLSSHSPVSRSAGPGVA

SEQ ID NO: 4 - protein, 207 aa, Macaca fascicularis
MRHNWTPDLSFLWVLLCAHIITLLVRATPVSQTTTAATASSRSTKDPCPSQPPVFPAAKQCPALEVTWPEVEMPLNGTLTLSCTACSRFPNFSMLYWLGNGSFIEHLPGQLWEGSTSREHGSTGTRLYKALVLEQLTPALHSTNFSCVLMDPEQVVQRHVILAQLWAGLRTTLPPTQEALPSSHSTGPQQPTAAGLRLSTGPAAARP

SEQ ID NO: 5 - protein, 193 aa, synthetic construct
GLNDIFEAQKIEWHEGGHHHHHHHHGSGGTPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQG

SEQ ID NO: 6 - protein, 194 aa, synthetic construct
GLNDIFEAQKIEWHEGGHHHHHHHHGSGGTSAPQTTATVLTGSSKDPCSSWSPAVPTKQYPALDVIWPEKEVPLNGTLTLSCTACSRFPYFSILYWLGNGSFIEHLPGRLKEGHTSREHRNTSTWLHRALVLEELSPTLRSTNFSCLFVDPGQVAQYHIILAQLWDGLKTAPSPSQETLSSHSPVSRSAGPGVA

SEQ ID NO: 7 - protein, 201 aa, synthetic construct
HHHHHHGLNDIFEAQKIEWHETPVSQTTTAATASSRSTKDPCPSQPPVFPAAKQCPALEVTWPEVEMPLNGTLTLSCTACSRFPNFSMLYWLGNGSFIEHLPGQLWEGSTSREHGSTGTRLYKALVLEQLTPALHSTNFSCVLMDPEQVVQRHVILAQLWAGLRTTLPPTQEALPSSHSTGPQQPTAAGLRLSTGPAAARP

SEQ ID NO: 8 - protein, 330 aa, Homo sapiens [sequence omitted]

SEQ ID NO: 9 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 17 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 18 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 23 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 24 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 25 - protein, 325 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 26 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - protein, 107 aa, Homo sapiens [sequence omitted]

SEQ ID NO: 29 - protein, 116 aa, Mus sp.
QVQLQQSGPEVVRPGVSVKISCKGSGYTFTDFAMYWVKQSHAKSLEWIGVISTDNGDTSYNQKFKGKATMTVDKSSSTAYMELDRLTSEDSAIYYCARSRGPFDYWGQGTALTVSS

SEQ ID NO: 30 - protein, 124 aa, Mus sp.
EVQLQQSGPELVKPGASVKIPCKASGYTFTDYNMDWVKQSHGKSLEWIGNINTNNGGTVYNQKFKGKATLTVDKSSSTAYMELRSLTSEDTAVYYCARSDTTVVASRYYAMDYWGQGTSVTVSL

SEQ ID NO: 31 - protein, 124 aa, Mus sp.
EVQLQQSGPELVKPGASVKIPCKASGYTFTDYNMDWVKQSHGKSLEWIGNINTDNGGTVYNQKFKGKATLTVDQSSSTAYMELRSLTSEDTAVYYCARSDTTVVASRYYAMDYWGQGTSVTVSS

SEQ ID NO: 32 - protein, 125 aa, Mus sp.
HVHLHQSGAELVRPGVSVKISCRGSGYTFTDYDVHWVKQSHAKSLEWIGVISTYYGDVGYNQEFKGKATMTLDKSSSTSYLELARLTSEDSAIYYCARSGTYYYGTRESHALDYWGQGTSVTVSS

SEQ ID NO: 33 - protein, 124 aa, Mus sp.
EVQLQQSGPELVKPGASVKIPCKASGYTFTDYNMDWVKQSHGKSLEWIGNINTNNGGTVYNQKFKDKATLTVDKSSSTAYMELRSLTSEDTAVYYCARSDTTVVASRYYALDYWGQGTSVTVSS

SEQ ID NO: 34 - protein, 117 aa, Mus sp.
QIQLQQSGPELVKPGASVKISCKASGYTFTDYYLNWVKQTPGQGLEWIGWIYPGSGNTKYTEKFKDKASLTVDTSSNTAYMQLSSLTSDDTAVYFCARRGVAWFAYWGQGTLVTVSA

SEQ ID NO: 35 - protein, 124 aa, Mus sp.
EVQLQQSGPELVKPGASVKISCRASGFTITDYNMDWVRQSHGKSLEWIGNINLNNGGSIYNQKFKGKATLTIDKSSSTAYMELRSLTSEDTAVYYCARSDTTVVASRYYAMDYWGQGTSVTVSS

SEQ ID NO: 36 - protein, 116 aa, Mus sp.
QVQLQQPGAELVKPGASVKLSCKVSGYTFTSYWMHWVKQRPGQGLEWIGMIDPNSDSSRYNEKFKNRATLTADKSSSTAFMQFSSLTSEDSAVYYCARSRLGQDYWGQGTTLTVSS

SEQ ID NO: 37 - protein, 116 aa, Mus sp.
QVQLQQSGAELVRPGASVTLSCKASGYRFTDYAMHWVKQTPVHGLQWIGIIDPETGDTAFNQKFKGKATLTADKSSSTAYMELSSLTSEDSAVYYCTRPRGAMDYWGQGTSVTVSS

SEQ ID NO: 38 - protein, 117 aa, Mus sp.
QVQLQQPGAERVEPGASVRLSCKTSGYTFTNYWMHWVKQRPGQGLEWIGMIHPESGSTNNNEKFKSKATLTVDKSSSTAYMQLSSLTSEDSAVYYCARWDRGYFDYWGQGTTLTVSS

SEQ ID NO: 39 - protein, 117 aa, Mus sp.
QVQLQQSGAELAKPGASVKMSCKASGYTFTNYWMHWVKQRPGQGLEWIGHIDPSTGYTEYNQKFKDKATLTADKSSSTAYMQLSSLTSEDSAVYYCARGLGGLFGYWGQGTLVTVSA

SEQ ID NO: 40 - protein, 116 aa, Mus sp.
EVQLQQSGPELVKPGASVKMSCKTSGYTFTEYTMHWVKQSHGKSLEWIGGVNPNNGGTTYNQKFRDKATLTVDKSSSTTYMEVRTLTSDDSAVYYCAPLWRVFDYWGQGTTLTVSS

SEQ ID NO: 41 - protein, 117 aa, Mus sp.
QVQLQQSGAELVRPGASVTLSCKASGYTFTDYEMHWVKQTPVHGLEWIGPIDPETGDTAYNQKFKGQATLTADKSSSTAYMELSSLTSEDSAVYYCTRGSRGVFDYWGQGTALTVSS

SEQ ID NO: 42 - protein, 118 aa, Mus sp.
QVQLVQSGAEVKKPGASVKVSCKASGYTFTGYYMHWVRQAPGQGLEWMGIINPRGGSTSHAQKFQGRVTMTRDTSTSTVYMELSSLRSEDTAVYYCARGLELHWFDPWGQGTLVTVSS

SEQ ID NO: 43 - protein, 118 aa, Mus sp.
QVQLVQSGAEVKKPGASVKVSCKASGYTFTTYDINWVRQAPGQGLEWMGIINPSGGSTSYAQKFQGRVTMTRDTSTSTVYMELSSLRSEDTAVYYCATGIGELDFDYWGQGTLVTVSS

SEQ ID NO: 44 - protein, 118 aa, Mus sp.
QVQLVQSGAEVKKPGASVKVSCKASGYTFTTYDINWVRQAPGQGLEWMGIIGSYGGATSYAQKFQGRVTMTRDTSTSTVYMELSSLRSEDTAVYYCATAEEELRFDYWGQGTLVTVSS

SEQ ID NO: 45 - protein, 107 aa, Mus sp.
DIVMTQSHKFKSTSVGDRVNITCKASQDVGTAVAWYQQKPGQSPKLLIYWASTRHIGVPDRFTGSGSGTDFTLTIRNVQSEDLADYFCQRYSSYPWTFGGGTKLEIK

SEQ ID NO: 46 - protein, 111 aa, Mus sp.
DIVLTQSPASLAMSLGKRATISCRASESVSIIATNLIHWYQQKPGQPPKLLIYHASNLETGVPARFSGSGSRTDFTLTIDPVEKDDVAIYYCLQSRKIPPTFGGGTKLEIK

SEQ ID NO: 47 - protein, 111 aa, Mus sp.
DIVLTQSPASLAMSLGKRATISCRASESVSIIGTNLIHWYQQKPGQPPKLLIYHASNLETGVPARFSGSGSRTDFTLTIDPVEEDDVAIYYCLQSRKIPPTFGGGTKLEIK

SEQ ID NO: 48 - protein, 107 aa, Mus sp.
DIQMTQSPASLSVSVGETVTITCRTSDNVYSNLAWYQQKQGKSPQLLVYGAINLADGVPSRFSGSGSDTQYSLKINNLQSEDFGSYYCQHFWGNPYTFGGGTKLEIK

SEQ ID NO: 49 - protein, 111 aa, Mus sp.
DIVLTQSPASLAMSLGKRATISCRASESVSIIGTDLIHWYQQKPGQPPKLLIYHASNLETGVPARFSGSGSRTDFTLTIDPVEEDDVAIYYCLQSRKIPPTFGGGTKLEIK

SEQ ID NO: 50 - protein, 107 aa, Mus sp.
DIKMTQSPSSIYASLGERVTITCKASQDINSYLSWFQQKPGKSPKTLIHRANRLVDGVPSRFSGSGSGQDYSLTISSLEYEDMGIYYCLQHDDFPPTFGAGTNLELK

SEQ ID NO: 51 - protein, 111 aa, Mus sp.
DIVLTQSPASLAMSLGKRATISCRASESVSIIGTNLIHWYQQKPGQPPKLLIYHASNLETGVPVRFSGSGSRTDFTLTIDPVEEDDVAIYYCLQSRKIPPTFGGGTKLEIK

SEQ ID NO: 52 - protein, 107 aa, Mus sp.
DIVMTQSHKFMSTSVGDRVSITCKASQDVSTAVAWYQQKPGQSPKLLIYWASTRHTGVPDRFTGSESGTDYTLIISSVQAEDLALYYCQQHFSTPYTFGGGTKLEIK

SEQ ID NO: 53 - protein, 107 aa, Mus sp.
DIVMTQSHKFMSTSGGDRVSITCKASQDVGTAVAWYQQKPGISPKLLIYWASTRHTGVPDRFTGSGSGTDFTLAISNVQSEDLADYFCQQYSSHPYTFGGGTKLEIK

SEQ ID NO: 54 - protein, 107 aa, Mus sp.
DIVMTQSHKFMSTSVGDRVSITCKASQDVRSTVGWYQQRPGQSPKLLIYWTSTRHTGVPDRFTGSGSGTDYTLTISSVQAEDLALYYCQQHYSTPYTFGGGTTLEIK

SEQ ID NO: 55 - protein, 107 aa, Mus sp.
DIQMTQSPSSLSASLGGKVTITCKASQDINKFIAWYQHKPGKGPRLLIHHTSTLMPGIPSRFSGSGSGREYSFSISNLEPEDIATYYCLQYDNLLWTFGGGTKLEIR

SEQ ID NO: 56 - protein, 107 aa, Mus sp.
DIVMTQSHKFMSSSVGDRVSITCKASQDVGTAVAWYQQKPGQSPKLLIYWASTRHTGVPDRFTGSGSGTDFTLTISNVQSEDLADYFCHQFSRYPLTFGAGTKLELK

SEQ ID NO: 57 - protein, 107 aa, Mus sp.
DIVMTQSHKFMSTSVGDRVSITCKASQDVGTAVAWYQQKPGQSPKLLIYWASARHTGVPDRFTGSGSGTDFTLTISNVQSEDLADYFCQHYFNFPRTFGGGTKLEIT

SEQ ID NO: 58 - protein, 107 aa, Mus sp.
DIQMTQSPSSLSASVGDRVTITCRASQNIRSWLAWYQQKPGKAPKLLIYAASSLQSGVPSRFSGSGSGTDFTLTISSLQPEDFATYYCQQANSFPYTFGQGTKLEIK

SEQ ID NO: 59 - protein, 107 aa, Mus sp.
DIQMTQSPSSLSASVGDRVTITCRASQSISSYLNWYQQKPGKAPKLLIYKASTLHNGVPSRFSGSGSGTDFTLTISNLQPEDFATYYCQQAISFPLTFGGGTKVEIK

SEQ ID NO: 60 - protein, 5 aa, Mus sp.
DFAMY

SEQ ID NO: 61 - protein, 5 aa, Mus sp.
DYNMD

SEQ ID NO: 62 - protein, 5 aa, Mus sp.
DYDVH

SEQ ID NO: 63 - protein, 5 aa, Mus sp.
DYYLN

SEQ ID NO: 64 - protein, 5 aa, Mus sp.
SYWMH

SEQ ID NO: 65 - protein, 5 aa, Mus sp.
DYAMH

SEQ ID NO: 66 - protein, 5 aa, Mus sp.
NYWMH

SEQ ID NO: 67 - protein, 5 aa, Mus sp.
EYTMH

SEQ ID NO: 68 - protein, 5 aa, Mus sp.
DYEMH

SEQ ID NO: 69 - protein, 5 aa, Mus sp.
GYYMH

SEQ ID NO: 70 - protein, 5 aa, Mus sp.
TYDIN

SEQ ID NO: 71 - protein, 17 aa, Mus sp.
VISTDNGDTSYNQKFKG

SEQ ID NO: 72 - protein, 17 aa, Mus sp.
NINTNNGGTVYNQKFKG

SEQ ID NO: 73 - protein, 17 aa, Mus sp.
NINTDNGGTVYNQKFKG

SEQ ID NO: 74 - protein, 17 aa, Mus sp.
VISTYYGDVGYNQEFKG

SEQ ID NO: 75 - protein, 17 aa, Mus sp.
NINTNNGGTVYNQKFKD

SEQ ID NO: 76 - protein, 17 aa, Mus sp.
WIYPGSGNTKYTEKFKD

SEQ ID NO: 77 - protein, 17 aa, Mus sp.
NINLNNGGSIYNQKFKG

SEQ ID NO: 78 - protein, 17 aa, Mus sp.
MIDPNSDSSRYNEKFKN

SEQ ID NO: 79 - protein, 17 aa, Mus sp.
IIDPETGDTAFNQKFKG

SEQ ID NO: 80 - protein, 17 aa, Mus sp.
MIHPESGSTNNNEKFKS

SEQ ID NO: 81 - protein, 17 aa, Mus sp.
HIDPSTGYTEYNQKFKD

SEQ ID NO: 82 - protein, 17 aa, Mus sp.
GVNPNNGGTTYNQKFRD

SEQ ID NO: 83 - protein, 17 aa, Mus sp.
PIDPETGDTAYNQKFKG

SEQ ID NO: 84 - protein, 17 aa, Mus sp.
IINPRGGSTSHAQKFQG

SEQ ID NO: 85 - protein, 17 aa, Mus sp.
IINPSGGSTSYAQKFQG

SEQ ID NO: 86 - protein, 17 aa, Mus sp.
IIGSYGGATSYAQKFQG

SEQ ID NO: 87 - protein, 7 aa, Mus sp.
SRGPFDY

SEQ ID NO: 88 - protein, 15 aa, Mus sp.
SDTTVVASRYYAMDY

SEQ ID NO: 89 - protein, 16 aa, Mus sp.
SGTYYYGTRESHALDY

SEQ ID NO: 90 - protein, 15 aa, Mus sp.
SDTTVVASRYYALDY

SEQ ID NO: 91 - protein, 8 aa, Mus sp.
RGVAWFAY

SEQ ID NO: 92 - protein, 7 aa, Mus sp.
SRLGQDY

SEQ ID NO: 93 - protein, 7 aa, Mus sp.
PRGAMDY

SEQ ID NO: 94 - protein, 8 aa, Mus sp.
WDRGYFDY

SEQ ID NO: 95 - protein, 8 aa, Mus sp.
GLGGLFGY

SEQ ID NO: 96 - protein, 7 aa, Mus sp.
LWRVFDY

SEQ ID NO: 97 - protein, 8 aa, Mus sp.
GSRGVFDY

SEQ ID NO: 98 - protein, 9 aa, Mus sp.
GLELHWFDP

SEQ ID NO: 99 - protein, 9 aa, Mus sp.
GIGELDFDY

SEQ ID NO: 100 - protein, 9 aa, Mus sp.
AEEELRFDY

SEQ ID NO: 101 - protein, 11 aa, Mus sp.
KASQDVGTAVA

SEQ ID NO: 102 - protein, 15 aa, Mus sp.
RASESVSIIATNLIH

SEQ ID NO: 103 - protein, 15 aa, Mus sp.
RASESVSIIGTNLIH

SEQ ID NO: 104 - protein, 11 aa, Mus sp.
RTSDNVYSNLA

SEQ ID NO: 105 - protein, 15 aa, Mus sp.
RASESVSIIGTDLIH

SEQ ID NO: 106 - protein, 11 aa, Mus sp.
KASQDINSYLS

SEQ ID NO: 107 - protein, 11 aa, Mus sp.
KASQDVSTAVA

SEQ ID NO: 108 - protein, 11 aa, Mus sp.
KASQDVRSTVG

SEQ ID NO: 109 - protein, 11 aa, Mus sp.
KASQDINKFIA

SEQ ID NO: 110 - protein, 11 aa, Mus sp.
RASQNIRSWLA

SEQ ID NO: 111 - protein, 11 aa, Mus sp.
RASQSISSYLN

SEQ ID NO: 112 - protein, 7 aa, Mus sp.
WASTRHI

SEQ ID NO: 113 - protein, 7 aa, Mus sp.
HASNLET

SEQ ID NO: 114 - protein, 7 aa, Mus sp.
GAINLAD

SEQ ID NO: 115 - protein, 7 aa, Mus sp.
RANRLVD

SEQ ID NO: 116 - protein, 7 aa, Mus sp.
WASTRHT

SEQ ID NO: 117 - protein, 7 aa, Mus sp.
WTSTRHT

SEQ ID NO: 118 - protein, 7 aa, Mus sp.
HTSTLMP

SEQ ID NO: 119 - protein, 7 aa, Mus sp.
WASARHT

SEQ ID NO: 120 - protein, 7 aa, Mus sp.
AASSLQS

SEQ ID NO: 121 - protein, 7 aa, Mus sp.
KASTLHN

SEQ ID NO: 122 - protein, 9 aa, Mus sp.
QRYSSYPWT

SEQ ID NO: 123 - protein, 9 aa, Mus sp.
LQSRKIPPT

SEQ ID NO: 124 - protein, 9 aa, Mus sp.
QHFWGNPYT

SEQ ID NO: 125 - protein, 9 aa, Mus sp.
LQHDDFPPT

SEQ ID NO: 126 - protein, 9 aa, Mus sp.
QQHFSTPYT

SEQ ID NO: 127 - protein, 9 aa, Mus sp.
QQYSSHPYT

SEQ ID NO: 128 - protein, 9 aa, Mus sp.
QQHYSTPYT

SEQ ID NO: 129 - protein, 9 aa, Mus sp.
LQYDNLLWT

SEQ ID NO: 130 - protein, 9 aa, Mus sp.
HQFSRYPLT

SEQ ID NO: 131 - protein, 9 aa, Mus sp.
QHYFNFPRT

SEQ ID NO: 132 - protein, 9 aa, Mus sp.
QQANSFPYT

SEQ ID NO: 133 - protein, 9 aa, Mus sp.
QQAISFPLT

SEQ ID NO: 134 - protein, 123 aa, synthetic construct
QVQLVQSGAEVKKPGASVKVSCKASGYTFTDYDMWVRQAPGQGLEWMGIISTYYGDVSYAIKFQGRVTMTRDKSTSTVYMELSSLRSEDTAVYYCARSGTYYYGTRESALDYWGQGTLVTVSS

SEQ ID NO: 135 - protein, 125 aa, synthetic construct
QVQLVQSGAEVKKPGASVKVSCKASGYTFTDYDLHWVRQAPGQGLEWMGIISTYYGDVSYAQKFQGRVTMTRDKSTSTVYMELSSLRSEDTAVYYCARSGMYYYGTRESHALDYWGQGTLVTVSS

SEQ ID NO: 136 - protein, 125 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSS

SEQ ID NO: 137 - protein, 125 aa, synthetic construct
QVQLVQSGAEVKKPGASVKVSCKASGYTFTDYDMHWVRQAPGQGLEWMGIISTYYGDVSYAQKKQGRVTMTRDKSTSTVYMELSSLRSEDTAVYYCARSGTYNYGTRESHPLDYWGQGTLVTVSS

SEQ ID NO: 138 - protein, 125 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSS

SEQ ID NO: 139 - protein, 125 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHPLDYWGQGTLVTVSS

SEQ ID NO: 140 - protein, 125 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSS

SEQ ID NO: 141 - protein, 125 aa, synthetic construct
QVQLVQSGAEVKKPGASVKVSCKASGYTFTDYDMHWVRQAPGQGLEWMGIYSTYYGDVSYAQKKQGRVTMTRDKSTSTVYMELSSLRSEDTAVYYCARSGTYNYGTRESHALDYWGQGTLVTVSS

SEQ ID NO: 142 - protein, 125 aa, synthetic construct
QVQLVQSGAEVKKPGASVKVSCKASGYTFTDYDMHWVRQAPGQGLEWMGIYSTYYGVVSYAQKFQGRVTMTRDKSTSTVYMELSSLRSEDTAVYYCARSGTYYYGTRESHAFDYWGQGTLVTVSS

SEQ ID NO: 143 - protein, 125 aa, synthetic construct
QVQLVQSGAEVKKPGASVKVSCKASGYTFTDYDMHWVRQAPGQGLEWMGIYSTYYGVVSYAQKFQGRVTMTRDKSTSTVYMELSSLRSEDTAVYYCARSGTYYYGTRESHPLDYWGQGTLVTVSS

SEQ ID NO: 144 - protein, 125 aa, synthetic construct
QVQLVQSGAEVKKPGASVKVSCKASGYTFTDYDMHWVRQAPGQGLEWMGIYSTYYGVVSYAQKFQGRVTMTRDKSTSTVYMELSSLRSEDTAVYYCARSGTYNYGTRESHALDYWGQGTLVTVSS

SEQ ID NO: 145 - protein, 125 aa, synthetic construct
QVQLVQSGAEVKKPGASVKVSCKASGYTFTDYDMHWVRQAPGQGLEWMGIYSTYYGVVSYAQKKQGRVTMTRDKSTSTVYMELSSLRSEDTAVYYCARSGTYYYGTRESHALDYWGQGTLVTVSS

SEQ ID NO: 146 - protein, 125 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSEDTAVYYCARSGTYYYGTRESHPFDYWGQGTLVTVSS

SEQ ID NO: 147 - protein, 125 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSEDTAVYYCARSGTYNYGTRESHAFDYWGQGTLVTVSS

SEQ ID NO: 148 - protein, 125 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSEDTAVYYCARSGTYNYGTRESHPLDYWGQGTLVTVSS

SEQ ID NO: 149 - protein, 125 aa, synthetic construct
QVQLVQSGAEVKKPGASVKVSCKASGYTFTDYDMHWVRQAPGQGLEWMGIISTYYGDVSYAQKKQGRVTMTRDKSTSTVYMELSSLRSEDTAVYYCARSGTYYYGTRESHAFDYWGQGTLVTVSS

SEQ ID NO: 150 - protein, 125 aa, synthetic construct
QVQLVQSGAEVKKPGASVKVSCKASGYTFTDYDMHWVRQAPGQGLEWMGIISTYYGDVSYAQKKQGRVTMTRDKSTSTVYMELSSLRSEDTAVYYCARSGTYYYGTRESHPLDYWGQGTLVTVSS

SEQ ID NO: 151 - protein, 125 aa, synthetic construct
QVQLVQSGAEVKKPGASVKVSCKASGYTFTDYDMHWVRQAPGQGLEWMGIISTYYGDVSYAQKKQGRVTMTRDKSTSTVYMELSSLRSEDTAVYYCARSGTYNYGTRESHALDYWGQGTLVTVSS

SEQ ID NO: 152 - protein, 125 aa, synthetic construct
QVQLVQSGAEVKKPGASVKVSCKASGYTFTDYDMHWVRQAPGQGLEWMGIISTYYGVVSYAQKFQGRVTMTRDKSTSTVYMELSSLRSEDTAVYYCARSGTYYYGTRESHAFDYWGQGTLVTVSS

SEQ ID NO: 153 - protein, 125 aa, synthetic construct
QVQLVQSGAEVKKPGASVKVSCKASGYTFTDYDMHWVRQAPGQGLEWMGIISTYYGVVSYAQKFQGRVTMTRDKSTSTVYMELSSLRSEDTAVYYCARSGTYYYGTRESHPLDYWGQGTLVTVSS

SEQ ID NO: 154 - protein, 125 aa, synthetic construct
QVQLVQSGAEVKKPGASVKVSCKASGYTFTDYDMHWVRQAPGQGLEWMGIISTYYGVVSYAQKFQGRVTMTRDKSTSTVYMELSSLRSEDTAVYYCARSGTYNYGTRESHALDYWGQGTLVTVSS

SEQ ID NO: 155 - protein, 125 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHALDYWGQGTLVTVSS

SEQ ID NO: 156 - protein, 125 aa, synthetic construct
QVQLVQSGAEVKKPGASVKVSCKASGYTFTDYDMHWVRQAPGQGLEWMGIYSTYYGDVSYAQKFQGRVTMTRDKSTSTVYMELSSLRSEDTAVYYCARSGTYYYGTRESHAFDYWGQGTLVTVSS

SEQ ID NO: 157 - protein, 125 aa, synthetic construct
QVQLVQSGAEVKKPGASVKVSCKASGYTFTDYDMHWVRQAPGQGLEWMGIYSTYYGDVSYAQKFQGRVTMTRDKSTSTVYMELSSLRSEDTAVYYCARSGTYYYGTRESHPLDYWGQGTLVTVSS

SEQ ID NO: 158 - protein, 125 aa, synthetic construct
QVQLVQSGAEVKKPGASVKVSCKASGYTFTDYDMHWVRQAPGQGLEWMGIYSTYYGDVSYAQKFQGRVTMTRDKSTSTVYMELSSLRSEDTAVYYCARSGTYNYGTRESHALDYWGQGTLVTVSS

SEQ ID NO: 159 - protein, 125 aa, synthetic construct
QVQLVQSGAEVKKPGASVKVSCKASGYTFTDYDMHWVRQAPGQGLEWMGIYSTYYGVVSYAQKFQGRVTMTRDKSTSTVYMELSSLRSEDTAVYYCARSGTYYYGTRESHALDYWGQGTLVTVSS

SEQ ID NO: 160 - protein, 125 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSEDTAVYYCARSGTYYYGTRESHAFDYWGQGTLVTVSS

SEQ ID NO: 161 - protein, 125 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSEDTAVYYCARSGTYYYGTRESHPLDYWGQGTLVTVSS

SEQ ID NO: 162 - protein, 125 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSEDTAVYYCARSGTYNYGTRESHALDYWGQGTLVTVSS

SEQ ID NO: 163 - protein, 125 aa, synthetic construct
QVQLVQSGAEVKKPGASVKVSCKASGYTFTDYDMHWVRQAPGQGLEWMGIISTYYGDVSYAQKKQGRVTMTRDKSTSTVYMELSSLRSEDTAVYYCARSGTYYYGTRESHALDYWGQGTLVTVSS

SEQ ID NO: 164 - protein, 125 aa, synthetic construct
QVQLVQSGAEVKKPGASVKVSCKASGYTFTDYDMHWVRQAPGQGLEWMGIISTYYGVVSYAQKFQGRVTMTRDKSTSTVYMELSSLRSEDTAVYYCARSGTYYYGTRESHALDYWGQGTLVTVSS

SEQ ID NO: 165 - protein, 125 aa, synthetic construct
QVQLVQSGAEVKKPGASVKVSCKASGYTFTDYDMHWVRQAPGQGLEWMGIYSTYYGDVSYAQKFQGRVTMTRDKSTSTVYMELSSLRSEDTAVYYCARSGTYYYGTRESHALDYWGQGTLVTVSS

SEQ ID NO: 166 - protein, 107 aa, synthetic construct
DIQMTQSPSSVSASVGDRVTITCRASDNVYSNLAWYQQKPGKAPKLLIYGAISLADGVPSRFSGSGSGTDFTLTISSLQPEDFATYYCQHFWGNPYTFGQGTKLEIK

SEQ ID NO: 167 - protein, 4 aa, synthetic construct
DYDM

SEQ ID NO: 168 - protein, 5 aa, synthetic construct
DYDLH

SEQ ID NO: 169 - protein, 5 aa, synthetic construct
DYDMH

SEQ ID NO: 170 - protein, 17 aa, synthetic construct
IISTYYGDVSYAIKFQG

SEQ ID NO: 171 - protein, 17 aa, synthetic construct
IISTYYGDVSYAQKFQG

SEQ ID NO: 172 - protein, 17 aa, synthetic construct
IISTYYGDVSYAQKKQG

SEQ ID NO: 173 - protein, 17 aa, synthetic construct
IISTYYGVVSYAQKKQG

SEQ ID NO: 174 - protein, 17 aa, synthetic construct
IYSTYYGDVSYAQKKQG

SEQ ID NO: 175 - protein, 17 aa, synthetic construct
IYSTYYGVVSYAQKFQG

SEQ ID NO: 176 - protein, 17 aa, synthetic construct
IYSTYYGVVSYAQKKQG

SEQ ID NO: 177 - protein, 17 aa, synthetic construct
IISTYYGVVSYAQKFQG

SEQ ID NO: 178 - protein, 17 aa, synthetic construct
IYSTYYGDVSYAQKFQG

SEQ ID NO: 179 - protein, 15 aa, synthetic construct
SGTYYYGTRESALDY

SEQ ID NO: 180 - protein, 16 aa, synthetic construct
SGMYYYGTRESHALDY

SEQ ID NO: 181 - protein, 16 aa, synthetic construct
SGTYYYGTREQHALDY

SEQ ID NO: 182 - protein, 16 aa, synthetic construct
SGTYNYGTRESHPLDY

SEQ ID NO: 183 - protein, 16 aa, synthetic construct
SGTYYYGTRESHAFDY

SEQ ID NO: 184 - protein, 16 aa, synthetic construct
SGTYYYGTRESHPLDY

SEQ ID NO: 185 - protein, 16 aa, synthetic construct
SGTYNYGTRESHALDY

SEQ ID NO: 186 - protein, 16 aa, synthetic construct
SGTYYYGTRESHALDY

SEQ ID NO: 187 - protein, 16 aa, synthetic construct
SGTYYYGTRESHPFDY

SEQ ID NO: 188 - protein, 16 aa, synthetic construct
SGTYNYGTRESHAFDY

SEQ ID NO: 189 - protein, 214 aa, synthetic construct
DIQMTQSPSSVSASVGDRVTITCRASDNVYSNLAWYQQKPGKAPKLLIYGAISLADGVPSRFSGSGSGTDFTLTISSLQPEDFATYYCQHFWGNPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 190 - protein, 455 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 455 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 455 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 455 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 455 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 195 - protein, 455 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 455 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 455 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 198 - protein, 455 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 455 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - protein, 455 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 455 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 455 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - protein, 452 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 204 - protein, 452 aa, synthetic construct
QVQLVQSGAEVKKPGASVKVSCKASGYTFTDYDMHWVRQAPGQGLEWMGIISTYYGDVSYAQKFQGRVTMTRDKSTSTVYMELSSLRSENTAVYYCARSGTYYYGTREQHALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 205 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 210 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 213 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 452 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 219 - protein, 452 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYYYGTRESHAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 220 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 225 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 228 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - protein, 455 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 452 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 234 - protein, 452 aa, synthetic construct
QVQLVQSGAEVKKPGASVKVSCKASGYTFTDYDMHWVRQAPGQGLEWMGIISTYYGVVSYAQKKQGRVTMTRDKSTSTVYMELSSLRSEDTAVYYCARSGTYNYGTRESHALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 235 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 237 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 239 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 241 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 243 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 245 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 246 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 247 - protein, 11 aa, synthetic construct
RASDNVYSNLA

SEQ ID NO: 248 - protein, 7 aa, synthetic construct
GAISLAD

SEQ ID NO: 249 - protein, 9 aa, synthetic construct
QHFWGNPYT